(19) United States
(12) Patent Application Publication
(10) Pub. No.: US 2024/0327384 A1
(43) Pub. Date: Oct. 3, 2024

(54) SALTS AND SOLID STATE FORMS OF A KIF18A INHIBITOR COMPOUND

(71) Applicant: Amgen Inc., Thousand Oaks, CA (US)

(72) Inventors: Tian WU, Thousand Oaks, CA (US); Prashant AGARWAL, Thousand Oaks, CA (US); Andreas R. ROTHELI, Thousand Oaks, CA (US); Hyunsoo PARK, Thousand Oaks, CA (US); Michael J. FROHN, Thousand Oaks, CA (US)

(73) Assignee: Amgen Inc., Thousand Oaks, CA (US)

(21) Appl. No.: 18/580,564

(22) PCT Filed: Jul. 21, 2022
(86) PCT No.: PCT/US2022/037928

§ 371 Date: Jan. 18, 2024

Related U.S. Application Data
(60) Provisional application No. 63/224,208, filed on Jul. 21, 2021.

(51) Int. Cl. C07D 401/14; A61K 31/506; C07C 15/06; C07C 31/02; C07C 31/04; C07C 31/08; C07C 43/04; C07C 43/18; C07C 49/08; C07C 55/02; C07C 55/22; C07C 69/003; C07C 233/05; C07C 255/03; C07C 275/06; C07C 309/04; C07C 309/65; C07C 309/73; C07D 211/90; C07D 275/06; C07D 307/06; C07D 319/12; C07D 487/04

(52) U.S. Cl. CPC C07D 401/14; A61K 31/506; C07C 15/06; C07C 31/02; C07C 31/04; C07C 31/08; C07C 43/046; C07C 43/18; C07C 49/08; C07C 55/02; C07C 55/22; C07C 69/003; C07C 233/05; C07C 255/03; C07C 275/06; C07C 309/04; C07C 309/65; C07C 309/73; C07D 211/90; C07D 275/06; C07D 307/06; C07D 319/12; C07D 487/04; C07B 2200/13; C07C 2601/08

(57) ABSTRACT

Disclosed herein is a salt, a crystalline anhydrous form, a hydrate, a solvate, or a co-crystal of a free base compound 2-(6-azaspiro[2.5]octan-6-yl)-N-[2-(4,4-difluoropiperidin-1-yl)-6-methylpyrimidin-4-yl]-4-[(2-hydroxyethanesulfonyl)amino]benzamide (Compound A); method of preparation, pharmaceutical compositions, and method of treating a disease mediated by a motor protein kinesin family member 18A (KIF18A) inhibition, wherein said disease is a neoplastic disease, including a cancer or a tumor.

### INCORPORATION BY REFERENCE OF MATERIAL SUBMITTED ELECTRONICALLY

Incorporated herein by reference in its entirety is a computer-readable nucleotide/amino acid sequence listing in ST.26 format submitted concurrently herewith and identified as follows: 137 KB XML format file named “A-2832-WO01-SEC_FromUS-PSP_Seq_Listing_ST26_072122b” and created on Jul. 21, 2022”.

### FIELD OF THE INVENTION

The present disclosure relates to a salt, a hydrate, a solvate, or a co-crystal of a free base compound 2-(6-azaspiro[2.5]octan-6-yl)-N-[2-(4,4-difluoropiperidin-1-yl)-6-methylpyrimidin-4-yl]-4-[(2-hydroxyethanesulfonyl)amino]benzamide (Compound A); or a solid form of the Compound A, including crystalline anhydrous forms, salt, hydrate, solvate, or co-crystal thereof; method of preparation, pharmaceutical compositions, and method of treating a disease mediated by a motor protein kinesin family member 18A (KIF18A) inhibition.

### BACKGROUND OF THE INVENTION

The free base compound 2-(6-azaspiro[2.5]octan-6-yl)-N-[2-(4,4-difluoropiperidin-1-yl)-6-methylpyrimidin-4-yl]-4-[(2-hydroxyethanesulfonyl)amino]benzamide (Compound A), is useful as an inhibitor of a motor protein kinesin family member 18A (KIF18A):

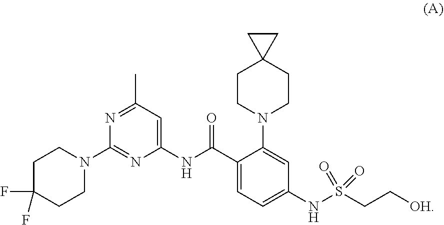 [SMILES: Cc1cc(NC(=O)c2ccc(NS(=O)(=O)CCO)cc2N2CCC3(CC2)CC3)nc(N2CCC(F)(F)CC2)n1]

Kinesins are molecular motors that play important roles in cell division and intracellular vesicles and organelle transport. Mitotic kinesin plays roles in several aspects of spindle assembly, chromosome segregation, centrosome separation, and dynamics. Human kinesins are categorized into 14 subfamilies based on sequence homology within the so-called “motor domain”; this domain's ATPase activity drives unidirectional movement along microtubules (MT). The nonmotor domain of these proteins is responsible for cargo attachment; a “cargo” can include any one of a variety of different membranous organelles, signal transduction scaffolding systems, and chromosomes. Kinesins use the energy of ATP hydrolysis to move cargo along polarized microtubules. Thus, kinesins are often called “plus-end” or “minus-end” directed motors.

KIF18A gene belongs to the Kinesin-8 subfamily and is a plus-end-directed motor. KIF18A is believed to influence dynamics at the plus end of kinetochore microtubules to control correct chromosome positioning and spindle tension. Depletion of human KIF18A leads to longer spindles, increased chromosome oscillation at metaphase, and activation of the mitotic spindle assembly checkpoint in HeLa cervical cancer cells. KIF18A appears to be a viable target for the treatment of cancer. KIF18A is overexpressed in various types of cancers, including but not limited to colon, breast, lung, pancreas, prostate, bladder, head, neck, cervix, and ovarian cancers. Further, genetic deletion or knockdown or inhibition of KIF18A affects mitotic spindle apparatus in cancer cell lines. Particularly, inhibition of KIF18A has been found to induce mitotic cell arrest, a known vulnerability that can promote cell death in mitosis via apoptosis, mitotic catastrophe, or multipolarity driven lethality or death after mitotic slippage in interphase.

The human KIF18A gene sequence, the human KIF18A mRNA sequence, and the encoded KIF18A protein are provided herein as SEQ ID NOs: 12, 13, and 11, respectively.

Compound A, as well as an exemplary method of making the same, is described in International Patent Application Publication No. WO2020/132648, which is incorporated herein by reference in its entirety. However, the stable salt, hydrate, solvate, or co-crystal of Compound A, along with the solid form of Compound A (including crystalline anhydrous Compound A or amorphous Compound A), the stable salt, hydrate, solvate, or co-crystal of Compound A are desired, particularly for the commercial pharmaceutical production of Compound A.

### SUMMARY OF THE INVENTION

In one aspect, disclosed herein is a salt, a hydrate, a solvate, or a co-crystal of Compound A having a structure of

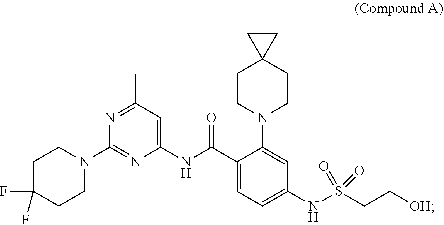 [SMILES: Cc1cc(NC(=O)c2ccc(NS(=O)(=O)CCO)cc2N2CCC3(CC2)CC3)nc(N2CCC(F)(F)CC2)n1]

which chemical name is 2-(6-azaspiro[2.5]octan-6-yl)-N-[2-(4,4-difluoropiperidin-1-yl)-6-methylpyrimidin-4-yl]-4-[(2-hydroxyethanesulfonyl)amino]benzamide; or is also known as N-(2-(4,4-difluoropiperidin-1-yl)-6-methylpyrimidin-4-yl)-4-((2-hydroxyethyl)sulfonamido)-2-(6-azaspiro[2.5]octan-6-yl)benzamide; or a solid form of Compound A (including crystalline anhydrous Compound A or amorphous Compound A), salt, hydrate, solvate, or co-crystal thereof.

In another aspect, disclosed herein is a solid form of the Compound A, including crystalline anhydrous forms, a salt, a hydrate, a solvate, or a co-crystal of Compound A. The solid form can be crystalline form or amorphous form.

In various embodiments, disclosed herein is the salt, anhydrous, hydrate, solvate, or co-crystal of Claim 1, selected from hydrochloride salt (Compound A-HCl), mesylate salt (Compound A-MsA), tosylate salt (Compound A-TsA), sulfate salt (Compound A-sulfate), variable hydrate (Compound A-variable hydrate), tetrahydrofuran solvate (Compound A-THF), ethanol solvate (Compound A-ethanol), 1-propanol solvate (Compound A-1-propanol), isopropyl alcohol solvate (Compound A-IPA), methanol solvate (Compound A-methanol), isopropyl acetate solvate (Compound A-IPAc), acetone solvate (Compound A-acetone), cyclopentyl methyl ether solvate (Compound A-CPME), dioxane solvate (Compound A-dioxane), ethyl acetate solvate (Compound A-EtOAc), acetonitrile solvate (Compound A-MeCN), methyl tert-butyl ether solvate (Compound A-MTBE), toluene solvate (Compound A-toluene), dodecyl sulfate (Compound A-dodecyl sulfate), dimethyl formamide (DMF) solvate hydrate (Compound A-DMF-hydrate), dimethylacetamide (DMAC) solvate (Compound A-DMAC), monobesylate hydrate (Compound A-besylate-hydrate), caffeine co-crystal (Compound A-caffeine), citric acid co-crystal (Compound A-citric acid), saccharin co-crystal (Compound A-saccharin), L-tartaric acid co-crystal (Compound A-L-tartaric acid), or urea co-crystal (Compound A-urea); or the solid form thereof.

In embodiment 1, the invention provides a hydrochloride salt of Compound A, having the structure:

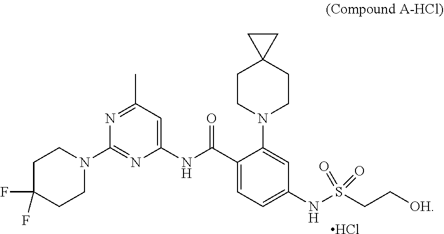 [SMILES: Cc1cc(NC(=O)c2ccc(NS(=O)(=O)CCO)cc2N2CCC3(CC2)CC3)nc(N2CCC(F)(F)CC2)n1 ; Cl]

In embodiment 1a, the invention provides a solid form of Compound A-HCl. In a sub-embodiment, the solid form is crystalline Form 1 (Compound A-HCl-Form 1). In another sub-embodiment, the solid form is crystalline Form 2 (Compound A-HCl-Form 2).

In embodiment 1 b, the invention provides a crystalline Compound A-HCl-Form 1, characterized by solid state ¹⁹F NMR peaks at −91 and −103 ppm.

In embodiment 1c, the invention provides a crystalline Compound A-HCl-Form 1, further characterized by X-Ray Powder Diffraction (XRPD) pattern peaks at 7.5, 16.9, and 20.2±0.2° 2θ using Cu Kα radiation.

In embodiment 1d, the invention provides a crystalline Compound A-HCl-Form 1, further characterized by X-Ray Powder Diffraction (XRPD) pattern peaks at 12.8, 18.2, 22.7, 23.6, 24.8 and 26.1±0.2° 2θ using Cu Kα radiation.

In embodiment 1e, the invention provides a crystalline Compound A-HCl-Form 1, further characterized by X-Ray Powder Diffraction (XRPD) pattern peaks at 10.9, 14.5, 15.7, 15.9, 19.8, 20.6, 21.6, 23.2, 26.1 and 26.8±0.2° 2θ using Cu Kα radiation.

In embodiment 1f, the invention provides a crystalline Compound A-HCl-Form 1, having an XRPD pattern substantially as shown in FIG. 1.

In embodiment 1g, the invention provides a crystalline Compound A-HCl-Form 1, having an endothermic transition at 268.5° C. to 274.5° C., as measured by Differential Scanning Calorimetry.

In embodiment 1h, the invention provides a crystalline Compound A-HCl-Form 1, wherein the endothermic transition is at 271.5° C.±3° C.

In embodiment 1i, the invention provides a crystalline Compound A-HCl-Form 1, having a Thermogravimetric Analysis (TGA) substantially as shown in FIG. 2.

In embodiment 1j, the invention provides a crystalline Compound A-HCl-Form 1, having a single crystal structure substantially as shown in FIG. 5.

In embodiment 2, the invention provides a mesylate salt of Compound A, having the structure:

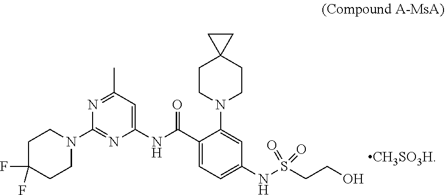

In embodiment 2a, the invention provides the invention provides a solid form of the Compound A-MsA. In a sub-embodiment, the solid form is crystalline Form 1 (Compound A-MsA-Form 1). In another sub-embodiment, the solid form is crystalline Form 2 (Compound A-MsA-Form 2).

In embodiment 2b, the invention provides a crystalline Compound A-MsA-Form 1, characterized by solid state ¹⁹F NMR peaks at −95.2 and −103.2±0.5 ppm. Spinning sidebands are indicated by (*).

In embodiment 2c, the invention provides a crystalline Compound A-MsA-Form 1, further characterized by X-Ray Powder Diffraction (XRPD) pattern peaks at 7.0, 16.5, and 23.9±0.2° 2θ using Cu Kα radiation.

In embodiment 2d, the invention provides a crystalline Compound A-MsA-Form 1, further characterized by XRPD pattern peaks at 12.6, 15.7, 17.4, 18.5, 20.0 and 21.0±0.2° 2θ using Cu Kα radiation.

In embodiment 2e, the invention provides a crystalline Compound A-MsA-Form 1, further characterized by XRPD pattern peaks at 5.8, 11.8, 13.5, 15.3, 16.1, 18.0, 20.6, 25.2, 28.0 and 30.5±0.2° 2θ using Cu Kα radiation.

In embodiment 2f, the invention provides a crystalline Compound A-MsA-Form 1, having an XRPD pattern substantially as shown in FIG. 10.

In embodiment 2g, the invention provides a crystalline Compound A-MsA-Form 1, having an endothermic transition at 247° C. to 253° C., as measured by Differential Scanning Calorimetry.

In embodiment 2h, the invention provides a crystalline Compound A-MsA-Form 1, wherein the endothermic transition is at 250° C.±3° C.

In embodiment 2i, the invention provides a crystalline Compound A-MsA-Form 1, having a Thermogravimetric Analysis (TGA) substantially as shown in FIG. 11.

In embodiment 3, the invention provides a tosylate salt of Compound A, having the structure:

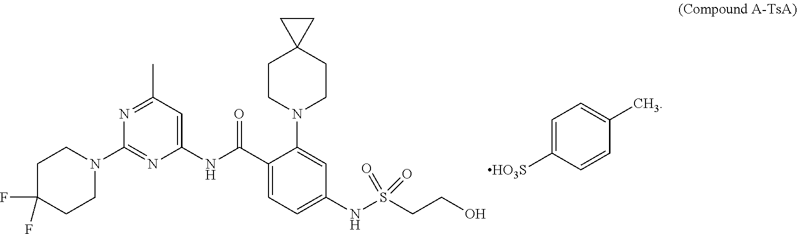

In embodiment 3a, the invention provides a solid form of the Compound A-TsA. In a sub-embodiment, the solid form is crystalline Form 1 (Compound A-TsA-Form 1). In another sub-embodiment, the solid form is crystalline Form 2 (Compound A-TsA-Form 2). In a sub-embodiment, the solid form is crystalline Form 3 (Compound A-TsA-Form 3). In another sub-embodiment, the solid form is crystalline Form 4 (Compound A-TsA-Form 4). In another sub-embodiment, the solid form is crystalline Form 5 (Compound A-TsA-Form 5). In yet another sub-embodiment, the solid form is a ditosylate salt crystalline Form 6 (Compound A-DiTsA-Form 6).

In embodiment 3b, the invention provides a crystalline Compound A-TsA-Form 4, characterized by X-Ray Powder Diffraction (XRPD) pattern peaks at 6.2, 14.7, and 23.5±0.2° 2θ using Cu Kα radiation.

In embodiment 3c, the invention provides a crystalline Compound A-TsA-Form 4, further characterized by XRPD pattern peaks at 10.5, 12.4, 14.2, 19.1, 21.5 and 29.0±0.2° 2θ using Cu Kα radiation.

In embodiment 3d, the invention provides a crystalline Compound A-TsA-Form 4, further characterized by XRPD pattern peaks at 15.5, 16.5, 17.7, 18.3, 18.6, 20.1, 20.8, 24.1, and 25.3±0.2° 2θ using Cu Kα radiation.

In embodiment 3e, the invention provides a crystalline Compound A-TsA-Form 4, having an XRPD pattern substantially as shown in FIG. 24a.

In embodiment 3f, the invention provides a crystalline Compound A-TsA-Form 4, having a single crystal structure substantially as shown in FIG. 24b.

In embodiment 3g, the invention provides a crystalline Compound A-TsA-Form 4, having an endothermic transition at 250° C. to 256° C., as measured by Differential Scanning Calorimetry.

In embodiment 3h, the invention provides a crystalline Compound A-TsA-Form 4, wherein the endothermic transition is at 253° C.±3° C.

In embodiment 3i, the invention provides a crystalline Compound A-TsA-Form 4, having a Thermogravimetric Analysis (TGA) substantially as shown in FIG. 25.

In embodiment 3j, the invention provides a crystalline Compound A-TsA-Form 4, characterized by solid state ¹⁹F NMR peaks at −96.93 and −101.60±0.5 ppm substantially as shown in FIG. 26. Spinning sidebands are indicated by (*).

In embodiment 4, the invention provides a solid form of the Compound A. In a sub-embodiment, the solid form is an amorphous form (Compound A-Amorphous). In another sub-embodiment, the solid form is crystalline Compound A-Form 1 (Compound A-Form 1).

In embodiment 4a, the invention provides Compound A-Amorphous, having an XRPD pattern substantially as shown in FIG. 33.

In embodiment 4b, the invention provides Compound A-Amorphous, having a melting onset at 88° C. to 94° C., as measured by Differential Scanning Calorimetry. In a sub-embodiment, the Compound A-Amorphous has the melting onset at 91° C.±3° C. In a sub-embodiment, the Compound A-Amorphous has a DSC thermograph pattern substantially as shown in FIG. 34.

In embodiment 4c, the invention provides Compound A-Amorphous, having a Thermogravimetric Analysis (TGA) substantially as shown in FIG. 35.

In embodiment 4d, the invention provides Crystalline Compound A-Form 1, having a Thermogravimetric Analysis (TGA) substantially as shown in FIG. 52.

In embodiment 5, the invention provides a sulfate salt of Compound A, having the structure:

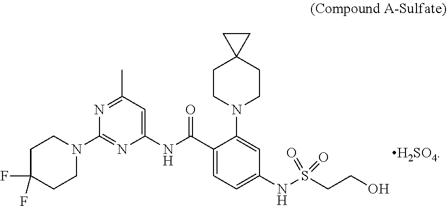

In embodiment 5a, the invention provides a solid form of the Compound A-sulfate. In a sub-embodiment, the solid form is crystalline Form 1 (Compound A-Sulfate-Form 1). In another sub-embodiment, the Compound A-Sulfate-Form 1 has an XRPD pattern substantially as shown in FIG. 30. In another sub-embodiment, the Compound A-Sulfate-Form 1 has an endothermic transition at 261° C. to 267° C., as measured by Differential Scanning Calorimetry. In yet another sub-embodiment, the Compound A-Sulfate-Form 1 has the endothermic transition at 264° C.±3° C. In yet another sub-embodiment, the Compound A-Sulfate-Form 1 has a Thermogravimetric Analysis (TGA) substantially as shown in FIG. 31.

In embodiment 6, the invention provides a hydrate of Compound A, having the structure:

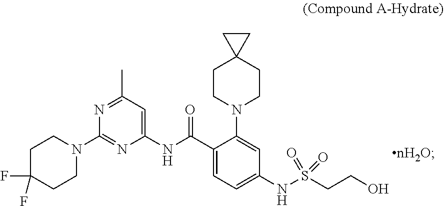

wherein n is a number in the range of 0.5 to 2, or variable (mixtures) thereof. The n value can vary as a result from various preparation methods and/or storage conditions.

In embodiment 6a, the invention provides a solid form of the Compound A-hydrate.

In embodiment 6b, the invention provides Compound A-Variable-Hydrate-Form 2, characterized by X-Ray Powder Diffraction (XRPD) pattern peaks at 13.9, 16.2, and 19.6±0.2° 2θ using Cu Kα radiation.

In embodiment 6c, the invention provides Compound A-Variable-Hydrate-Form 2, further characterized by XRPD pattern peaks at 3.5, 17.4, 18.4, 18.7, 20.0, 20.2, 22.6, 22.9, 27.5, and 30.8±0.2° 2θ using Cu Kα radiation.

In embodiment 6d, the invention provides Compound A-Variable-Hydrate-Form 2, further characterized by XRPD pattern peaks at 3.5, 10.1, 11.2, 13.9, 16.2, 18.2, 19.2, 23.2, and 26.0 t 0.2° 26 using Cu Kα radiation.

In embodiment 6e, the invention provides Compound A-Variable Hydrate Form 2, having an XRPD pattern substantially as shown in FIG. 36.

In embodiment 6f, the invention provides the Compound A-Variable-Hydrate-Form 2 having a dehydration onset at 48° C. to 54° C. and a melting point of 136° C., as measured by Differential Scanning Calorimetry. In a sub-embodiment, the Compound A-Variable-Hydrate-Form 2 has a DSC thermograph pattern substantially as shown in FIG. 37.

In embodiment 6g, the invention provides the Compound A-Variable-Hydrate-Form 2 having the endothermic transition at 51° C.±3° C.

In embodiment 6h, the invention provides the Compound A-Variable-Hydrate-Form 2 having a Thermogravimetric Analysis (TGA) substantially as shown in FIG. 38.

In embodiment 7, the invention provides a crystalline anhydrous Form of Compound A (Compound A-Anhydrous).

In embodiment 7a, the solid form is crystalline Anhydrous Form 3 (Compound A-Anhydrous-Form 3). In a sub-embodiment, the Compound A-Anhydrous-Form 3 has an XRPD pattern substantially as shown in FIG. 40. In another sub-embodiment, the Compound A-Anhydrous-Form 3 has a melting onset at 193.5° C. to 199.5° C., as measured by Differential Scanning Calorimetry. In yet another sub-embodiment, the Compound A-Anhydrous-Form 3 has the melting onset at 196.5° C.±3° C. In yet another sub-embodiment, the Compound A-Anhydrous-Form 3 has a Dynamic Vapor Sorption (DVS) substantially as shown in FIG. 42.

In embodiment 7b, the solid form is crystalline Anhydrous Form 4 (Compound A-Anhydrous-Form 4). In a sub-embodiment, the Compound A-Anhydrous-Form 4 has an XRPD pattern substantially as shown in FIG. 43.

In embodiment 7c, the solid form is crystalline Anhydrous Form 5 (Compound A-Anhydrous-Form 5). In a sub-embodiment, the Compound A-Anhydrous-Form 5 has an XRPD pattern substantially as shown in FIG. 44. In another sub-embodiment, the Compound A-Anhydrous-Form 5 has a melting onset at 188.5° C. to 194.5° C., as measured by Differential Scanning Calorimetry, substantially as shown in FIG. 45. In yet another sub-embodiment, the Compound A-Anhydrous-Form 5 has the melting onset at 191.5° C.±3° C. In yet another sub-embodiment, the Compound A-Anhydrous-Form 5 has a Dynamic Vapor Sorption (DVS) substantially as shown in FIG. 46, which showed that the Anhydrous Form 5 rehydrated to Compound A-Monohydrate.

In embodiment 7d, the solid form is crystalline Anhydrous Form 6 (Compound A-Anhydrous-Form 6). In a sub-embodiment, the Compound A-Anhydrous-Form 6 has an XRPD pattern substantially as shown in FIG. 47. In another sub-embodiment, the Compound A-Anhydrous-Form 6 has a melting onset at 183.4° C. to 189.4° C., as measured by Differential Scanning Calorimetry. In yet another sub-embodiment, the Compound A-Anhydrous-Form 6 has the melting onset at 186.4° C. t 3° C.

In embodiment 7e, the solid form is crystalline Anhydrous Form 7 (Compound A-Anhydrous-Form 7). In a sub-embodiment, the Compound A-Anhydrous-Form 7 has an XRPD pattern substantially as shown in FIG. 49.

In embodiment 7f, the solid form is crystalline Anhydrous Form 8 (Compound A-Anhydrous-Form 8). In a sub-embodiment, the Compound A-Anhydrous-Form 8 has an XRPD pattern substantially as shown in FIG. 50.

In embodiment 8, the invention provides a tetrahydrofuran (THF) solvate of Compound A, having the structure:

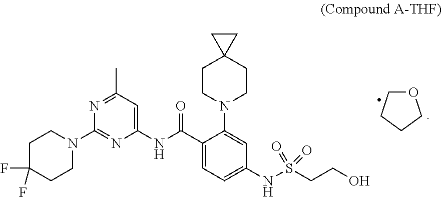

In embodiment 8a, the invention provides a solid form of the Compound A-THF. In a sub-embodiment, the Compound A-THF has an XRPD pattern substantially as shown in FIG. 53. In another sub-embodiment, the Compound A-THF has a melting onset at 188.5° C. to 194.5° C., as measured by Differential Scanning Calorimetry. In yet another sub-embodiment, the Compound A-THF has the melting onset at 191.5° C.±3° C. In yet another sub-embodiment, the Compound A-THF has a Thermogravimetric Analysis (TGA) substantially as shown in FIG. 54.

In embodiment 9, the invention provides an ethanol solvate of Compound A. In embodiment 9a, the invention provides a solid form of the Compound A-ethanol. In a sub-embodiment, the Compound A-ethanol has an XRPD pattern substantially as shown in FIG. 55. In another sub-embodiment, the Compound A-ethanol has a melting onset at 162.6° C. to 168.6° C., as measured by Differential Scanning Calorimetry. In yet another sub-embodiment, the Compound A-ethanol has the melting onset at 165.6° C.±3° C. In yet another sub-embodiment, the Compound A-ethanol has a Thermogravimetric Analysis (TGA) substantially as shown in FIG. 56.

In embodiment 10, the invention provides a 1-propanol solvate (Compound A-1-propanol). In embodiment 10a, the invention provides a solid form of the Compound A-1-propanol. In a sub-embodiment, the Compound A-1-propanol has an XRPD pattern substantially as shown in FIG. 58.

In another sub-embodiment, the Compound A-1-propanol has a melting onset at 191.2° C. to 197.2° C., as measured by Differential Scanning Calorimetry. In yet another sub-embodiment, the Compound A-1-propanol has the melting onset at 194.2° C.±3° C. In yet another sub-embodiment, the Compound A-1-propanol has a Thermogravimetric Analysis (TGA) substantially as shown in FIG. 59.

In embodiment 11, the invention provides an isopropyl alcohol solvate of Compound A. (Compound A-IPA). In embodiment 11a, the invention provides a solid form of the Compound A-IPA. In a sub-embodiment, the Compound A-IPA has an XRPD pattern substantially as shown in FIG. 60. In another sub-embodiment, the Compound A-IPA has a melting onset at 155.7° C. to 161.7° C., as measured by Differential Scanning Calorimetry. In yet another sub-embodiment, the Compound A-IPA has the melting onset at 158.7° C.±3° C. In yet another sub-embodiment, the Compound A-IPA has a Thermogravimetric Analysis (TGA) substantially as shown in FIG. 61.

In embodiment 12, the invention provides a methanol solvate of Compound A (Compound A-methanol). In embodiment 12a, the invention provides a solid form of the Compound A-methanol. In a sub-embodiment, the Compound A-methanol has an XRPD pattern substantially as shown in FIG. 62.

In embodiment 13, the invention provides an isopropyl acetate solvate of Compound A (Compound A-IPAc). In embodiment 13a, the invention provides a solid form of the Compound A-IPAc. In a sub-embodiment, the Compound A-IPAc has an XRPD pattern substantially as shown in FIG. 63.

In embodiment 14, the invention provides an acetone solvate of Compound A (Compound A-acetone). In embodiment 14a, the invention provides a solid form of the Compound A-acetone. In a sub-embodiment, the Compound A-acetone has an XRPD pattern substantially as shown in FIG. 64.

In embodiment 15, the invention provides a cyclopentyl methyl ether solvate of Compound A (Compound A-CPME). In embodiment 15a, the invention provides a solid form of the Compound A-CPME. In a sub-embodiment, the Compound A-CPME has an XRPD pattern substantially as shown in FIG. 65.

In embodiment 16, the invention provides a dioxane solvate of Compound A (Compound A-dioxane). In embodiment 16a, the invention provides a solid form of the Compound A-dioxane. In a sub-embodiment, the Compound A-dioxane has an XRPD pattern substantially as shown in FIG. 66.

In embodiment 17, the invention provides an ethyl acetate solvate of Compound A (Compound A-EtOAc). In embodiment 17a, the invention provides a solid form of the Compound A-EtOAc. In a sub-embodiment, the Compound A-EtOAc has an XRPD pattern substantially as shown in FIG. 67.

In embodiment 18, the invention provides an acetonitrile solvate of Compound A (Compound A-MeCN). In embodiment 18a, the invention provides a solid form of the Compound A-MeCN. In a sub-embodiment, the Compound A-MeCN has an XRPD pattern substantially as shown in FIG. 68.

In embodiment 19, the invention provides a methyl tert-butyl ether solvate of Compound A (Compound A-MTBE). In embodiment 19a, the invention provides a solid form of the Compound A-MTBE. In a sub-embodiment, the Compound A-MTBE has an XRPD pattern substantially as shown in FIG. 69.

In embodiment 20, the invention provides a toluene solvate of Compound A (Compound A-toluene). In embodiment 20a, the invention provides a solid form of the Compound A-toluene. In a sub-embodiment, the Compound A-toluene has an XRPD pattern substantially as shown in FIG. 70.

In embodiment 21, the invention provides a dodecyl sulfate salt of Compound A (Compound A-dodecyl sulfate). In embodiment 21a, the invention provides a solid form of the dodecyl sulfate (Compound A-dodecyl sulfate). In a sub-embodiment, the Compound A-dodecyl sulfate has an XRPD pattern substantially as shown in FIG. 71.

In embodiment 22, the invention provides a dimethyl formamide (DMF) solvate hydrate of Compound A (Compound A-DMF-hydrate). In embodiment 22a, the invention provides a solid form of the Compound A-DMF-hydrate. In a sub-embodiment, the Compound A-DMF-hydrate has an XRPD pattern substantially as shown in FIG. 73. In another sub-embodiment, the Compound A-DMF-hydrate has a melting onset at 104.8° C. to 110.8° C., as measured by Differential Scanning Calorimetry. In yet another sub-embodiment, the Compound A-DMF-hydrate has the melting onset at 107.8° C.±3° C. In yet another sub-embodiment, the Compound A-DMF-hydrate has a DSC pattern substantially as shown in FIG. 74.

In embodiment 23, the invention provides a dimethylacetamide (DMAC) solvate of Compound A (Compound A-DMAC). In embodiment 23a, the invention provides a solid form of the Compound A-DMAC. In a sub-embodiment, the Compound A-DMAC has an XRPD pattern substantially as shown in FIG. 75. In another sub-embodiment, the Compound A-DMAC has a melting onset at 147° C. to 153° C., as measured by Differential Scanning Calorimetry. In yet another sub-embodiment, the Compound A-DMAC has the melting onset at 150° C.±3° C. In yet another sub-embodiment, the Compound A-DMAC has a DSC pattern substantially as shown in FIG. 76.

In embodiment 24, the invention provides a monobesylate hydrate of Compound A (Compound A-besylate-hydrate). In embodiment 24a, the invention provides a solid form of the Compound A-besylate-hydrate. In embodiment 24b, the invention provides a solid form of the Compound A-besylate-hydrate Form 1. In a sub-embodiment, the Compound A-besylate-hydrate Form 1 has an XRPD pattern substantially as shown in FIG. 77. In yet another sub-embodiment, the Compound A-besylate-hydrate Form 1 has a DSC pattern substantially as shown in FIG. 78.

In embodiment 25, the invention provides a caffeine co-crystal of Compound A (Compound A-caffeine). In embodiment 25a, the invention provides a solid form of the Compound A-caffeine. In embodiment 25b, the solid form of the Compound A-caffeine is crystalline Compound A-caffeine Co-Crystal Form 1. In a sub-embodiment, the Compound A-caffeine Co-Crystal Form 1 has an XRPD pattern substantially as shown in FIG. 79. In yet another sub-embodiment, the Compound A-caffeine Co-Crystal Form 1 has a DSC pattern substantially as shown in FIG. 80. In yet another sub-embodiment, the Compound A-caffeine Co-Crystal Form 1 has a DVS pattern substantially as shown in FIG. 81.

In embodiment 26, the invention provides a citric acid co-crystal of Compound A (Compound A-citric acid). In embodiment 26a, the invention provides a solid form of the Compound A-citric acid. In embodiment 26b, the solid form of the Compound A-citric acid is crystalline Compound A-Citric Acid Co-Crystal Form 1. In a sub-embodiment, the Compound A-Citric Acid Co-Crystal Form 1 has an XRPD pattern substantially as shown in FIG. 82. In yet another sub-embodiment, the Compound A-Citric Acid Co-Crystal Form 1 has a DSC pattern substantially as shown in FIG. 83.

In embodiment 26c, the solid form of the Compound A-citric acid is crystalline Compound A Citric Acid Co-Crystal Form 2. In a sub-embodiment, the Compound A-Citric Acid Co-Crystal Form 2 has an XRPD pattern substantially as shown in FIG. 84. In yet another sub-embodiment, the Compound A-Citric Acid Co-Crystal Form 2 has a DSC and TGA pattern substantially as shown in FIG. 85.

In embodiment 27, the invention provides a saccharin co-crystal of Compound A (Compound A-saccharin). In embodiment 27a, the invention provides a solid form of the Compound A-saccharin. In embodiment 27b, the solid form of the Compound A-saccharin is crystalline Compound A-saccharin Co-Crystal Form 1. In a sub-embodiment, the Compound A-saccharin Co-crystal Form 1 has an XRPD pattern substantially as shown in FIG. 86. In yet another sub-embodiment, the Compound A-saccharin Co-Crystal Form 1 has a DSC pattern substantially as shown in FIG. 87. In yet another sub-embodiment, the Compound A-saccharin Co-Crystal Form 1 has a DVS pattern substantially as shown in FIG. 88.

In embodiment 28, the invention provides an L-tartaric acid co-crystal (Compound A-L-tartaric acid). In embodiment 28a, the invention provides a solid form of the Compound A-L-tartaric acid. In embodiment 28b, the solid form of the Compound A-L-tartaric acid is crystalline Compound A-L-tartaric acid Co-Crystal Form 1. In a sub-embodiment, the Compound A-L-tartaric acid Co-crystal Form 1 has an XRPD pattern substantially as shown in FIG. 89. In yet another sub-embodiment, the Compound A-L-tartaric acid Co-Crystal Form 1 has a DSC pattern substantially as shown in FIG. 90. In yet another sub-embodiment, the Compound A-L-tartaric acid Co-Crystal Form 1 has a DVS pattern substantially as shown in FIG. 91.

In embodiment 29, the invention provides a urea co-crystal (Compound A-Urea). In embodiment 29a, the invention provides a solid form of the Compound A-Urea. In embodiment 29b, the solid form of the Compound A-Urea is crystalline Compound A-Urea Co-Crystal Form 1. In a sub-embodiment, the Compound A-Urea Co-crystal Form 1 has an XRPD pattern substantially as shown in FIG. 92. In yet another sub-embodiment, the Compound A-Urea Co-Crystal Form 1 has a DSC pattern substantially as shown in FIG. 93. In yet another sub-embodiment, the Compound A-Urea Co-Crystal Form 1 has a DVS pattern substantially as shown in FIG. 94.

In embodiment 30, the invention provides a pharmaceutical composition comprising a salt, a hydrate, a solvate, or a co-crystal of Compound A; or a solid form of Compound A, salt, hydrate, solvate, or co-crystal thereof.

In embodiment 30a, the invention provides a pharmaceutical composition comprising a solid form of the Compound A, a salt, a hydrate, a solvate, or a co-crystal of Compound A. In a sub-embodiment, the solid form is crystalline or amorphous. In a sub-embodiment, the solid form is crystalline Compound A-Form 1. In another sub-embodiment, the solid form is a crystalline form of Anhydrous Compound A, including crystalline anhydrous forms 3, 4, 5, 6, 7, or 8.

In embodiment 30b, the invention provides a pharmaceutical composition comprising a salt, a hydrate, a solvate, or a co-crystal of Compound A, selected from hydrochloride salt (Compound A-HCl), mesylate salt (Compound A-MsA), tosylate salt (Compound A-TsA), sulfate salt (Compound A-sulfate), variable hydrate (Compound A-variable hydrate), tetrahydrofuran solvate (Compound A-THF), ethanol solvate (Compound A-ethanol), 1-propanol solvate (Compound A-1-propanol), isopropyl alcohol solvate (Compound A-IPA), methanol solvate (Compound A-methanol), isopropyl acetate solvate (Compound A-IPAc), acetone solvate (Compound A-acetone), cyclopentyl methyl ether solvate (Compound A-CPME), dioxane solvate (Compound A-dioxane), ethyl acetate solvate (Compound A-EtOAc), acetonitrile solvate (Compound A-MeCN), methyl tert-butyl ether solvate (Compound A-MTBE), toluene solvate (Compound A-toluene), dodecyl sulfate (Compound A-dodecyl sulfate), dimethyl formamide (DMF) solvate hydrate (Compound A-DMF-hydrate), dimethylacetamide (DMAC) solvate (Compound A-DMAC), monobesylate hydrate (Compound A-besylate-hydrate), caffeine co-crystal (Compound A-caffeine), citric acid co-crystal (Compound A-citric acid), saccharin co-crystal (Compound A-saccharin), L-tartaric acid co-crystal (Compound A-L-tartaric acid), or urea co-crystal (Compound A-urea); or the solid form thereof.

In embodiment 30c, the invention provides a pharmaceutical composition comprising a solid form of the Compound A-HCl of any of embodiments 1a-1j or any sub-embodiments thereof, and a pharmaceutically acceptable excipient. Preferably, the solid form of the Compound A-HCl is crystalline Form 1 of the Compound A-HCl having an XRPD pattern substantially as shown in FIG. 1.

In embodiment 30d, the invention provides a pharmaceutical composition comprising the solid form of the Compound A-MsA of any of embodiments 2a-2j or any sub-embodiments thereof, and a pharmaceutically acceptable excipient. Preferably, the solid form of the Compound A-MsA is crystalline Form 1 of the Compound A-MsA having an XRPD pattern substantially as shown in FIG. 10.

In embodiment 30e, the invention provides a pharmaceutical composition comprising the solid form of the Compound A-TsA of any of embodiments 3a-3j or any sub-embodiments thereof, and a pharmaceutically acceptable excipient. Preferably, the solid form of the Compound A-TsA is crystalline Form 4 of the Compound A-TsA having an XRPD pattern substantially as shown in FIG. 20.

In embodiment 30f, the invention provides a pharmaceutical composition comprising the solid form of the Compound A-Variable Hydrate of any of embodiments 6a-6e or any sub-embodiments thereof, and a pharmaceutically acceptable excipient. Preferably, the Compound A-Variable Hydrate is Compound A-Variable Hydrate Form 2 having an XRPD pattern substantially as shown in FIG. 36.

In embodiment 30g, the invention provides a pharmaceutical composition comprising the crystalline anhydrous form of Compound A, and a pharmaceutically acceptable excipient. Preferably, the crystalline anhydrous form of Compound A has an XRPD pattern substantially as shown in any one of FIG. 40, 43, 44, 47, 49, or 50.

In embodiment 30h, the invention provides a pharmaceutical composition comprising the solid form of the Compound A-Citric Acid Co-Crystal Form 1, and a pharmaceutically acceptable excipient. Preferably, the Compound A-Citric Acid Co-Crystal Form 1 has an XRPD pattern substantially as shown in FIG. 82.

In embodiment 30i, the invention provides a pharmaceutical composition comprising the solid form of the Compound A-Citric Acid Co-Crystal Form 2, and a pharmaceutically acceptable excipient. Preferably, the Compound A-Citric Acid Co-Crystal Form 2 has an XRPD pattern substantially as shown in FIG. 84.

In embodiment 30j, the invention provides a pharmaceutical composition comprising the solid form of the Compound A-dodecyl sulfate, and a pharmaceutically acceptable excipient. Preferably, the Compound A-dodecyl sulfate has an XRPD pattern substantially as shown in FIG. 71.

In embodiment 31, the invention provides a method of treating a subject suffering from a disease mediated by KIF18A inhibition, comprising administering to a subject in need thereof a pharmaceutically effective amount of the pharmaceutical composition of any one of embodiments 30-30j.

In embodiment 31a, the invention provides a method of embodiment 31, wherein said disease mediated by KIF18A inhibition is a neoplastic disease. In a sub-embodiment, the neoplastic disease is a cancer or a tumor. In a further sub-embodiment, the cancer is ovarian cancer, breast cancer, lung cancer, or endometrial cancer. In a further sub-embodiment, the ovarian cancer is high grade serous ovarian cancer (HGSOC), optionally, metastatic, or unresectable HGSOC. In a further sub-embodiment, the HGSOC is platinum-resistant HGSOC or wherein the HGSOC progressed during or within 6 months of a platinum-containing regimen. In a further sub-embodiment, the cancer is primary peritoneal cancer and/or cancer of the fallopian tube. In a further sub-embodiment, the breast cancer is triple negative breast cancer. In a further sub-embodiment, the endometrial cancer is serous endometrial cancer. In a further sub-embodiment, the serous endometrial cancer is metastatic or recurrent serous endometrial cancer. In a further sub-embodiment, the serous endometrial cancer has relapsed or is refractory to at least one line of systemic chemotherapy. In a further sub-embodiment, the serous endometrial cancer has relapsed or is refractory to at least one line of systemic chemotherapy. In a further sub-embodiment, the lung cancer is non small cell lung cancer. In a further sub-embodiment, the tumor is an advanced solid tumor. In a further sub-embodiment, the tumor is non-resectable, metastatic and/or non-localized. In a further sub-embodiment, the tumor has relapsed or is refractory to at least one line of systemic chemotherapy.

In embodiment 31 b, the invention provides a method of embodiment 31, 31a or any sub-embodiment thereof, wherein the subject has relapsed or is refractory to at least one line of systemic chemotherapy. In a sub-embodiment, the systemic chemotherapy comprises taxane, gemcitabine, or doxorubicin. In a further sub-embodiment, the systemic chemotherapy comprises cisplatin, carboplatin or lenvatinib.

In embodiment 31c, the invention provides a method of embodiment 31, 31a, 31b, or any sub-embodiment thereof, wherein the cancer or tumor comprises cells that are positive for an inactivated TP53 gene and/or positive for at least one of an inactivated Rb gene, (ii) an amplified CCNE1 gene or overexpressed CCNE1 gene product, (iii) an inactivated BRCA gene or (iv) a combination thereof.

In embodiment 31c, the invention provides a method of embodiment 31, 31a, 31b, 31c, or any sub-embodiment thereof, wherein the subject is an adult human.

In embodiment 32, the invention provides a method for preparing the Compound A-HCl of any of embodiments 1-1j or any sub-embodiments thereof, the method comprising: combining hydrochloric acid, Compound A, and a suitable solvent to form the Compound A-HCl salt or the solid form thereof. In a sub-embodiment, the suitable solvent is selected from acetonitrile/water, acetonitrile/1,4-dioxane, tetrahydrofuran/water, N-Methyl-2-pyrrolidonelethanol or acetone/water.

In embodiment 33, the invention provides a method for preparing the Compound A-MsA of any of embodiments 2-2j or any sub-embodiments thereof, the method comprising: combining methanesulfonic acid, Compound A, and a suitable solvent to form the Compound A-MsA salt or the solid form thereof. In a sub-embodiment, the suitable solvent is selected from acetonitrile or ethyl acetate.

In embodiment 34, the invention provides a method for preparing the Compound A-TsA of any of embodiments 3-3i or any sub-embodiments thereof, the method comprising: combining p-toluenesulfonic acid, Compound A, and a suitable solvent to form the Compound A-TsA salt or the solid form thereof. In a sub-embodiment, the suitable solvent is selected from ethanol or isopropanol.

In embodiment 35, the invention provides a method for preparing the solid form of Compound A-Variable-Hydrate-Form 2 of any of embodiments 6b-6h or any sub-embodiments thereof, the method comprising: (a) combining water and a mixture of Compound A-methanol solvate form 1 and Compound A-ethanol solvate form 1 to form the Compound A-Variable-Hydrate-Form 2; or (b) combining Compound A in alcohol solvent, followed by water, filtration, and drying at elevated temperature to remove the alcohol solvent. In a sub-embodiment, the alcohol solvent is a mixture of methanol and ethanol. In another sub-embodiment, the elevated temperature is 50° C.

In embodiment 36, the invention provides a hydrochloride salt of Compound A, having the structure:

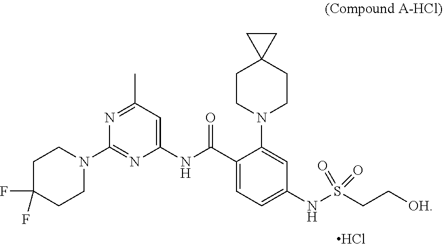 [SMILES: Cc1cc(NC(=O)c2ccc(NS(=O)(=O)CCO)cc2N2CCC3(CC2)CC3)nc(N2CCC(F)(F)CC2)n1 ; Cl]

In embodiment 37, the invention provides a mesylate salt of Compound A, having the structure:

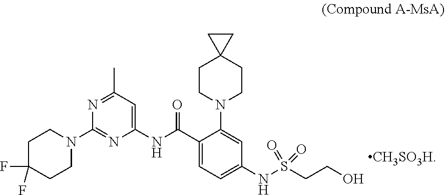

In embodiment 38, the invention provides a tosylate salt of Compound A, having the structure:

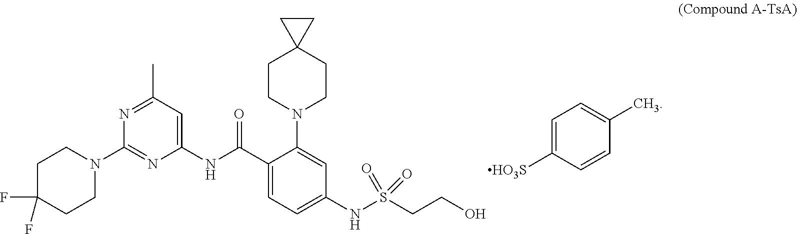

In embodiment 39, the invention provides a hydrate of Compound A, having the structure:

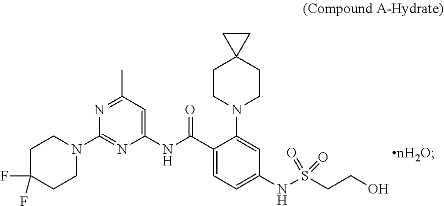

wherein n is in the range of 0.5 and 2.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Further aspects and advantages will be apparent to those of ordinary skill in the art from a review of the following detailed description, taken in conjunction with the drawings. The description hereafter includes specific embodiments with the understanding that the disclosure is illustrative and is not intended to limit the invention to the specific embodiments described herein.

### BRIEF DESCRIPTION OF THE DRAWINGS

FIG. 1 depicts an X-ray powder diffraction (“XRPD”) pattern of the crystalline Compound A-HCl Form 1.

FIG. 2 depicts a Differential Scanning Calorimetry (DSC) thermograph and Thermogravimetric analysis (TGA) of the crystalline Compound A-HCl Form 1.

FIG. 3 depicts a Dynamic Vapor Sorption (DVS) profile of the crystalline Compound A-HCl Form 1.

FIG. 4 depicts a solid state ¹⁹F NMR of the crystalline Compound A-HCl Form 1.

FIG. 5 depicts a single crystal X-Ray crystal structure of the crystalline Compound A-HCl Form 1.

FIG. 6 depicts powder dissolution of Compound A-Anhydrous-Form 3, Compound A-Variable-Hydrate Form 2, Compound A-HCl Form 1, and Compound A-Amorphous Form.

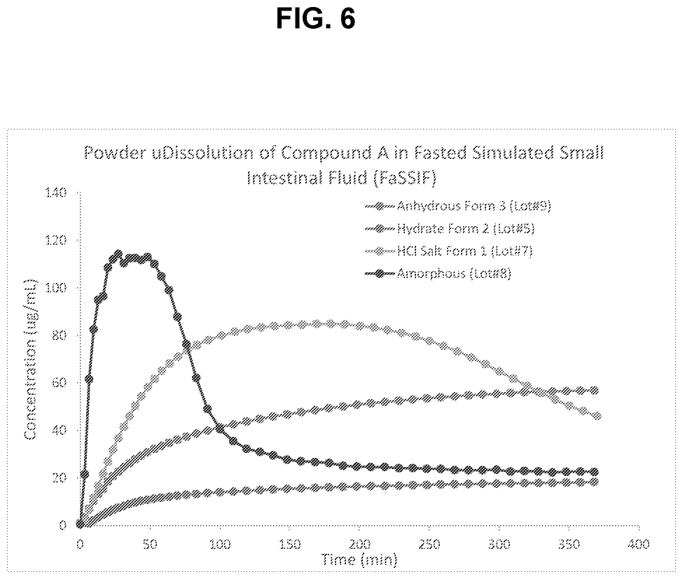

FIG. 7 depicts an XRPD pattern of the crystalline Compound A-HCl Form 2.

FIG. 8 depicts DSC thermograph of the crystalline Compound A-HCl Form 2.

FIG. 9 depicts modulated DSC of amorphous form of Compound A-HCl.

FIG. 10 depicts an XRPD pattern of the crystalline Compound A-MsA Form 1.

FIG. 11 depicts a DSC thermograph and TGA of the crystalline Compound A-MsA Form 1.

FIG. 12 depicts a DVS moisture sorption profile of crystalline Compound A-MsA Form 1.

FIG. 13 depicts a solid state ¹⁹F NMR of the crystalline Compound A-MsA Form 1.

FIG. 14 depicts an XRPD pattern of the crystalline Compound A-MsA Form 2.

FIG. 15 depicts a DSC thermograph of the crystalline Compound A-MsA Form 2.

FIG. 16 depicts TGA of the crystalline Compound A-MsA Form 2.

FIG. 17 depicts an XRPD pattern of the crystalline Compound A-TsA Form 1.

FIG. 18 depicts Variable Temperature X-Ray Diffraction (VTXRD) of the crystalline Compound A-TsA Form 1 showing a recrystallization at ≥180° C. and forming a new crystalline Compound A-TsA Form 5.

FIG. 19 depicts DSC thermograph and TGA of the crystalline Compound A-TsA Form 1

FIG. 20 depicts an XRPD pattern of the crystalline Compound A-TsA Form 2.

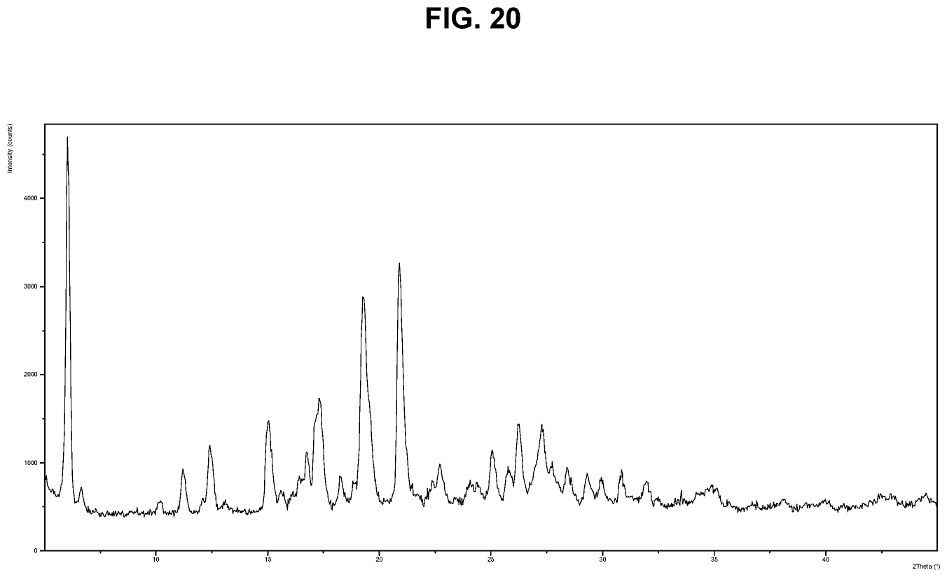

FIG. 21 depicts a DSC thermograph and TGA of the crystalline Compound A-TsA Form 2.

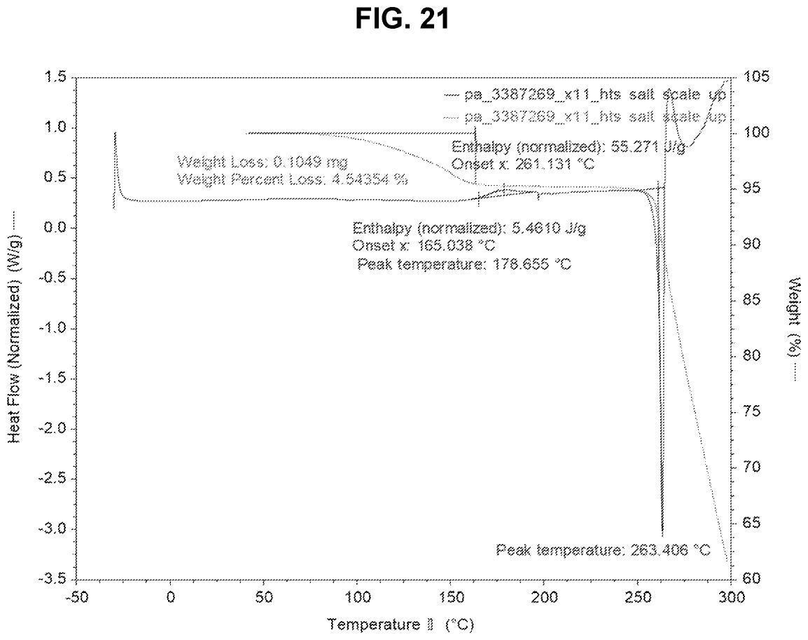

FIG. 22 depicts an XRPD pattern of the crystalline Compound A-TsA Form 3.

FIG. 23 depicts a DSC thermograph and TGA of the crystalline Compound A-TsA Form 3.

FIG. 24A depicts an XRPD pattern of the crystalline Compound A-TsA Form 4.

FIG. 24B depicts a single crystal X-ray crystal structure of the crystalline Compound A-TsA Form 4.

FIG. 25 depicts a DSC thermograph and TGA of the crystalline Compound A-TsA Form 4.

FIG. 26 a solid state ¹⁹F NMR of the crystalline Compound A-TsA Form 4.

FIG. 27 depicts an XRPD pattern of the crystalline Compound A-TsA Form 5.

FIG. 28 depicts an XRPD pattern of the crystalline Compound A-DiTsA Form 6.

FIG. 29 depicts powder dissolution and kinetic solubility of Compound A-HCl Salt Form 1, Compound A-Mesylate Form 1, and Compound A-Tosylate Form 4 in Fasted Simulated Small Intestinal Fluid (FaSSIF).

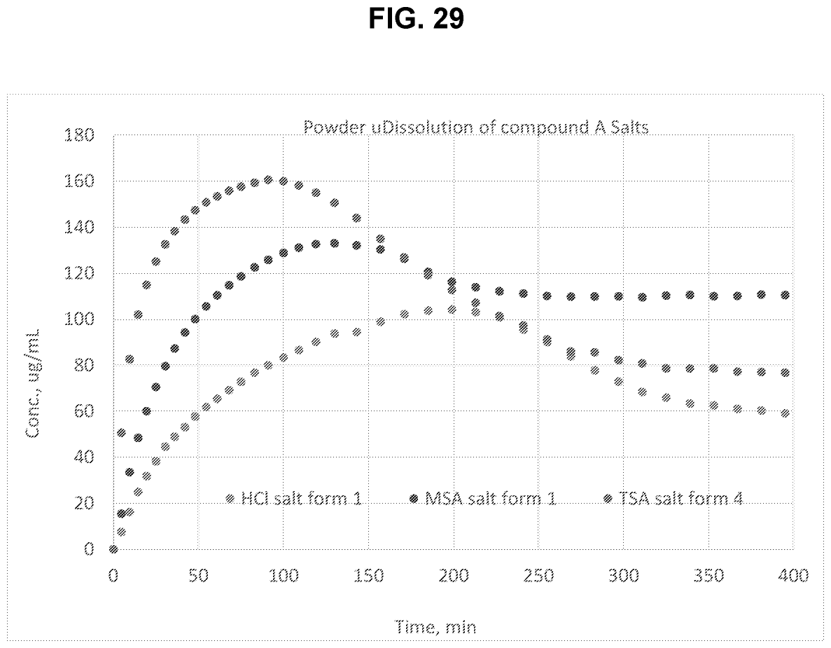

FIG. 30 depicts an XRPD pattern of the crystalline Compound A-sulfate Form 1.

FIG. 31 depicts a DSC thermograph and TGA of the crystalline Compound A-sulfate Form 1.

FIG. 32 depicts a DVS of the crystalline Compound A-sulfate Form 1.

FIG. 33 depicts an XRPD pattern of the Compound A-Amorphous form.

FIG. 34 depicts a DSC thermograph indicating a glass transition temperature (Tg) at 91° C. of the Compound A-Amorphous form.

FIG. 35 depicts TGA-IR showing about 1.05% weight loss of water from the Compound A amorphous form below 100° C.

FIG. 36 depicts an XRPD pattern of the crystalline Compound A-Variable Hydrate Form 2.

FIG. 37 depicts a DSC thermograph of the crystalline Compound A-Variable Hydrate Form 2.

FIG. 38 depicts TGA of the crystalline Compound A-Variable Hydrate Form 2.

FIG. 39 depicts a DVS moisture sorption profile of crystalline Compound A-Variable Hydrate Form 2.

FIG. 40 depicts an XRPD pattern of the crystalline Compound A-Anhydrous Form 3.

FIG. 41 depicts a DSC thermograph of the crystalline Compound A-Anhydrous Form 3.

FIG. 42 depicts a DVS profile of the crystalline Compound A-Anhydrous Form 3.

FIG. 43 depicts an XRPD pattern of the crystalline Compound A-Anhydrous Form 4.

FIG. 44 depicts an XRPD pattern of the crystalline Compound A-Anhydrous Form 5.

FIG. 45 depicts a DSC thermograph and TGA of the crystalline Compound A-Anhydrous Form 5.

FIG. 46 depicts a DVS profile of the crystalline Compound A-Anhydrous Form 5 wherein the Anhydrous Form 5 rehydrates to Compound A-Monohydrate.

FIG. 47 depicts an XRPD pattern of the crystalline Compound A-Anhydrous Form 6.

FIG. 48 depicts a DSC thermograph and TGA of the crystalline Compound A-Anhydrous Form 6.

FIG. 49 depicts an XRPD pattern of the crystalline Compound A-Anhydrous Form 7.

FIG. 50 depicts an XRPD pattern of the crystalline Compound A-Anhydrous Form 8.

FIG. 51 depicts a DSC thermograph and TGA of the crystalline Compound A-Anhydrous Form 8.

FIG. 52 depicts an XRPD pattern of the Crystalline Compound A Form 1.

FIG. 53 depicts an XRPD pattern of the crystalline Compound A-THF solvate.

FIG. 54 depicts a DSC thermograph and TGA of the crystalline Compound A-THF solvate.

FIG. 55 depicts an XRPD pattern of the crystalline Compound A-ethanol solvate.

FIG. 56 depicts TGA of the crystalline Compound A-ethanol solvate.

FIG. 57 depicts a DSC thermograph of the crystalline Compound A-ethanol solvate.

FIG. 58 depicts an XRPD pattern of the crystalline Compound A-1-propanol solvate.

FIG. 59 depicts a DSC thermograph and TGA of the crystalline Compound A-1-propanol solvate.

FIG. 60 depicts an XRPD pattern of the crystalline Compound A-isopropyl alcohol (IPA) solvate.

FIG. 61 depicts a DSC thermograph and TGA of the crystalline Compound A-IPA solvate.

FIG. 62 depicts an XRPD pattern of the crystalline Compound A-Methanol solvate.

FIG. 63 depicts an XRPD pattern of the crystalline Compound A-Isopropyl Acetate (IPAc) solvate.

FIG. 64 depicts an XRPD pattern of the crystalline Compound A-Acetone solvate.

FIG. 65 depicts an XRPD pattern of the crystalline Compound A-Cyclopentyl Methyl Ether (CPME) solvate.

FIG. 66 depicts an XRPD pattern of the crystalline Compound A-Dioxane solvate.

FIG. 67 depicts an XRPD pattern of the crystalline Compound A-Ethyl Acetate (EtOAc) solvate.

FIG. 68 depicts an XRPD pattern of the crystalline Compound A-Acetonitrile (MeCN) solvate.

FIG. 69 depicts an XRPD pattern of the crystalline Compound A-Methyl Tert-Butyl Ether (MTBE) solvate.

FIG. 70 depicts an XRPD pattern of the crystalline Compound A-Toluene solvate.

FIG. 71 depicts an XRPD pattern of the crystalline Compound A-Dodecyl Sulfate solvate.

FIG. 72 depicts a DSC thermograph and TGA of the crystalline Compound A-Dodecyl Sulfate solvate.

FIG. 73 depicts an XRPD pattern of the crystalline Compound A-Dimethyl Formamide (DMF) solvate hydrate.

FIG. 74 depicts a DSC thermograph of the crystalline Compound A-Dimethyl Formamide (DMF) solvate hydrate.

FIG. 75 depicts an XRPD pattern of the crystalline Compound A-Dimethylacetamide (DMAC) solvate.

FIG. 76 depicts a DSC thermograph of the crystalline Compound A-Dimethylacetamide (DMAC) solvate.

FIG. 77 depicts an XRPD pattern of the crystalline Compound A-MonoBesylate Hydrate Form 1.

FIG. 78 depicts a DSC thermograph and TGA of the crystalline Compound A-MonoBesylate Hydrate Form 1.

FIG. 79 depicts an XRPD pattern of the crystalline Compound A-Caffeine Co-Crystal Form 1.

FIG. 80 depicts a DSC thermograph and TGA of the crystalline Compound A-Caffeine Co-Crystal Form 1.

FIG. 81 depicts a DVS profile of the crystalline Compound A-Caffeine Co-Crystal Form 1.

FIG. 82 depicts an XRPD pattern of the crystalline Compound A-Citric Acid Co-Crystal Form 1.

FIG. 83 depicts a DSC thermograph and TGA of the crystalline Compound A-Citric Acid Co-Crystal Form 1.

FIG. 84 depicts an XRPD pattern of the crystalline Compound A-Citric Acid Co-Crystal Form 2.

FIG. 85 depicts a DSC thermograph and TGA of the crystalline Compound A-Citric Acid Co-Crystal Form 2.

FIG. 86 depicts an XRPD pattern of the crystalline Compound A-Saccharin Co-Crystal Form 1.

FIG. 87 depicts a DSC thermograph and TGA of the crystalline Compound A-Saccharin Co-Crystal Form 1.

FIG. 88 depicts a DVS profile of the crystalline Compound A-Saccharin Co-Crystal Form 1.

FIG. 89 depicts an XRPD pattern of the crystalline Compound A-L-Tartaric Acid Co-Crystal Form 1.

FIG. 90 depicts a DSC thermograph and TGA of the crystalline Compound A-L-Tartaric Acid Co-Crystal Form 1

FIG. 91 depicts a DVS profile of the crystalline Compound A-L-Tartaric Acid Co-Crystal Form 1.

FIG. 92 depicts an XRPD pattern of the crystalline Compound A-Urea Co-Crystal Form 1.

FIG. 93 depicts a DSC thermograph and TGA of the crystalline Compound A-Urea Co-Crystal Form 1.

FIG. 94 depicts a DVS profile of the crystalline Compound A-Urea Co-Crystal Form 1.

FIG. 95 depicts dog cross-over PK Study of Compound A-HCl Form 1, Compound A-Anhydrous Form 3, and Compound A-Amorphous.

### DETAILED DESCRIPTION

Provided herein is a salt, a hydrate, a solvate, or a co-crystal of Compound A; a solid form of the Compound A, salt, hydrate, solvate, or co-crystal thereof; pharmaceutical compositions thereof; and methods of treating a subject suffering from cancer, comprising administering to the subject a therapeutically effective amount of the pharmaceutical compositions.

Compound A is a KIF18A inhibitor and, in various aspects, has a KIF18A ATPase IC50 of about 0.071 μM. The KIF18A gene belongs to Kinesin-8 subfamily and is a plus-end-directed motor. KIF18A is believed to influence dynamics at the plus end of kinetochore microtubules to control correct chromosome positioning and spindle tension. Depletion of human KIF18A leads to longer spindles, increased chromosome oscillation at metaphase, and activation of the mitotic spindle assembly checkpoint in HeLa cervical cancer cells (MI Mayr et al, Current Biology 17, 488-98, 2007). KIF18A is overexpressed in various types of cancers, including but not limited to colon, breast, lung, pancreas, prostate, bladder, head, neck, cervix, and ovarian cancers. Overexpression of KIF18A dampens sister chromatid oscillation resulting in tight metaphase plates. Inactivation of KIF18A motor function in KIF18A knockout mice or by mutagenic ethylmethanosulfonate (EMS) treatment in KIF18A^{gcd2/gcd2} mice (missense mutation (R308K) in the motor domain) result in viable mice with no gross abnormalities in major organs except for clear testis atrophy and sterility (J Stumpff et al Developmental Cell. 2008; 14:252-262; J Stumpff et al Developmental Cell. 2012; 22:1017-1029; XS Liu et al. Genes & Cancer. 2010; 1:26-39; CL Fonseca et al J Cell Biol. 2019;1-16; A Czechanski et al Developmental Biology. 2015; 402:253-262. O Rath, F Kozielski. Nature Reviews Cancer. 2012; 12:527-539). Normal human and mouse KIF18A-deficient somatic cells were shown to complete cell division with relatively normal mitotic progression but without proper chromosome alignment resulting in daughter cells with a normal karyotype, some defects in exit from mitosis were noted in a subset of normal cells resulting in micronuclei formation on slower proliferation (CL Fonseca et al J Cell Biol. 2019;1-16). These genetic studies suggest that normal germ and somatic cells have different dependency on requirements for chromosome alignment and indicate that KIF18A may be dispensable in normal euploidy somatic cell division (X S Liu et al Genes & Cancer. 2010; 1:26-39; A Czechanski et al Developmental Biology. 2015; 402:253-262). In normal human tissues, expression of KIF18A is elevated in tissues with actively cycling cells, with highest expression in the testis (GTEx Portal, GTEx Portal, J Lonsdale et al Nature Genetics. 2013:29; 45:580). In various aspects, Compound A inhibits ATPase activity. For example, Compound A inhibits MT-ATPase activity and not basal ATPase activity.

The compounds disclosed herein may be identified either by their chemical structure and/or chemical name herein. When the chemical structure and chemical name conflict, the chemical structure is determinative of the identity of the compound.

When ranges are used herein for physical properties, such as molecular weight, or chemical properties, such as chemical formulae, all combinations and subcombinations of ranges and specific embodiments therein are intended to be included.

As used herein, chemical structures which contain one or more stereocenters depicted with dashed and bold bonds (i.e., [symbol] and [symbol] ) are meant to indicate absolute stereochemistry of the stereocenter(s) present in the chemical structure. As used herein, bonds symbolized by a simple line do not indicate a stereo-preference. Unless otherwise indicated to the contrary, chemical structures that include one or more stereocenters which are illustrated herein without indicating absolute or relative stereochemistry encompass all possible stereoisomeric forms of the compound (e.g., diastereomers, enantiomers) and mixtures thereof. Structures with a single bold or dashed line, and at least one additional simple line, encompass a single enantiomeric series of all possible diastereomers.

The term “about” is meant to account for variations due to experimental error. All measurements reported herein are understood to be modified by the term “about,” whether the term is explicitly used, unless explicitly stated otherwise. As used herein, the singular forms “a,” “an,” and “the” include plural referents unless the context clearly dictates otherwise.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The term “hydrate” refers to the chemical entity formed by the interaction of water and a compound, including, for example, hemi-hydrates, monohydrates, dihydrates, trihydrates, etc. A hydrate, as used herein, can have a variable amount of water, usually from 0.5 to 2 water molecules, such as, 0.5, 1, 1.5, or 2 water molecules per compound A molecule, referred to as “variable hydrate”. The number of water molecules can vary depending on various method of preparations and storage conditions of the hydrate forms.

The term “solid form” and “physical form” are meant to include all crystalline and amorphous forms of the compound, including, for example, polymorphs, pseudopolymorphs, solvates, hydrates, unsolvated polymorphs (including anhydrous), conformational polymorphs, and amorphous forms, as well as mixtures thereof, unless a particular crystalline or amorphous form is referred to.

The term “co-crystal” as used herein, refers to a crystalline complex of a neutral molecular constituent and Compound A bound together in the crystal lattice through noncovalent interactions, often including hydrogen bonding. Examples of co-crystals include caffeine co-crystal (Compound A-caffeine), citric acid co-crystal (Compound A-citric acid), saccharin co-crystal (Compound A-saccharin), L-tartaric acid co-crystal (Compound A-L-tartaric acid), or urea co-crystal (Compound A-urea.

The term “glass transition temperature” refers to a range of temperature in which an amorphous solid form experiences a gradual and reversible transition from a hard and relatively brittle “glassy” state into a viscous or rubbery state as the temperature is increased.

Isolation and Purification of Salts. Hydrates, Solvates, Co-Crystals of Compound A; and a Solid Form of the Compound A, Including Crystalline Anhydrous Forms, Salts, Solvates, and Co-Crystals Thereof.

Compound A has ionizable functional groups with one weakly basic pKa value of 3.9 and one weakly acidic pKa value of 7.3. From high-throughput and manual polymorph screening, the present inventors generated various salts, hydrates, solvates, co-crystals of compound A; and a solid form of the Compound A, including crystalline anhydrous forms, salts, hydrates, solvates, and co-crystals thereof. The desolvation of the ethanol solvate through drying generated the relatively stable Compound A-Hydrate-Form 2, which dehydrated starting at 25° C. and had a very low solubility. The desolvation of the Compound A-THF solvate generated an Anhydrous-Compound-A-Form 3, which quickly converted to the Compound A Hydrate-Form 2 in the aqueous media or upon moisture uptake. Based on the solid-state properties of the free base forms of Compound A, and their inclinations to form solvates, the present inventors generated various salts, hydrates, solvates, and co-crystals of compound A that may be suitable for drug substance scale up and crystallization for pharmaceutical development.

From high-throughput and manual salt screenings, various counterions and solvents were tested and generated crystalline salts and solvates of Compound A. Several salts, hydrates, solvates of Compound A, including sulfate, besylate, mesylate and tosylate salts, were formed in multiple polymorphs. After further solubility and stability testings, Compound A-HCl Salt Form 1 was prioritized for further evaluation because of its acceptable solubility and stability profiles, improved biopharmaceutical properties and favorable crystallization process. Polymorph screening of Compound A-HCl Salt Form 1 recovered a total of 126 crystalline samples from 384 crystallization conditions. Of those, the XRPD patterns of 90 samples were the same as that of HCl salt Form 1. The others may include disproportionated Compound A or Compound A Solvate forms. Polymorph screening of the Compound-A-HCl Salts revealed that Compound-A-HCl-Form 1 was the most thermodynamic stable form. Multiple co-crystals of Compound A were also generated from co-crystal screening, e.g. citric acid, tartaric acid, caffeine, and urea co-crystals.

### Crystalline Compound A-Hydrochloride Salt Form 1 (Compound A-HCl Salt Form 1).

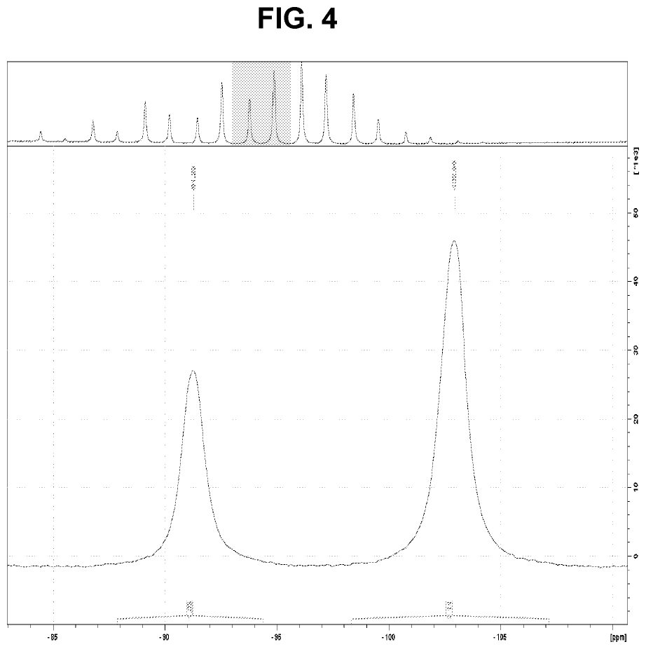

Also provided herein is a crystalline Compound A-HCl Salt Form 1. The crystalline Compound A-HCl Salt Form 1 can be characterized by solid state ¹⁹F NMR, obtained as set forth in the Examples, having peaks at −91 and −103±0.5 ppm. In some embodiments, the crystalline Compound A-HCl Salt Form 1 has a solid state ¹⁹F NMR substantially as shown in FIG. 4, wherein by “substantially” is meant that the reported peaks can vary by ±0.5 ppm.

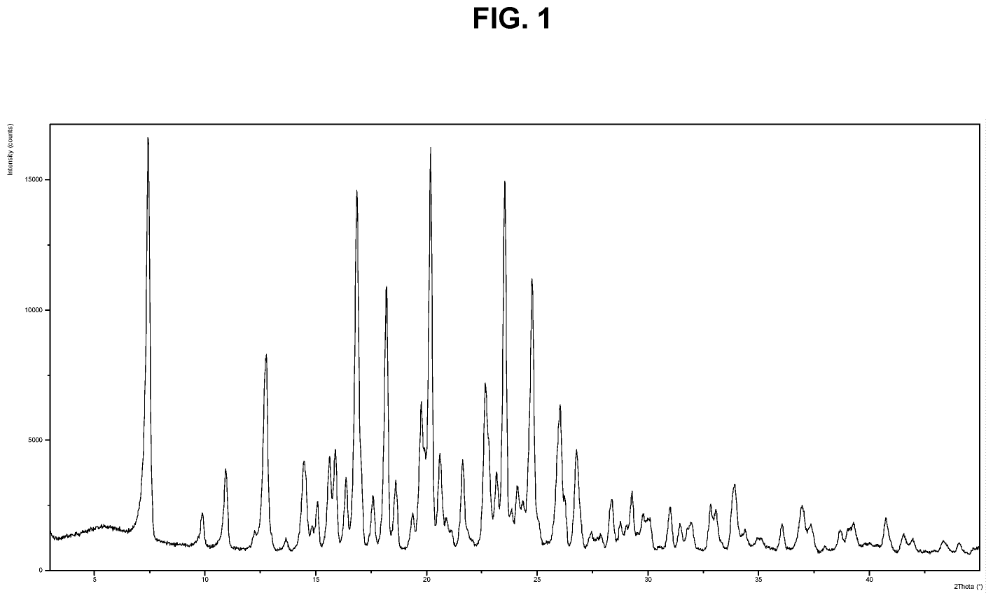

The crystalline Compound A-HCl Salt Form 1 can be further characterized by an X-ray powder diffraction pattern, obtained as set forth in the Examples, having peaks at 7.5, 16.9, and 20.2±0.2° 2θ using Cu Kα radiation. The crystalline Compound A-HCl Salt Form 1 optionally can be further characterized by an X-ray powder diffraction pattern having additional peaks at 12.8, 18.2, 22.7, 23.6, 24.8 and 26.1±0.2° 2θ using Cu Kα radiation. The crystalline Compound A-HCl Salt Form 1 optionally can be further characterized by an X-ray powder diffraction pattern having additional peaks at 10.9, 14.5, 15.7, 15.9, 19.8, 20.6, 21.6, 23.2, 26.1 and 26.8±0.2° 2θ using Cu Kα radiation. In some embodiments, crystalline Compound A-HCl Salt Form 1 has an X-ray powder diffraction pattern substantially as shown in FIG. 1, wherein by “substantially” is meant that the reported peaks can vary by ±0.2°. Those skilled in the art know that in the field of XRPD, while relative peak heights in spectra are dependent on several factors, such as sample preparation and instrument geometry, peak positions are relatively insensitive to experimental details.

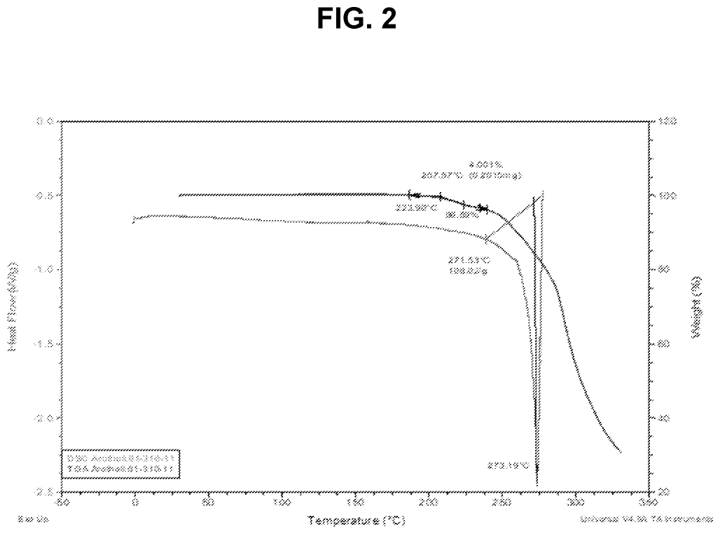

Differential scanning calorimetry (DSC) thermographs were obtained, as set forth in the Examples, for the crystalline Compound A-HCl Salt Form 1. The DSC curve indicated an endothermic transition at 271.5° C.±3° C. Thus, in some embodiments, the crystalline Compound A-HCl Salt Form 1 can be characterized by a DSC thermograph having a transition endotherm with an onset of 268.5° C. to 274.5° C. For example, in some embodiments the crystalline Compound A-HCl Salt Form 1 is characterized by DSC, as shown in FIG. 2.

The crystalline Compound A-HCl Salt Form 1 also can be characterized by thermogravimetric analysis (TGA). Thus, the crystalline Compound A-HCl Salt Form 1 can be characterized by a weight loss of about 4% with an onset temperature of 268.3° C. to 273.7° C. For example, the crystalline Compound A-HCl Salt Form 1 can be characterized by a weight loss of about 4%, up to about 271° C. In some embodiments, the crystalline Compound A-HCl Salt Form 1 has a thermogravimetric analysis substantially as depicted in FIG. 2, wherein by “substantially” is meant that the reported TGA features can vary by ±1% of the about 4% weight loss.

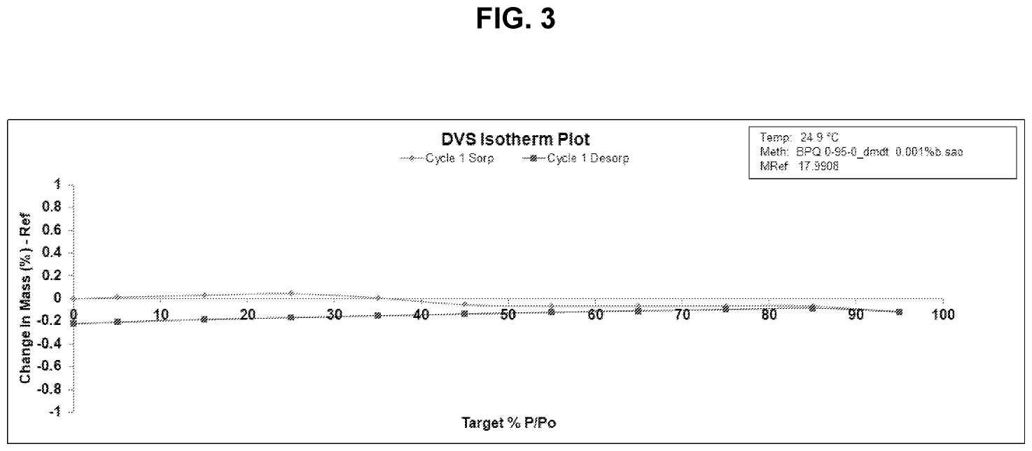

The crystalline Compound A-HCl Salt Form 1 can be characterized by a moisture sorption profile. For example, in some embodiments the crystalline Compound A-HCl Salt Form 1 is characterized by the moisture sorption profile (DVS) as shown in FIG. 3, showing a weight gain of less than 0.5% by 95% RH.

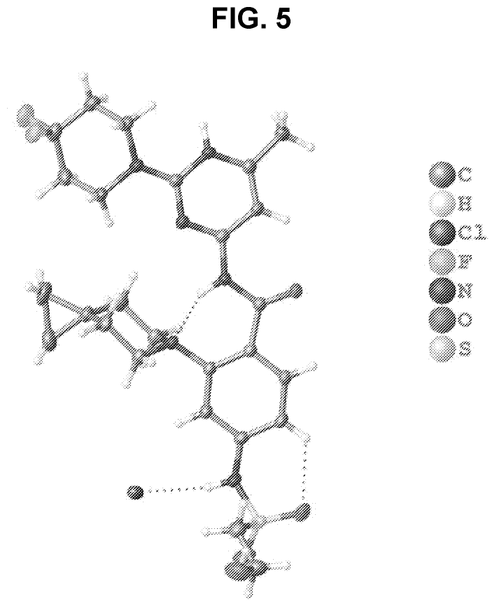

The crystalline Compound A-HCl Salt Form 1 is further characterized by a single crystal structure substantially as shown in FIG. 5, or as set forth in the Examples.

Further provided herein are pharmaceutical compositions comprising the crystalline Compound A-HCl Salt Form 1 as described herein and a pharmaceutically acceptable excipient.

### Crystalline Compound A-Mesylate Salt Form 1 (Compound A-MsA Salt Form 1).

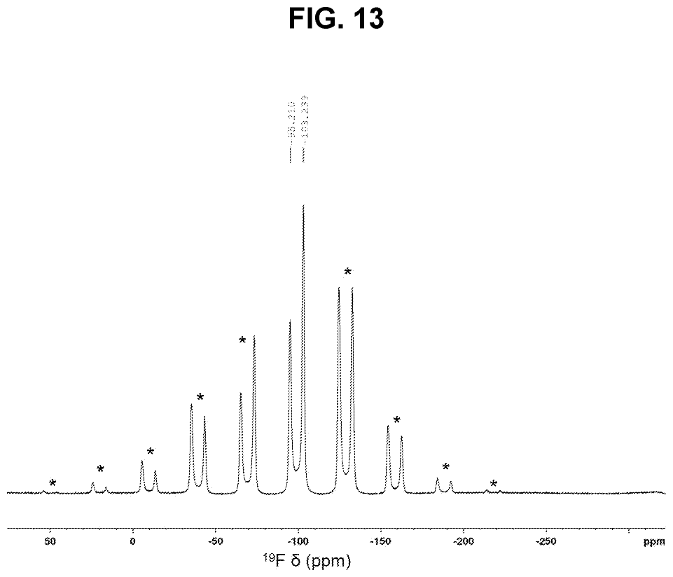

Also provided herein is a crystalline Compound A-MsA Salt Form 1. The crystalline Compound A-MsA Salt Form 1 can be characterized by solid state ¹⁹F NMR, obtained as set forth in the Examples, having peaks at −95.2 and −103.2±0.5 ppm. In some embodiments, the crystalline Compound A-MsA Salt Form 1 has a solid state ¹⁹F NMR substantially as shown in FIG. 13, wherein by “substantially” is meant that the reported peaks can vary by ±0.5 ppm.

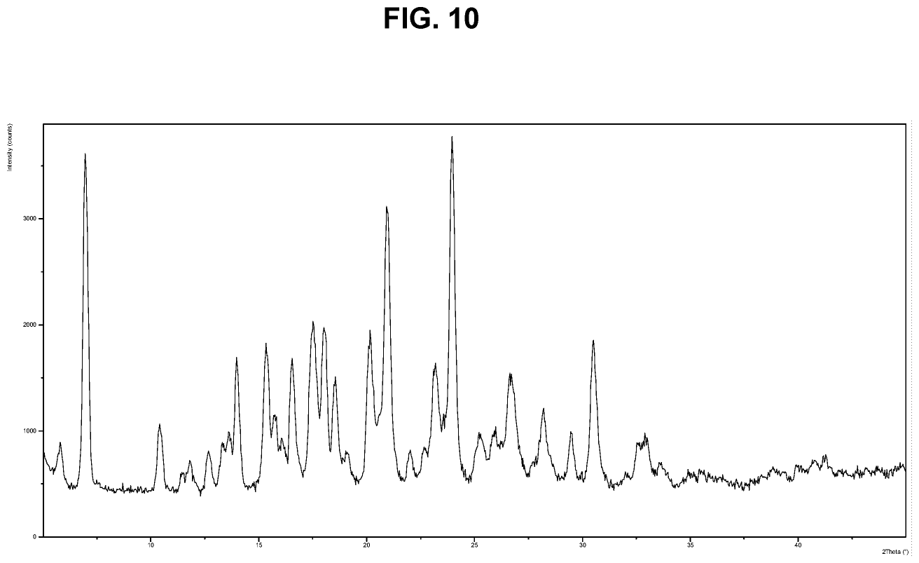

The crystalline Compound A-MsA Salt Form 1 can be further characterized by an X-ray powder diffraction pattern, obtained as set forth in the Examples, having peaks at 7.0, 16.5, and 23.9±0.2° 2θ using Cu Kα radiation. The crystalline Compound A-MsA Salt Form 1 optionally can be further characterized by an X-ray powder diffraction pattern having additional peaks at 12.6, 15.7, 17.4, 18.5, 20.0 and 21.0±0.2° 2θ using Cu Kα radiation. The crystalline Compound A-MsA Salt Form 1 optionally can be further characterized by an X-ray powder diffraction pattern having additional peaks at 5.8, 11.8, 13.5, 15.3, 16.1, 18.0, 20.6, 25.2, 28.0 and 30.5±0.2° 2θ using Cu Kα radiation. In some embodiments, crystalline Compound A-MsA Salt Form 1 has an X-ray powder diffraction pattern substantially as shown in FIG. 10, wherein by “substantially” is meant that the reported peaks can vary by ±0.2°. Those skilled in the art know that in the field of XRPD, while relative peak heights in spectra are dependent on several factors, such as sample preparation and instrument geometry, peak positions are relatively insensitive to experimental details.

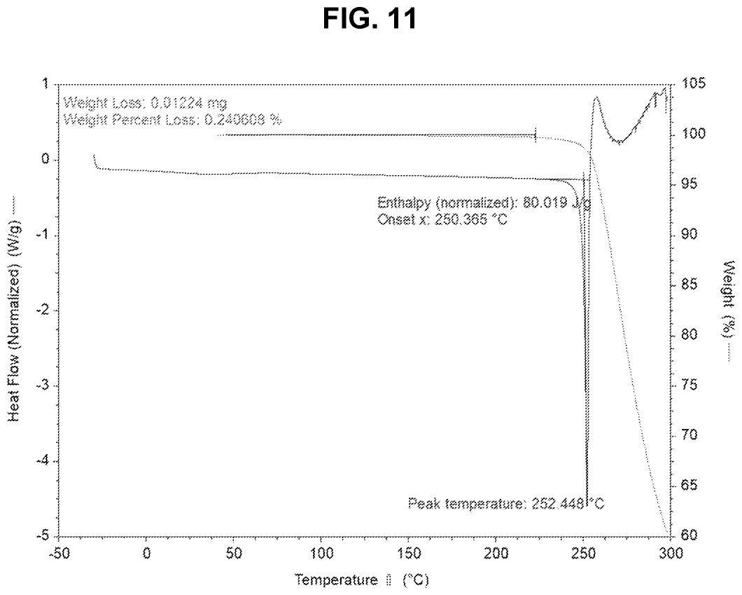

Differential scanning calorimetry (DSC) thermographs were obtained, as set forth in the Examples, for the crystalline Compound A-MsA Salt Form 1. The DSC curve indicates an endothermic transition at 250° C.±3° C. Thus, in some embodiments, the crystalline Compound A-MsA Salt Form 1 can be characterized by a DSC thermograph having a transition endotherm with an onset of 247° C. to 253° C. For example, in some embodiments the crystalline Compound A-MsA Salt Form 1 is characterized by DSC, as shown in FIG. 11.

The crystalline Compound A-MsA Salt Form 1 also can be characterized by thermogravimetric analysis (TGA). Thus, the crystalline Compound A-MsA Salt Form 1 can be characterized by a weight loss of about 0.2% with an onset temperature of 247° C. to 253° C. For example, the crystalline Compound A-MsA Salt Form 1 can be characterized by a weight loss of about 0.2%, up to about 250° C. In some embodiments, the crystalline Compound A-MsA Salt Form 1 has a thermogravimetric analysis substantially as depicted in FIG. 11, wherein by “substantially” is meant that the reported TGA features can vary by ±1% of the 0.2% weight loss.

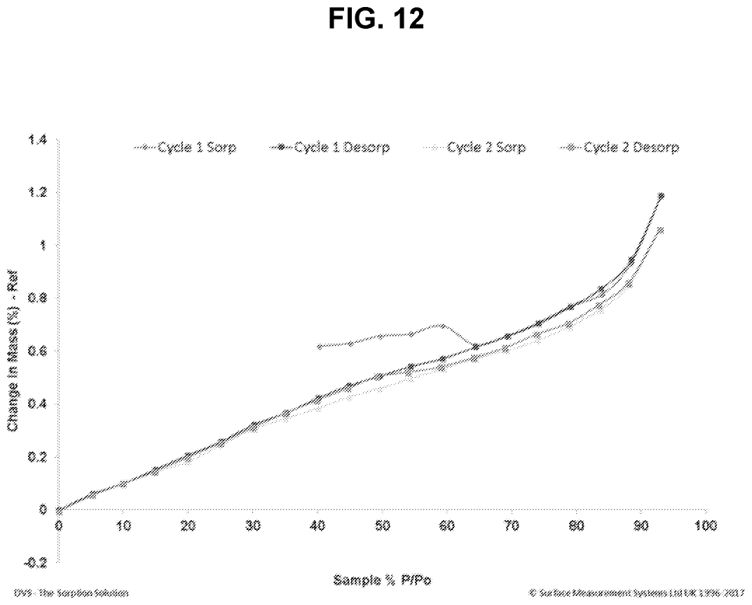

The crystalline Compound A-MsA Salt Form 1 can be characterized by a moisture sorption profile. For example, in some embodiments the crystalline Compound A-MsA Salt Form 1 is characterized by the moisture sorption profile as shown in FIG. 12, showing a weight gain of less than 1.2% by 95% RH.

Further provided herein are pharmaceutical compositions comprising the crystalline Compound A-MsA Salt Form 1 as described herein and a pharmaceutically acceptable excipient.

### Crystalline Compound A-Tosylate Salt Form 1 (Compound-A-TsA Form 4).

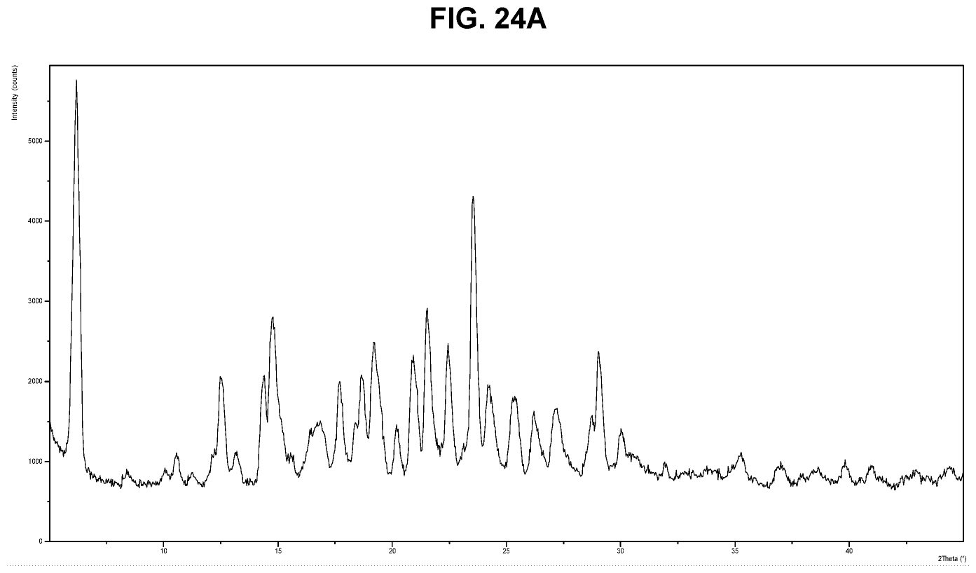

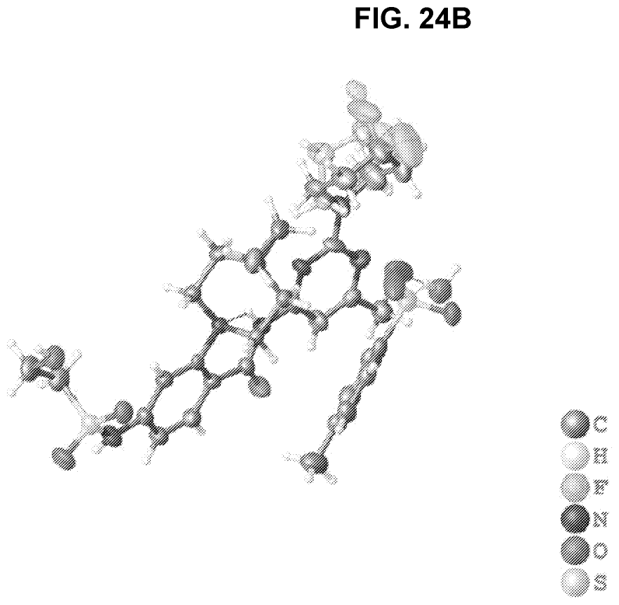

Also provided herein is a crystalline Compound A-TsA Salt Form 4. The crystalline Compound A-TsA Salt Form 4 can be characterized by an X-ray powder diffraction pattern, obtained as set forth in the Examples, having peaks at 6.2, 14.7, and 23.5±0.2° 2θ using Cu Kα radiation. The crystalline Compound A-TsA Salt Form 4 optionally can be further characterized by an X-ray powder diffraction pattern having additional peaks at 10.5, 12.4, 14.2, 19.1, 21.5 and 29.0±0.2° 2θ using Cu Kα radiation. The crystalline Compound A-TsA Salt Form 4 optionally can be further characterized by an X-ray powder diffraction pattern having additional peaks at 15.5, 16.5, 17.7, 18.3, 18.6, 20.1, 20.8, 24.1, and 25.3±0.2° 2θ using Cu Kα radiation. In some embodiments, crystalline Compound A-TsA Salt Form 4 has an X-ray powder diffraction pattern substantially as shown in FIG. 24a, wherein by “substantially” is meant that the reported peaks can vary by ±0.2°. Those skilled in the art know that in the field of XRPD, while relative peak heights in spectra are dependent on several factors, such as sample preparation and instrument geometry, peak positions are relatively insensitive to experimental details.

The crystalline Compound A-TsA Salt Form 4 is further characterized by a single crystal structure substantially as shown in FIG. 24b, or as set forth in the Examples.

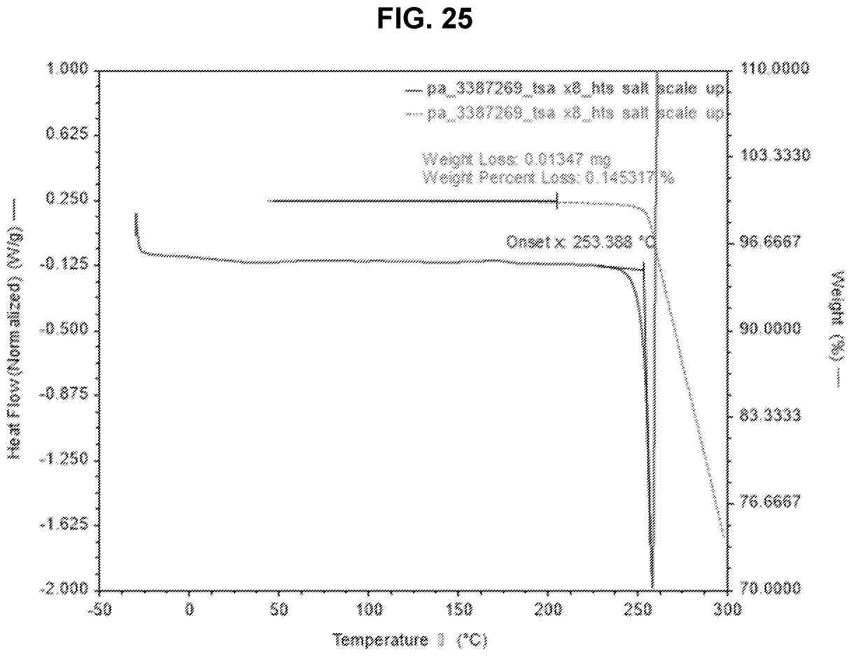

Differential scanning calorimetry (DSC) thermographs were obtained, as set forth in the Examples, for the crystalline Compound A-TsA Salt Form 4. The DSC curve indicated an endothermic transition at 253° C.±3° C. Thus, in some embodiments, the crystalline Compound A-TsA Salt Form 4 can be characterized by a DSC thermograph having a transition endotherm with an onset of 250° C. to 256° C. For example, in some embodiments the crystalline Compound A-TsA Salt Form 4 is characterized by DSC, as shown in FIG. 25.

The crystalline Compound A-TsA Salt Form 4 also can be characterized by thermogravimetric analysis (TGA). Thus, the crystalline Compound A-TsA Salt Form 4 can be characterized by a weight loss of about 0.07%, with an onset temperature of 250° C. to 256° C. For example, the crystalline Compound A-TsA Salt Form 4 can be characterized by a weight loss of about 0.07%, up to about 253° C. In some embodiments, the crystalline Compound A-TsA Salt Form 4 has a thermogravimetric analysis substantially as depicted in FIG. 25, wherein by “substantially” is meant that the reported TGA features can vary by ±1% of the 0.07% weight loss.

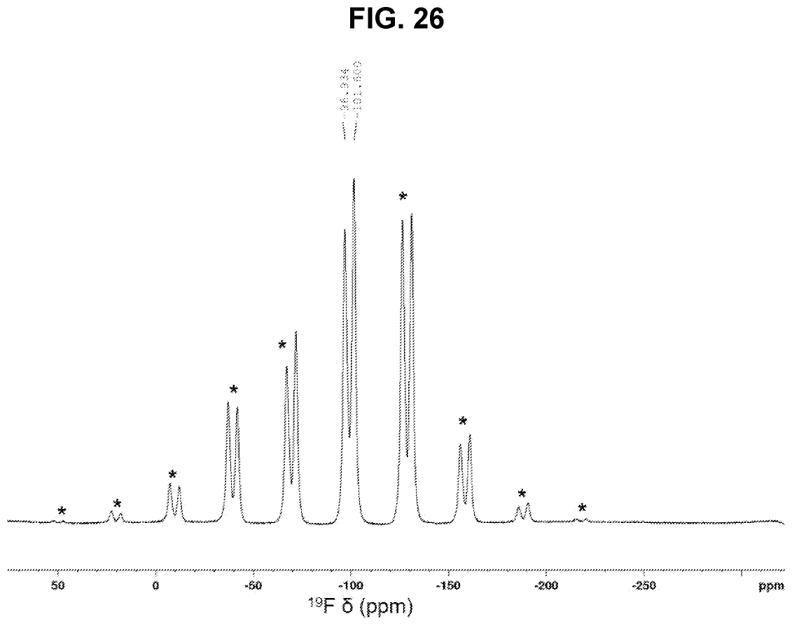

The crystalline Compound A-TsA Salt Form 4 also has a solid state ¹⁹F NMR substantially as shown in FIG. 26, having peaks at −96.93 and −101.60 ppm, wherein by “substantially” is meant that the reported peaks can vary by ±0.5 ppm.

Further provided herein are pharmaceutical compositions comprising the crystalline Compound A-HCl Salt Form 4 as described herein and a pharmaceutically acceptable excipient.

### Pharmaceutical Compositions

Compound A, as described in any of the above embodiments and sub-embodiments thereof, can be combined with a pharmaceutically acceptable excipient to provide a pharmaceutical formulation (also referred to, interchangeably, as a composition). The excipient can be a diluent or carrier. Formulations may influence the physical state, stability, rate of in vivo release and rate of in vivo clearance of the administered agents. The phrases “pharmaceutically acceptable” or “pharmacologically acceptable” refer to molecular entities and compositions that do not produce adverse, allergic, or other untoward reactions when administered to an animal or a human. As used herein, “pharmaceutically acceptable” includes any and all solvents, dispersion media, coatings, antibacterial and antifungal agents, isotonic and absorption delaying agents and the like. The use of such excipients for pharmaceutically active substances is well known in the art. Except insofar as any conventional media or agent is incompatible with the therapeutic compositions, its use in therapeutic compositions is contemplated. Supplementary active ingredients also can be incorporated into the compositions. In exemplary embodiments, the formulation may comprise corn syrup solids, high-oleic safflower oil, coconut oil, soy oil, L-leucine, calcium phosphate tribasic, L-tyrosine, L-proline, L-lysine acetate, DATEM (an emulsifier), L-glutamine, L-valine, potassium phosphate dibasic, L-isoleucine, L-arginine, L-alanine, glycine, L-asparagine monohydrate, L-serine, potassium citrate, L-threonine, sodium citrate, magnesium chloride, L-histidine, L-methionine, ascorbic acid, calcium carbonate, L-glutamic acid, L-cystine dihydrochloride, L-tryptophan, L-aspartic acid, choline chloride, taurine, m-inositol, ferrous sulfate, ascorbyl palmitate, zinc sulfate, L-carnitine, alpha-tocopheryl acetate, sodium chloride, niacinamide, mixed tocopherols, calcium pantothenate, cupric sulfate, thiamine chloride hydrochloride, vitamin A palmitate, manganese sulfate, riboflavin, pyridoxine hydrochloride, folic acid, beta-carotene, potassium iodide, phylloquinone, biotin, sodium selenate, chromium chloride, sodium molybdate, vitamin D3 and cyanocobalamin.

For oral administration, suitable compositions can be formulated by combining Compound A with pharmaceutically acceptable excipients such as carriers well known in the art. Such excipients and carriers enable Compound A to be formulated as tablets, pills, dragees, capsules, liquids, gels, syrups, slurries, suspensions, and the like, for oral ingestion by a patient to be treated. Pharmaceutical preparations for oral use can be obtained by adding Compound A with a solid excipient, optionally grinding a resulting mixture, and processing the mixture of granules, after adding suitable auxiliaries, if desired, to obtain tablets or dragee cores. Suitable excipients include, for example, fillers and cellulose preparations. If desired, disintegrating agents can be added. Pharmaceutically acceptable ingredients are well known for the various types of formulation and may be for example binders (e.g., natural or synthetic polymers), lubricants, surfactants, sweetening and flavoring agents, coating materials, preservatives, dyes, thickeners, adjuvants, antimicrobial agents, antioxidants, and carriers for the various formulation types.

When a therapeutically effective amount of Compound A is administered orally, the composition typically is in the form of a solid (e.g., tablet, capsule, pill, powder, or troche) or a liquid formulation (e.g., aqueous suspension, solution, elixir, or syrup).

When administered in tablet form, the composition can additionally contain a functional solid and/or solid carrier, such as a gelatin or an adjuvant.

When administered in liquid or suspension form, a functional liquid and/or a liquid carrier such as water, petroleum, or oils of animal or plant origin can be added. The liquid form of the composition can further contain physiological saline solution, sugar alcohol solutions, dextrose or other saccharide solutions, or glycols. In one embodiment contemplated, the liquid carrier is non-aqueous or substantially non-aqueous. For administration in liquid form, the composition may be supplied as a rapidly-dissolving solid formulation for dissolution or suspension immediately prior to administration.

For administration by inhalation, Compound A can be delivered in the form of an aerosol spray presentation from pressurized packs or a nebulizer, with the use of a suitable propellant. In the embodiment of a pressurized aerosol, the dosage unit can be determined by providing a valve to deliver a metered amount. Capsules and cartridges of, e.g., gelatin, for use in an inhaler or insufflator can be formulated containing a powder mix of the compound and a suitable powder base such as lactose or starch.

In particular, Compound A can be administered orally, buccally, or sublingually in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents.

### Safety

In some aspects, the method comprises administering Compound A, or the pharmaceutically acceptable salt thereof, in amount that does not lead to a dose limiting toxicity (DLT) during treatment with Compound A or the salt thereof. Optionally, the subject does not exhibit a DLT associated with Compound A treatment during the treatment period. In various instances, the subject does not exhibit any grade 3 or grade 4 adverse events associated with Compound A treatment during the treatment period. In various instances, the treatment period is at least two weeks or at least one month, if not longer, e.g., 2 months, 3, months, 4 months, 5 months, 6 months, 7 months, 8 months, 9 months, 10 months, 11 months, 12 months, 1 year, 1.5 years, 2 years.

In exemplary aspects, the method further comprises monitoring the subject's complete blood count before, during or after Compound A treatment. In various aspects, the complete blood count includes a count of the number of one or more of: red blood cells, white blood cells, platelets, and neutrophils. Optionally, the complete blood count includes a measurement of hematocrit and/or hemoglobin. In exemplary aspects, the monitoring occurs once a week for about two months. In various aspects, the subject's platelet count is greater than about 100,000 per μL blood during Compound A treatment.

In exemplary embodiments, the methods of the present disclosure are advantageously highly specific to cells of the neoplastic disease. In various aspects, Compound A effectively treats the neoplastic disease, induces or increases tumor regression, reduces tumor or cancer growth, or induces or increases death of a tumor or cancer cell, with little to no toxicity to normal somatic cells in the subject. In various aspects, Compound A, or a pharmaceutically acceptable salt thereof, is administered in an amount effective to treat the neoplastic disease, induce or increase tumor regression, reduce tumor or cancer growth, and/or induce or increase death of a tumor or cancer cell, without a substantial decrease in the proliferation of normal somatic cells in the subject. In exemplary instances, Compound A, or a pharmaceutically acceptable salt thereof, is administered in an amount effective to treat the neoplastic disease, induce or increase tumor regression, reduce tumor or cancer growth, or induce or increase death of a tumor or cancer cell, without a substantial increase in the apoptosis of normal somatic cells. As used herein, the term “normal” in reference to cells means cells that are not neoplastic and/or not diseased. In various aspects, the normal somatic cells are human bone marrow mononuclear cells or T cells. In various instances, the normal somatic cells are not genetically characterized as TP53^{MUT} or are genetically characterized as TP53^{WT}. In various aspects, Compound A, or a pharmaceutically acceptable salt thereof, causes not more than a 25% increase in the apoptosis of normal somatic cells. In various aspects, Compound A, or a pharmaceutically acceptable salt thereof, causes not more than a 25% decrease in the proliferation of normal somatic cells in the subject. Optionally, the increase in the apoptosis of normal somatic cells or the decrease in the proliferation of normal somatic cells is less than about 20%, less than about 15%, less than about 10%, less than about 9%, less than about 8%, less than about 7%, less than about 6%, less than about 5%, less than about 4%, less than about 3%, less than about 2%, or less than about 1%.

The primary side effect of taxanes is myelosuppression, primarily neutropenia, while other side effects include peripheral edema, and neurotoxicity (peripheral neuropathy). In exemplary aspects, the methods of the present disclosure treat the neoplastic disease in the subject without causing any of these side effects observed in patients treated with taxanes or treats the neoplastic disease wherein such side effects are lessened in severity compared to that observed in patients treated with taxanes.

### Treatment Efficacy

As used herein, the term “treat,” as well as words related thereto, do not necessarily imply 100% or complete treatment. Rather, there are varying degrees of treatment of which one of ordinary skill in the art recognizes as having a potential benefit or therapeutic effect. In this respect, the methods of treating a neoplastic disease of the present disclosure can provide any amount or any level of treatment. Furthermore, the treatment provided by the methods of the present disclosure can include treatment of one or more conditions or symptoms or signs of the neoplastic disease being treated. Also, the treatment provided by the methods of the present disclosure can encompass slowing the progression of the neoplastic disease. For example, the methods can treat neoplastic disease by virtue of enhancing the T cell activity or an immune response against the neoplastic disease, reducing tumor or cancer growth or tumor burden, reducing metastasis of tumor cells, increasing cell death of tumor or cancer cells, or increasing tumor regression, and the like. In accordance with the foregoing, provided herein are methods of reducing tumor growth, tumor volume, or tumor burden or increasing tumor regression in a subject. In exemplary embodiments, the method comprises administering to the subject Compound A, or a pharmaceutically acceptable salt thereof. The terms “treat”, “treating” and “treatment” as used herein refer to therapy, including without limitation, curative therapy, prophylactic therapy, and preventative therapy. Prophylactic treatment generally constitutes either preventing the onset of disorders altogether or delaying the onset of a pre-clinically evident stage of disorders in individuals.

In various aspects, the methods treat by way of delaying the onset or recurrence of the neoplastic disease or delaying the occurrence or onset of metastasis. In various aspects, the methods treat by way increasing the survival of the subject. In exemplary instances, the onset or recurrence or the occurrence is delayed by at least 1 day, 2 days, 4 days, 6 days, 8 days, 10 days, 15 days, 30 days, two months, 3 months, 4 months, 6 months, 1 year, 2 years, 3 years, 4 years, or more.

In various aspects, the treatment provided by the methods of the present disclosure provides a therapeutic response as per Response Evaluation Criteria in Solid Tumors (RECIST) or other like criteria. RECIST is a set of criteria to evaluate the progression, stabilization, or responsiveness of tumors and/or cancer cells jointly created by the National Cancer Institute of the United States, the National Cancer Institute of Canada Clinical Trials Group and the European Organisation for Research and Treatment of Cancer. According to RECIST, certain tumors are measured in the beginning of an evaluation (e.g., a clinical trial), to provide a baseline for comparison after treatment with a drug. The response assessment and evaluation criteria for tumors are published in Eisenhauer et. al., Eur J Cancer 45:228-247 (2009) and Litiere et. al., Journal of Clinical Oncology 37(13): 1102-1110 (2019) DOI: 10.1200/JCO.18.01100. In various instances, the treatment provided by the methods of the present disclosure provides a therapeutic response as per a modified RECIST tumor response assessment, as follows:

Summary of Measurement and Tumor Response
Assessment Based on Modified RECIST 1.1

| | |
|---|---|
| Measurable lesions | Non-nodal lesions: ≥10 mm (unidimensional measurement) |
| | Pathologic lymph nodes: longest diameter short axis ≥15 mm |
| Measurement of each | Non-nodal lesions: the longest diameter (mm) in the axial plane |
| lesion | Pathologic lymph nodes: longest diameter: short axis (mm) |
| Tumor burden | Sum of the longest diameter (SLD) in all index lesions |
| | Up to 5 lesions per organ, up to 10 total |
| Response assessment: | CR: Disappearance of all lesions. Pathologic lymph nodes |
| index lesions | short axis <10 mm. |
| (calculated from % | PR: ≥30% decrease from baseline |
| change in tumor | SD: Does not meet criteria for CR, PR, or progressive disease |
| burden) | Progressive disease: ≥20% increase (and ≥5 mm absolute increase) |
| | from nadir |
| Response assessment: | CR: Disappearance of all lesions. Pathologic lymph nodes |
| non-index lesions | short axis <10 mm. |
| | SD: Persistence of one or more non-index lesion(s) |
| | Progressive disease: Unequivocal progression of existing non-index |
| | lesions |
| New Lesions | The presence of new lesion(s) defines progression |
| Confirmation | Confirmation by subsequent assessment after ≥4 weeks required for |
| | CR, PR, and progressive disease |

Summary of Modified RECIST 1.1 Overall Response Assessment

| Index lesions | Non-Index | | Overall Response using |
|---|---|---|---|
| (tumor burden)^{a}, % | lesions | New lesions | Modified RECIST 1.1 |
| ↓ 100% | Absent | Absent | CR^{b} |
| None^{d} | Absent | Absent | CR^{b} |
| ↓ 100% | Present | Absent | PR^{b} |
| ↓ ≥ 30% | Absent/Present | Absent | PR^{b} |
| ↓ < 30% to ↑ < 20% | Absent/Present | Absent | SD |
| None^{d} | Present | Absent | SD |
| ↑ ≥ 20% | Any | Any | Progressive disease^{c} |
| Any | Unequivocal progression | Any | Progressive disease^{c} |
| Any | Any | Present | Progressive disease^{c} |
| NA/ND/UE | Absent/Present | Absent | UE |
| None^{d} | NA/ND/UE | Absent | UE |

CR = complete response;
NA = not available;
ND = not done;
PR = partial response;
RECIST = Response Evaluation Criteria in Solid Tumors;
UE = unable to evaluate
^{a}Decrease assessed relative to baseline. Increase assessed relative to nadir.
^{b}Response: CR and PR require a confirmation assessment after ≥4 weeks, may also wait until the next scheduled imaging
^{c}Progression: Progressive disease requires a confirmation assessment 4 to 6 weeks after initial radiographic progressive disease is observed
^{d}Subjects with non-index lesions only

In various instances, the subject exhibits at least a stable disease (SD) after treatment with Compound A or the pharmaceutically acceptable salt thereof. In various aspects, the subject exhibits at least a partial response (PR) after treatment with Compound A or the pharmaceutically acceptable salt thereof. The subject exhibits at least a 10%, at least a 15%, at least a 25%, at least a 30%, at least a 40%, or at least a 50% decrease in Cancer Antigen 125 (CA125) levels compared to baseline, in various aspects. The subject in exemplary instances exhibits at least a 10%, at least a 15%, at least a 25%, at least a 30%, at least a 40%, or at least a 50% decrease in tumor volume after treatment with Compound A.

### Neoplastic Disease

As used herein, the term “neoplastic disease” refers to any condition that causes growth of a tumor. In exemplary aspects, the tumor is a benign tumor. In exemplary aspects, the tumor is a malignant tumor. In various aspects, the neoplastic disease is a tumor or a cancer. The cancer in various aspects is acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, gastrointestinal carcinoid tumor, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer, peritoneum, omentum, mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, or urinary bladder cancer. In particular aspects, the cancer is head and neck cancer, ovarian cancer, cervical cancer, bladder cancer, oesophageal cancer, pancreatic cancer, gastrointestinal cancer, gastric cancer, breast cancer, endometrial cancer, colorectal cancer, hepatocellular carcinoma, glioblastoma, bladder cancer, lung cancer, e.g., non-small cell lung cancer (NSCLC), or bronchioloalveolar carcinoma. In particular embodiments, the tumor is non-small cell lung cancer (NSCLC), head and neck cancer, renal cancer, triple negative breast cancer, or gastric cancer. In exemplary aspects, the subject has a tumor (e.g., a solid tumor, a hematological malignancy, or a lymphoid malignancy) and the pharmaceutical composition is administered to the subject in an amount effective to treat the tumor in the subject. In other exemplary aspects, the tumor is non-small cell lung cancer (NSCLC), small cell lung cancer (SCLC), head and neck cancer, renal cancer, breast cancer, melanoma, ovarian cancer, liver cancer, pancreatic cancer, colon cancer, prostate cancer, gastric cancer, lymphoma or leukemia, and the pharmaceutical composition is administered to the subject in an amount effective to treat the tumor in the subject.

The terms “cancer” and “cancerous” when used herein refer to or describe the physiological condition in mammals that is typically characterized by unregulated cell growth. Examples of cancer include, without limitation, carcinoma, lymphoma, sarcoma, blastoma and leukemia. More particular examples of such cancers include squamous cell carcinoma, lung cancer, pancreatic cancer, cervical cancer, bladder cancer, hepatoma, breast cancer, colon carcinoma, and head and neck cancer, ovarian cancer, and endometrial cancer. While the term “cancer” as used herein is not limited to any one specific form of the disease, it is believed that the methods of the invention will be particularly effective for cancers which are found to be accompanied by unregulated levels of KIF18A or dependent on KIF18A for proper chromosome segregation and survival in the mammal.

In various aspects, the cancer is metastatic, the tumor is unresectable, or a combination thereof. In various instances, the cancer is a chromosomally unstable aneuploid cancer. In various aspects, the neoplastic disease (e.g., the cancer or tumor) comprises cells that are positive for an inactivated TP53 gene and/or positive for at least one of (i) an inactivated Rb1 gene, (ii) an amplified CCNE1 gene, gene copy number gain of the CCNE1 gene, or overexpression of a CCNE1 gene product, (iii) an inactivated BRCA gene or (iv) a combination thereof. In various aspects, the neoplastic disease is triple negative breast cancer (TNBC), nonluminal breast cancer (e.g., basal like mesenchymal), or high grade serous ovarian cancer (HGSOC). In various aspects, the neoplastic disease is resistant or not sensitive (insensitive) to treatment with a CDK4/6 inhibitor. In various aspects, the neoplastic disease is resistant or not sensitive (insensitive) to treatment with a CDK4/6 inhibitor and is Rb1 proficient (vs. Rb1 deficient). In various aspects, the neoplastic disease is resistant to treatment with a KIF18A inhibitor. In various aspects, the neoplastic disease is resistant to treatment with a KIF18A inhibitor and Rb1 deficient (vs. Rb1 proficient).

In exemplary aspects, the neoplastic disease is a breast cancer, optionally, luminal breast cancer or TNBC. In various aspects, the breast cancer has been (a) histologically or cytologically confirmed metastatic or locally recurrent estrogen receptor (ER)-negative (e.g., <1% by immunohistochemistry [IHC]), (b) progesterone receptor (PR)-negative (e.g., <1% IHC) and (c) human epidermal growth factor receptor 2 (Her2)-negative (either fluorescent in situ hybridisation [FISH] negative, 0 or 1+ by IHC, or IHC2+ and FISH negative per ASCO/CAP definition). In exemplary aspects, the neoplastic disease is relapsed and/or refractory to at least one line of systemic chemotherapy in the metastatic setting or intolerant of existing therapy(ies) known to provide clinical benefit for the neoplastic disease. In exemplary instances, the cancer has been treated with an immune checkpoint inhibitor. In various instances, the breast cancer is hormone receptor (HR)-positive and/or HER2-negative. In various aspects, the breast cancer is advanced breast cancer and/or metastatic breast cancer. In various aspects, the breast cancer is HR+/HER2− advanced or metastatic breast cancer that has progressed after endocrine therapy. In some aspects, the breast cancer is a hormone receptor-positive (HR+)/HER2-negative (HER2−) advanced or metastatic breast cancer previously treated with endocrine therapy and chemotherapy after the cancer has spread/metastasized. In various instances, the cancer is an HR+/HER2− advanced or metastatic breast cancer that has not been treated with hormonal therapy (Arimidex (chemical name: anastrozole), Aromasin (chemical name: exemestane), and Femara (chemical name: letrozole). In various instances, the breast cancer is HR+/HER2− advanced or metastatic breast cancer that has grown after being treated with hormonal therapy. In various instances, the breast cancer is a HER2− positive breast cancer, including but not limited to those that are similar to the HER2-positive breast cancer cells of Table 2. Optionally, the breast cancer is a HER2-positive, estrogen receptor (ER)-negative breast cancer. In various aspects, the neoplastic disease is ovarian cancer, optionally, high grade serous ovarian cancer (HGSOC). Optionally, the ovarian cancer is platinum-resistant HGSOC. In exemplary aspects, the ovarian cancer is primary peritoneal cancer or fallopian-tube cancer. In various aspects, the neoplastic disease is metastatic or unresectable HGSOC, with platinum-resistance defined as progression during or within 6 months of a platinum-containing regimen. In various aspects, the ovarian cancer has been or is being treated with platinum-resistant recurrence therapy. In various aspects, the neoplastic disease is serous endometrial cancer. Optionally, the neoplastic disease is metastatic or recurrent serous endometrial cancer. In various instances, the endometrial cancer is relapsed and/or refractory to at least one line of systemic therapy in the metastatic/recurrent setting or intolerant of existing therapy(ies) known to provide clinical benefit for the neoplastic disease. In various instances, the neoplastic disease is an advanced or metastatic solid tumor that is unresectable and relapsed and/or refractory to at least one line of systemic chemotherapy or intolerant. Optionally, the advanced or metastatic solid tumor is TP53^{MUT}.

In various aspects, the cancer is ovarian cancer, breast cancer, or endometrial cancer. In various aspects, the ovarian cancer is clear cell ovarian cancer or high grade serous ovarian cancer (HGSOC), optionally, metastatic or unresectable HGSOC. Optionally, the HGSOC is platinum-resistant HGSOC or wherein the HGSOC progressed during or within 6 months of a platinum-containing regimen. In various instances, the cancer is primary peritoneal cancer and/or cancer of the fallopian tube. In exemplary instances, the breast cancer is triple negative breast cancer. In some aspects, the subject has relapsed or is refractory to at least one line of systemic chemotherapy. Optionally, the systemic chemotherapy comprises taxane, gemcitabine, or doxorubicin. In various instances, the endometrial cancer is serous endometrial cancer, optionally, metastatic, or recurrent serous endometrial cancer. In certain aspects, the serous endometrial cancer has relapsed or is refractory to at least one line of systemic chemotherapy, e.g., cisplatin, carboplatin or lenvatinib.

In various aspects, the tumor is an advanced solid tumor. The tumor is non-resectable, metastatic and/or non-localized in various instances. The tumor in exemplary aspects, has relapsed or is refractory to at least one line of systemic chemotherapy.

In various instances, the neoplastic disease is resistant to treatment with one or more drugs. In various aspects, the neoplastic disease exhibits reduced sensitivity to treatment with one or more drugs. Optionally, the neoplastic disease is a multidrug resistant neoplastic disease. In exemplary instances, the tumor or cancer cells (e.g., of the neoplastic disease) are multidrug resistant tumor or cancer cells and/or exhibit increased expression of the Multidrug resistance 1 (MDR-1) gene and/or a gene product thereof. In exemplary instances, the tumor or cancer cells (e.g., of the neoplastic disease) exhibit increased expression of a P-glycoprotein (P-gp) encoded by MDR-1 gene. In various aspects, the neoplastic disease exhibits reduced sensitivity or resistance to treatment with an anti-mitotic agent or anthracycline antibiotic, optionally, paclitaxel or doxorubicin. In various aspects, the tumor or cancer cells (e.g., of the neoplastic disease) exhibit mutations in a tubulin gene, overexpression of tubulin, tubulin amplification, and/or isotype switched tubulin expression. In various aspects, the mutations in α- or β-tubulin inhibit the binding of taxanes to the correct place on the microtubules, thereby rendering the taxane ineffective. In exemplary aspects, the neoplastic disease exhibits reduced sensitivity or resistance to treatment with any one or more of a platinum agent an anthracycline, a targeted therapy (e.g. TKI, PARP inhibitors).

In various aspects, the neoplastic disease is a cancer comprising one or more whole genome duplication or whole genome doubling (WGD) events. WGD in the context of cancer is discussed in Lens and Hemdema, Nature Reviews Cancer 19: 32-45 (2019); Ganem et. al., Current Opinion in Genetics & Development 17, 157-162, and Davoli et. al., Annual Review of Cell and Developmental Biology 27, 585-610.

### Inactivated Genes, Amplified Genes, and Expression Levels

As used herein, the term “inactivated” in the context of a gene refers to a reduction or loss of function of the gene or gene product encoded by the gene. The inactivation of a gene may be caused by one or more known mechanisms. For example, the inactivation of the gene may be caused by a variation in (including, e.g., a loss of) DNA sequence, RNA sequence or protein sequence, relative to the corresponding wild-type gene, RNA, or protein or may be caused by an epigenetic variation that does not involve any alterations in the DNA sequence of the gene.

In various aspects, cells of the cancer comprise a variation or anomaly in a gene or a gene product encoded by the gene, which variation or anomaly is relative to the corresponding wild-type gene or gene product, and which presence of the variation leads to or is associated with a silencing of the gene, a reduction or loss of expression of the gene or gene product encoded by the gene, a reduction or loss of function of the gene or gene product encoded by the gene, or a combination thereof. In various instances, the gene product is an RNA transcript or a protein. In various instances, the variation leads to at least a reduction or loss of function of the gene or gene product encoded by the gene. In various instances, the variation leads to at least a reduction or loss of function of the TP53 gene or gene product encoded by the TP53 gene. In various instances, the variation leads to at least a reduction or loss of function of the Rb1 gene or gene product encoded by the Rb1 gene. In various instances, the variation leads to at least a reduction or loss of function of the BRCA gene or gene product encoded by the BRCA gene.

The variation in the gene may be present anywhere in the gene, e.g., within an intron or exon, within a 5′-untranslated region (5′-UTR), or a 3′-untranslated region (3′-UTR). The variation may be present within or at any part of the transcript (e.g., RNA transcript, primary transcript, pre-mRNA, mRNA) encoded by the gene, or may be present within or at any part of the protein encoded by the gene.

In various aspects, the variation is a difference in DNA sequence, RNA sequence or protein sequence, relative to the corresponding wild-type gene, RNA, or protein. In various aspects, the inactivated gene is detected by analyzing the nucleotide sequence of the gene, analyzing the nucleotide sequence of an RNA encoded by the gene, or analyzing the amino acid sequence of the protein encoded by the gene and comparing the sequence of gene of the sample to the corresponding wild-type human sequence of the gene, RNA, or protein. In exemplary aspects, the variation comprises a deletion, insertion, or substitution of one or more nucleotides in the DNA sequence or RNA sequence, a deletion, insertion, or substitution of one or more amino acids in the protein sequence, relative to the corresponding wild-type gene, RNA, or protein. In exemplary aspects, the variation comprises a deletion, insertion, or substitution of one or more nucleotides in the DNA sequence or RNA sequence, a deletion, insertion, or substitution of one or more amino acids in the protein sequence, relative to the corresponding wild-type gene, RNA, or protein that may result in a gene copy number gain or amplification of the DNA, RNA, or protein. In various aspects, cells of the cancer comprise a gene mutation in the gene. In various aspects, cells of the cancer comprise a gene mutation in the gene or loss of nucleotides in the gene. In exemplary instances, the gene mutation is a missense mutation, nonsense mutation, insertion, deletion, duplication, frameshift mutation, truncation, or a repeat expansion. In various instances, the inactivated TP53 gene comprises a mutation, deletion, or truncation, the inactivated Rb1 gene comprises a mutation, deletion, or truncation, and/or the inactivated BRCA gene comprises a mutation, deletion, or truncation. As used herein, the term “BRCA gene” refers to the BRCA1 or the BRCA2 gene. In exemplary instances, the BRCA gene is BRCA1. In exemplary aspects, the BRCA gene is BRCA2.

In various instances, the variation is epigenetic and does not involve any alterations in the DNA sequence of the gene. In exemplary aspects, the inactivated gene is epigenetically silenced and optionally involves a covalent modification of the DNA or histone proteins. The covalent modification of the DNA may be, for example, a cytosine methylation or hydroxymethylation. The covalent modification of the histone protein may be, for example, a lysine acetylation, lysine or arginine methylation, serine or threonine phosphorylation, or lysine ubiquitination or sumoylation. Mechanisms of gene silencing can occur during transcription or translation. Exemplary mechanisms of gene silencing include but are not limited to DNA methylation, histone modification, and RNA interference (RNAi). In various aspects, the inactivated gene is an epigenetically silenced gene having an epigenetically silenced promoter. Optionally, the inactivated TP53 gene has an epigenetically silenced TP53 promoter or the inactivated Rb1 gene has an epigenetically silenced Rb1 promoter or the inactivated BRCA gene has an epigenetically silenced BRCA promoter. Suitable techniques to assay for epigenetic silencing include but are not limited to chromatin immunoprecipitation (ChIP-on chip, ChIP-Seq) fluorescent in situ hybridization (FISH), methylation-sensitive restriction enzymes, DNA adenine methyltransferase identification (DamID) and bisulfite sequencing. See, e.g., Verma et. al., Cancer Epidemiology, Biomarkers, and Prevention 23: 223-233 (2014).

In various aspects, the inactivated gene is inactivated by a virus-induced gene silencing (VIGS). In various instances, the inactivated TP53 gene is inactivated by a viral protein, e.g., human papillomavirus (HPV) E6 protein. Optionally, the HPV E6 protein interacts with the p53 protein encoded by the TP53 gene and renders the p53 protein inactive. In various instances, the inactivated Rb1 gene is inactivated by a viral protein, e.g., HPV E7 protein. Optionally, the HPV E7 protein interacts with the Rb protein encoded by the Rb1 gene and renders the Rb protein inactive. Such modes of silencing are known in the art. See, e.g., Jiang and Milner, Oncogene 21: 6041-6048 (2002).

In various embodiments of the methods of the present disclosure, cells of the cancer comprise a gene amplification, e.g., CCNE1 amplification, or an increase in the number of copies of a gene, e.g., a gene copy number gain of the gene. In various instances, cells of the cancer comprise a gene copy number gain or amplified gene which can be detected by DNA- or RNA-based techniques (gene expression analysis [comparative genomic hybridization, RNA-based hybridization], NGS, PCR, or Southern blot) or by molecular cytogenetic techniques (FISH2 with gene-specific probes, CISH (chromogenic in situ hybridization). In various aspects, competitive or quantitative PCR, genomic hybridization to cDNA microarrays, hybridization and quantification of gene probes to RNA are carried out to detect the gene amplification or gene copy number gain. See, e.g., Harlow and Stewart, Genome Res 3: 163-168 (1993); Heiskanen et. al., Cancer Res 60(4): 799-802 (2000). In various instances, cells of the cancer comprise a gene copy number gain or amplification of an MDM2 gene and/or a gene copy number gain or amplification or mutation of an FBXW7 gene. In exemplary aspects, cells of the cancer comprise a gene copy number gain or amplification of an MDM2 gene and a reduction in p53 protein levels. In exemplary aspects, cells of the cancer comprise a mutation in an FBXW7 gene, and an overexpression of a gene product encoded by the CCNE1 gene. Next Generation Sequencing (NGS) may also be employed as a method by which to detect a gene copy number gain or loss or a gene amplification whereby genetic areas are sequenced, and sequencing reads are compared to other genes to deduce gain or loss of the gene of interest.

In exemplary aspects, the inactivated TP53 gene (i) comprises a TP53 gene mutation, deletion, truncation, and/or an epigenetically silenced TP53 promoter, (ii) is inactivated by a viral protein or via gene amplification of an MDM2 gene, or (iii) a combination thereof. Optionally, the viral protein is a Human Papillomavirus (HPV) E6 protein. In exemplary aspects, the inactivated Rb1 gene (i) comprises an Rb1 gene mutation, deletion, truncation, and/or an epigenetically silenced Rb1 promoter, (ii) is inactivated by a viral protein or (iii) a combination thereof. Optionally, the viral protein is a Human Papillomavirus (HPV) E7 protein. In exemplary aspects, the inactivated BRCA gene (i) comprises a BRCA gene mutation, deletion, truncation, and/or an epigenetically silenced BRCA promoter. Optionally, the BRCA gene is a BRCA1 gene. Alternatively, the BRCA gene is a BRCA2 gene.

In various aspects, the inactivated TP53 gene, inactivated Rb1 gene, CCNE1 gene copy number gain or amplification and/or inactivated BRCA gene is present in the germline cells of the neoplastic disease (e.g., cancer). In various aspects, the inactivated TP53 gene, inactivated Rb1 gene, CCNE1 gene copy number gain or amplification and/or inactivated BRCA gene is present in the germline cells of the neoplastic disease (e.g., cancer) and absent from somatic cells of the neoplastic disease (e.g., cancer). Optionally, due to somatic mutations of the neoplastic disease, the somatic cells of the neoplastic disease have reverted back to wild-type genotype and thus do not exhibit the inactivated TP53 gene, inactivated Rb1 gene, CCNE1 gene copy number gain or amplification and/or inactivated BRCA gene, though the germline cells of the neoplastic disease still demonstrate inactivated TP53 gene, inactivated Rb1 gene, CCNE1 gene copy number gain or amplification and/or inactivated BRCA gene. For example, the neoplastic disease may be a PARP inhibitor-resistant cancer and only the germline cells of the cancer have an inactivated BRCA1 gene, whereas the somatic cells of the cancer exhibit a restored BRCA1 coding region and function.

A cytogenetics method and/or molecular method may be used for detecting the presence of an inactivated or amplified gene or gene copy number gain, e.g., an inactivated TP53 gene, inactivated Rb1 gene, amplified CCNE1 gene or inactivated BRCA gene. In exemplary aspects, direct DNA sequencing, DNA hybridization and/or restriction enzyme digestion are used. Optionally, the cytogenetics method comprises karyotyping, fluorescence in situ hybridization (FISH), comparative genomic hybridization (CGH), or a combination thereof. In various instances, the molecular method comprises restriction fragment length polymorphism (RFLP), amplification refractory mutation system (ARMS), polymerase chain reaction (PCR), multiplex ligation dependent probe amplification (MLPA), denaturing gradient gel electrophoresis (DGGE), single strand conformational polymorphism (SSCP), heteroduplex analysis, chemical cleavage of mismatch (CCM), protein truncation test (PTT), oligonucleotide ligation assay (OLA), or a combination thereof. Optionally, the PCR is a multiplex PCR, nested PCR, RT-PCR, or real time quantitative PCR. In various aspects, expression levels of RNA or protein encoded by the TP53 gene, Rb1 gene, CCNE1 gene, and/or the BRCA gene are assayed. In various aspects, ARMS, FISH, IHC, or NGS are employed. Such techniques are described in Su et al., J Experimental Clin Cancer Research 36: 121 (2017) and He et al., Blood 127(24): 3004-3014 (2016). In various instances, whole-exome sequencing or whole genome sequencing is used. In exemplary aspects, the assaying comprises a liquid biopsy. Liquid biopsies are described in detail in the art. See, e.g., Poulet et al., Acta Cytol 63(6): 449-455 (2019), Chen and Zhao, Hum Genomics 13(1): 34 (2019).

In various aspects, the gene copy number gain or amplification leads to overexpressed or increased levels of the gene products (e.g., RNA and/or protein) encoded by the gene. Methods of detecting increased levels in RNA and/or protein are known in the art. In exemplary aspects, the gene copy number gain or amplification of the CCNE1 gene leads to overexpressed or increased levels of the gene products encoded by the CCNE1 gene. In exemplary aspects, the overexpression of the CCNE1 gene product is caused by a mutation in an FBXW7 gene. In various aspects, the sample is positive for overexpression of the CCNE1 gene products and a mutation in an FBXW7 gene.

Suitable methods of determining expression levels of nucleic acids (e.g., genes, RNA, mRNA) are known in the art and include but not limited to, quantitative polymerase chain reaction (qPCR) (e.g., quantitative real-time PCR (qRT-PCR)), RNAseq, Nanostring, and Northern blotting. Techniques for measuring gene expression also include, for example, gene expression assays with or without the use of gene chips or gene expression microarrays are described in Onken et. al., J Molec Diag 12(4): 461-468 (2010); and Kirby et. al., Adv Clin Chem 44: 247-292 (2007). Affymetrix gene chips and RNA chips and gene expression assay kits (e.g., Applied Biosystems™ TaqMan® Gene Expression Assays) are also commercially available from companies, such as ThermoFisher Scientific (Waltham, MA), and Nanostring (Geiss et. al., Nature Biotechnology 26: 317-325 (2008)). Suitable methods of determining expression levels of proteins are known in the art and include immunoassays (e.g., Western blotting, an enzyme-linked immunosorbent assay (ELISA), a radioimmunoassay (RIA), and immunohistochemical assay) or bead-based multiplex assays, e.g., those described in Djoba Siawaya J F, Roberts T, Babb C, Black G, Golakai H J, Stanley K, et al. (2008) An Evaluation of Commercial Fluorescent Bead-Based Luminex Cytokine Assays. PLoS ONE 3(7): e2535. Proteomic analysis which is the systematic identification and quantification of proteins of a particular biological system are known. Mass spectrometry is typically the technique used for this purpose.

In exemplary aspects, the method comprises measuring the level of a complementary DNA (cDNA) based on the RNA encoded by said gene. Briefly, the method comprises extracting or isolating RNA from the sample (e.g., from the tumor cell(s) of the sample) and synthesizing cDNA based on RNA isolated from the sample. Alternatively, or additionally, in some aspects, measuring the expression level comprises isolating RNA from the sample, producing complementary DNA (cDNA) from the RNA, amplifying the cDNA, and hybridizing the cDNA to a gene expression microarray. Accordingly, in some aspects, measuring the expression level comprises isolating RNA from the sample and quantifying the RNA by RNA-Seq. In alternative or additional aspects, the level of expression is determined via an immunohistochemical assay. In exemplary aspects, measuring the expression level comprises contacting the sample with a binding agent to TP53, Rb1, BRCA, or CCNE1, or a gene product thereof, or a combination thereof. In some aspects, the binding agent is an antibody, or antigen-binding fragment thereof. In some aspects, the binding agent is a nucleic acid probe specific for TP53, Rb1, BRCA, or CCNE1, or an RNA transcript thereof, or a complement thereof.

Once the expression level of TP53, Rb1, BRCA, or CCNE1, or the gene product thereof, is measured from a sample obtained from the subject, the measured expression level may be compared to a reference level, normalized to a housekeeping gene, mathematically transformed. In exemplary instances, the measured expression level of TP53, Rb1, BRCA, or CCNE1, or the gene product thereof, is centered and scaled. Suitable techniques of centering and scaling biological data are known in the art. See, e.g., van den Berg et. al., BMC Genomics 7: 142 (2006).

The wild-type TP53, Rb1, CCNE1, and BRCA genes, as well as the RNA and proteins encoded by these genes, are known in the art. Exemplary sequences of each are available at the website for the National Center for Biotechnology Information (NCBI) and provided in the sequence listing submitted herewith.

TABLE A

| Gene name | NCBI, | mRNA | | Protein | |
|---|---|---|---|---|---|
| (abbreviation, | HUGO Gene | Accession | SEQ | Accession | SEQ |
| full) | ID No. | No. | ID NO: | No. | ID NO: |
| TP53 | 7157, 11998 | NM_000546.6 | 1 | NP_000537.3 | 2 |
| RB1 | 5925, 9884 | NM_000321.3 | 3 | NP_000312.2 | 4 |
| CCNE1 | 898, 1589 | NM_001238.4 | 5 | NP_001229.1 | 6 |
| BRCA1 | 672, 1100 | NM_007294.4 | 7 | NP_009225.1 | 7 |
| BRCA2 | 675, 1101 | NM_000059.4 | 9 | NP_000050.3 | 10 |

The cells of the cancer may be identified as “positive” or “negative” for (a) an inactivated TP53 gene and/or (b) at least one of: (i) an inactivated Rb1 gene, (ii) an amplified CCNE1 gene, gene copy number gain of the CCNE1 gene, or overexpression of a CCNE1 gene product, (iii) an inactivated BRCA gene or (iv) a combination thereof. As used herein, the term “positive” in the context of a sample means that an inactivated TP53 gene and/or (b) at least one of: (i) an inactivated Rb1 gene, (ii) an amplified CCNE1 gene, gene copy number gain of the CCNE1 gene, or overexpression of a CCNE1 gene product, (iii) an inactivated BRCA gene or (iv) a combination thereof is/are present in the sample. As used herein, the term “negative” in the context of a sample means that an inactivated TP53 gene and/or (b) at least one of: (i) an inactivated Rb1 gene, (ii) an amplified CCNE1 gene, gene copy number gain of the CCNE1 gene, or overexpression of a CCNE1 gene product, (iii) an inactivated BRCA gene or (iv) a combination thereof is/are absent from the sample, e.g., the sample does not have an inactivated TP53 gene and/or (b) at least one of: (i) an inactivated Rb1 gene, (ii) an amplified CCNE1 gene, gene copy number gain of the CCNE1 gene, or overexpression of a CCNE1 gene product, (iii) an inactivated BRCA gene or (iv) a combination thereof is/are present in the sample.

### Subjects

In exemplary embodiments of the present disclosure, the subject is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammals are of the order Primates, Ceboids, or Simoids (monkeys) or of the order Anthropoids (humans and apes). In some aspects, the mammal is a human. In various aspects, the subject has a neoplastic disease, e.g., any one of those described herein. The term “patient”, “subject”, or “mammal” as used herein refers to any “patient”, “subject”, or “mammal” including humans, cows, horses, dogs, and cats. In one embodiment of the invention, the mammal is a human. In various aspects, the subject is an adult human. Optionally, the subject has received prior treatment with at least one chemotherapeutic agent.

In exemplary aspects, the subject has cancer with a metastasis, an unresectable tumor, or a combination thereof. In various aspects, the cancer or tumor exhibits or has exhibited a resistance or reduced sensitivity to treatment with a CDK4/6 inhibitor. In exemplary aspects, the subject has breast cancer, optionally, luminal breast cancer or triple negative breast cancer (TNBC). In various aspects, the breast cancer has been (a) histologically or cytologically confirmed metastatic or locally recurrent estrogen receptor (ER)-negative (e.g., <1% by immunohistochemistry [IHC]), (b) progesterone receptor (PR)-negative (e.g., <1% IHC) and (c) human epidermal growth factor receptor 2 (Her2)-negative (either fluorescent in situ hybridisation [FISH] negative, 0 or 1+ by IHC, or IHC2+ and FISH negative per ASCO/CAP definition). In exemplary aspects, the subject is relapsed and/or refractory to at least one line of systemic chemotherapy in the metastatic setting or intolerant of existing therapy(ies) known to provide clinical benefit for their condition. In exemplary instances, the subject has prior exposure to an immune checkpoint inhibitor. In various instances, the breast cancer hormone receptor (HR)-positive and/or HER2-negative. In various aspects, the breast cancer is advanced breast cancer and/or metastatic breast cancer. In various aspects, the subject has HR+/HER2− advanced or metastatic breast cancer that has progressed after taking endocrine therapy. In some aspects, the subject is a hormone receptor-positive (HR+)/HER2-negative (HER2−) advanced or metastatic breast cancer patient previously treated with endocrine therapy and chemotherapy after cancer has spread/metastasized. In various instances, the subject has HR+/HER2− advanced or metastatic breast cancer that has not been treated with hormonal therapy before in postmenopausal women (Arimidex (chemical name: anastrozole), Aromasin (chemical name: exemestane), and Femara (chemical name: letrozole). In various instances, the subject is a postmenopausal woman with HR+/HER2− advanced or metastatic breast cancer that has grown after being treated with hormonal therapy. In certain aspects, the subject is a pre/perimenopausal or postmenopausal woman with HR+, human epidermal growth factor receptor 2 (HER2)-negative advanced or metastatic breast cancer and has received endocrine-based therapy. Optionally, the subject is a postmenopausal woman with HR+, HER2− advanced or metastatic breast cancer, and has received initial endocrine-based therapy or has disease progression upon treatment with the endocrine therapy. In various aspects, the subject has ovarian cancer, optionally, high grade serous ovarian cancer (HGSOC). Optionally, the ovarian cancer is platinum-resistant HGSOC. In exemplary aspects, the subject has primary peritoneal cancer and/or fallopian-tube cancer. In various aspects, the subject has a histologically or cytologically confirmed diagnosis of metastatic or unresectable HGSOC, with platinum-resistance defined as progression during or within 6 months of a platinum-containing regimen. In various aspects, the subject has ovarian cancer and has received or is receiving platinum-resistant recurrence therapy. In various aspects, the subject has serous endometrial cancer. Optionally, the subject has a histologically or cytologically confirmed diagnosis of metastatic or recurrent serous endometrial cancer. In various instances, the subject is relapsed and/or refractory to at least one line of systemic therapy in the metastatic/recurrent setting or intolerant of existing therapy(ies) known to provide clinical benefit for their condition. In various instances, the subject has an advanced or metastatic solid tumor that is unresectable and relapsed and/or refractory to at least one line of systemic chemotherapy or intolerant. Optionally, the advanced or metastatic solid tumor is TP53^{MUT}.

In exemplary aspects, the subject does not have any of the following: (a) active brain metastases, (b) primary central nervous system (CNS) tumor, hematological malignancies, or lymphoma, (c) uncontrolled pleural effusions(s), pericardial effusion, or ascites, (d) gastrointestinal (GI) tract disease causing the inability to take oral medication.

### Subject Selection and Therapeutic Results

In some embodiments, the subject being treated by Compound A in the disclosed methods is one who has undergone one or more prior systemic cancer therapies (e.g., Compound A is a second or third line therapy). Prior systemic cancer therapies can be any therapy approved by a regulatory authority (e.g., the FDA or EMA) as treatment given type and stage of cancer. In some cases, the prior systemic cancer therapy is a cancer therapy not yet approved by a regulatory authority but undergoing clinical trials. If a subject has had a prior systemic cancer therapy, in some cases, the subject has not undergone any systemic cancer therapy for at least one month, at least two months, at least three months, at least four months, at least five months, or at least six months prior to starting therapy as disclosed herein with Compound A.

A subject undergoing a therapy is monitored for adverse events (AE) during the course of the therapy. A treatment related AE is an AE that is related to the treatment drug. A treatment emergent AE is one that a subject develops undergoing the treatment that was not present prior to start of therapy. In some cases, the treatment emergent AE is not or suspected not to be related to the treatment itself. AEs are characterized as one of five grades—grade 1 is a mile AE; grade 2 is a moderate AE; grade 3 is a severe AE; grade 4 is a life-threatening or disabling AE; and grade 5 is death related to AE. In some cases, the subject does not exhibit any grade 3 AE that is treatment related. In some cases, the subject does not exhibit any grade 3 AE. In some cases, the subject does not exhibit any grade 4 AE that is treatment related. In some cases, the subject does not exhibit any grade 4 AE. In various cases, the subject does not exhibit a grade 3 or grade 4 AE that is treatment related after administration of Compound A for at least one month, or at least three months.

In various cases, the subject being treated with Compound A in the methods disclosed herein, does not exhibit any dose limiting toxicities (DLT) at the dose administered. A DLT is any AE meeting the criteria listed below occurring during the first treatment cycle of Compound A (day 1 through day 21) where relationship to the drug cannot be ruled out. The grading of AEs is based on the guidelines provided in the CTCAE version 5.0. AEs for DLT assessment: Hematological toxicity: Febrile neutropenia; Neutropenic infection; Grade 4 neutropenia; Grade ≥3 thrombocytopenia for >7 days; Grade 3 thrombocytopenia with grade ≥2 bleeding; Grade 4 thrombocytopenia; Grade 4 Anemia Non-hematological toxicity Grade ≥4, vomiting or diarrhea; Grade 3 diarrhea or grade 3 vomiting lasting more than 3 days despite optimal medical support; Grade ≥3 nausea for 3 days or more despite optimal medical support; Any other grade >3 AE.

In various cases, the subject of the disclosed methods exhibits a response to the therapy. In some cases, the subject exhibits at least a stable disease (SD) due to administration of Compound A. In some cases, the subject exhibits at least a partial response (PR) due to administration of Compound A. The response of a subject is assessed by the criteria as defined by RECIST 1.1, e.g., as discussed in Eisenhauer et al., Eur J Cancer, 45:228-247 (2009). A complete response (CR) is disappearance of all target lesions and any pathological lymph nodes have a reduction in short axis to less than 10 mm. A partial response (PR) is at least a 30% decrease in the sum of diameters of target lesions, taking as reference the baseline sum diameters. A progressive disease is at least a 20% increase in the sum of diameters of target lesions, taking as reference the smallest sum on study (including the baseline sum if that is the smallest on study), and there must be an absolute increase of at least 5 mm in addition to the relative increase of 20%. A stable disease is neither sufficient shrinkage to qualify for PR nor sufficient increase to qualify for PD. A controlled disease state is when a patient may alternate between exhibiting a stable disease and a partial response. The tumor size can be measured by radiographic scan.

### Other Embodiments

Further provided herein are any amorphous or crystalline form of any salt, hydrate, solvate, or co-crystal of Compound A, selected from hydrochloride salt (Compound A-HCl), mesylate salt (Compound A-MsA), tosylate salt (Compound A-TsA), sulfate salt (Compound A-sulfate), variable hydrate (Compound A-variable hydrate), tetrahydrofuran solvate (Compound A-THF), ethanol solvate (Compound A-ethanol), 1-propanol solvate (Compound A-1-propanol), isopropyl alcohol solvate (Compound A-IPA), methanol solvate (Compound A-methanol), isopropyl acetate solvate (Compound A-IPAc), acetone solvate (Compound A-acetone), cyclopentyl methyl ether solvate (Compound A-CPME), dioxane solvate (Compound A-dioxane), ethyl acetate solvate (Compound A-EtOAc), acetonitrile solvate (Compound A-MeCN), methyl tert-butyl ether solvate (Compound A-MTBE), toluene solvate (Compound A-toluene), dodecyl sulfate (Compound A-dodecyl sulfate), dimethyl formamide (DMF) solvate hydrate (Compound A-DMF-hydrate), dimethylacetamide (DMAC) solvate (Compound A-DMAC), monobesylate hydrate (Compound A-besylate-hydrate), caffeine co-crystal (Compound A-caffeine), citric acid co-crystal (Compound A-citric acid), saccharin co-crystal (Compound A-saccharin), L-tartaric acid co-crystal (Compound A-L-tartaric acid), or urea co-crystal (Compound A-urea), as characterized by any of XRPD, DSC, TGA, moisture sorption (DVS) in the figures and examples herein; and the pharmaceutical compositions comprising any of the salt, solvate, or co-crystal of Compound A and a pharmaceutically acceptable excipient.

In various embodiments, the organic solvent can be selected from the group consisting of an ether solvent, a nonpolar solvent, and any combination thereof. In some cases, the organic solvent can be an ether solvent. Suitable ether solvents can include, for example, tetrahydrofuran (THF), 2-methyltetrahydrofuran (MeTHF), cyclopentyl methyl ether, tert-butyl methyl ether, 1,2-dimethoxyethane, 1,4-dioxane, diethyl ether, diisopropyl ether, bis(2-methoxyethyl) ether, propylene glycol methyl ether, and any combination thereof. In embodiments, the ether solvent can be THE or 2-methyltetrahydrofuran. In some cases, the organic solvent can be a nonpolar solvent. Suitable nonpolar solvents can include, for example, hexane, pentane, toluene, benzene, heptane, xylene, and any combination thereof. In embodiments, the nonpolar solvent can be toluene, hexane, heptane, or any combination thereof. In embodiments, the organic solvent can be selected from the group consisting of THF, 2-methyltetrahydrofuran, cyclopentyl methyl ether, tert-butyl methyl ether, 1,2-dimethoxyethane, toluene, hexane, heptane, 1,4-dioxane, and any combination thereof. In some embodiments, the organic solvent is THF.

It is to be understood that while the disclosure is read in conjunction with the detailed description thereof, the foregoing description is intended to illustrate and not limit the scope of the disclosure, which is defined by the scope of the appended claims. Other aspects, advantages, and modifications are within the scope of the following claims. For example, as shown in Examples 1-45.

### EXAMPLES

The following examples are provided for illustration and are not intended to limit the scope of the invention.

### Materials and Methods

Commercially available reagents are used as is without further purification unless specified. The 1.0 M Mel in THF solution is prepared by weight. The batch and flow chemistry equipment (reactors, tubing, pumps, connections, and fittings) are from commercially available sources.

The synthesis of the starting material (Compound A) for the following synthetic methods is disclosed in U.S. Non-Provisional patent application Ser. No. 16/724,119, filed on Dec. 20, 2019, published on Jul. 30, 2020, as U.S. 2020-0239441. The starting materials, the intermediates, and final products of the reactions may be isolated and purified, if desired, using conventional techniques, including but not limited to filtration, distillation, crystallization, chromatography, and the like. Such materials may be characterized using conventional means, including physical constants and spectral data.

Unless specified to the contrary, the reactions described herein take place at atmospheric pressure and a temperature in a range of about −78° C. to about 150° C., or about 0° C. to about 50° C., or about 15° C. to about 25° C.

### PANalytical X'Pert PRO MPD Diffractometer—Transmission Geometry

Unless specified to the contrary, XRPD patterns were collected with a PANalytical X'Pert PRO MPD diffractometer using an incident beam of Cu radiation produced by an Optix long, fine-focus source. An elliptically graded multilayer mirror was used to focus Cu Kα X-rays through the specimen and onto the detector. Prior to the analysis, a silicon specimen (NIST SRM 640f) was analyzed to verify the Si 111 peak position. A specimen of the sample was sandwiched between 3 μm thick films and analyzed in transmission geometry. A beam-stop and short antiscatter extension were used to minimize the background generated by air. Soller slits for the incident and diffracted beams were used to minimize broadening from axial divergence. Diffraction patterns were collected using a scanning position-sensitive detector (X'Celerator) located 240 mm from the specimen and Data Collector software v.5.5 (except for the as received materials where Data Collector software v.2.2b was used). The data-acquisition parameters for each pattern are displayed above the image in the Data section of this report.

### PANalytical X'Pert PRO MPD Diffractometer—Reflection Geometry

Unless specified to the contrary, XRPD patterns were collected with a PANalytical X'Pert PRO MPD diffractometer using an incident beam of Cu Kα radiation produced using a long, fine-focus source and a nickel filter. The diffractometer was configured using the symmetric Bragg-Brentano geometry. Data were collected and analyzed using Data Collector software v. 2.2b. Prior to the analysis, a silicon specimen (NIST SRM 640) was analyzed to verify the observed position of the Si 111 peak is consistent with the NIST-certified position. A specimen of the sample was packing in a nickel-coated copper well. Antiscatter slits (SS) were used to minimize the background generated by air. Soller slits for the incident and diffracted beams were used to minimize broadening from axial divergence. Diffraction patterns were collected using a scanning position-sensitive detector (X'Celerator) located 240 mm from the sample and Data Collector software v. 2.2b. The data acquisition parameters for each pattern are displayed above the image in the Data section of this report including the divergence slit (DS) and the incident-beam antiscatter slit (SS)

X-ray powder diffraction (XRPD) data were obtained on a PANalytical X'Pert PRO X-ray diffraction system with RTMS detector. Samples were scanned at ambient temperature in a continuous mode from 5 to 450 (2θ) with step size of 0.0334° at a time per step of 50 s at 45 kV and 40 mA with CuKα radiation (1.541874 Å).

XRPD indexing was conducted with proprietary SSCI software, TRIADS™ is covered by U.S. Pat. No. 8,576,985.

Differential scanning calorimetry (DSC) was performed using a Mettler-Toledo DSC3+ differential scanning calorimeter. A tau lag adjustment is performed with indium, tin, and zinc. The temperature and enthalpy are adjusted with octane, phenyl salicylate, indium, tin, and zinc. The adjustment is then verified with octane, phenyl salicylate, indium, tin, and zinc. The sample was placed into a hermetically sealed aluminum DSC pan, and the weight was accurately recorded. The pan lid was pierced by the instrument and then inserted into the DSC cell for analysis. A weighed aluminum pan configured as the sample pan was placed on the reference side of the cell.

Alternatively, Differential scanning calorimetry (DSC) analysis was also conducted on a TA Instruments Q and Discovery Series calorimeter at 10° C./min from 25° C. to 250° C. to 350° C. in an aluminum pan under dry nitrogen at 50 ml/min.

Thermal gravimetric analysis (TGA) and TGA/DSC Combo analyses were performed using a Mettler-Toledo TGA/DSC3+ analyzer. Temperature and enthalpy adjustments were performed using indium, tin, and zinc, and then verified with indium. The balance was verified with calcium oxalate. The sample was placed in an open aluminum pan. The pan was hermetically sealed, the lid pierced, then inserted into the TG furnace. A weighed aluminum pan configured as the sample pan was placed on the reference platform. The furnace was heated under nitrogen.

Thermal gravimetric analysis (TGA) was performed on a TA Instruments Q and Discovery Series analyzer at 10° C./min from ambient temperature between 250° C. to 350° C. in a platinum pan under dry nitrogen at 25 ml/min.

Moisture sorption data was collected using a VTI SGA 100 symmetrical vapor sorption analyzer. A sample size of approximately 5 mg to 10 mg was used in a platinum pan. Hygroscopicity was evaluated from 5% RH to 95% RH in increments of 5% RH. Data for adsorption and desorption cycles were collected. Equilibrium criteria were set at 0.001% weight change in 10 minutes with a maximum equilibration time of 180 minutes.

Solution proton NMR spectra were acquired by Spectral Data Services of Champaign (SSCI), IL at 25° C. with a Varian UNITY/NOVA-400 spectrometer. Samples were dissolved in DMSO-d6. In some cases, the solution NMR spectra were also acquired at SSCI with an Agilent DD2-400 spectrometer using deuterated DMSO or methanol.

¹⁹F SSNMR data was collected on a Bruker DSX spectrometer operating at 600 Mhz (¹H). A 4 mm H/F/X spinning probe operating at a spinning frequency of 14 kHz was used for all experiments. HPDEC program was used with a recycle delay of 10 s and referenced to Teflon. A ¹H 90° pulse of 2.5 μs and ¹⁹F 90° pulse of 5 μs were used. Decoupling was carried out using a spinal64 sequence. 256 transients were acquired for signal averaging. The data was processed with Topspin 3.0 software.

### Example 1: Crystalline Compound A-HCl Form 1

The crystallization of Compound A-HCl Form 1 can be achieved in multiple solvent systems following in situ protonation of the Compound A with hydrochloric acid. Initially, Compound A-HCl Form 1 was prepared by slurring one equivalent of HCl in Acetonitrile/water 90/10 at ambient condition. Later, an anhydrous process, treating the Compound A in acetonitrile/1,4-dioxane system with hydrochloric acid at elevated temperature, was used (Table 1, entry no. 1). Alternative reactive crystallization processes using different sources of hydrochloric acid were developed in NMP/EtOH, THE/water, and acetone/water (Table 1, entries nos. 2-5). Acetone/water was selected as the final crystallization system due to consistent high purity of drug substance and control over residual solvent amounts according to ICH guideline limits. The characterization results of these batches and were summarized in Table 1.

TABLE 1
Various crystallization processes to produce Compound A-HCl Form 1

| | | | | | Equiv. | Yield | HPLC | Residual | Chloride |
|---|---|---|---|---|---|---|---|---|---|
| No. | Scale | | Solvent(s) | HCl source | HCl | (%) | purity | Solvent | Content |
| 1. | 2.5 | g | MeCN/ | 4M HCl in | 1.0 | 95 | 97.98 | 6136 ppm | 5.5 |
| | | | 1,4-dioxane | 1,4-dioxane | | | LCAP | MeCN | |
| 2. | 5 | g | THF/water | 0.5M HCl | 1.5 | 82 | 98.85 | 3649 ppm | 5.7 |
| | | | | (aq.) | | | LCAP | THF | |
| 3. | 5 | g | NMP/EtOH | 1.25M HCl in | 1.5 | 84 | 99.07 | 15112 | 5.7 |
| | | | | EtOH | | | LCAP | ppm NMP | |
| | | | | | | | | 1205 ppm | |
| | | | | | | | | EtOH | |
| 4. | 0.5 | g | Acetone/ | 1.5M HCl | 2.0 | 89 | 98.97 | 4830 ppm | N/A |
| | | | water | (aq.) | | | LCAP | acetone | |
| 5. | 1 | kg | Acetone/ | 1.5M HCl | 2.0 | 78 | 98.99 | 1346 ppm | 5.9 |

| | | | |
|---|---|---|---|
| water | (aq.) | LCAP | acetone |

Compound A was dissolved in 30 vol. of acetone, followed by polish filtration, addition of 5 vol. of water and 2.0 equiv. of hydrochloric acid (2.5 vol. of aq. 1.5N HCl solution) at ambient temperature. The final solvent composition for crystallization and slurry aging is 80/20 (v/v) acetone/water, which offered appropriate solubility for both the Compound A (i.e. about 18 mg/mL) and the Compound A-HCl Form 1 (i.e. about 8 mg/mL) to achieve crystal growth and impurity rejection. The process was unseeded and crystal growth occurs during the addition of hydrochloric acid to the Compound A solution. The final slurry was aged at ambient temperature for 10 h, then cooled to 10° C. prior to wet milling. Isolation of the milled material occurred at 10° C. followed by washing of the filter cake with 8 volumes of acetone. The material was dried at 40° C. under vacuum. Wet milling experiments in both THE/water and acetone/water showed particle size reduction to the specified target range as summarized in Table 2 and the form purity was 95% by XRPD, solid state NMR and DSC.

TABLE 2
Comparison of unmilled and milled Compound A HCl particle
sizes obtained from THF/water and acetone/water slurries.

| Solvent(s) | |
|---|---|
| THF/water | Acetone/water |

| Particle | Before | After | Before | After |
|---|---|---|---|---|
| size | milling | milling | milling | milling |
| D[4,3], μm | 54.2 | 28.1 | 50.6 | 26.9 |
| D10, μm | 13.7 | 6.7 | 8.9 | 4.5 |
| D50, μm | 47.6 | 26.2 | 41.7 | 24.6 |
| D90, μm | 106.0 | 52.5 | 106.0 | 53.3 |
| Span | 1.9 | 1.8 | 2.3 | 2.0 |

X-Ray Powder Diffraction: X-ray powder diffraction data were obtained on a PANalytical X'Pert PRO X-ray diffraction system with RTMS detector. Samples were scanned in continuous mode from 5-450 (2θ) with step size of 0.0334° at 45 kV and 40 mA with CuKα radiation (1.54 Å). The incident beam path was equipped with a 0.02 rad soller slit, 15 mm mask, 4° fixed anti-scatter slit and a programmable divergence slit. The diffracted beam was equipped with a 0.02 rad soller slit, programmable anti-scatter slit and a 0.02 mm nickel filter. Samples were prepared on a low background sample holder and placed on a spinning stage with a rotation time of 2 s. For variable-temperature studies, samples were prepared on a flat plate sample holder and placed in a TTK-450 temperature control stage. For variable-humidity studies, modular humidity generator (ProUmid) was used to control atmosphere in THC humidity sample chamber. The XRPD pattern of the Crystalline Compound A-HCl Form 1 material is shown in FIG. 1 and the XRPD peaks are listed in Table 3.

TABLE 3
XRPD Data of Crystalline Compound A-HCl Form 1

| Pos. [°2θ] | FWHM [°2θ] | d-spacing [Å] | Height [cts] | Rel. Int. [%] |
|---|---|---|---|---|
| 7.46 | 0.12 | 11.85 | 4968.86 | 98.51 |
| 9.92 | 0.05 | 8.92 | 392.44 | 7.78 |
| 10.93 | 0.15 | 8.10 | 999.90 | 19.82 |
| 12.22 | 0.10 | 7.24 | 215.45 | 4.27 |
| 12.80 | 0.22 | 6.92 | 2194.48 | 43.51 |
| 13.65 | 0.17 | 6.49 | 156.86 | 3.11 |
| 14.45 | 0.13 | 6.13 | 1141.27 | 22.63 |
| 15.08 | 0.12 | 5.87 | 608.16 | 12.06 |
| 15.65 | 0.12 | 5.66 | 1160.06 | 23.00 |
| 15.91 | 0.13 | 5.57 | 1267.80 | 25.14 |
| 16.38 | 0.12 | 5.41 | 906.12 | 17.96 |
| 16.86 | 0.13 | 5.26 | 4628.60 | 91.77 |
| 17.60 | 0.13 | 5.04 | 694.56 | 13.77 |
| 18.22 | 0.17 | 4.87 | 3169.70 | 62.84 |
| 18.61 | 0.13 | 4.77 | 878.26 | 17.41 |
| 19.37 | 0.10 | 4.58 | 432.63 | 8.58 |
| 19.77 | 0.12 | 4.49 | 1876.76 | 37.21 |
| 20.19 | 0.15 | 4.40 | 5043.90 | 100.00 |
| 20.61 | 0.08 | 4.31 | 1142.70 | 22.66 |
| 20.93 | 0.10 | 4.24 | 357.31 | 7.08 |
| 21.15 | 0.10 | 4.20 | 195.64 | 3.88 |
| 21.63 | 0.15 | 4.11 | 1043.33 | 20.69 |
| 22.65 | 0.12 | 3.93 | 2116.42 | 41.96 |
| 23.15 | 0.08 | 3.84 | 876.92 | 17.39 |
| 23.55 | 0.15 | 3.78 | 4553.07 | 90.27 |
| 24.11 | 0.10 | 3.69 | 733.98 | 14.55 |
| 24.77 | 0.13 | 3.59 | 3403.62 | 67.48 |
| 25.94 | 0.14 | 3.43 | 1409.32 | 27.94 |
| 26.05 | 0.12 | 3.42 | 1700.20 | 33.71 |
| 26.25 | 0.08 | 3.39 | 608.23 | 12.06 |
| 26.76 | 0.10 | 3.33 | 1195.89 | 23.71 |
| 27.40 | 0.13 | 3.26 | 136.88 | 2.71 |
| 27.88 | 0.10 | 3.20 | 164.48 | 3.26 |
| 28.39 | 0.10 | 3.14 | 618.61 | 12.26 |
| 28.72 | 0.10 | 3.11 | 307.94 | 6.11 |
| 29.29 | 0.10 | 3.05 | 720.00 | 14.27 |
| 29.77 | 0.12 | 3.00 | 427.49 | 8.48 |
| 30.12 | 0.20 | 2.97 | 320.39 | 6.35 |
| 31.02 | 0.17 | 2.88 | 501.91 | 9.95 |
| 31.46 | 0.17 | 2.84 | 301.61 | 5.98 |
| 32.00 | 0.13 | 2.80 | 319.71 | 6.34 |
| 32.84 | 0.10 | 2.73 | 585.65 | 11.61 |
| 33.09 | 0.08 | 2.71 | 505.83 | 10.03 |
| 33.94 | 0.10 | 2.64 | 832.84 | 16.51 |
| 34.39 | 0.13 | 2.61 | 255.97 | 5.07 |
| 35.05 | 0.27 | 2.56 | 139.37 | 2.76 |
| 36.08 | 0.12 | 2.49 | 307.30 | 6.09 |
| 36.95 | 0.23 | 2.43 | 567.46 | 11.25 |
| 37.35 | 0.20 | 2.41 | 318.94 | 6.32 |
| 37.98 | 0.20 | 2.37 | 39.69 | 0.79 |
| 38.67 | 0.17 | 2.33 | 252.64 | 5.01 |
| 39.30 | 0.10 | 2.29 | 353.28 | 7.00 |
| 40.76 | 0.12 | 2.21 | 443.34 | 8.79 |
| 41.52 | 0.20 | 2.17 | 230.44 | 4.57 |
| 41.97 | 0.17 | 2.15 | 176.56 | 3.50 |
| 43.31 | 0.17 | 2.09 | 161.12 | 3.19 |
| 44.03 | 0.16 | 2.06 | 141.80 | 2.81 |

TABLE 4
Solid State ¹⁹F NMR Data of the
Crystalline Compound A-HCl Form 1

| Peak | v(F1) [ppm] |
|---|---|
| 1 | −91 |
| 2 | −103 |

Thermal Analysis: Differential scanning calorimetry (DSC) was performed on a TA Instruments Q1000/2000 calorimeter at in an aluminum Tzero pan under dry nitrogen, flowing at 50 ml/min. Thermogravimetric analysis (TGA) was performed on a TA Instruments Q500 analyzer in a platinum pan under dry nitrogen, flowing at 60 ml/min. The DSC and TGA of the Crystalline Compound A-HCl Form 1 are shown in FIG. 2. Typical DSC and TGA of Crystalline Compound A-HCl Form 1 indicated a melting onset of 271.5° C. and about 4% weight loss before melting and decomposition.

Dynamic Vapor Sorption (DVS): Moisture sorption data was collected using a Surface Measurement Systems DVSAdvantage instrument. Equilibrium criteria were set at ±0.001% weight change in 10 minutes with a maximum equilibrium time of 360 minutes. The moisture sorption profile of the Crystalline Compound A-HCl Form 1 is shown in FIG. 3. Typical DVS of Crystalline Compound A-HCl Form 1 showed a weight gain of less than about 0.5% by 95% RH.

Single Crystal Data: single crystals of the Crystalline Compound A-HCl Form 1 were grown from DMF, DMAC or NMP with excess of HCl at room temperature. A single colourless needle-shaped crystals of Compound A-HCl Form 1 was used for single crystal structure determination. The specimen chosen for data collection was a needle with the approximate dimensions 0.29×0.08×0.06 mm³. The crystal was mounted on a nylon loop with paratone oil on a Bruker APEX-II CCD diffractometer. The crystal was kept at a steady T=173(2) K during data collection. The structure was solved with the SheIXT (Sheldrick, G. M. (2015). Acta Cryst. A71, 3-8) structure solution program using the Intrinsic Phasing solution method and by using Olex2 (Dolomanov et al., 2009) as the graphical interface. The model was refined with version 2018/3 of SheIXL (Sheldrick, Acta Cryst. A64 2008, 112-122) using Least Squares minimization. Table 5 shows the Crystallographic data summary of the Crystalline Compound A-HCl Form 1. The molecular structure of Crystalline Compound A-HCl Form 1 as found from X-ray crystal structure determination is shown in FIG. 5.

TABLE 5
X-ray Single Crystallographic data summary
of the Crystalline Compound A-HCl Form 1:

| Wavelength | 1.5418 | Å |
|---|---|---|
| Crystal System | Monoclinic | |
| Space Group | P2₁/n | |
| Unit Cell | a = 8.9026 Å | |
| | b = 13.8562 Å | |
| | c = 23.5472 Å | |
| | α = 90° | |
| | β = 94.987° | |
| | γ = 90° | |
| Volume | 2893.7 | Å³ |
| Z | 4 | |
| Density (calculated) | 1.380 | Mg/m³ |

| | |
|---|---|
| Absolute structure parameter | NA* |

*centrosymmetic

### Example 2: Crystalline Compound A-HCl Form 2

Crystalline Compound A-HCl Form 2 was generated under high throughput slurrying condition with one equivalent of HCl in 90/10 acetone/water solvent. This metastable form has low melting point and was not able to be scaled up or reproduced.

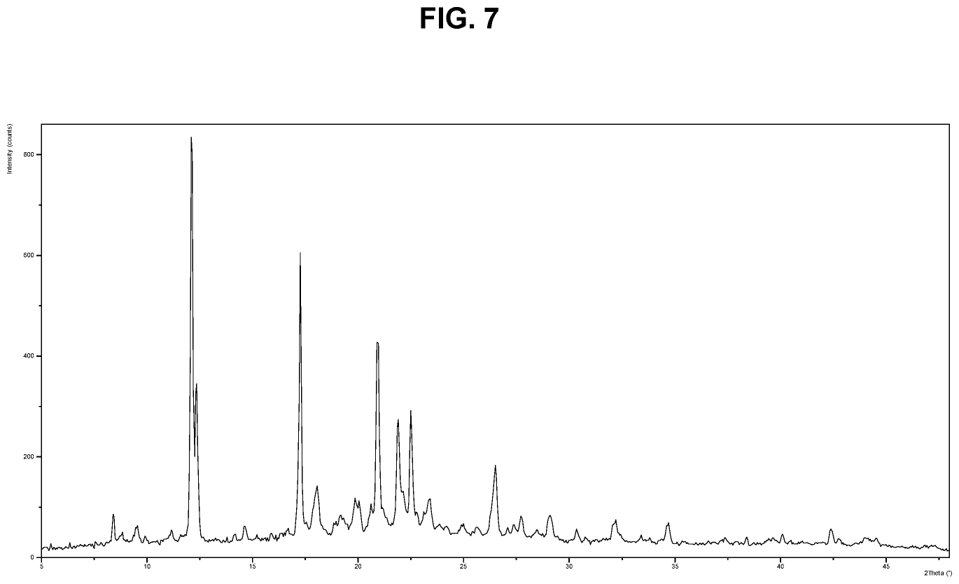

X-Ray Powder Diffraction: The XRPD pattern of the Crystalline Compound A-HCl Form 2 material is shown in FIG. 7.

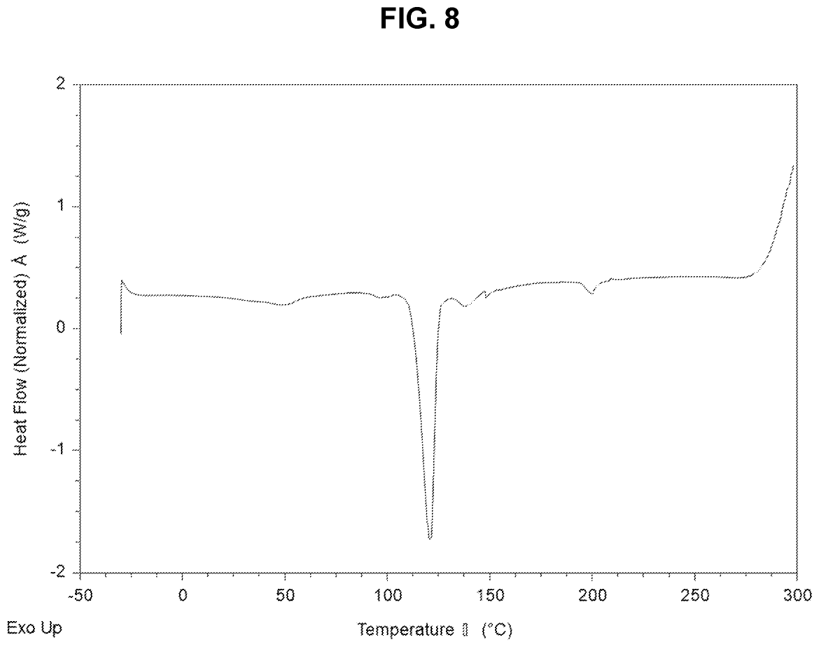

Thermal Analysis: The DSC of the Crystalline Compound A-HCl Form 2 is shown in FIG. 8. Typical DSC of Crystalline Compound A-HCl Form 2 indicated a melting onset of 113.2° C.

### Example 3: Amorphous Compound A-HCl

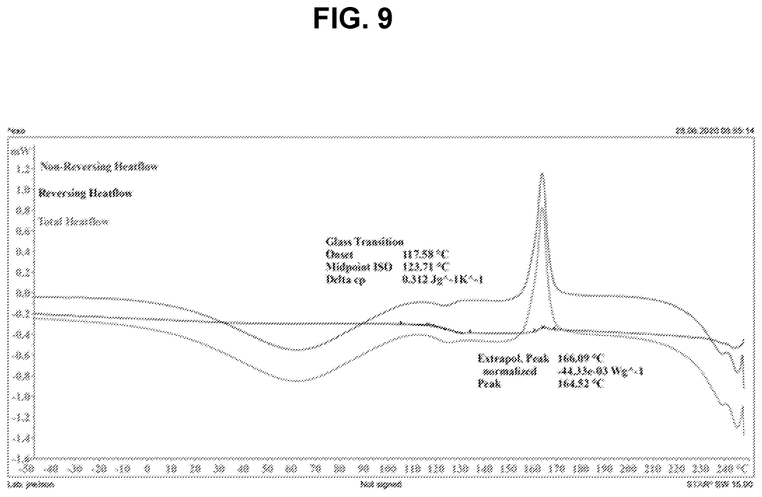

Amorphous Compound A-HCl was isolated from rotary evaporation in methanol and showed X-ray amorphous with broad peak(s). The glass transition temperature (T_{g}) was 124° C. as shown by modulated DSC analysis (MDSC) (FIG. 9). The compound was converted to Crystalline Compound A-HCl Form 1 upon heating at 165° C. to 180° C. The compound was converted to Crystalline Compound A-HCl Form 1 and Compound A Hydrate Form 2 upon stressing with water.

### Example 4: Crystalline Compound A-MsA Form 1

Crystalline Compound A-MsA Form 1 was prepared by slurring one molar equivalent of methanesulfonic acid and Compound A in acetonitrile at ambient condition. The gram level was prepared at a larger scale by dissolving 3 g of Compound A in ethyl acetate (30 ml) at 60° C. in a Mettler Toledo EasyMax controlled lab reactor with an overhead stirrer. One molar equivalent of methanesulfonic acid (350 μl) was added and precipitation was observed. The slurry was aged at 60° C. for 8 hours and then cooled to 20° C. at 0.1° C./min. The solids were isolated by vacuum filtration after aging at 20° C. overnight. The wet cake was washed with ethyl acetate (15 ml). XRPD analysis indicated the wet cake was Compound A-MsA Form 1. The wet cake was then vacuum dried at ambient temperature for 4 days and characterized. The yield is 89%.

X-Ray Powder Diffraction: The XRPD pattern of the Crystalline Compound A-MsA Form 1 material is shown in FIG. 10 and the XRPD peaks are listed in Table 6.

TABLE 6
XRPD Data of Crystalline Compound A-MsA Form 1:

| Pos. [°2θ] | FWHM [°2θ] | d-spacing [Å] | Height [cts] | Rel. Int. [%] |
|---|---|---|---|---|
| 5.7617 | 0.1171 | 15.33923 | 3563.27 | 31.19 |
| 6.9624 | 0.1506 | 12.69635 | 7042.91 | 61.65 |
| 10.3789 | 0.1506 | 8.5234 | 1384.44 | 12.12 |
| 11.4149 | 0.1004 | 7.75206 | 1349.12 | 11.81 |
| 11.757 | 0.1004 | 7.52727 | 2469.54 | 21.62 |
| 12.6462 | 0.1171 | 6.99991 | 4221.79 | 36.96 |
| 13.2625 | 0.184 | 6.676 | 1562.02 | 13.67 |
| 13.5157 | 0.184 | 6.55151 | 2979.83 | 26.09 |
| 13.9748 | 0.1506 | 6.33726 | 1876.93 | 16.43 |
| 15.2984 | 0.2509 | 5.79185 | 4150.19 | 36.33 |
| 15.7192 | 0.1171 | 5.63773 | 6578.37 | 57.59 |
| 16.0526 | 0.1338 | 5.52139 | 3477.55 | 30.44 |
| 16.5226 | 0.1338 | 5.36536 | 11423.3 | 100 |
| 17.3718 | 0.1673 | 5.10495 | 6589.9 | 57.69 |
| 18.0232 | 0.1673 | 4.92188 | 3151.82 | 27.59 |
| 18.4749 | 0.1506 | 4.80257 | 4712.45 | 41.25 |
| 19.0636 | 0.1338 | 4.65555 | 986.66 | 8.64 |
| 20.0335 | 0.184 | 4.43231 | 6106.14 | 53.45 |
| 20.5715 | 0.1171 | 4.31758 | 3925.51 | 34.36 |
| 20.9518 | 0.2007 | 4.24007 | 5418.76 | 47.44 |
| 22.0197 | 0.1338 | 4.03679 | 1294.16 | 11.33 |
| 22.5862 | 0.1004 | 3.9368 | 1497.18 | 13.11 |
| 22.9684 | 0.1338 | 3.87216 | 1723.17 | 15.08 |
| 23.1804 | 0.2007 | 3.83722 | 2033.44 | 17.8 |
| 23.9206 | 0.184 | 3.72013 | 7336.95 | 64.23 |
| 25.1798 | 0.2007 | 3.53687 | 2531.92 | 22.16 |
| 25.9079 | 0.2007 | 3.43911 | 1506.44 | 13.19 |
| 26.783 | 0.368 | 3.32869 | 1541.01 | 13.49 |
| 28.0224 | 0.1673 | 3.18422 | 1898.47 | 16.62 |
| 28.5556 | 0.1338 | 3.12597 | 899.77 | 7.88 |
| 29.4476 | 0.2676 | 3.03328 | 499.68 | 4.37 |
| 30.4832 | 0.368 | 2.93255 | 1823.75 | 15.97 |
| 31.9437 | 0.2007 | 2.80173 | 433.97 | 3.8 |
| 32.4773 | 0.1338 | 2.7569 | 977.85 | 8.56 |
| 32.8882 | 0.2007 | 2.72339 | 757.89 | 6.63 |
| 33.4218 | 0.1338 | 2.68112 | 398.32 | 3.49 |
| 34.7631 | 0.4015 | 2.58069 | 207.81 | 1.82 |
| 36.3258 | 0.5353 | 2.47317 | 143.17 | 1.25 |
| 37.1502 | 0.2007 | 2.42017 | 108.9 | 0.95 |
| 38.7259 | 0.3346 | 2.32525 | 207.39 | 1.82 |

Indexing Solution of Crystalline Compound A-MsA Form 1: XRPD indexing is a method that can be used to extract information and aid the interpretation of XRPD patterns. XRPD indexing is the process of determining the size, shape, and symmetry of the crystallographic unit cell for a crystalline component responsible for a set of peaks in an XRPD pattern. Crystalline Compound A-MsA Form 1 was collected with Cu-Kα radiation, and the indexing results are tabulated in Table 7 below.

TABLE 7
Indexing Result for XRPD Data of Crystalline
Compound A-MsA Form 1:

| Bravais Lattice Type | Primitive Monoclinic |
|---|---|
| a [Å] | 16.157 |
| b [Å] | 7.860 |
| c [Å] | 26.462 |
| α [deg] | 90 |
| β [deg] | 106.71 |
| γ [deg] | 90 |
| Volume [Å³/cell] | 3,218.6 |
| Chiral Contents? | Not specified |
| Extinction Symbol | P 1 2₁/n 1 |
| Space Group(s) | P2₁/n (14) |

Thermal Analysis: The DSC and TGA of the Crystalline Compound A-MsA Form 1 are shown in FIG. 11. Typical DSC of the Crystalline Compound A-MsA Form 1 indicated a melting onset at 250° C. TGA of the Crystalline Compound A-MsA Form 1 showed a weight loss of 0.2% prior decomposition.

Hygroscopicity Analysis: The hygroscopic profile of the Crystalline Compound A-MsA Form 1 is shown in FIG. 12. Typical DVS of Crystalline Compound A-MsA Form 1 showed a weight gain of about 1.2% by 95% RH.

### Example 5: Crystalline Compound A-MsA Form 2

The Crystalline Compound A-MsA Form 2 was prepared by slurring one equivalent of MSA and Compound A in 90/10 THF/water v/v solvent at ambient condition.

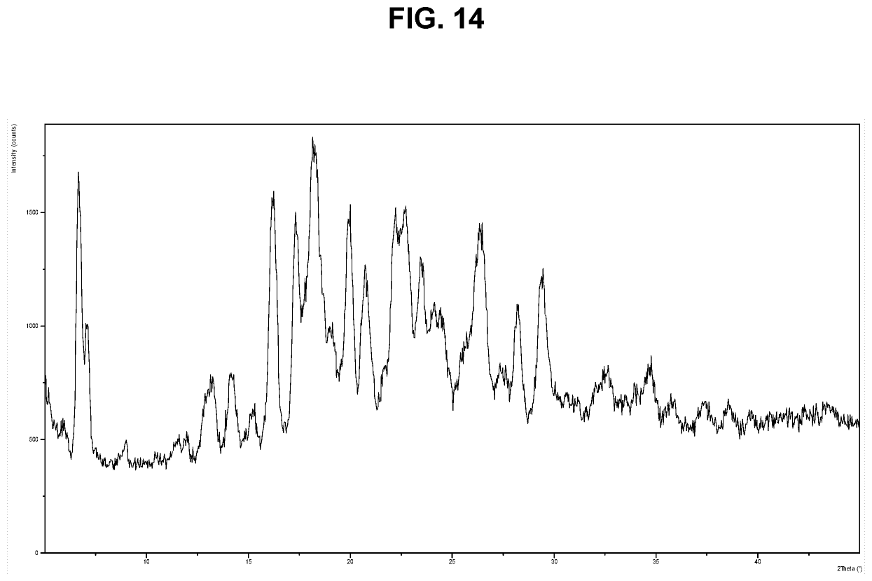

X-Ray Powder Diffraction: The XRPD pattern of the Crystalline Compound A-MsA Form 2 material is shown in FIG. 14.

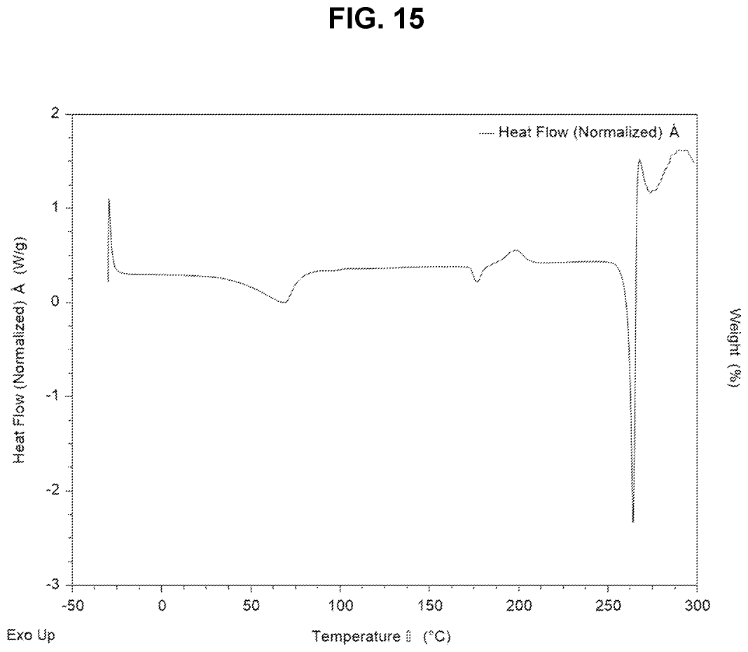

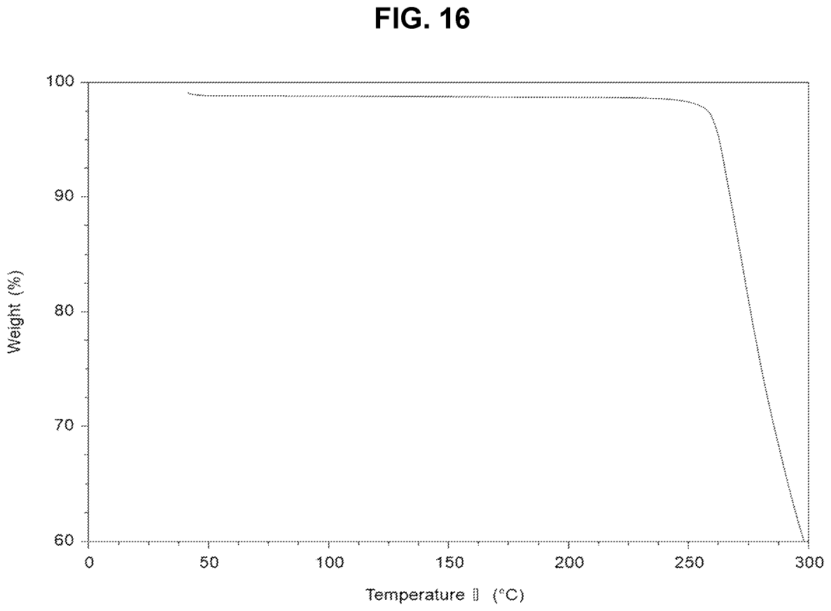

Thermal Analysis: The DSC of the Crystalline Compound A-MsA Form 2 is shown in FIG. 15. Typical DSC of Crystalline Compound A-MsA Form 2 indicated a melting onset of 38.0° C. and 177.1° C. endothermic events. TGA of the Crystalline Compound A-MsA Form 2 showed a weight loss of about 0.3% prior decomposition (see FIG. 16).

### Example 6: Crystalline Compound A-TsA Form 1 and Form 5

The Crystalline Compound A-TsA Form 1 was prepared by slurring one molar equivalent of p-Toluenesulfonic acid and Compound A in Acetonitrile at ambient condition.

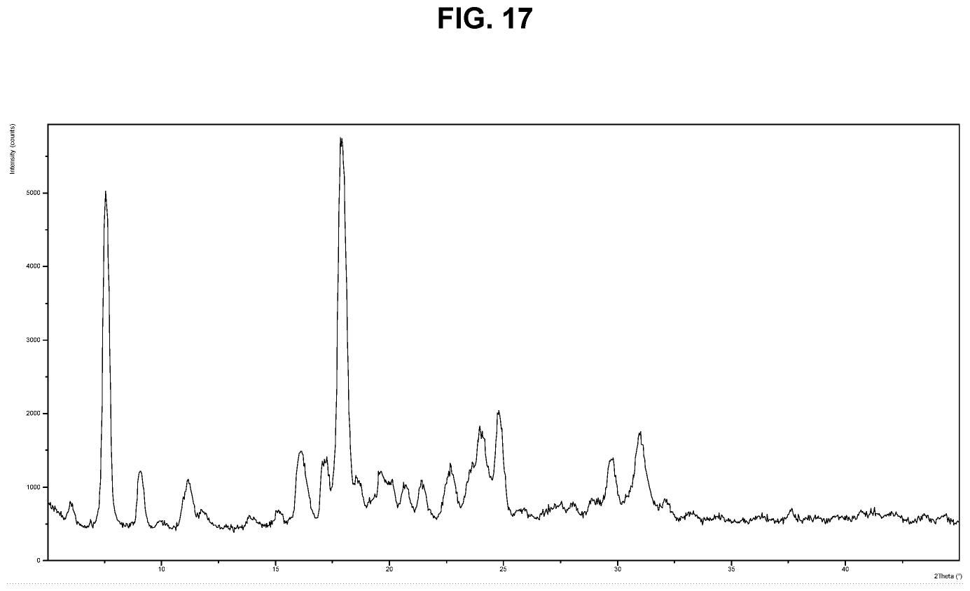

X-Ray Powder Diffraction: The XRPD pattern of the Crystalline Compound A-TsA Form 1 material is shown in FIG. 17.

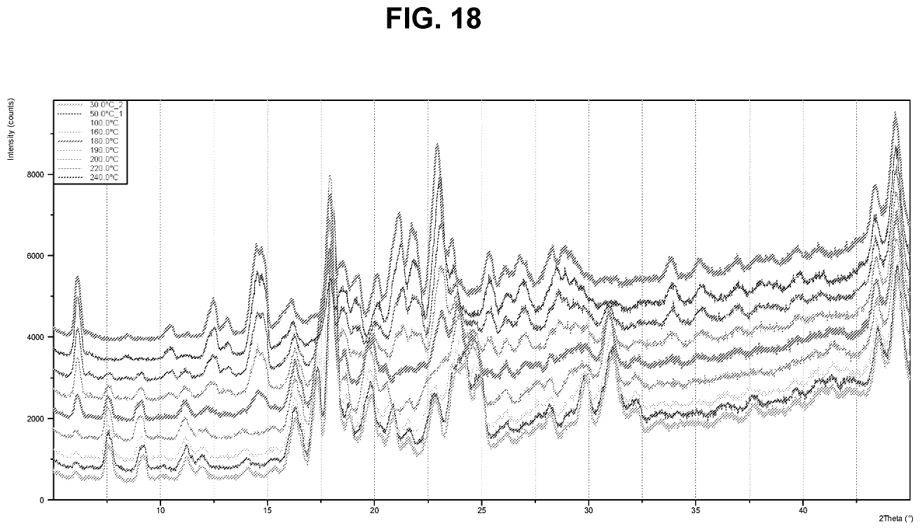

The variable temperature X-ray diffraction (VTXRD) of Crystalline Compound A-TsA Form 1 showed a recrystallization at a temperature of ≥180° C. and the new crystalline form was assigned as Crystalline Compound A-TsA Form 5. The VTXRD pattern is shown in FIG. 18.

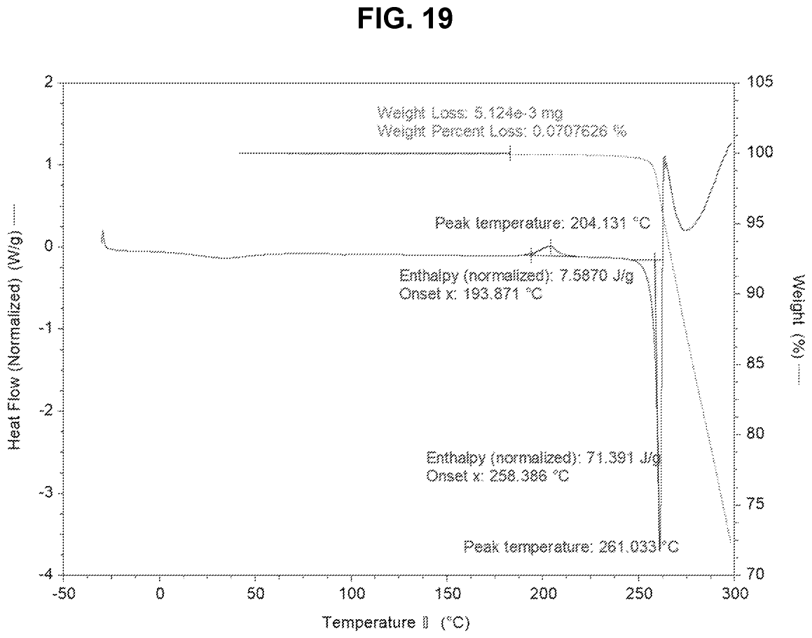

Thermal Analysis: The DSC and TGA patterns of the Crystalline Compound A-TsA Form 1 shown in FIG. 19. Typical DSC of Crystalline Compound A-TsA Form 1 indicated onsets of 193.9° C. and 258.4° C. endothermic events. TGA of the Crystalline Compound A-TsA Form 1 showed a weight loss of about 0.07% prior decomposition.

### Example 7: Crystalline Compound A-TsA Form 3

The Crystalline Compound A-TsA Form 3 was prepared by slurring one molar equivalent of p-Toluenesulfonic acid and Compound A in 90/10 EtOH/water v/v at ambient condition.

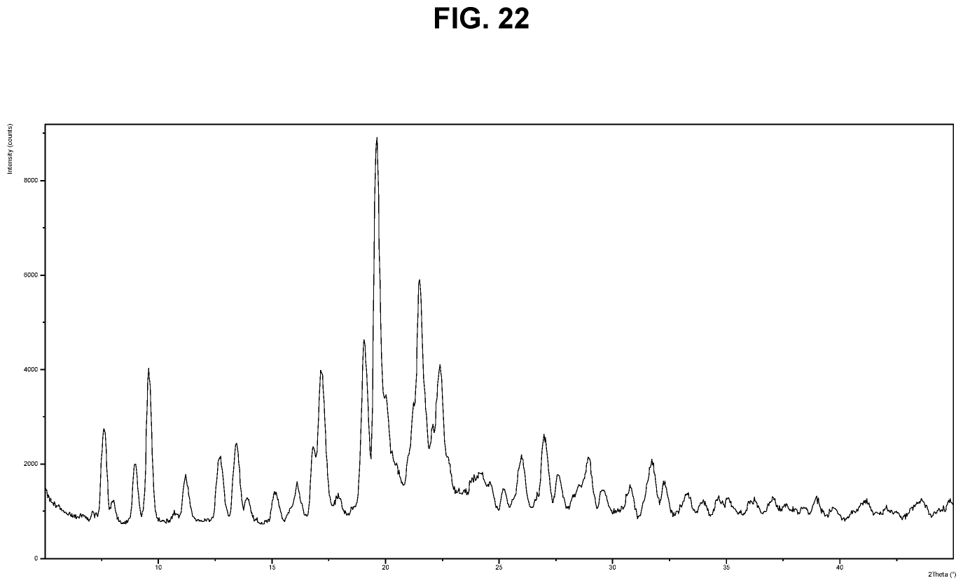

X-Ray Powder Diffraction: The XRPD pattern of the crystalline Compound A-TsA Form 3 is shown in FIG. 22.

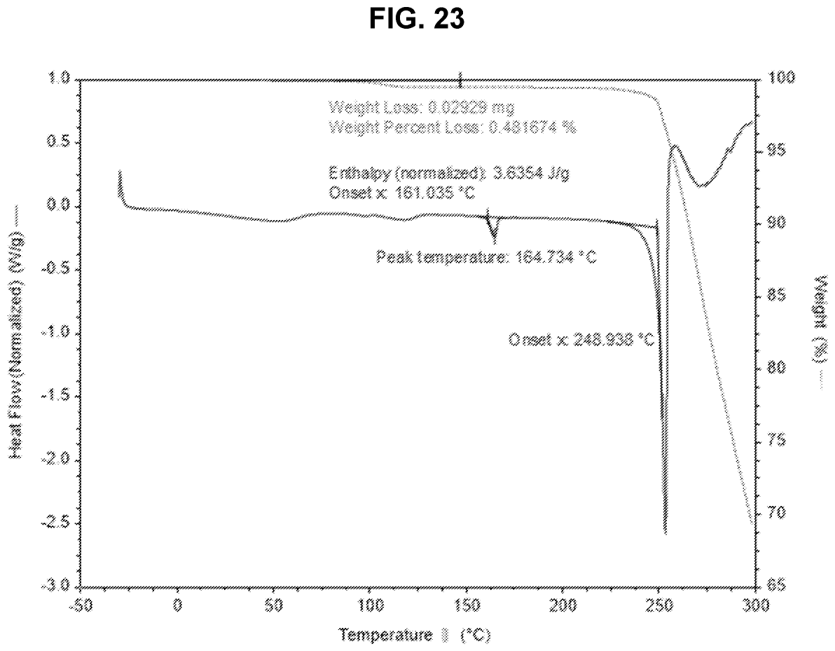

Thermal Analysis: The DSC and TGA patterns of the Crystalline Compound A-TsA Form 3 are shown in FIG. 23. Typical DSC of Crystalline Compound A-TsA Form 3 indicated onsets of 161.0° C. and 248.9° C. endothermic events. TGA of the Crystalline Compound A-TsA Form 3 showed a weight loss of about 0.48% prior decomposition.

### Example 8: Crystalline Compound A-TsA Form 4

The Crystalline Compound A-TsA Form 4 was prepared by slurring one molar equivalent of p-Toluenesulfonic acid and Compound A in EtOH at ambient condition. Alternatively, the compound was also generated from vacuum drying of Compound A-TsA Form 1 at a temperature of 95° C. to 103° C. for 1 day and then 107° C. to 109° C. for 3 days; or at 150° C. to 170° C. for 1 day.

The scale up of Compound A-TsA Salt Form 4 was prepared by desolvation of the Compound A-isopropanol solvate of TSA salt Form 1. The procedure involved stirring 3.5 g of Compound A and 1 molar equivalent of p-toluenesulfonic acid (1.08 g) in isopropanol (60 ml) at 60° C. in a Mettler Toledo EasyMax controlled lab reactor with an overhead stirrer. The slurry was stirred for 1 day at 60° C. and then cooled to 20° C. at 0.1° C./min. Solids were isolated by vacuum filtration and washed twice with isopropanol (10 ml). XRPD analysis indicated the material was composed of a mixture of Tosylate Salt Form 1 and minor free form isopropanol solvate. To attempt to complete the reaction, the solids were re-slurried in isopropanol (30 ml) with about 0.15 molar equivalents of p-toluenesulfonic acid (0.21 g) at ambient temperature for 4 days. The solids were isolated by vacuum filtration and washed with twice with isopropanol (10 ml). XRPD analysis indicated the solids were composed of Tosylate Salt Form 1 and still contained a trace amount of free form isopropanol solvate. The solids were again re-slurried with 0.25 molar equivalents of p-toluenesulfonic acid (0.34 g) in isopropanol (50 ml) at 60° C. The solids were isolated by vacuum filtration after stirring for 1 day. The wet cake was washed with isopropanol (15 ml) and was analyzed by XRPD. The XRPD pattern was consistent with Compound A-TsA Salt Form 1 and minor Compound A-TsA Salt Form 4. Vacuum drying of the material at 145° C. resulted in complete conversion to Compound A-TsA Salt Form 4 by XRPD. The XRPD pattern of the Crystalline Compound A-TsA Form 4 material is shown in FIG. 24a and the XRPD peaks are listed in Table 7.

TABLE 7
XRPD Data of Crystalline Compound A-TsA Form 4

| Pos. [°2θ] | FWHM [°2θ] | d-spacing [Å] | Height [cts] | Rel. Int. [%] |
|---|---|---|---|---|
| 6.202 | 0.0836 | 14.25126 | 11841.62 | 100 |
| 8.3596 | 0.0669 | 10.57727 | 862.73 | 7.29 |
| 10.485 | 0.1171 | 8.4374 | 1865.49 | 15.75 |
| 11.1414 | 0.0836 | 7.94174 | 551.39 | 4.66 |
| 12.1296 | 0.0669 | 7.29686 | 779.83 | 6.59 |
| 12.4404 | 0.0836 | 7.11527 | 1853.58 | 15.65 |
| 12.9743 | 0.0669 | 6.82362 | 774.13 | 6.54 |
| 13.129 | 0.0669 | 6.74358 | 977.91 | 8.26 |
| 14.2446 | 0.1004 | 6.21785 | 3903.03 | 32.96 |
| 14.6709 | 0.0836 | 6.03811 | 9322.96 | 78.73 |
| 14.8356 | 0.0502 | 5.97147 | 2122.89 | 17.93 |
| 15.5616 | 0.1004 | 5.69447 | 1034.9 | 8.74 |
| 16.4028 | 0.1004 | 5.40427 | 1127.73 | 9.52 |
| 16.7652 | 0.0836 | 5.28828 | 347.28 | 2.93 |
| 17.0513 | 0.0669 | 5.20018 | 378.6 | 3.2 |
| 17.6527 | 0.1004 | 5.02433 | 3101.72 | 26.19 |
| 18.331 | 0.1004 | 4.83993 | 1556.97 | 13.15 |
| 18.6311 | 0.1171 | 4.76264 | 2952.46 | 24.93 |
| 19.0787 | 0.1004 | 4.65191 | 4994.87 | 42.18 |
| 20.1393 | 0.1171 | 4.40924 | 1475.66 | 12.46 |
| 20.8195 | 0.1338 | 4.2667 | 1419.22 | 11.98 |
| 21.47 | 0.1004 | 4.13888 | 3668.99 | 30.98 |
| 21.8269 | 0.0836 | 4.07201 | 644.54 | 5.44 |
| 22.3718 | 0.1506 | 3.97404 | 2361.38 | 19.94 |
| 23.0541 | 0.0669 | 3.85796 | 516.62 | 4.36 |
| 23.4894 | 0.1338 | 3.78744 | 4553.16 | 38.45 |
| 24.1138 | 0.1673 | 3.69076 | 1930.58 | 16.3 |
| 24.3771 | 0.1171 | 3.65149 | 717.86 | 6.06 |
| 25.1314 | 0.0836 | 3.54357 | 990.68 | 8.37 |
| 25.2663 | 0.0836 | 3.52496 | 1313.91 | 11.1 |
| 25.4626 | 0.1004 | 3.49823 | 879.29 | 7.43 |
| 26.1153 | 0.1171 | 3.41226 | 751.94 | 6.35 |
| 26.5717 | 0.0836 | 3.35468 | 434.08 | 3.67 |
| 26.9761 | 0.0836 | 3.3053 | 711.31 | 6.01 |
| 28.6593 | 0.0836 | 3.11489 | 1044.32 | 8.82 |
| 29.0205 | 0.1506 | 3.07694 | 1315.89 | 11.11 |
| 29.5827 | 0.1004 | 3.01973 | 526.68 | 4.45 |
| 29.932 | 0.2007 | 2.98528 | 624.8 | 5.28 |
| 30.3779 | 0.2007 | 2.94247 | 321.63 | 2.72 |
| 30.7184 | 0.1171 | 2.91063 | 272.55 | 2.3 |
| 31.1915 | 0.1338 | 2.86755 | 235.81 | 1.99 |
| 32.7035 | 0.2342 | 2.73835 | 148.16 | 1.25 |
| 33.71 | 0.1171 | 2.65885 | 200.45 | 1.69 |
| 34.1773 | 0.1673 | 2.62357 | 235.65 | 1.99 |
| 35.1349 | 0.0836 | 2.55422 | 478.92 | 4.04 |
| 36.7215 | 0.1673 | 2.44742 | 226.97 | 1.92 |
| 37.7829 | 0.2007 | 2.38108 | 112.12 | 0.95 |
| 38.3197 | 0.2342 | 2.34895 | 204.82 | 1.73 |
| 38.6344 | 0.2007 | 2.33054 | 201.61 | 1.7 |
| 39.7904 | 0.0669 | 2.26546 | 288.15 | 2.43 |

Single Crystal Data: Table 8 shows the Crystallographic data summary of the Crystalline Compound A-TsA Form 4. The molecular structure of Crystalline Compound A-TsA Form 4 as found from X-ray crystal structure determination is shown in FIG. 24b.

TABLE 8
X-ray Single Crystallographic data summary
of the Crystalline Compound A-TsA Form 4:

| Wavelength | _1.542 | Å |
|---|---|---|
| Crystal System | Orthorhombic | |
| Space Group | Pbcn | |
| Unit Cell | a = 24.7407 Å | |
| | b = 10.7567 Å | |
| | c = 26.4846 Å | |
| | α = 90° | |
| | β = 90° | |
| | γ = 90° | |
| Volume | 7048.3 | Å³ |
| Z | 8 | |
| Density (calculated) | 1.391 | Mg/m³ |

Thermal Analysis: The DSC and TGA patterns of the Crystalline Compound A-TsA Form 4 are shown in FIG. 25. Typical DSC of the Crystalline Compound A-TsA Form 4 indicated a melting onset at 253° C. TGA of the Crystalline Compound A-TsA Form 4 showed a weight loss of 0.145% prior decomposition.

Solid State NMR: A solid state ¹⁹F NMR spectrum of the crystalline Compound A-TsA Form 4 is shown in FIG. 26 indicating 2 peaks at −96.93 and −101.60 ppm.

### Example 9: Crystalline Compound A-TsA Form 5

The Crystalline Compound A-TsA Form 5 was prepared by heating Crystalline Compound A-TsA Form 1 to above 180° C.

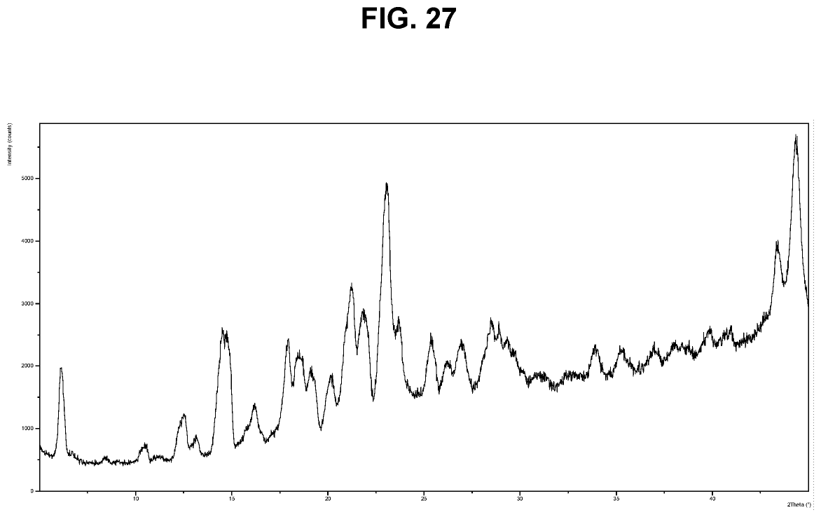

X-Ray Powder Diffraction: The XRPD pattern is shown in FIG. 27.

### Example 10: Crystalline Compound A-DiTsA Form 6

The Crystalline Compound A-DiTsA Form 6 was prepared by slurring two molar equivalents of p-Toluenesulfonic acid and Compound A in Acetonitrile in high throughput settings. Scale-up effort of the compound was not successful.

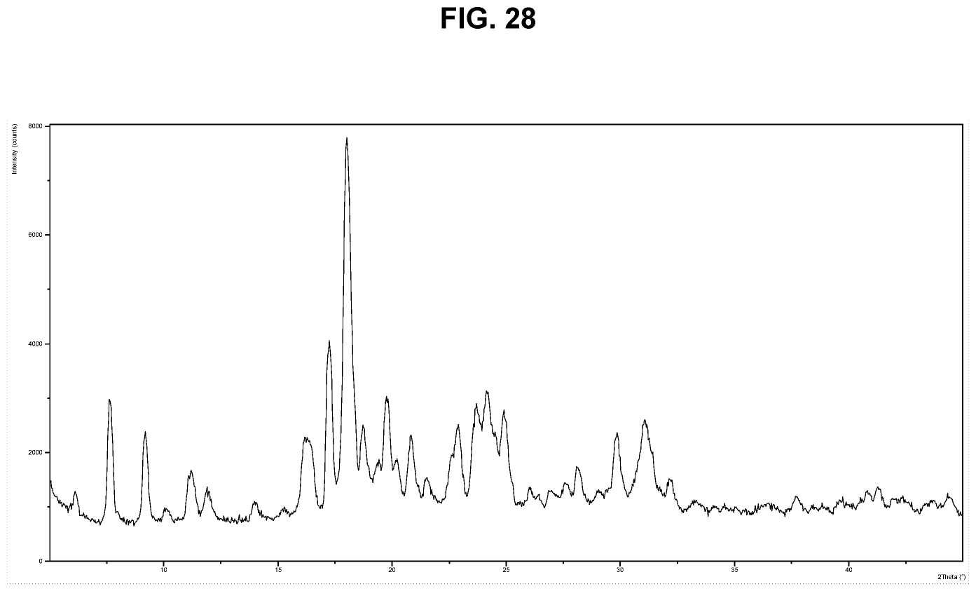

X-Ray Powder Diffraction: The XRPD pattern is shown in FIG. 28.

### Example 11: Crystalline Compound A-Sulfate Form 1

The Crystalline Compound A-Sulfate Form 1 was prepared by slurring one equivalent of sulfuric acid and Compound A in Acetonitrile at ambient condition.

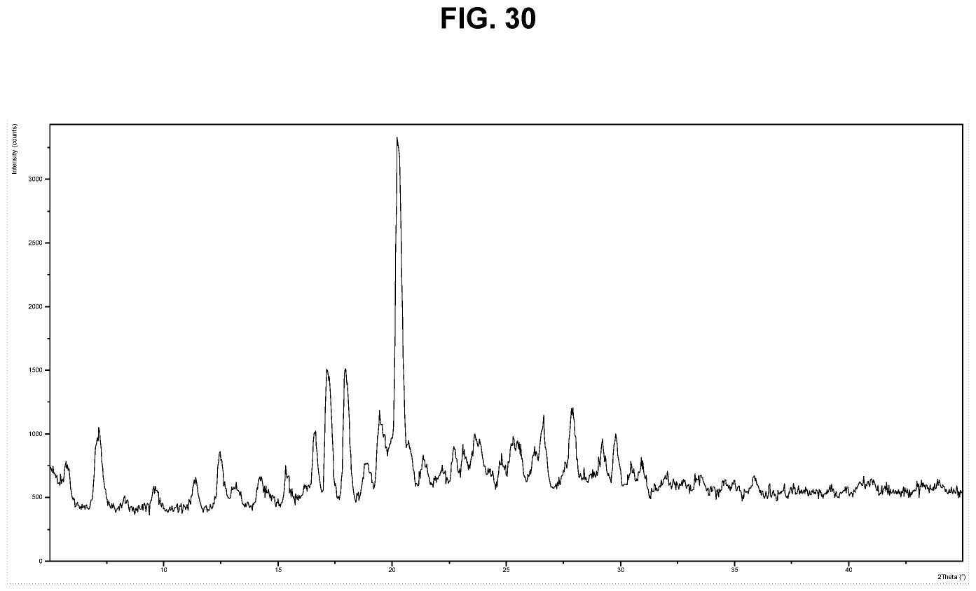

X-Ray Powder Diffraction: The XRPD pattern is shown in FIG. 30.

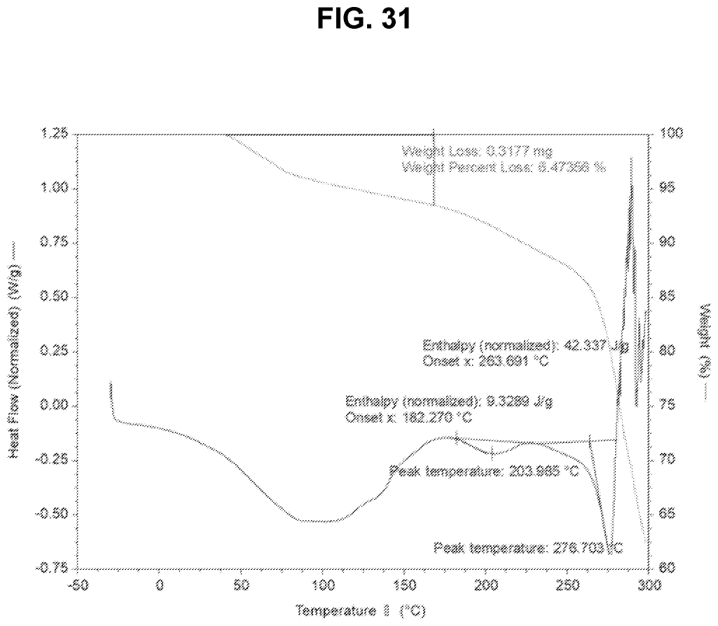

Thermal Analysis: The DSC and TGA of the Crystalline Compound A-Sulfate Form 1 are shown in FIG. 31. Typical DSC of the Crystalline Compound A-Sulfate Form 1 indicated onsets at 182.3° C. and 263.7° C. endothermic events. TGA of the Crystalline Compound A-Sulfate Form 1 showed a weight loss of 6.47% prior to decomposition.

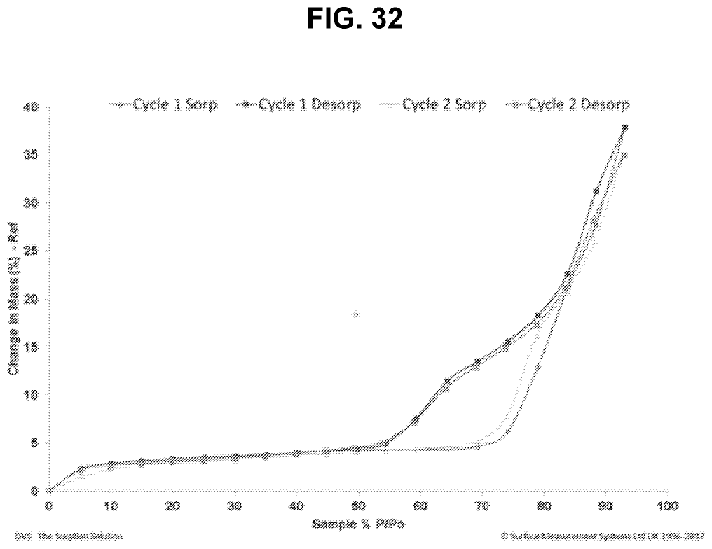

Hygroscopicity Analysis: The hygroscopic profile of the Crystalline Compound A-Sulfate Form 1 is shown in FIG. 32. Dynamic Vapor Sorption (DVS) of the Crystalline Compound A-Sulfate Form 1 suggests the sulfate salt deliquesces at 90% RH.

### Example 12: Amorphous Compound A

The Amorphous Compound A was prepared by dissolving 1.99 g of Compound A-Variable Hydrate Form 2 (See Example #13) in 100 mL acetone and shaking to form a yellow solution. The solution was then spray dried at a spray rate of 2 mL/min with an inlet temperature of 54° C., outlet temperature of 54° C., aspirator at 95%, drying air flow at 0.55 kg/min, nozzle air at 6.0 sL/min, and nozzle cool at 20° C. The amorphous product was collected and dried under vacuum oven at 40° C. with −10 bar pressure for 2.5 hours to remove the residual acetone.

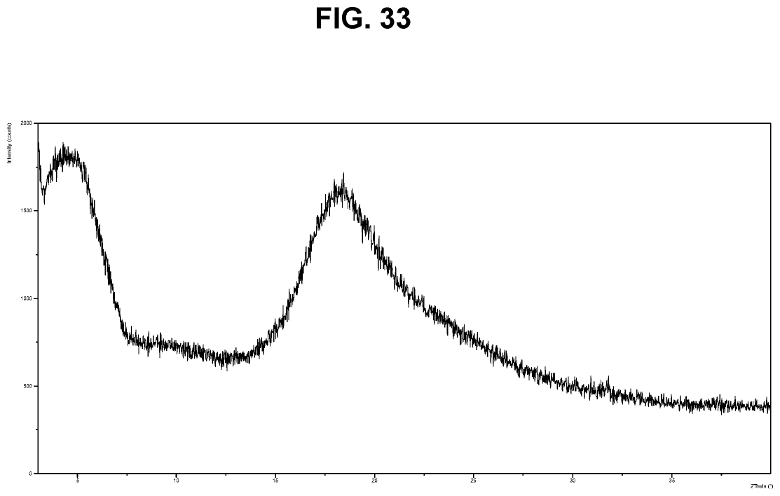

X-Ray Powder Diffraction: The XRPD pattern of the Amorphous Compound A is shown in FIG. 33.

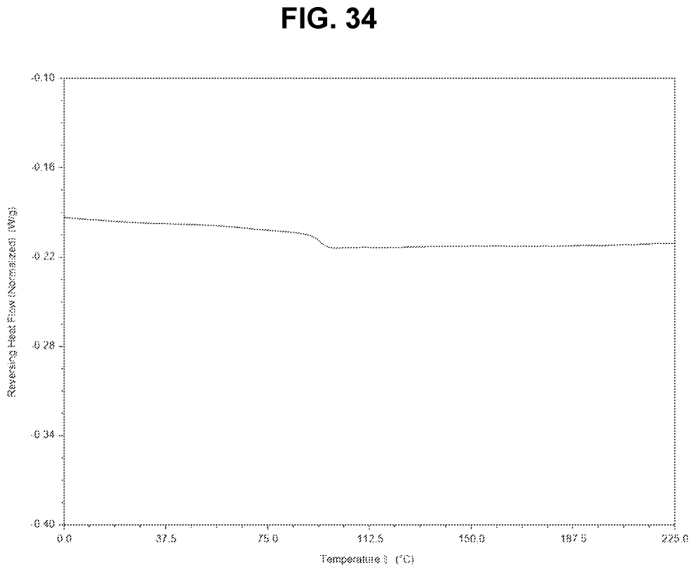

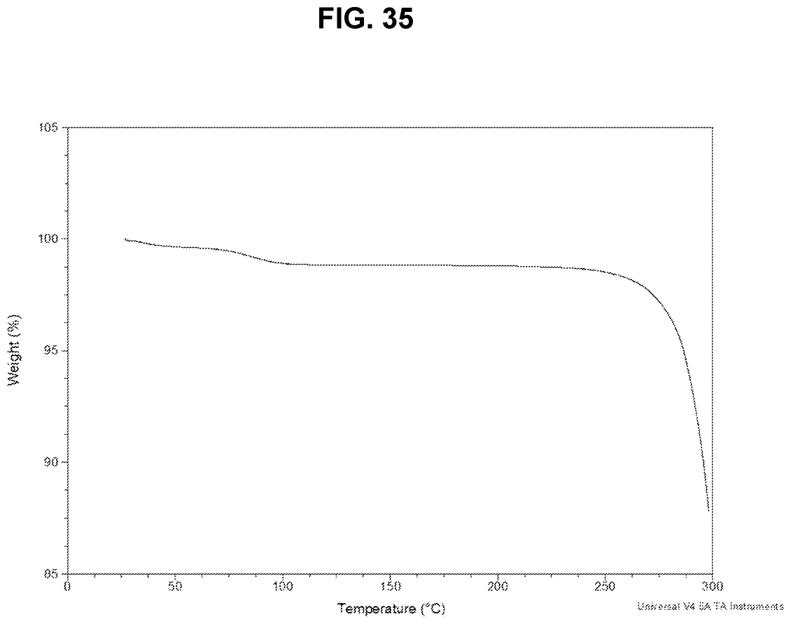

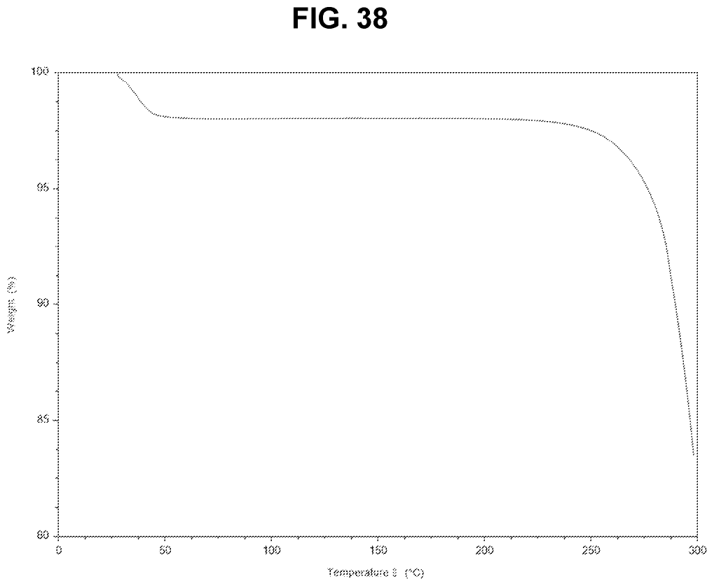

Thermal Analysis: The DSC of the Amorphous Compound A is shown in FIG. 38. Typical DSC of the Amorphous Compound A indicated a glass transition temperature (T_{g}) at 91° C. The TGA-IR of the Amorphous Compound A is shown in FIG. 34. TGA-IR of the Amorphous Compound A showed a 1.05% weight loss of water molecule below 100° C. as shown in FIG. 35.

### Example 13: Compound A-Variable Hydrate Form 2

The Compound A-Variable Hydrate Form 2 was prepared by slurring a mixture of Compound A-Methanol Form 1 and Compound A-Ethanol Form 1 product in water for 24 hours. The product was then filtered and air dried.

Alternatively, Compound A-Variable Hydrate Form 2 was prepared by mixing Compound A in methanol and ethanol solvent mixture. The compound A first formed Compound A-Methanol and Compound A-Ethanol solvates mixture, which was then slurried in water to initiate the conversion to Compound A-Variable Hydrate Form 2 product. To achieve complete conversion, the Compound A-Variable Hydrate Form 2 product was filtered and dried at elevated temperature (e.g. 50 C) overnight to remove all remaining organic solvents.

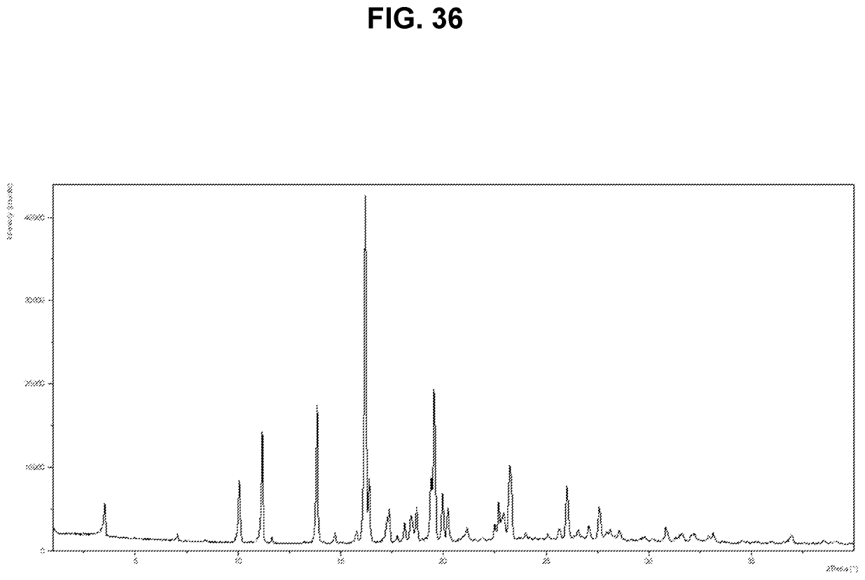

X-Ray Powder Diffraction: The XRPD pattern of the Compound A-Variable Hydrate Form 2 is shown in FIG. 36 and the XRPD peaks are listed in Table 9.

TABLE 9
XRPD Data of Crystalline Compound A-Variable Hydrate Form 2.

| Pos. [°2θ] | FWHM [°2θ] | d-spacing [Å] | Height [cts] | Rel. Int. [%] |
|---|---|---|---|---|
| 3.5259 | 0.0465 | 25.05944 | 3874.4 | 9.47 |
| 7.0727 | 0.0465 | 12.49861 | 817.33 | 2 |
| 8.4034 | 0.0697 | 10.52216 | 249.56 | 0.61 |
| 10.0657 | 0.0465 | 8.78788 | 7370.33 | 18.02 |
| 10.6175 | 0.0697 | 8.33241 | 210.38 | 0.51 |
| 11.1919 | 0.0465 | 7.90602 | 13007.2 | 31.81 |
| 11.6612 | 0.0465 | 7.58891 | 766.31 | 1.87 |
| 13.1983 | 0.0697 | 6.70835 | 236.22 | 0.58 |
| 13.858 | 0.0581 | 6.39044 | 16389.41 | 40.08 |
| 14.7185 | 0.0581 | 6.01871 | 1238.31 | 3.03 |
| 15.7803 | 0.0465 | 5.61604 | 1430.57 | 3.5 |
| 16.2049 | 0.0581 | 5.46983 | 40895.73 | 100 |
| 16.3899 | 0.0465 | 5.40851 | 7422.06 | 18.15 |
| 17.2502 | 0.0348 | 5.14065 | 3019.89 | 7.38 |
| 17.3745 | 0.0465 | 5.10417 | 3630.09 | 8.88 |
| 17.7541 | 0.0581 | 4.99588 | 717.93 | 1.76 |
| 18.1174 | 0.0581 | 4.8965 | 2212.42 | 5.41 |
| 18.4171 | 0.0697 | 4.81749 | 3123.43 | 7.64 |
| 18.6966 | 0.0581 | 4.74611 | 4195.83 | 10.26 |
| 19.3851 | 0.0465 | 4.57908 | 7495.87 | 18.33 |
| 19.5557 | 0.0581 | 4.53951 | 18007.58 | 44.03 |
| 19.9635 | 0.0581 | 4.44769 | 5786.34 | 14.15 |
| 20.2172 | 0.0581 | 4.39244 | 3988.37 | 9.75 |
| 20.7733 | 0.0465 | 4.27609 | 400.1 | 0.98 |
| 21.1641 | 0.0929 | 4.19801 | 1496.44 | 3.66 |
| 21.9302 | 0.0929 | 4.05307 | 400.41 | 0.98 |
| 22.4926 | 0.0465 | 3.95297 | 2032.91 | 4.97 |
| 22.6875 | 0.0465 | 3.91945 | 4501.02 | 11.01 |
| 22.9495 | 0.0465 | 3.8753 | 3327.64 | 8.14 |
| 23.2126 | 0.0708 | 3.8288 | 8736.19 | 21.36 |
| 23.2929 | 0.0425 | 3.82526 | 8096.88 | 19.8 |
| 24.0078 | 0.1133 | 3.70374 | 821.54 | 2.01 |
| 25.0842 | 0.0708 | 3.5472 | 683.37 | 1.67 |
| 25.6238 | 0.0992 | 3.47371 | 1376.56 | 3.37 |
| 26.0061 | 0.0992 | 3.4235 | 6312.46 | 15.44 |
| 26.5611 | 0.0708 | 3.35322 | 1159.46 | 2.84 |
| 27.0761 | 0.085 | 3.29059 | 1684.78 | 4.12 |
| 27.5641 | 0.085 | 3.23343 | 3930.25 | 9.61 |
| 27.9337 | 0.0708 | 3.19149 | 961.99 | 2.35 |
| 28.1181 | 0.0708 | 3.17097 | 1269.02 | 3.1 |
| 28.5516 | 0.0992 | 3.12381 | 1112.73 | 2.72 |
| 29.8219 | 0.1417 | 2.99358 | 411.91 | 1.01 |
| 30.2018 | 0.1133 | 2.95677 | 311.5 | 0.76 |
| 30.8316 | 0.0992 | 2.8978 | 1695.28 | 4.15 |
| 31.2945 | 0.085 | 2.85598 | 483.95 | 1.18 |
| 31.6143 | 0.085 | 2.82782 | 997.71 | 2.44 |
| 32.0573 | 0.0567 | 2.78974 | 929.07 | 2.27 |
| 32.2146 | 0.0567 | 2.77648 | 1013.24 | 2.48 |
| 32.9146 | 0.0567 | 2.71901 | 791.51 | 1.94 |
| 33.1229 | 0.0567 | 2.70239 | 1184.3 | 2.9 |
| 33.5895 | 0.1133 | 2.66591 | 219.49 | 0.54 |
| 34.5176 | 0.0708 | 2.59633 | 365.15 | 0.89 |
| 35.286 | 0.2267 | 2.54153 | 211.54 | 0.52 |
| 35.9712 | 0.1133 | 2.49467 | 323.7 | 0.79 |
| 36.9248 | 0.0567 | 2.43241 | 1163.96 | 2.85 |
| 37.5393 | 0.1417 | 2.39398 | 184.66 | 0.45 |
| 38.5019 | 0.0567 | 2.33632 | 401.22 | 0.98 |
| 39.0919 | 0.1133 | 2.3024 | 351.71 | 0.86 |

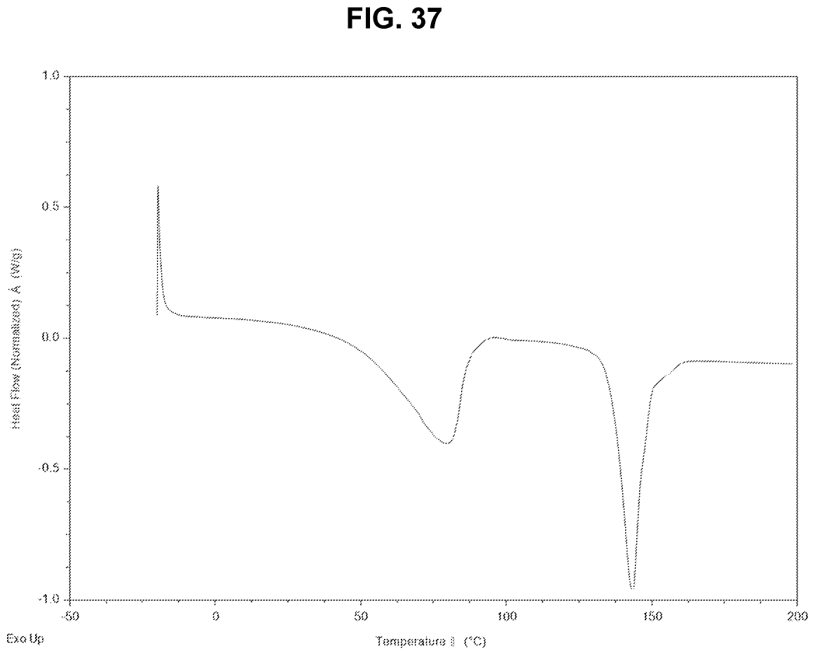

Thermal Analysis: The DSC of the Compound A-Variable Hydrate Form 2 is shown in FIG. 37. Typical DSC of the Compound A-Variable Hydrate Form 2 indicated dehydration onset of 51° C. and a melting point of 136° C. The TGA of the Compound A-Variable Hydrate Form 2 is shown in FIG. 38. TGA of the Compound A-Variable Hydrate Form 2 showed a 2.0% weight loss of water molecule below 100° C.

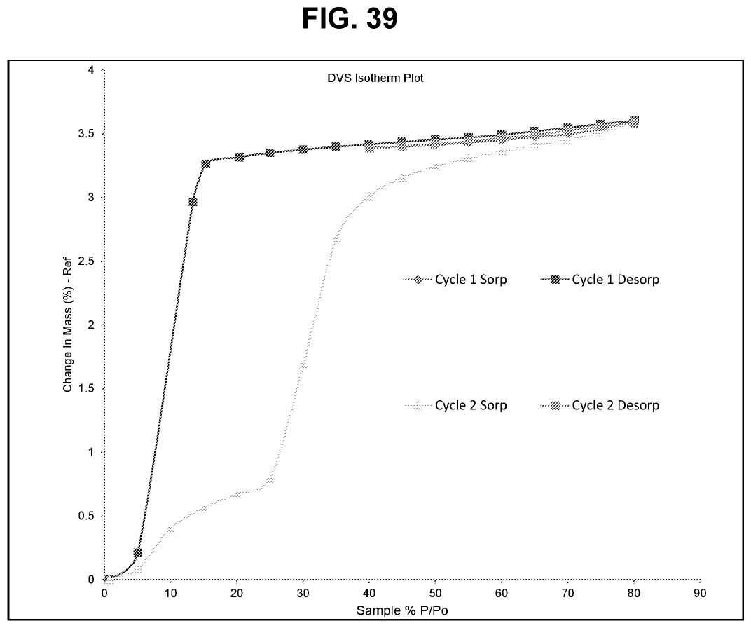

Hygroscopicity Analysis: The hygroscopic profile of the Compound A-Variable Hydrate Form 2 is shown in FIG. 39. Dynamic Vapor Sorption (DVS) of the Compound A-Variable Hydrate Form 2 showed a weight gain of about 3.4% by 95% RH.

### Example 14: Anhydrous Compound a Form 3

Anhydrous Compound A Form 3 was obtained by heating Compound A-THF solvate to a temperature of 150° C., holding for 3 minutes, then equilibrating at RT.

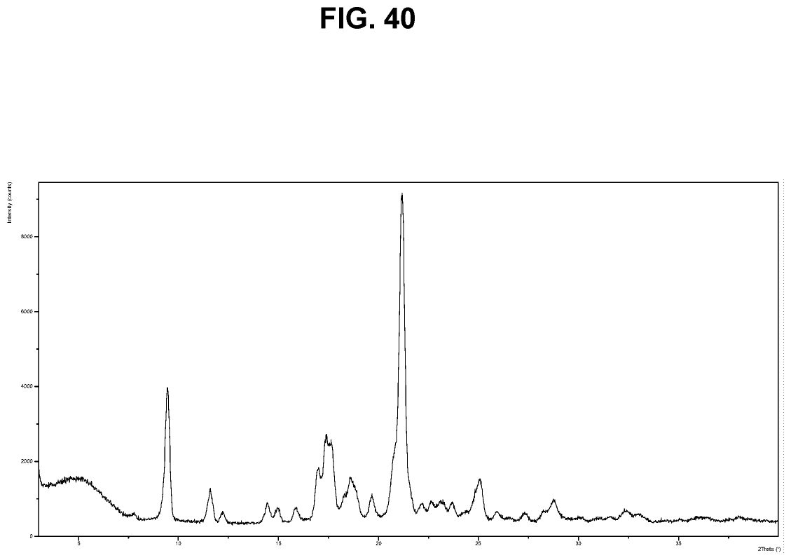

X-Ray Powder Diffraction: The XRPD pattern of Anhydrous Compound A Form 3 is shown in FIG. 40.

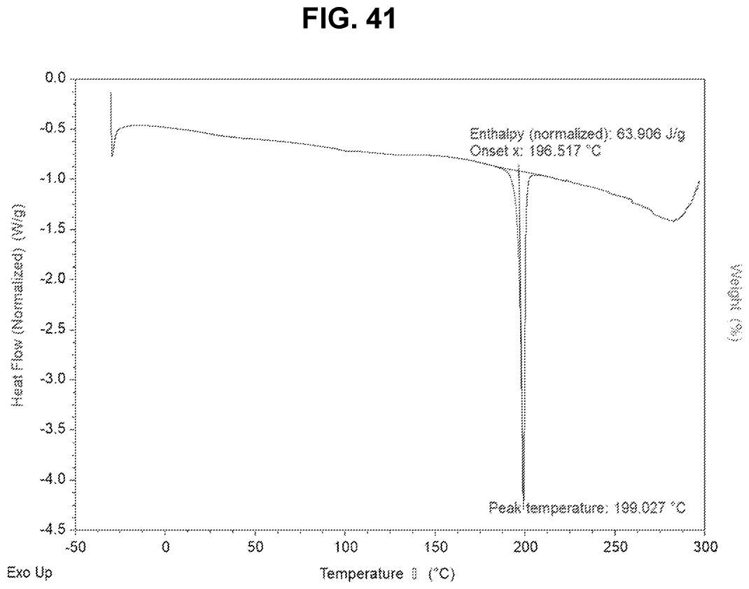

Thermal Analysis: The DSC of the Anhydrous Compound A Form 3 is shown in FIG. 41. Typical DSC of the Anhydrous Compound A Form 3 indicated a melting onset of 196.5° C.

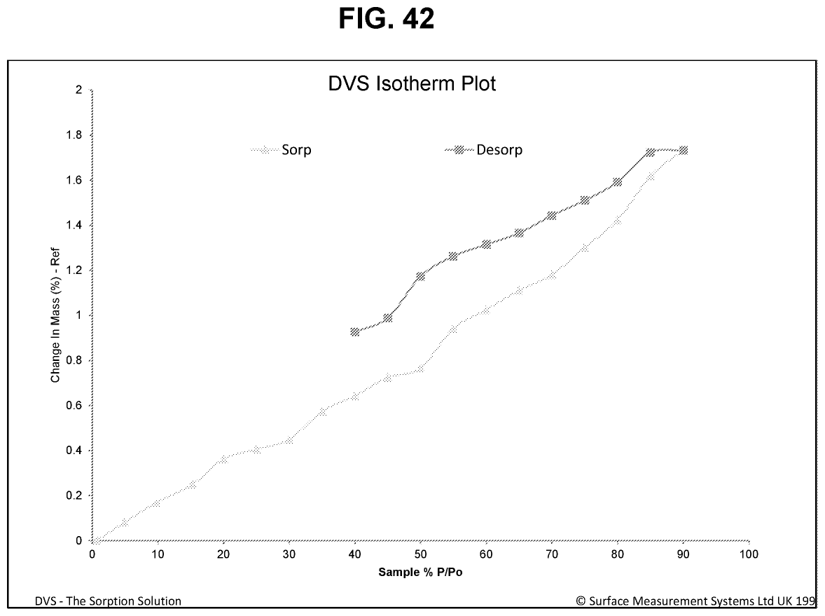

Hygroscopicity Analysis: The hygroscopic profile of the Anhydrous Compound A Form 3 is shown in FIG. 42. Dynamic Vapor Sorption (DVS) of the Anhydrous Compound A Form 3 showed a weight gain of about 1.5% by 95% RH.

### Example 15: Anhydrous Compound A Form 4

Anhydrous Compound A Form 4 was obtained by slurring mixed Anhydrous Compound A Form 3 and Compound A-Variable Hydrate Form 2 (Example 13) in heptane at 40° C. for 5 days.

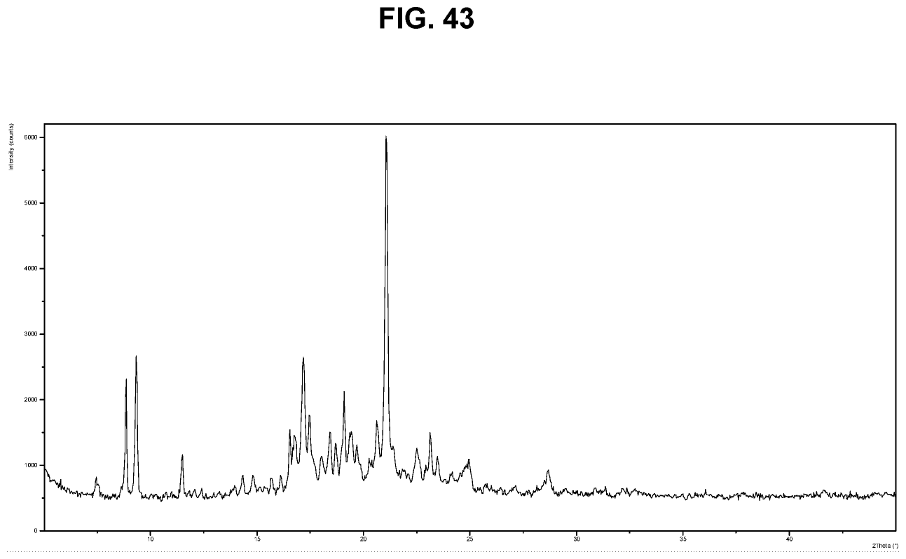

X-Ray Powder Diffraction: The XRPD pattern of Anhydrous Compound A Form 4 is shown in FIG. 43.

### Example 16: Anhydrous Compound A Form 5

Anhydrous Compound A Form 5 was obtained by slurrying 350 mg of Anhydrous Compound A Form 3 and Compound A-Variable Hydrate Form 2 (Example 13) mixture in 18 mL of heptane at a temperature of 70° C. for one day. The solid was then removed from the hot plate and filtered and washed with 5 mL heptane; then dried with a bleed of nitrogen overnight.

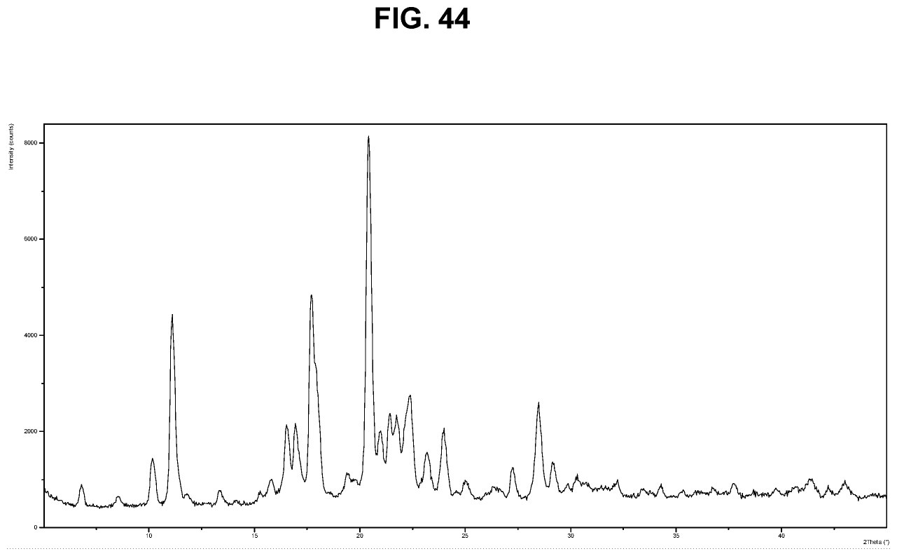

X-Ray Powder Diffraction: The XRPD pattern of Anhydrous Compound A Form 5 is shown in FIG. 44.

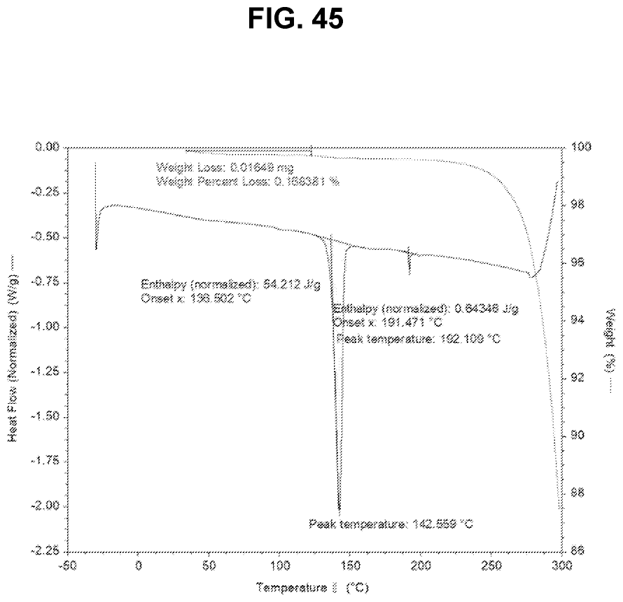

Thermal Analysis: The DSC and TGA patterns of the Anhydrous Compound A Form 5 are shown in FIG. 45. Typical DSC of the Anhydrous Compound A Form 5 indicated melting onset of 136.5° C. TGA of the Anhydrous Compound A Form 5 showed a 0.17% weight loss.

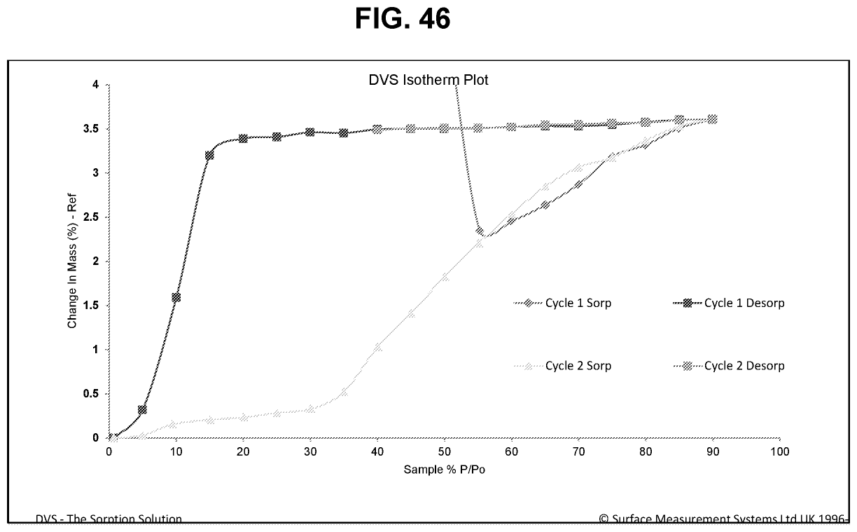

Hygroscopicity Analysis: The hygroscopic profile of the Anhydrous Compound A Form 5 is shown in FIG. 46. Dynamic Vapor Sorption (DVS) of the Anhydrous Compound A Form 5 showed the compound rehydrated to the Compound A-Variable Hydrate Form 2 (Example 13).

### Example 17: Anhydrous Compound A Form 6

Anhydrous Compound A Form 6 was obtained by slurrying Anhydrous Compound A Form 3 and Compound A-Variable Hydrate Form 2 (Example 13) mixture in heptane at a temperature of 80° C. overnight.

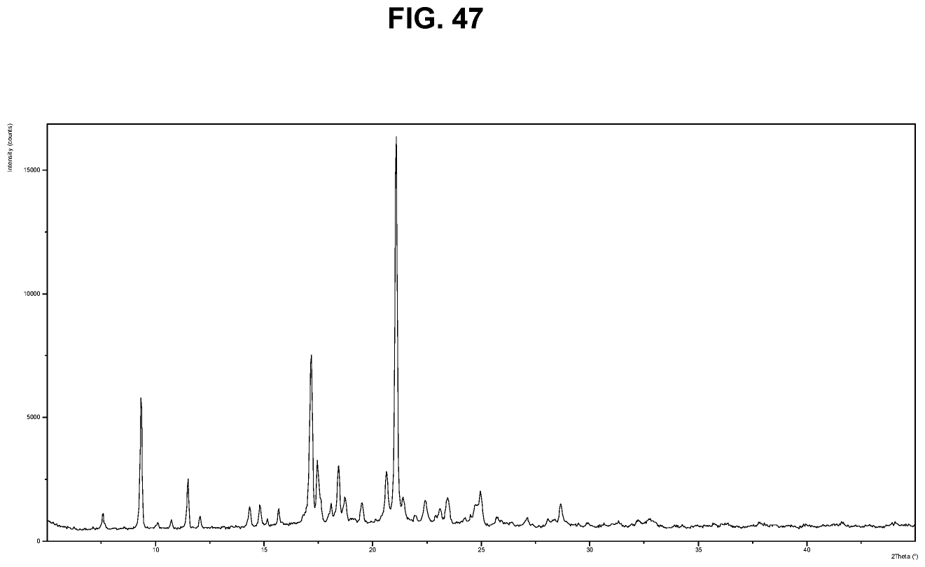

X-Ray Powder Diffraction: The XRPD pattern of Anhydrous Compound A Form 6 is shown in FIG. 47.

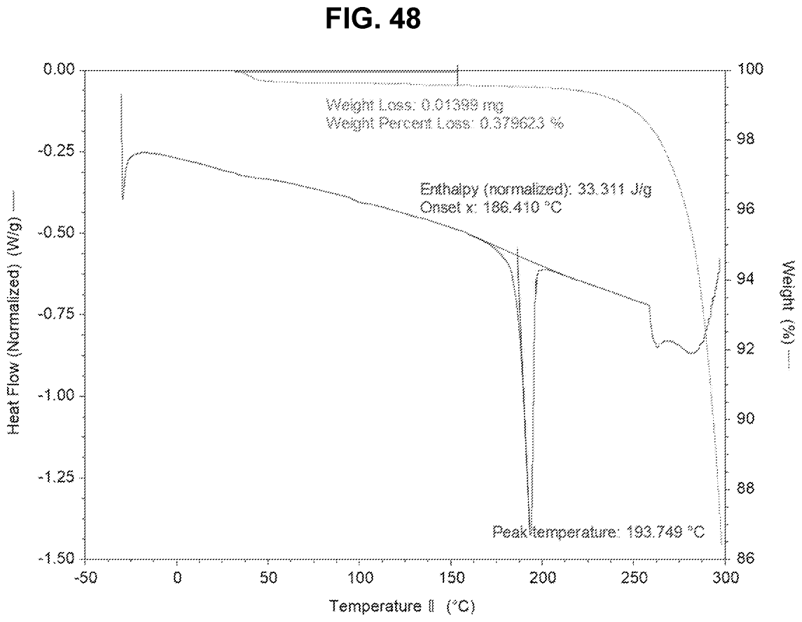

Thermal Analysis: The DSC and TGA of the Anhydrous Compound A Form 6 are shown in FIG. 48. Typical DSC of the Anhydrous Compound A Form 6 indicated onset of 186.4° C. TGA of the Anhydrous Compound A Form 6 showed a 0.38% weight loss.

### Example 18: Anhydrous Compound A Form 7

Anhydrous Compound A Form 7 was obtained by slurrying Anhydrous Compound A Form 3 and Compound A-Variable-Hydrate Form 2 (Example 13) in heptane at a temperature of 70° C. for 3 days.

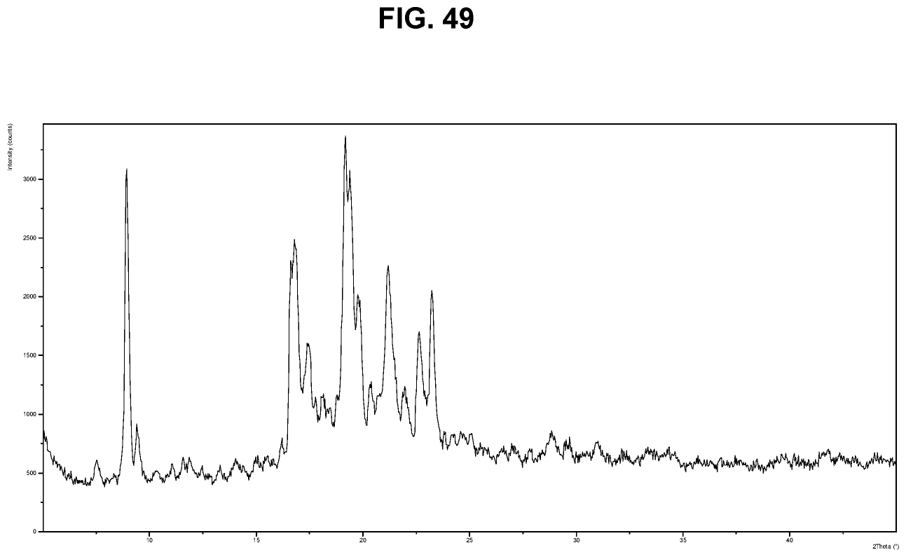

X-Ray Powder Diffraction: The XRPD pattern of Anhydrous Compound A Form 7 is shown in FIG. 49.

### Example 19: Anhydrous Compound A Form 8

Anhydrous Compound A Form 8 was obtained by slurrying Anhydrous Compound A Form 3 and Compound A-Variable-Hydrate Form 2 (Example 13) in toluene at a temperature of 50° C. for 3 days.

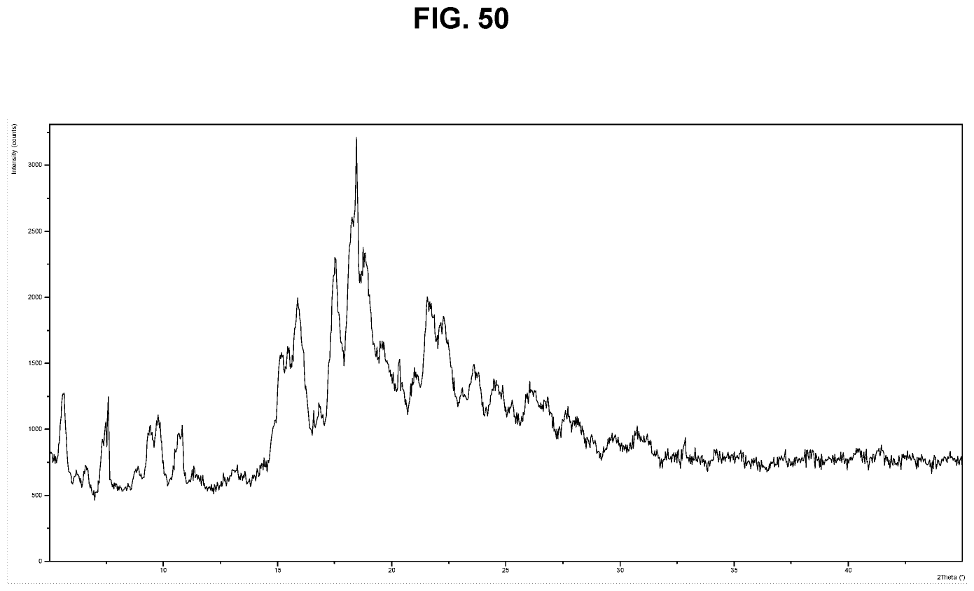

X-Ray Powder Diffraction: The XRPD pattern of Anhydrous Compound A Form 8 is shown in FIG. 50.

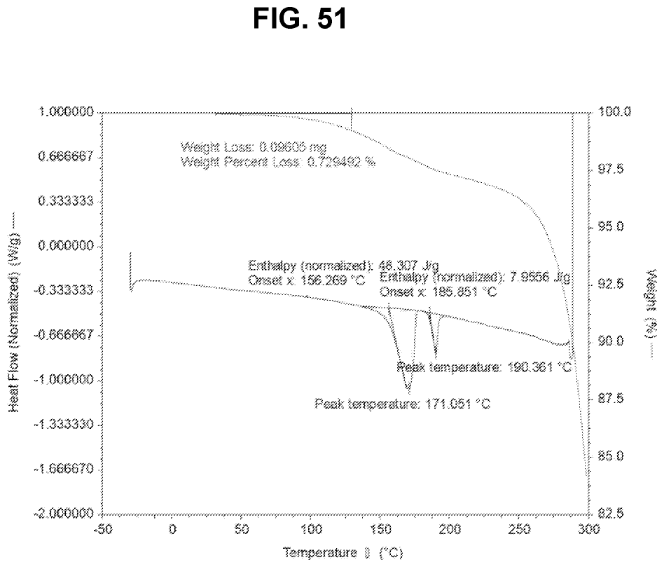

Thermal Analysis: The DSC and TGA of the Anhydrous Compound A Form 8 are shown in FIG. 51. Typical DSC of the Anhydrous Compound A Form 8 indicated melting onset of 156.3° C. and 185.9° C. TGA of the Anhydrous Compound A Form 8 showed a 0.73% weight loss.

### Example 20: Crystalline Compound A Form 1

To obtain the Crystalline Compound A Form 1, the Compound A was purified by Silica Gel column chromatography in combi-flash using a pre-packed Redi Sep column (12 g) and 20% to 100% EtOH in hexane as eluent. Thereafter, the fraction with desired product was concentrated under reduced pressure and the residue was dissolved in acenitrile/water solvent mixture and lyophilized.

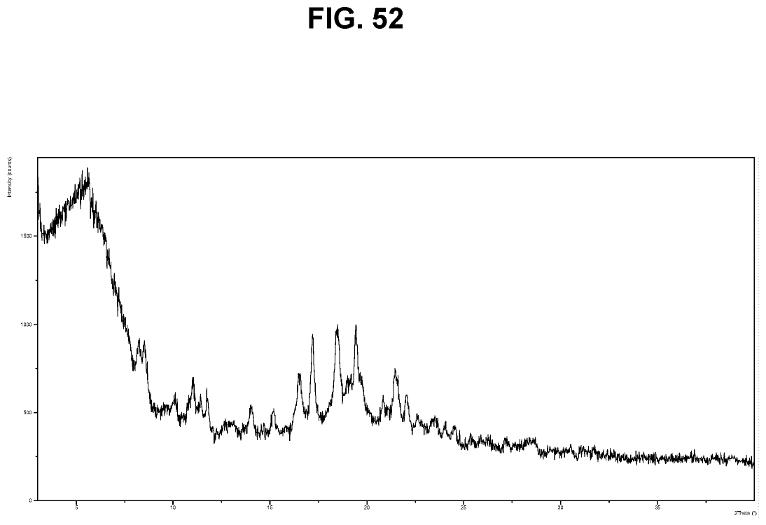

X-Ray Powder Diffraction: The XRPD pattern of Crystalline Compound A Form 1 is shown in FIG. 52.

### Example 21: Crystalline Compound A-THF Solvate

Crystalline Compound A-THF Solvate was prepared by slurrying Compound A in variety of solvents, i.e., a) 50 mg/mL of THF solution; b) 50-50 THF/water mixture; c) 50-50 THF/Methanol mixture; or d) 50-25-25 THF-NMP-water mixture.

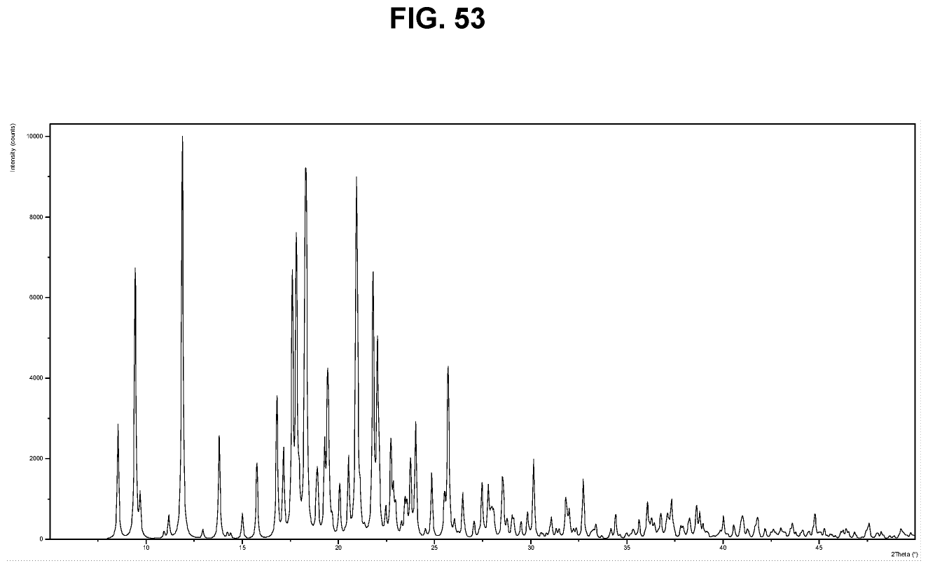

X-Ray Powder Diffraction: The XRPD pattern of Crystalline Compound A-THF Solvate is shown in FIG. 53.

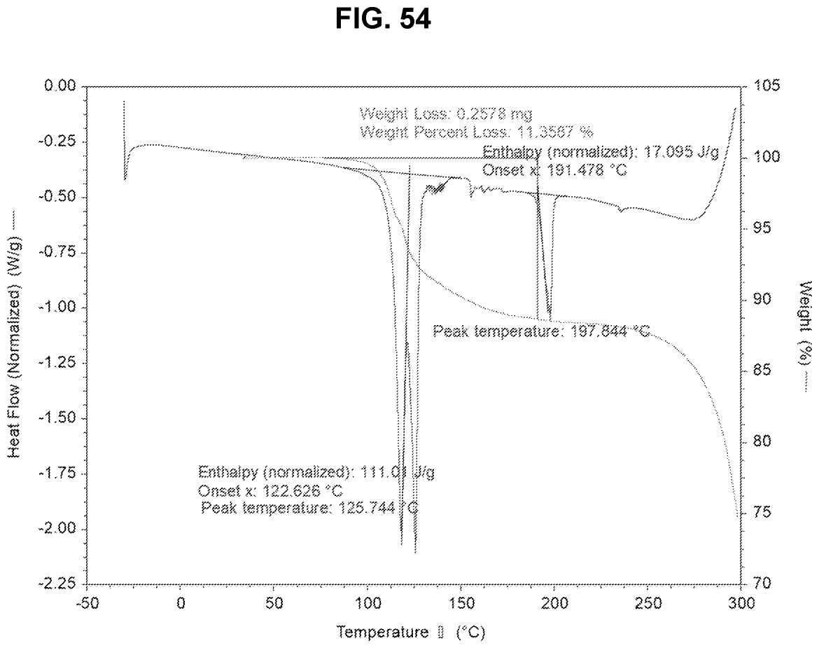

Thermal Analysis: The DSC and TGA of the Crystalline Compound A-THF Solvate are shown in FIG. 54. Typical DSC of the Crystalline Compound A-THF Solvate indicated melting onset of 122.6° C. and desolvated onset at 191.5° C. TGA of the Crystalline Compound A-THF Solvate showed a 11.4% weight loss which corresponded to a desolvation endotherm of 1 molar equivalent of THF molecule.

Single Crystal Data: Table 10 shows the Crystallographic data summary of the Crystalline Compound A-THF Solvate.

TABLE 10
X-ray Single Crystallographic data summary
of the Crystalline Compound A-THF Solvate:

| Wavelength | 0.71073 | Å |
|---|---|---|
| Crystal System | Triclinic | |
| Space Group | P-1 | |
| Unit Cell | a = 9.36460(10) Å | |
| | b = 10.6617(2) Å | |
| | c = 16.2424(3) Å | |
| | α = 79.3910(10)° | |
| | β = 80.7010(10)° | |
| | γ = 79.6150(10)° | |
| Volume | 1553.94(4) | Å³ |
| Z | 2 | |
| Density (calculated) | 1.357 | Mg/m³ |

### Example 22: Crystalline Compound A-Ethanol Solvate

Crystalline Compound A-Ethanol Solvate was prepared by slurrying the Compound A in ethanol.

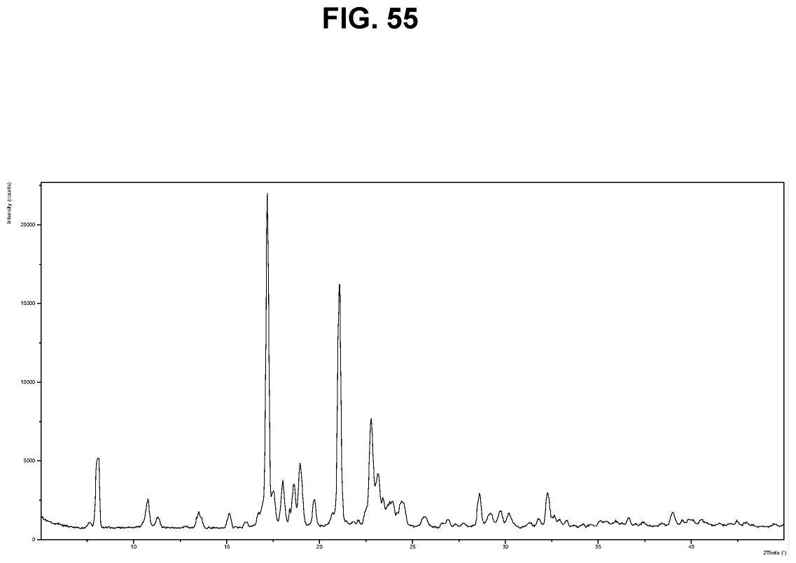

X-Ray Powder Diffraction: The XRPD pattern of Crystalline Compound A-Ethanol Solvate is shown in FIG. 55.

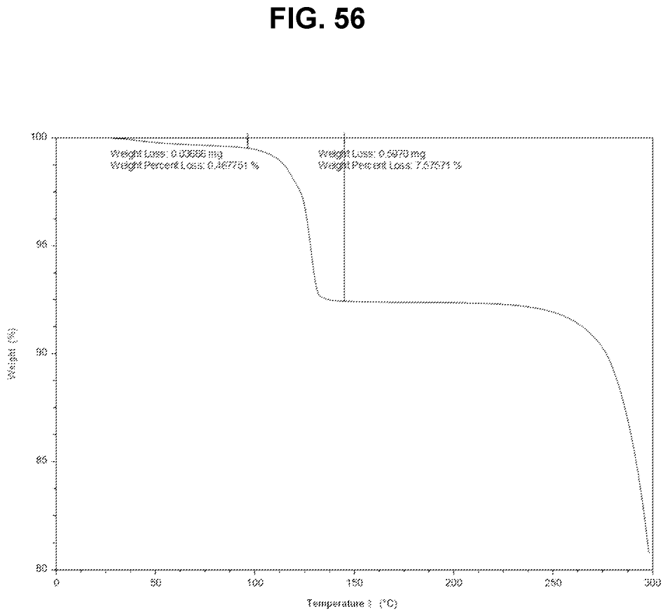

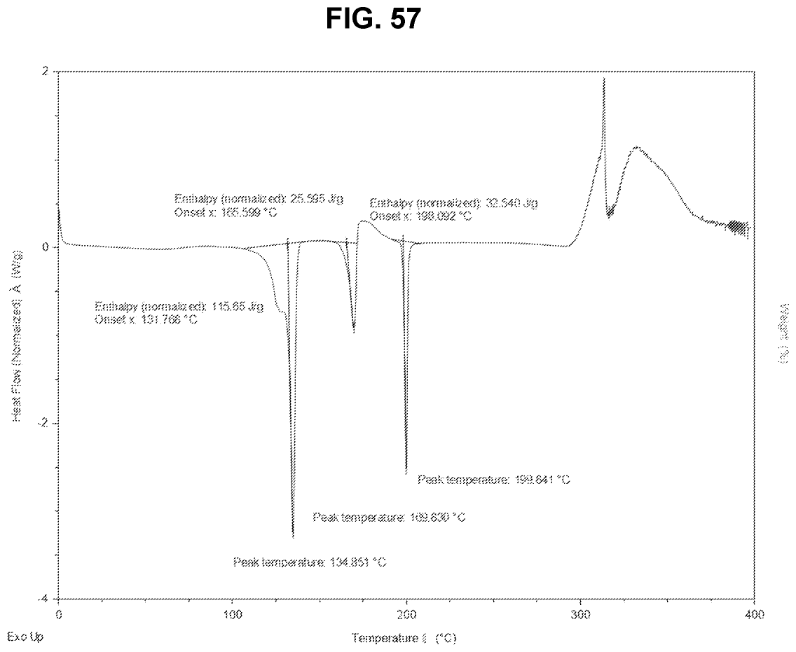

Thermal Analysis: The TGA of the Crystalline Compound A-Ethanol Solvate is shown in FIG. 56. TGA of the Crystalline Compound A-Ethanol Solvate showed a 7.58% weight loss which corresponded to loss of one molar equivalent of ethanol molecule. The DSC of the Crystalline Compound A-Ethanol Solvate is shown in FIG. 57. Typical DSC of the Crystalline Compound A-Ethanol Solvate indicated onsets of 131.8° C., 165.6° C., and 198.1° C. endothermic events.

### Example 23: Crystalline Compound A-Propanol Solvate

Crystalline Compound A-Propanol Solvate was prepared by slurrying the Compound A in 1-propanol.

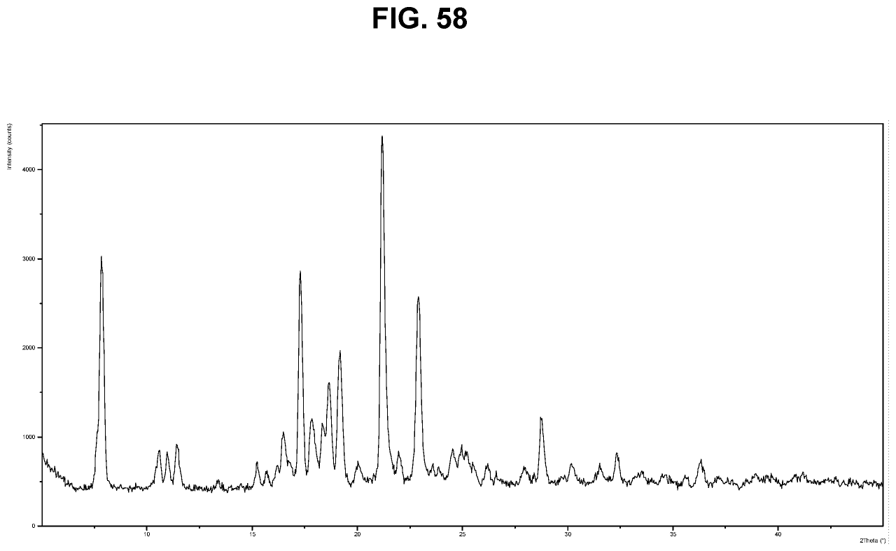

X-Ray Powder Diffraction: The XRPD pattern of Crystalline Compound A-Propanol Solvate is shown in FIG. 58.

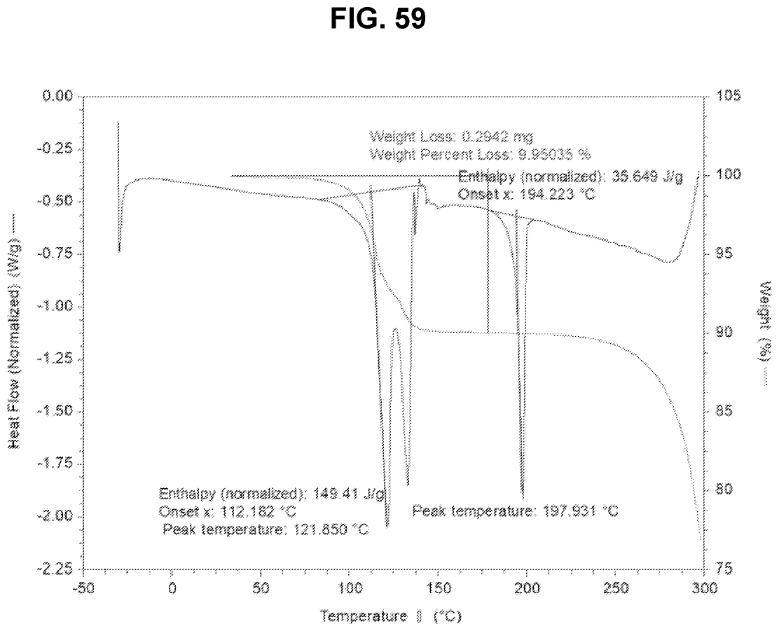

Thermal Analysis: The TGA and DSC of the Crystalline Compound A-Propanol Solvate are shown in FIG. 59. TGA of the Crystalline Compound A-Propanol Solvate showed a 9.95% weight loss which corresponded to loss of one molar equivalent of 1-propanol molecule. Typical DSC of the Crystalline Compound A-Propanol Solvate indicated melting onsets of 112.2° C. and 194.2° C.

### Example 24: Crystalline Compound A-Isopropyl Alcohol (IPA) Solvate

Crystalline Compound A-IPA Solvate was prepared by slurrying the Compound A in 50-50 1-propanol/water mixture.

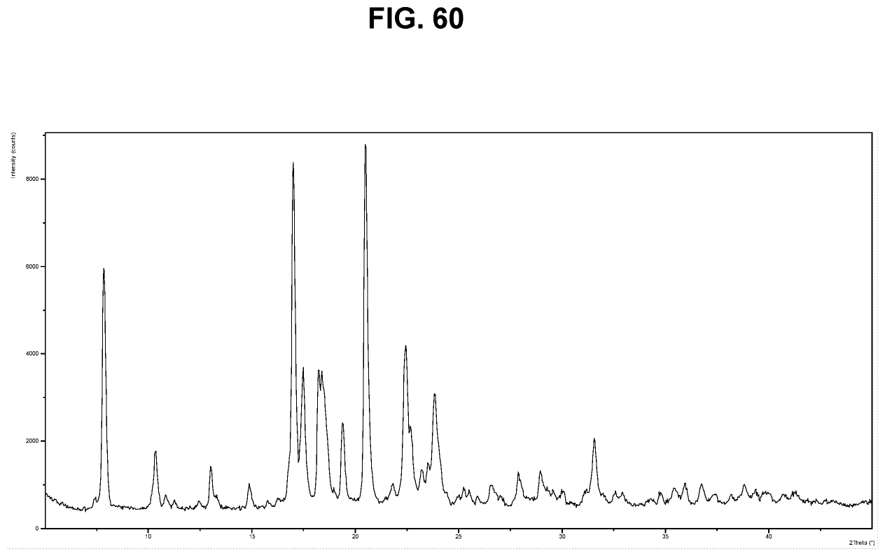

X-Ray Powder Diffraction: The XRPD pattern of Crystalline Compound A-IPA Solvate is shown in FIG. 60.

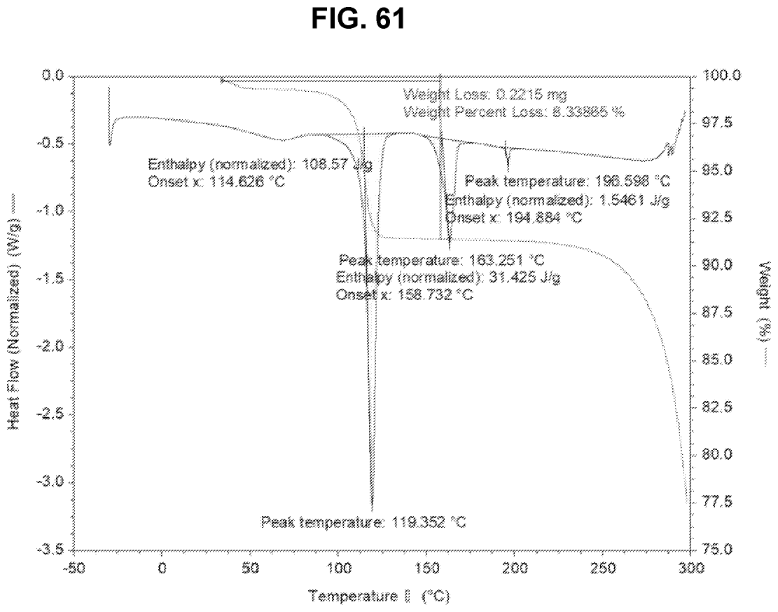

Thermal Analysis: The TGA and DSC of the Crystalline Compound A-IPA Solvate are shown in FIG. 61. TGA of the Crystalline Compound A-IPA Solvate showed an 8.5% weight loss which corresponded to loss of one molar equivalent of isopropyl alcohol molecule. Typical DSC of the Crystalline Compound A-IPA Solvate indicated onsets of 114.6° C.; 158.7° C.; and 194.9° C. endothermic events.

### Example 25: Crystalline Compound A-Methanol Solvate

Crystalline Compound A-Methanol Solvate was prepared by slurrying the Compound A in methanol.

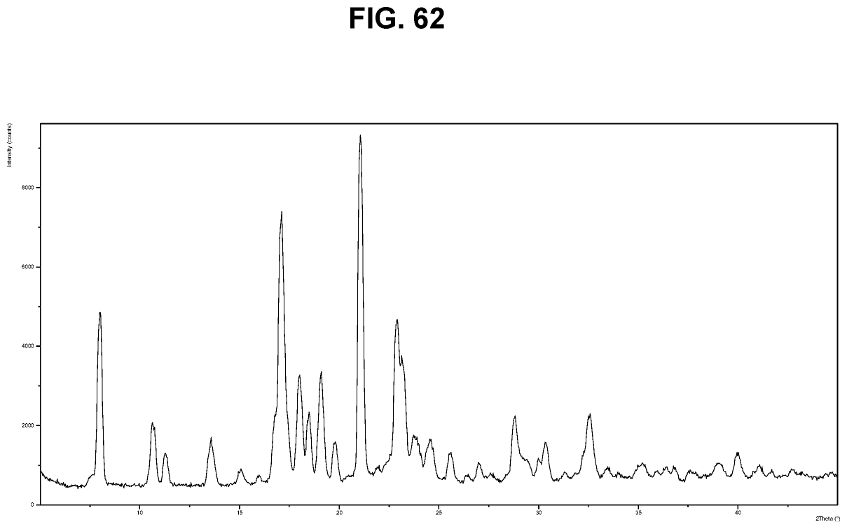

X-Ray Powder Diffraction: The XRPD pattern of Crystalline Compound A-Methanol Solvate is shown in FIG. 62.

### Example 26: Crystalline Compound A-Isopropyl Acetate (IPAc) Solvate

Crystalline Compound A-IPAc Solvate was prepared by slurrying the Compound A in isopropyl acetate.

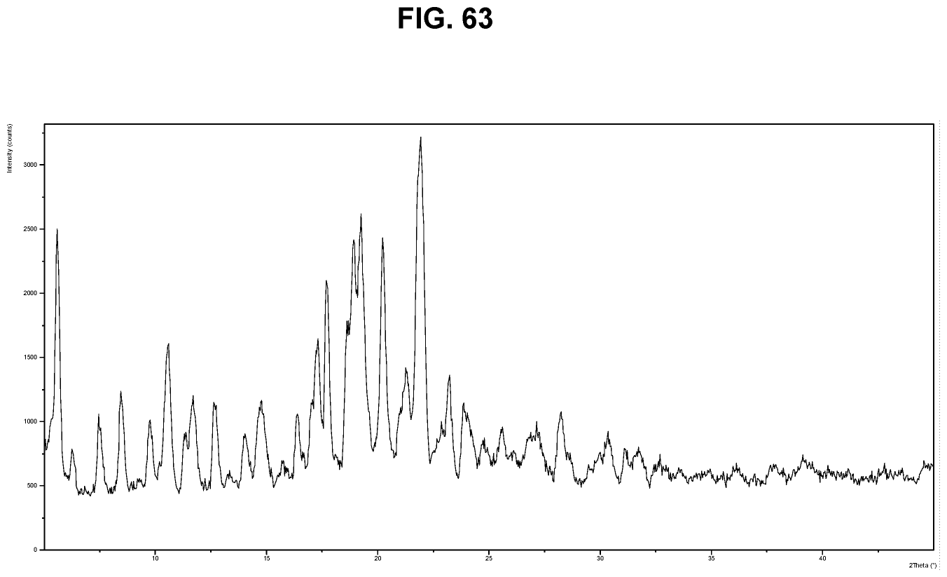

X-Ray Powder Diffraction: The XRPD pattern of Crystalline Compound A-IPAc Solvate is shown in FIG. 63.

### Example 27: Crystalline Compound A-Acetone Solvate

Crystalline Compound A-Acetone Solvate was prepared by slurrying the Compound A in acetone.

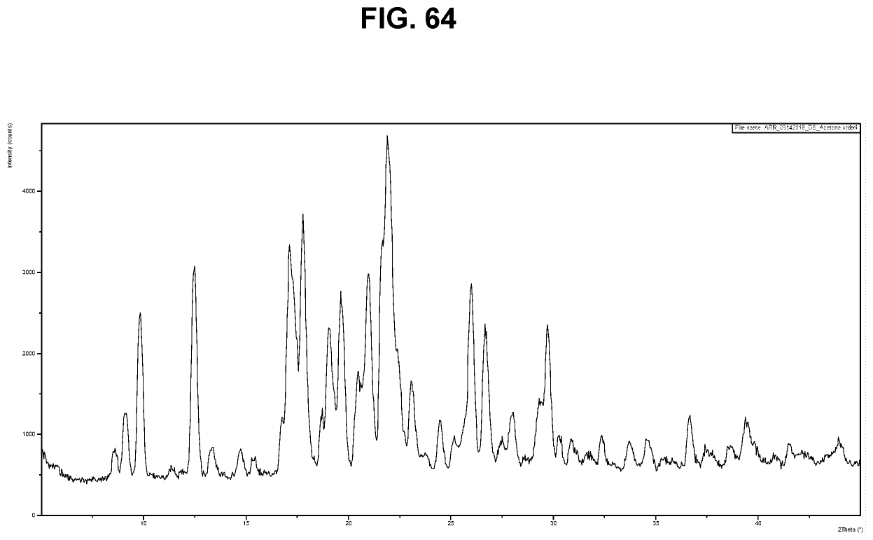

X-Ray Powder Diffraction: The XRPD pattern of Crystalline Compound A-Acetone Solvate is shown in FIG. 64.

### Example 28: Crystalline Compound A-Cyclopentyl Methyl Ether (CPME) Solvate

Crystalline Compound A-CPME Solvate was prepared by slurrying the Compound A in cyclopentyl methyl ether.

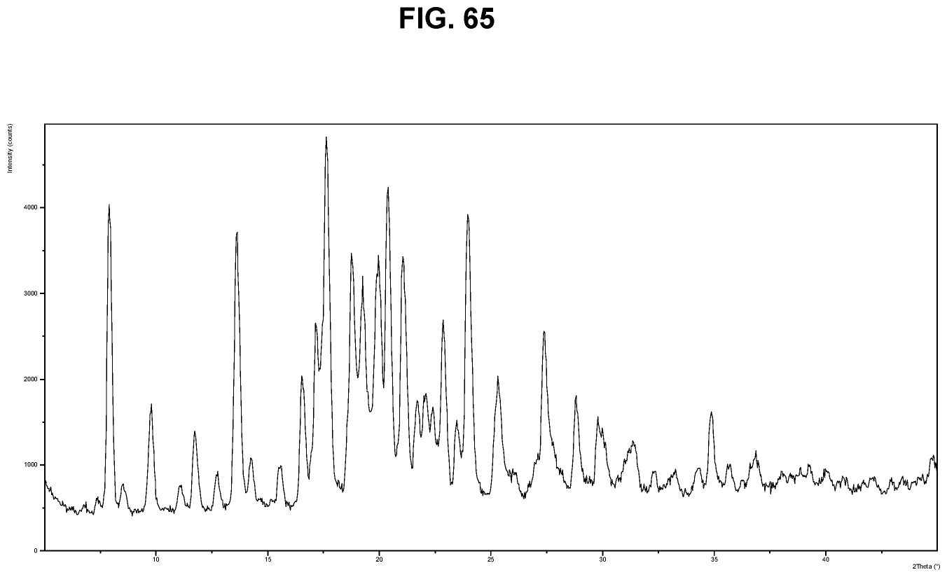

X-Ray Powder Diffraction: The XRPD pattern of Crystalline Compound A-CPME Solvate is shown in FIG. 65.

### Example 29: Crystalline Compound A-Dioxane Solvate

Crystalline Compound A-Dioxane Solvate was prepared by slurrying the Compound A in dioxane.

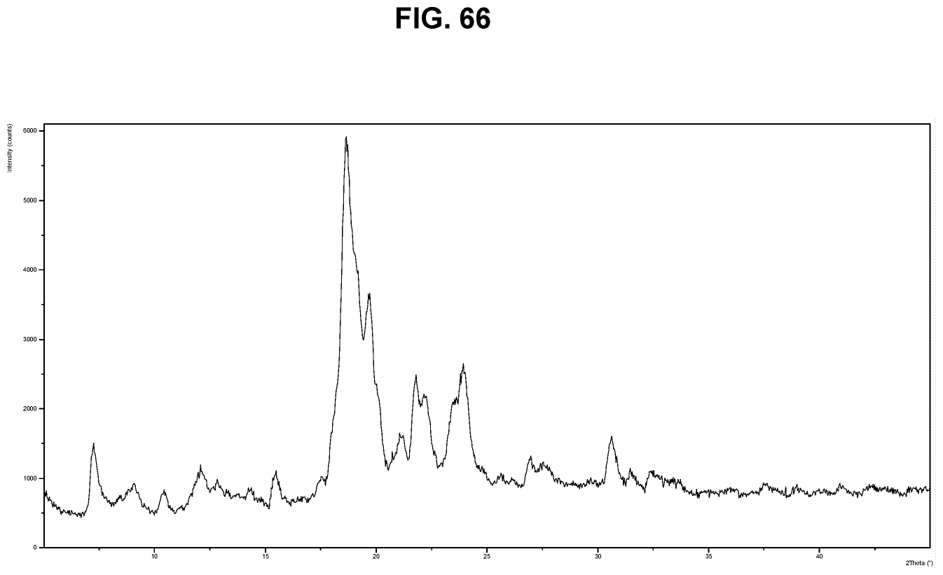

X-Ray Powder Diffraction: The XRPD pattern of Crystalline Compound A-Dioxane Solvate is shown in FIG. 66.

### Example 30: Crystalline Compound A-Ethyl Acetate (EtoAc) Solvate

Crystalline Compound A-EtOAc Solvate was prepared by slurrying the Compound A in ethyl acetate.

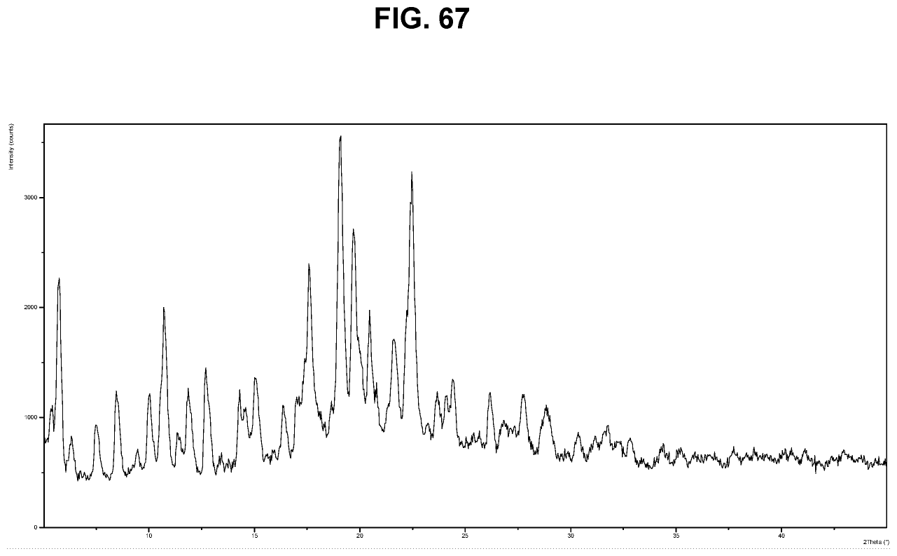

X-Ray Powder Diffraction: The XRPD pattern of Crystalline Compound A-EtOAc Solvate is shown in FIG. 67.

### Example 31: Crystalline Compound A-Acetonitrile (MeCN) Solvate

Crystalline Compound A-MeCN Solvate was prepared by slurrying the Compound A in acetonitrile.

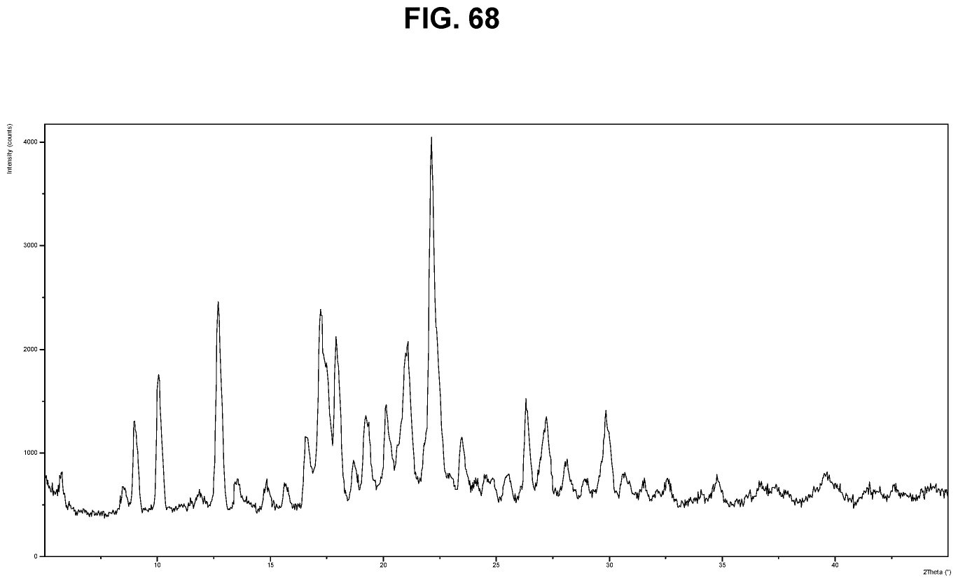

X-Ray Powder Diffraction: The XRPD pattern of Crystalline Compound A-MeCN Solvate is shown in FIG. 68.

### Example 32: Crystalline Compound A-Methyl Tert Butyl Ether (MTBE) Solvate

Crystalline Compound A-MTBE Solvate was prepared by slurrying the Compound A in methyl tert-butyl ether.

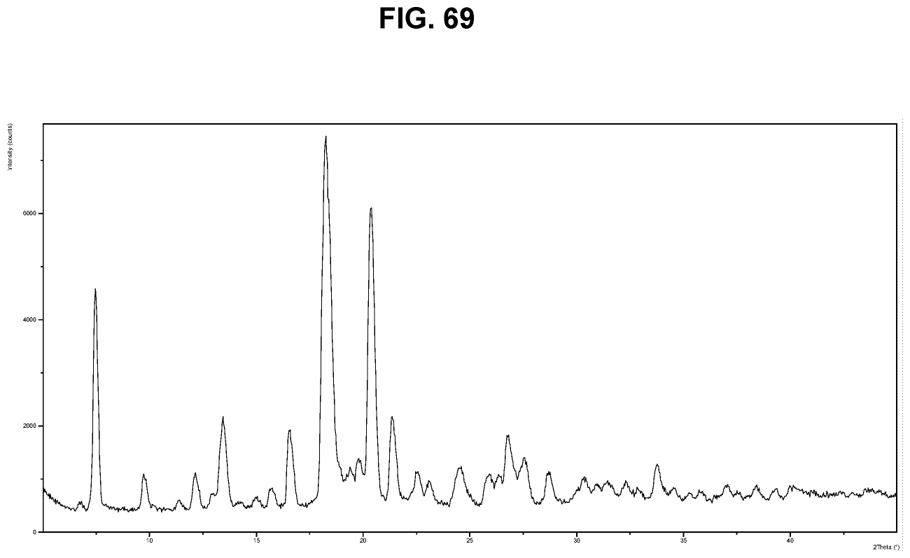

X-Ray Powder Diffraction: The XRPD pattern of Crystalline Compound A-CMTBE Solvate is shown in FIG. 69.

### Example 33: Crystalline Compound A-Toluene Solvate

Crystalline Compound A-Toluene Solvate was prepared by slurrying the Compound A in toluene at 25° C. for 18 hours.

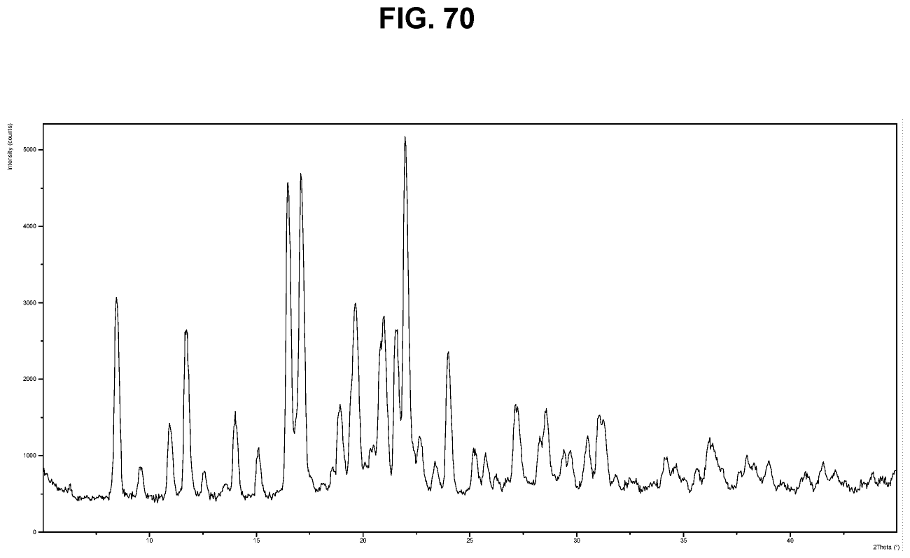

X-Ray Powder Diffraction: The XRPD pattern of Crystalline Compound A-Toluene Solvate is shown in FIG. 70.

### Example 34: Crystalline Compound A-Dodecyl Sulfate

Crystalline Compound A-Dodecyl Sulfate was prepared by slurrying 100 mg of Compound A-HCl in 0.5% sodium dodecyl sulfate (SDS) with or without 0.01N HCl at 37° C. for three hours. The solid was then removed and filtered, then washed with 1 mL DI water, and dried with a bleed of nitrogen overnight. A new crystal form was obtained, and the solution NMR analysis indicated a 1:1 API:dodecyl sulfate ratio, and assay confirmed 69% Compound A content, which correlated to one equivalent dodecyl sulfate.

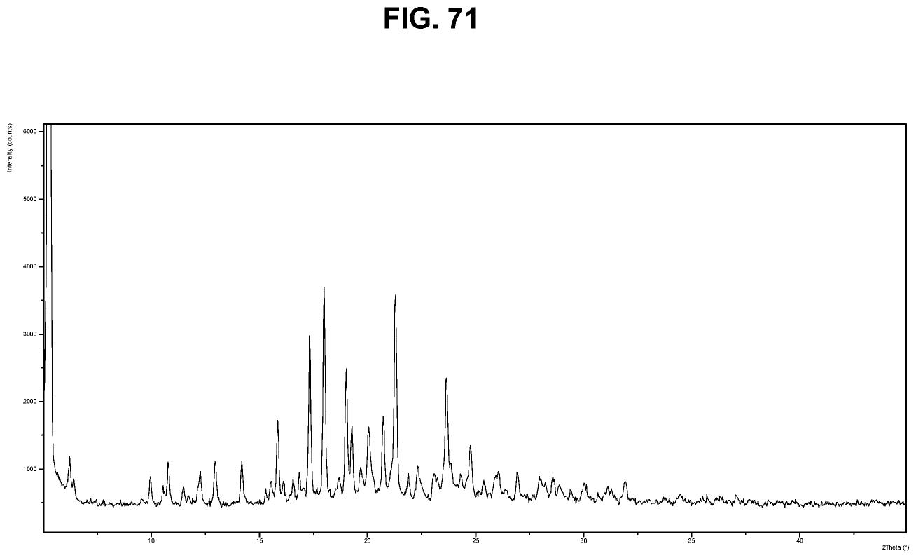

X-Ray Powder Diffraction: The XRPD pattern of Crystalline Compound A-Dodecyl Sulfate is shown in FIG. 71.

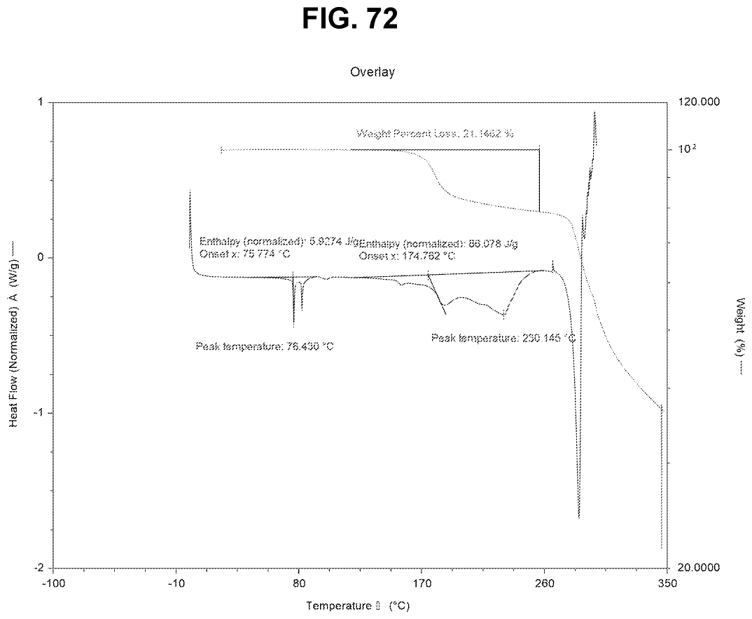

Thermal Analysis: The TGA and DSC of the Crystalline Compound A-Dodecyl Sulfate are shown in FIG. 72. TGA of the Crystalline Compound A-Dodecyl Sulfate showed a 21.1% weight loss. Typical DSC of the Crystalline Compound A-Dodecyl Sulfate indicated melting onsets of 75.8° C. and a decomposition at 174.8° C.

### Example 35: Crystalline Compound A-Dimethylformamide (DMF) Solvate Hydrate

Crystalline Compound A-DMF Solvate Hydrate was prepared by dissolving Compound A-HCl Form 1 in DMF solvent. The solution was then filtered to remove remaining solid particle from the solution. The clear solution was left for slow solvent evaporation in a fume hood at room temperature. The single crystals were observed after a week.

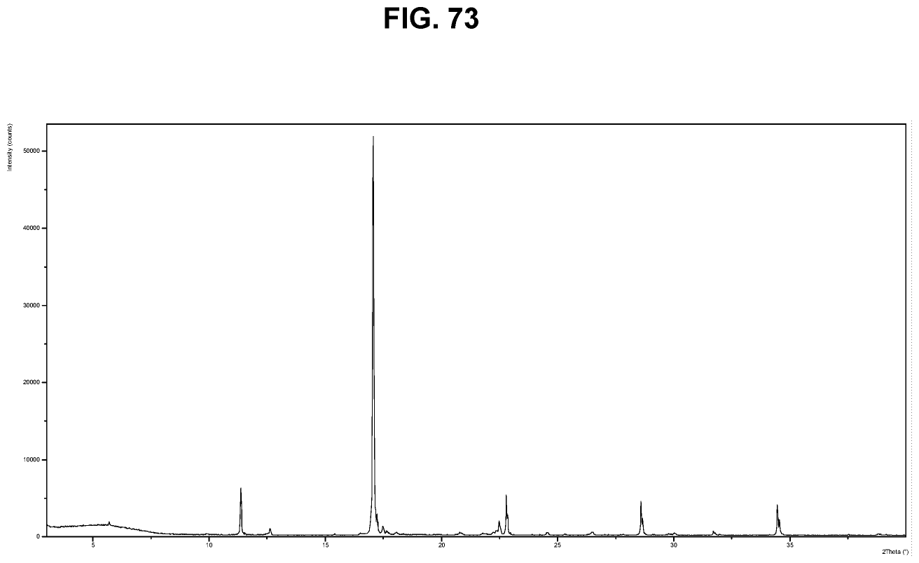

X-Ray Powder Diffraction: The XRPD pattern of Crystalline Compound A-DMF Solvate Hydrate is shown in FIG. 73.

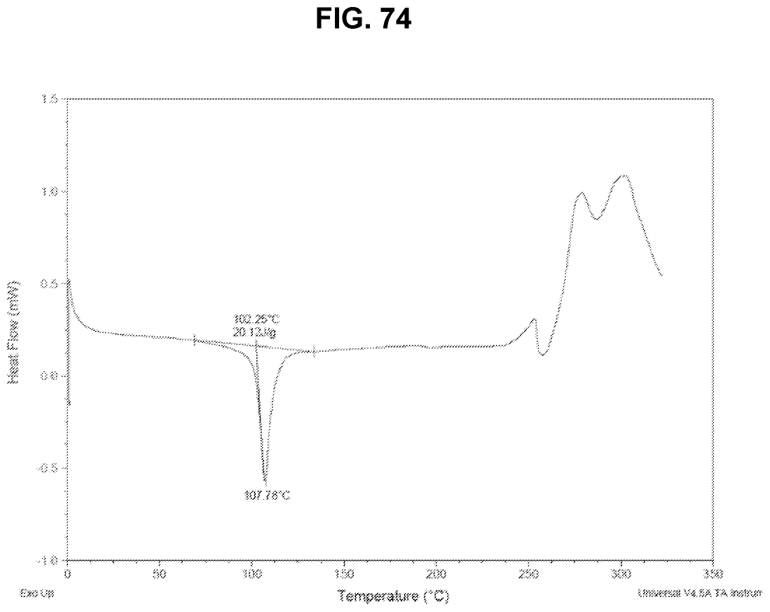

Thermal Analysis: The DSC of the Crystalline Compound A-DMF Solvate Hydrate is shown in FIG. 74. Typical DSC of the Crystalline Compound A-DMF Solvate Hydrate indicated melting onset of 107.8° C.

Single Crystal Data: In the Crystal structure of crystals provided, the DMF molecule was shown to be disordered, and the water molecule refined partial occupancy of 0.25. The DMF molecule was shown to not hydrogen bonded to the Compound A. Table 11 shows the Crystallographic data summary of the Crystalline Compound A-DMF Solvate Hydrate.

TABLE 11
X-ray Single Crystallographic data summary of
the Crystalline Compound A-DMF Solvate Hydrate:

| Wavelength | 0.710730 | Å |
|---|---|---|
| Crystal System | Triclinic | |
| Space Group | P-1 | |
| Unit Cell | a = 9.289(2) Å | |
| | b = 10.897(2) Å | |
| | c = 16.619(4) Å | |
| | α = 100.605(2)° | |
| | β = 106.204(2)° | |
| | γ = 99.216(6)° | |
| Volume | 1547.5(6) | Å³ |
| Z | 2 | |
| Density (calculated) | 1.378 | Mg/m³ |

### Example 36: Crystalline Compound A-Dimethylacetamide (DMAC) Solvate

Crystalline Compound A-DMAC Solvate was prepared by dissolving Compound A-HCl Form 1 in DMAC solvent. The solution was then filtered to remove remaining solid particle from the solution. The clear solution was left for slow solvent evaporation in a fume hood at room temperature. The single crystals were observed after a week.

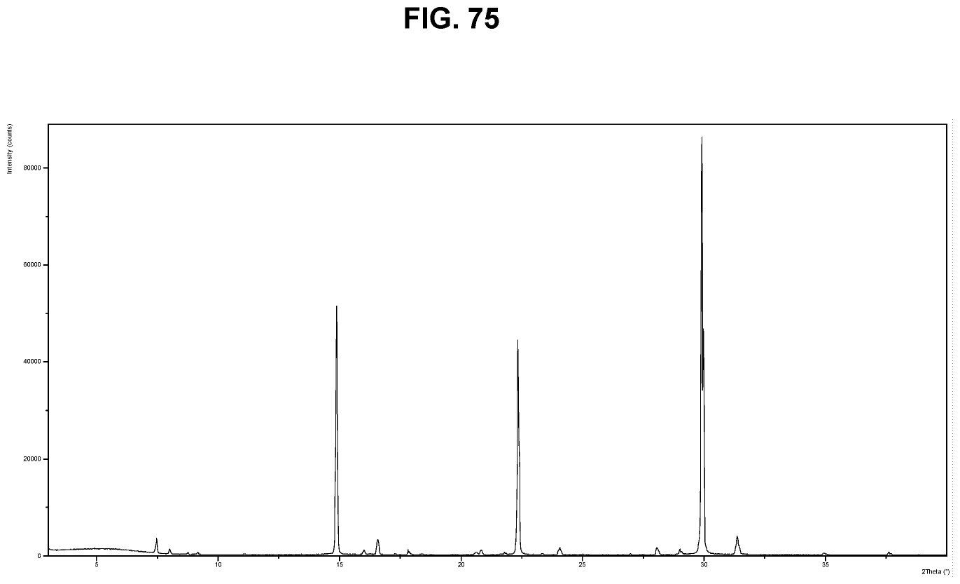

X-Ray Powder Diffraction: The XRPD pattern of Crystalline Compound A-DMAC Solvate is shown in FIG. 75.

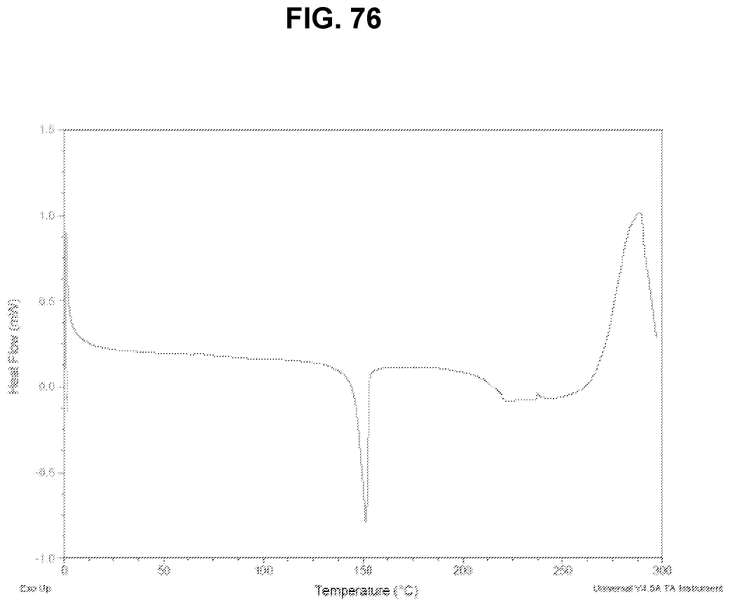

Thermal Analysis: The DSC of the Crystalline Compound A-DMAC Solvate is shown in FIG. 76. Typical DSC of the Crystalline Compound A-DMAC Solvate indicated a melting onset of about 150° C.

Single Crystal Data: In the Crystal structure of crystals provided, the DMAC molecule was shown to be disordered. However, the DMAC molecule was shown to still be hydrogen bonded to the Compound A. Table 12 shows the Crystallographic data summary of the Crystalline Compound A-DMAC Solvate.

TABLE 12
X-ray Single Crystallographic data summary
of the Crystalline Compound A-DMAC Solvate:

| Wavelength | 0.710730 | Å |
|---|---|---|
| Crystal System | Triclinic | |
| Space Group | P-1 | |
| Unit Cell | a = 12.5010(15) Å | |
| | b = 12.5268(15) Å | |
| | c = 13.2651(15 Å | |
| | α = 77.2990(10)° | |
| | β = 65.6460(10)° | |
| | γ = 61.1020(3)° | |
| Volume | 1656.2(3) | Å³ |
| Z | 2 | |
| Density (calculated) | 1.3333 | Mg/m³ |

### Example 37: Crystalline Compound A-Mono Besylate Hydrate Form 1

Crystalline Compound A-Mono Besylate Hydrate Form 1 was prepared by dissolving 92.6 mg of Compound A and 29.3 mg of benzenesulfonic acid in 1 mL methanol solvent. The solution was then stirred at 60° C. for 1 day. A slurry resulted and the solids were isolated by vacuum filtration. The solids were air dried for 1 hour and then analyzed.

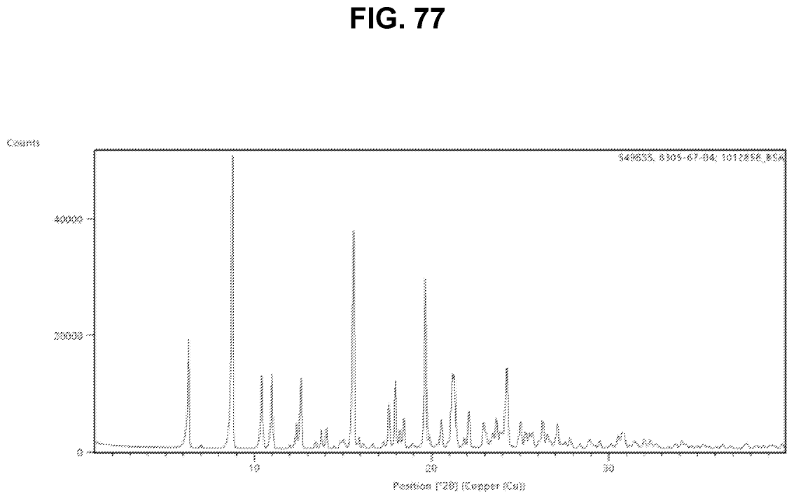

X-Ray Powder Diffraction: The XRPD pattern of Crystalline Compound A-Mono Besylate Hydrate Form 1 is shown in FIG. 77.

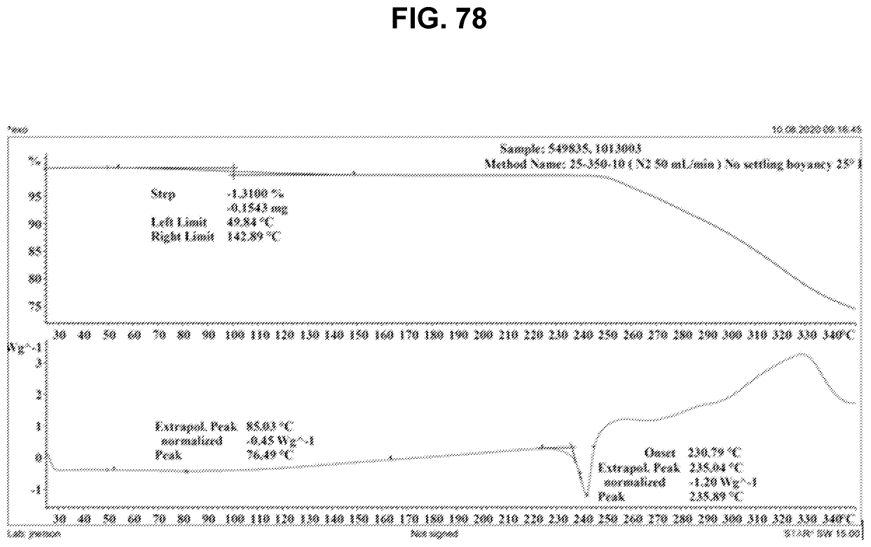

Thermal Analysis: The DSC and TGA of the Crystalline Compound A-Mono Besylate Hydrate Form 1 are shown in FIG. 78, which indicated a melting onset of about 230.8° C. TGA of the Crystalline Compound A-Mono Besylate Hydrate Form 1 showed about 1.3% weight loss up to 142.3° C.

### Example 38: Crystalline Compound A-Caffeine Co-Crystal Form 1

Compound A-Caffeine Co-crystal Form 1 was prepared by a slow cooling experiment in acetonitrile from 70° C. to 5° C. using a 1:1 Compound A:Caffeine molar ratio. The resulting product contains the remaining Compound A starting material mixed with the Caffeine Co-crystal Form 1 along with other impurities, which were not further identified. The resulting product was then further purified by heating the mixture to 167° C. in DSC furnace under nitrogen flow to form pure Compound A-Caffeine Co-crystal Form 1.

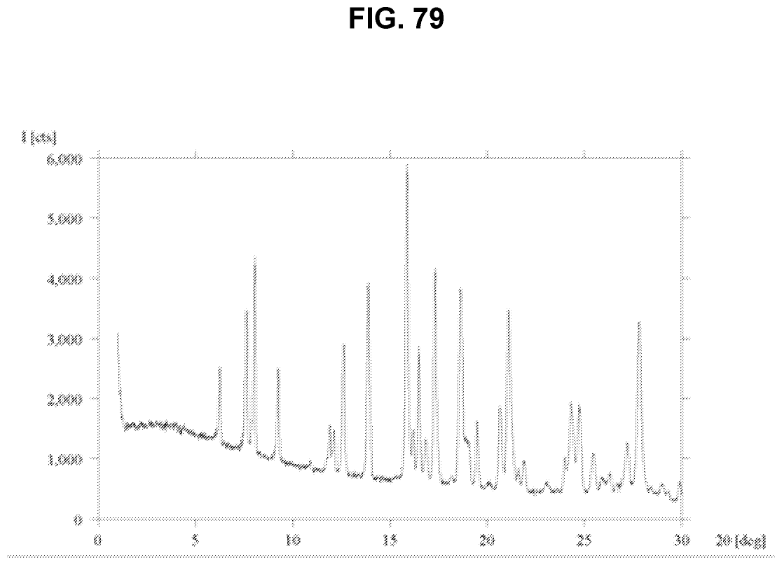

X-Ray Powder Diffraction: The XRPD pattern of Crystalline Compound A-Caffeine Co-crystal Form 1 is shown in FIG. 79. Single Crystal Structure data of the Crystalline Compound A-Caffeine Co-crystal Form 1 is tabulated in Table 13 below.

TABLE 13
Single Crystal Data of Crystalline Compound
A-Caffeine Co-crystal Form 1:

| Wavelength | 1.5418 | Å |
|---|---|---|
| Crystal System | Triclinic | |
| Space Group | P-1 | |
| Unit Cell | a = 11.2224 Å | |
| | b = 11.5160 Å | |
| | c = 14.1909 Å | |
| | α = 96.317° | |
| | β = 96.403° | |
| | γ = 100.279° | |
| Volume | 1777.40 | Å³ |
| Z | 4 | |
| Density (calculated) | 1.418 | Mg/m³ |

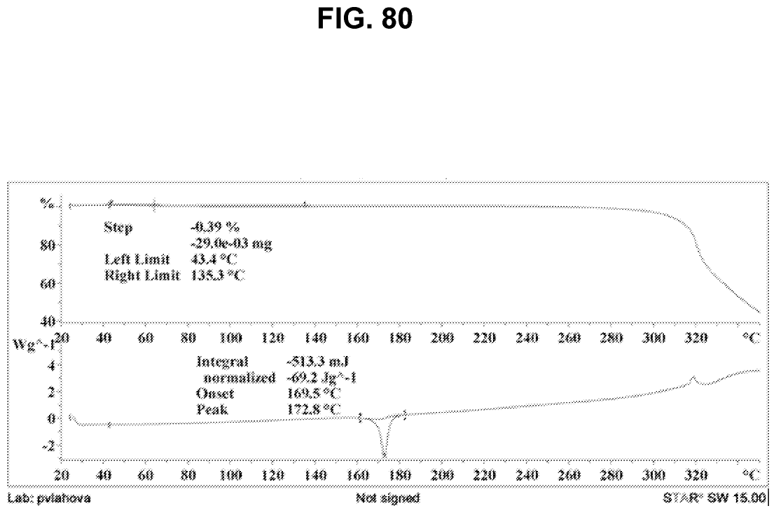

Thermal Analysis: The DSC and TGA patterns of the Crystalline Compound A-Caffeine Co-crystal Form 1 are shown in FIG. 80. The DSC indicated a melting onset of about 169.5° C. TGA of the Crystalline Compound A-Caffeine Co-crystal Form 1 showed about 0.39% weight loss of up to 135.3° C.

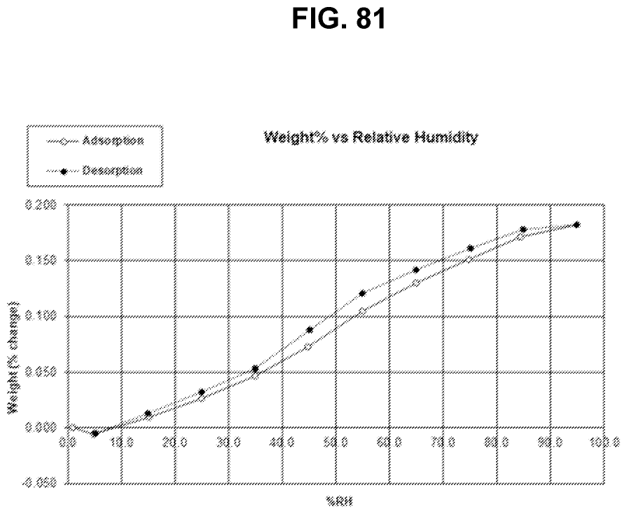

Hygroscopicity Analysis: The hygroscopic profile of the Crystalline Compound A-Caffeine Co-crystal Form 1 is shown in FIG. 81. Dynamic Vapor Sorption (DVS) of the Crystalline Compound A-Caffeine Co-crystal Form 1 showed a weight gain of lower than 0.20% at about 95% RH.

### Example 39: Crystalline Compound A-Citric Acid Co-Crystal Form 1

Crystalline Compound A-Citric Acid Co-crystal Form 1 was obtained by a slow cooling experiment in ethyl acetate from 70° C. to 5° C. using a 1:1 Compound A:citric acid molar ratio.

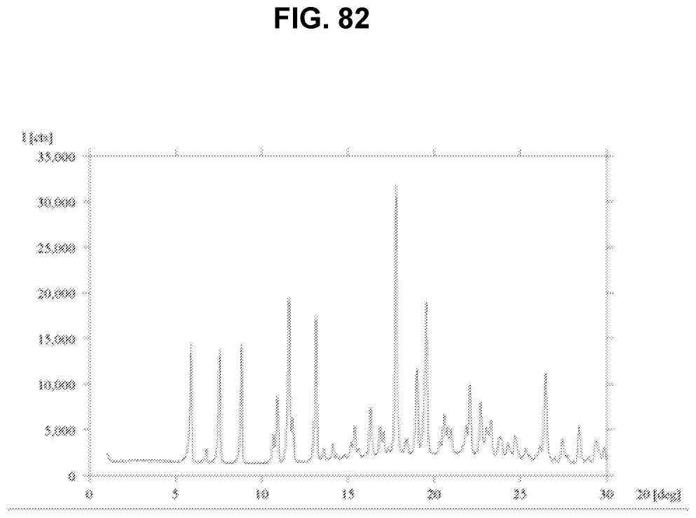

X-Ray Powder Diffraction: The XRPD pattern of Crystalline Compound A-Citric Acid Co-crystal Form 1 is shown in FIG. 82. Crystalline Compound A-Citric Acid Co-crystal Form 1 was collected with Cu-Kα radiation, and the indexing results are tabulated in Table 14 below.

TABLE 14
Indexing Result for XRPD Data of Crystalline
Compound A-Citric Acid Co-crystal Form 1:

| Bravais Lattice Type | Triclinic |
|---|---|
| a [Å] | 10.062 |
| b [Å] | 13.643 |
| c [Å] | 15.685 |
| α [deg] | 107.03 |
| β [deg] | 91.86 |
| γ [deg] | 94.05 |
| Volume [Å³/cell] | 2,505.5 |
| Chiral Contents? | Not specified |
| Extinction Symbol | P - |
| Space Group(s) | P1 (1), PĪ (2) |

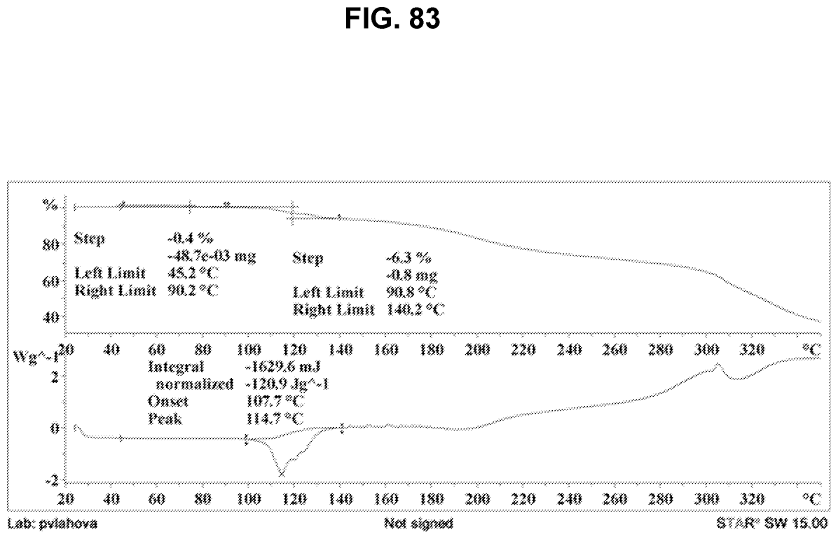

Thermal Analysis: The DSC and TGA of the Crystalline Compound A-Citric Acid Co-crystal Form 1 are shown in FIG. 83, which indicated a melting onset of about 107.7° C. TGA of the Crystalline Compound A-Citric Acid Co-crystal Form 1 showed about 6.3% weight loss of 0.8 mg up to 140.2° C.

### Example 40: Crystalline Compound A-Citric Acid Co-Crystal Form 2

Crystalline Compound A-Citric Acid Co-crystal Form 2 was obtained by a slow cooling experiment in acetonitrile from 70° C. to refrigerator temperature using a 1:2 Compound A:citric acid molar ratio. The sample initially oiled out and was stirred at 5° C. for 3 days producing an off-white precipitate.

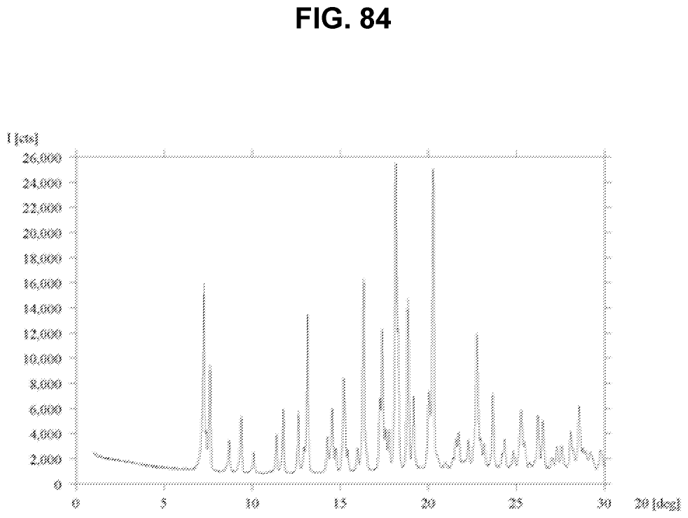

X-Ray Powder Diffraction: The XRPD pattern of Crystalline Compound A-Citric Acid Co-crystal Form 2 is shown in FIG. 84. Crystalline Compound A-Citric Acid Co-crystal Form 2 was collected with Cu-Kα radiation, and the indexing results are tabulated in Table 15 below.

TABLE 15
Indexing Result for XRPD Data of Crystalline
Compound A-Citric Acid Co-crystal Form 2:

| Bravais Lattice Type | Triclinic |
|---|---|
| a [Å] | 12.564 |
| b [Å] | 13.497 |
| c [Å] | 14.075 |
| α [deg] | 115.41 |
| β [deg] | 103.54 |
| γ [deg] | 93.81 |
| Volume [Å³/cell] | 2,057.8 |
| Chiral Contents? | Not specified |
| Extinction Symbol | P - |
| Space Group(s) | P1 (1), PĪ (2) |

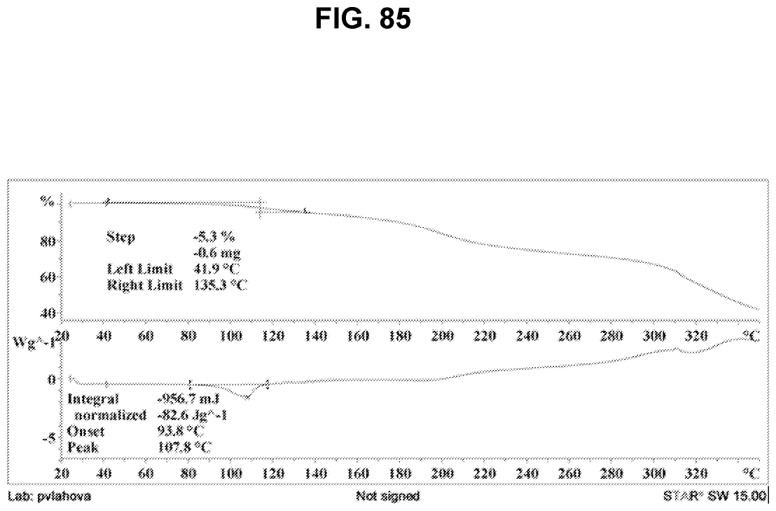

Thermal Analysis: The DSC and TGA patterns of the Crystalline Compound A-Citric Acid Co-crystal Form 2 are shown in FIG. 85. The DSC indicated an endothermic onset of about 93.8° C. TGA of the Crystalline Compound A-Citric Acid Co-crystal Form 2 showed about 5.3% weight loss of 0.6 mg up to 135.3° C.

### Example 41: Crystalline Compound A-Saccharin-Co-Crystal Form 1

Crystalline Compound A-Saccharin Co-crystal Form 1 was prepared by a slow cooling experiment in acetonitrile from 70° C. to 5° C. using a 1:1 Compound A:Saccharin molar ratio.

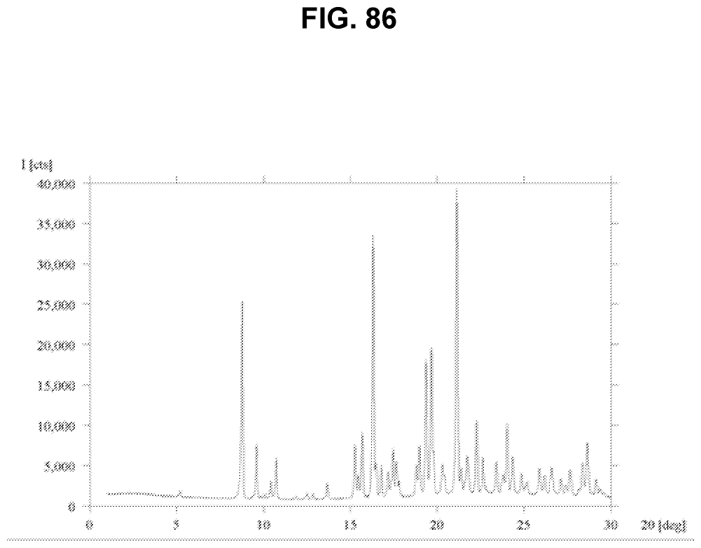

X-Ray Powder Diffraction: The XRPD pattern of Crystalline Compound A-Saccharin Co-crystal Form 1 is shown in FIG. 86. Crystalline Compound A-Saccharin Co-crystal Form 1 was collected with Cu-Kα radiation, and the indexing results are tabulated in Table 16 below.

TABLE 16
Indexing Result for XRPD Data of Crystalline
Compound A-Saccharin Co-crystal Form 1:

| Bravais Lattice Type | Triclinic |
|---|---|
| a [Å] | 10.249 |
| b [Å] | 11.000 |
| c [Å] | 17.389 |
| α [deg] | 91.62 |
| β [deg] | 101.30 |
| γ [deg] | 113.03 |
| Volume [Å³/cell] | 1,757.2 |
| Chiral Contents? | Not specified |
| Extinction Symbol | P - |
| Space Group(s) | P1 (1), PĪ (2) |

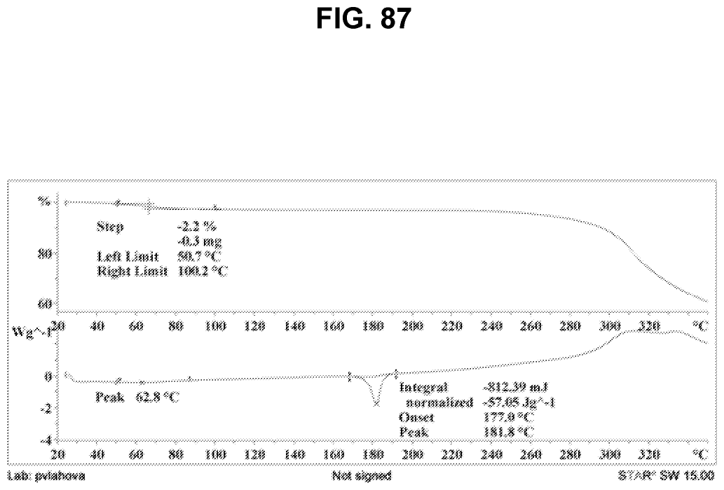

Thermal Analysis: The DSC and TGA of the Crystalline Compound A-Saccharin Co-crystal Form 1 are shown in FIG. 87. The DSC indicated a melting onset of about 177.0° C. TGA of the Crystalline Compound A-Saccharin Co-crystal Form 1 showed about 2.2% weight loss of 0.3 mg up to 100.2° C.

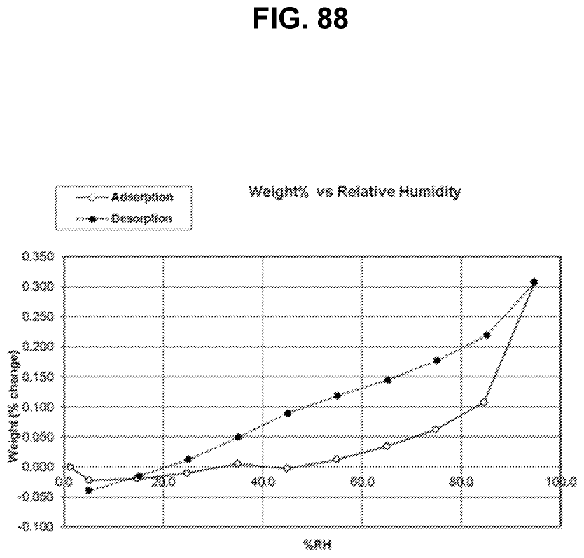

Hygroscopicity data: The hygroscopic profile of the Crystalline Compound A-Saccharin Co-crystal Form 1 is shown in FIG. 88. Dynamic Vapor Sorption (DVS) of the Crystalline Compound A-Saccharin Co-crystal Form 1 showed a weight gain of about 0.3% by 95% RH.

### Example 42: Crystalline Compound A-L-Tartaric Acid Co-Crystal Form 1

Crystalline Compound A-L-Tartaric Acid Co-crystal Form 1 as prepared by a slow cooling experiment in acetonitrile from 70° C. to 5° C. using a 1:1 of the Compound A:L-tartaric acid molar ratio.

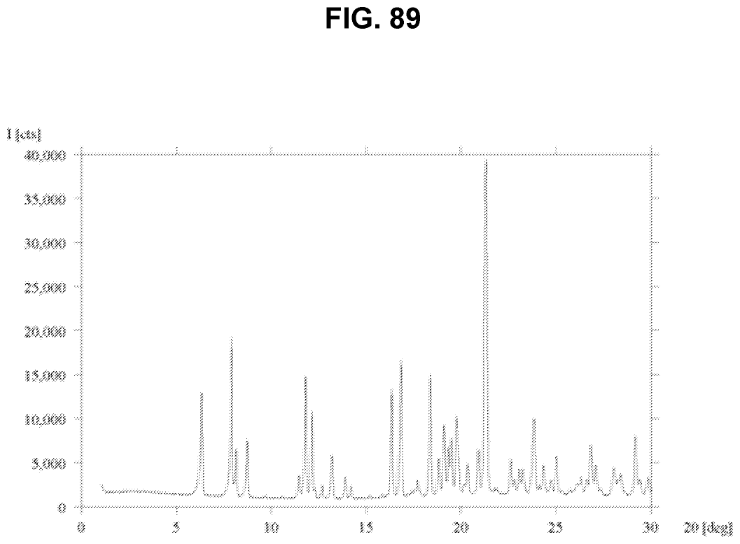

X-Ray Powder Diffraction: The XRPD pattern of Crystalline Compound A-L-Tartaric Acid Co-crystal Form 1 is shown in FIG. 89. Crystalline Compound A-L-Tartaric Acid Co-crystal Form 1 was collected with Cu-Kα radiation, and the indexing results are tabulated in Table 17 below.

TABLE 17
Indexing Result for XRPD Data of Crystalline
Compound A-L-Tartaric Acid Co-crystal Form 1:

| Bravais Lattice Type | Triclinic |
|---|---|
| a [Å] | 10.417 |
| b [Å] | 12.106 |
| c [Å] | 15.398 |
| α [deg] | 67.51 |
| β [deg] | 76.14 |
| γ [deg] | 81.86 |
| Volume [Å³/cell] | 1,739.2 |
| Chiral Contents? | Chiral |
| Extinction Symbol | P - |
| Space Group(s) | P1 (1) |

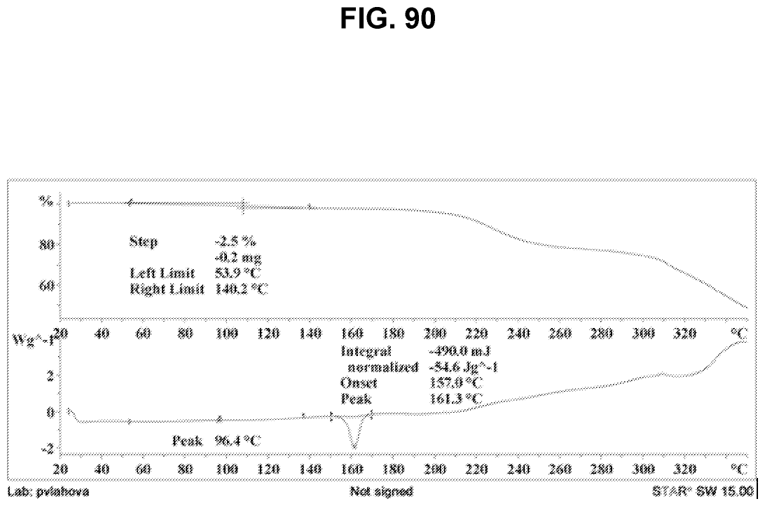

Thermal Analysis: The DSC and TGA of the Crystalline Compound A-L-Tartaric Acid Co-crystal Form 1 are shown in FIG. 90. The DSC indicated an onset of about 157.0° C. TGA of the Crystalline Compound A-Tartaric Acid Co-crystal Form 1 showed about 2.5% weight loss of 0.2 mg up to 140.2° C.

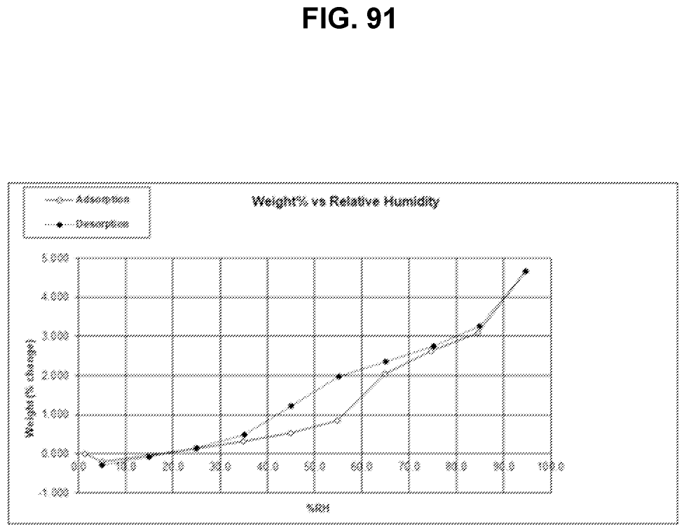

Hygroscopicity data: The hygroscopic profile of the Crystalline Compound A-L-Tartaric Acid Co-crystal Form 1 is shown in FIG. 91. Dynamic vapor sorption of the Crystalline Compound A-L-Tartaric Acid Co-crystal Form 1 showed a weight gain of about 4.75% by 95% RH.

### Example 43: Crystalline Compound A-Urea Co-Crystal Form 1

Crystalline Compound A-Urea Co-crystal Form 1 was prepared by a slow cooling experiment in acetonitrile from 70° C. to freezer temperature between −15° C. to −25° C., using a 2:1 of the Compound A:Urea molar ratio.

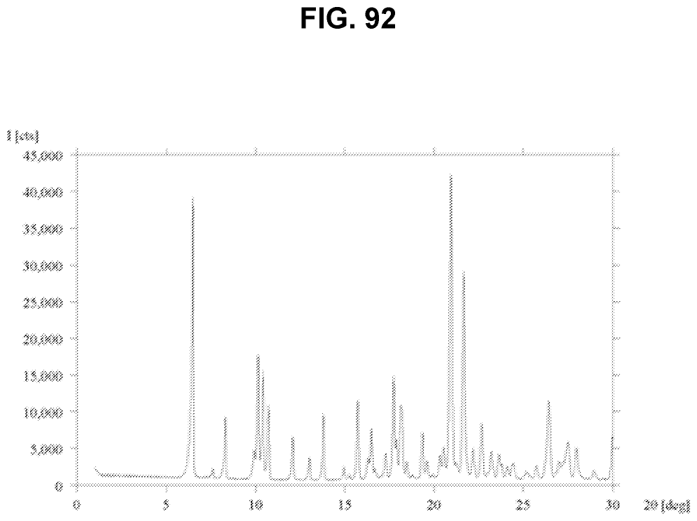

X-Ray Powder Diffraction: The XRPD pattern of Crystalline Compound A-Urea Co-crystal Form 1 is shown in FIG. 92. Crystalline Compound A-Urea Co-crystal Form 1 was collected with Cu-Kα radiation, and the indexing results are tabulated in Table 18 below.

TABLE 18
Indexing Result for XRPD Data of Crystalline
Compound A-Urea Co-crystal Form 1:

| Bravais Lattice Type | Triclinic |
|---|---|
| a [Å] | 10.754 |
| b [Å] | 11.715 |
| c [Å] | 13.612 |
| α [deg] | 88.37 |
| β [deg] | 87.73 |
| γ [deg] | 81.51 |
| Volume [Å³/cell] | 1,694.3 |
| Chiral Contents? | Not specified |
| Extinction Symbol | P - |
| Space Group(s) | P1 (1), PĪ (2) |

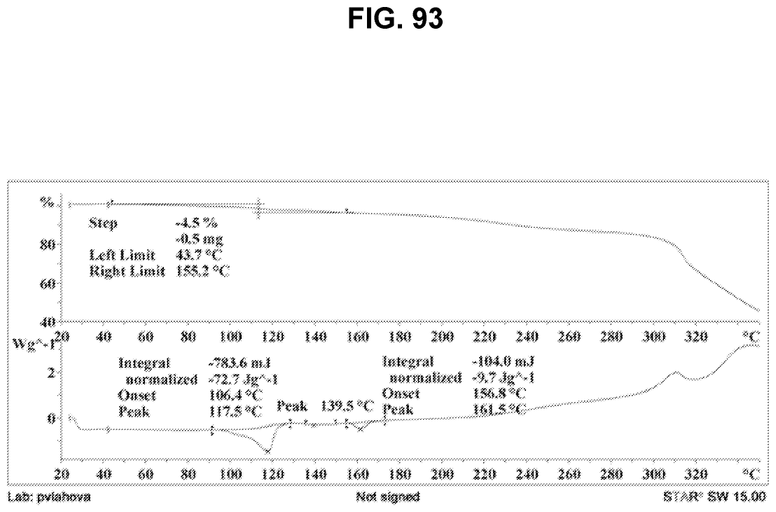

Thermal Analysis: The DSC and TGA of the Crystalline Compound A-Urea Co-crystal Form 1 are shown in FIG. 93. The DSC indicated a first endothermic onset of about 106.4° C. and a second endothermic onset of about 156.8° C. TGA of the Crystalline Compound A-Urea Co-crystal Form 1 showed about 4.5% weight loss of 0.5 mg up to 155.2° C.

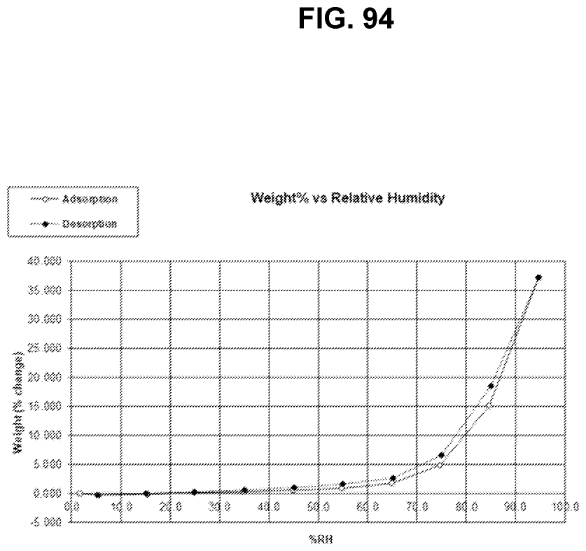

Hygroscopicity data: The hygroscopic profile of the Crystalline Compound A-Urea Co-crystal Form 1 is shown in FIG. 94. Dynamic Vapor Sorption (DVS) of the Crystalline Compound A-Urea Co-crystal Form 1 showed a weight gain of less than 40% by 95% RH.

### Solubility, Powder Dissolution (PD) and Intrinsic Dissolution Rate (IDR) Test

### Example 44: PD and IDR Tests of Compound A-HCl Form 1 Compared to Various Forms of Non-Salt Compound A

Solubilities of various forms of the Compound A and the Compound A-HCl Form 1 were measured in Fasted State Simulated Gastric Fluid (FaSSGF), Fasted State Simulated Intestinal Fluid (FaSSIF), fed state simulated intestinal fluid (FaSSIF), and water. The powder dissolution measurement test results showed Crystalline Compound A-HCl Form 1 exhibited a faster dissolution than the Compound A-Variable-Hydrate Form 2, or Compound A-Anhydrous Form 3, but a slower dissolution than the Amorphous Compound A. The solubility and IDR data are listed in Tables 19 and 20, respectively. The data shows that Crystalline Compound A-HCl Form 1 has solubility and IDR advantages compared to any of the forms tested here.

TABLE 19
Solubility Test Result of Compound A-HCl Form 1 compared to various Compound A Forms.

| | Media | | | | | | | | |
|---|---|---|---|---|---|---|---|---|---|
| | FaSSGF | | FaSSIF | | FeSSIF | Water | | | |
| Forms | Sol (pH), μg/mL | | Sol (pH), μg/mL | | Sol (pH), μg/mL | Sol (pH), μg/mL | | | |
| Cmp A1-HCl Form 1 | 6.3 ± 0.5 | (1.6)\hat{ } | 62.7 ± 9.0 | (6.5)\hat{ } | 437.1 ± 16.3 (5.0)\hat{ } | 7.9 | (1.14) | 13 | (1.58) |
| Cmp A-Variable- | 20 | (1.6)\hat{ } | 10.6 | (6.5)\hat{ } | 144.5(5.0)\hat{ } | 243 | (1.08) | 23 | (1.64) |
| Hydrate Form 2 | | | | | | | | | |
| Cmp A-Anhydrous | 99.3 | (1.6)* | 87 | (6.5)* | 352 (5.0)* | 5.4 (8.0)* | | | |
| Form 3 | | | | | | | | | |

Cmp = Compound.
\hat{ }Solubility from powder dissolution measurements.
*Solubility from 2 hr time point, pH from the end of experiments

TABLE 20
IDR Test Result of Compound A-HCl Form 1 compared
to Compound A Variable Hydrate Form 2 (See
Example 13) and Amorphous Compound A Form.

| Forms | IDR (ug/min*cm²) |
|---|---|
| Cmp A-HCl Form 1 | 2.14 |
| Cmp A-Variable Hydrate Form 2 | 0.42 |
| Amorphous Cmp A | 36.2 |

Cmp = Compound.

### Example 45: Biological Data

Dog Cross-over PK Study of Compound A-HCl Form 1, Compound A-Anhydrous Form 3, and Amorphous Compound A.

A total of 3 male dogs were initially assigned to study. All animals were fasted for at least eight hours prior to dosing and through the first four hours of blood sample collection (food was returned within 30 minutes following the collection of the last blood sample at the 4 hour collection interval, if applicable).

Each animal receiver an oral gavage dose (PO) of the appropriate test article solution containing Compound A as outlined in the following study design table. Oral gavage dosing solutions were continuously stirred throughout dosing. The gavage tubes were rinsed with approximately 10 mL of tap water following dosing (prior to removal of the gavage tube). There was a minimum of 10-day washout period between doses for each phase.

TABLE 21
Dog Cross-Over PK Study Protocol Summary

| | | | | | |
|---|---|---|---|---|---|
| | | | Dose | Dose | |
| Test | Dose | | Level | Volume | Collection |
| Article | Route | Vehicle | (mg/kg) | (mL/kg) | Intervals |

Phase 1

| | | | | | |
|---|---|---|---|---|---|
| Cmp A-HCl | PO | A | 10 | 5 | Blood^{a} |
| Form 1 | | | | | |

Phase 2

| | | | | | |
|---|---|---|---|---|---|
| Cmp A- | PO | A | 10 | 5 | Blood^{a} |
| Anhydrous | | | | | |
| Form 3 | | | | | |

Phase 3

| | | | | | |
|---|---|---|---|---|---|
| Amorphous | PO | a | 10 | 5 | Blood^{a} |
| Cmp A | | | | | |

^{a}Blood samples for plasma were collected predose and at 0.25, 0.5, 1, 2, 4, 6, 8, 24, 48, and 72 hours postdose.

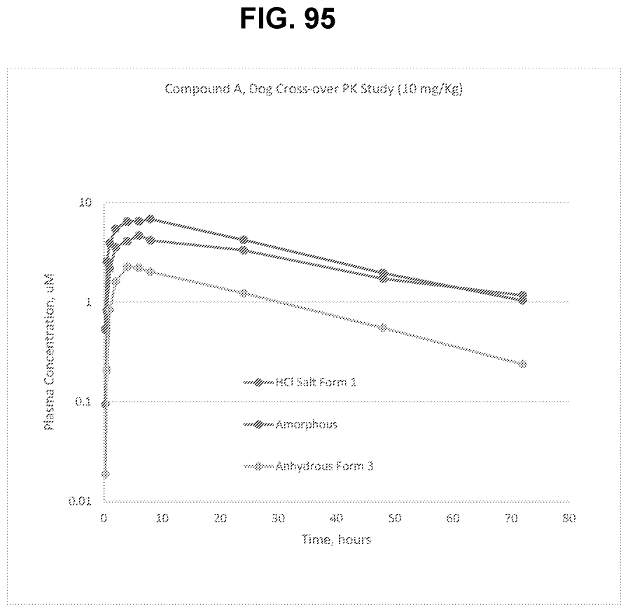

The dog PK cross-over study result is listed in Table 22. The data, as shown in FIG. 95, shows that Compound A-HCl Form 1 had a lower exposure than the Amorphous Compound A form. However, the Compound A-HCl Form 1 exhibited about a 2-fold increase in exposure compared to the Compound A-Anhydrous Form 3, which suggested a higher solubility than Compound A-Anhydrous Form 3.

TABLE 22
Dog Cross-Over PK Study Result:

| | D₅₀/D₉₀ | Cₘₐₓ | AUCₜ | Tₘₐₓ |
|---|---|---|---|---|
| Forms | (μm) | (μM) | (μM*Hr) | (hr) |
| Cmp A1-HCl Form 1 | 19.6/36 | 4.7 ± 1.4 | 181 ± 79 | 6.0 ± 0 |
| Cmp A-Anhydrous | 5.0/13.7 | 2.3 ± 0.2 | 69 ± 6 | 4.7 ± 1.2 |
| Form 3 | | | | |
| Amorphous Cmp A | 9.9/20 | 6.9 ± 1.3 | 237 ± 68 | 6.7± 2.3 |

Cmp = Compound.

### Example 46: PD and IDR Tests of Compound A-HCl Form 1 Compared to Compound A-MsA and A1-TsA

Solubilities of the Compound A-HCl Form 1, Compound A-MsA Form 1, and Compound A-TsA Form 4 were measured in Fed State Simulated Intestinal Fluid (FaSSIF) at pH 6.5.

All three salts showed greater kinetic solubility and faster dissolution than the Compound A in FaSSIF. The dissolution rate of the Tosylate (A-TsA or A-TSA) Salt Form 4 is better than the Mesylate (A-MsA or A-MSA) Salt Form 1, which is better than the HCl Salt Form 1. All three salts can convert to the free base, but maintain supersaturation in FaSSIF for some time, which indicated a potential good absorption in vivo if used in a pharmaceutical dosage form. Solubility Test Result data is listed in Table 23.

TABLE 23
Solubility Test Result of Compound A-HCl Form 1, Compound
A-MsA (MSA) Form 1, and Compound A-TsA (TSA) Form 4.

| | Media |
|---|---|
| | FaSSIF |
| Forms | Sol (pH), μg/mL |
| Cmp A-HCl Form 1 | 104.2 (6.5) |
| Cmp A-MSA Form 1 | 133.0 (6.5) |
| Cmp A-TSA Form 4 | 160.6 (6.5) |

Cmp = Compound.

The foregoing description is given for clearness of understanding only, and no unnecessary limitations should be understood therefrom, as modifications within the scope of the invention may be apparent to those having ordinary skill in the art.

Throughout this specification and the claims which follow, unless the context requires otherwise, the word “comprise” and variations such as “comprises” and “comprising” will be understood to imply the inclusion of a stated integer or step or group of integers or steps but not the exclusion of any other integer or step or group of integers or steps.

Throughout the specification, where compositions are described as including components or materials, it is contemplated that the compositions can also consist essentially of, or consist of, any combination of the recited components or materials, unless described otherwise. Likewise, where methods are described, it is contemplated that the methods can also consist essentially of, or consist of, any combination of the recited steps, unless described otherwise. The invention illustratively disclosed herein suitably may be practiced in the absence of any element or step which is not specifically disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

The practice of a method disclosed herein, and individual steps thereof, can be performed manually and/or with the aid of or automation provided by electronic equipment. Although processes have been described with reference to particular embodiments, a person of ordinary skill in the art will readily appreciate that other ways of performing the acts associated with the methods may be used. For example, the order of various of the steps may be changed without departing from the scope or spirit of the method, unless described otherwise. In addition, some of the individual steps can be combined, omitted, or further subdivided into additional steps.

The use of the terms “a,” “an,” “the,” and similar referents in the context of the disclosure herein (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the disclosure herein and is not a limitation on the scope of the disclosure herein unless otherwise indicated. No language in the specification should be construed as indicating any non-claimed element as essential to the practice of the disclosure herein.

All patents, publications and references cited herein are hereby fully incorporated by reference. In case of conflict between the present disclosure and incorporated patents, publications and references, the present disclosure should control.

We claim:

1. A salt, a hydrate, a solvate, or a co-crystal of Compound A having a chemical structure: 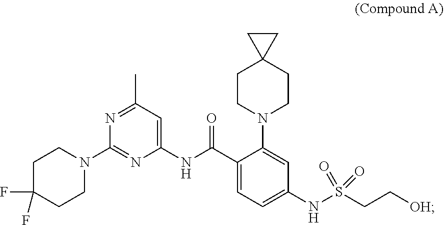 [SMILES: Cc1cc(NC(=O)c2ccc(NS(=O)(=O)CCO)cc2N2CCC3(CC2)CC3)nc(N2CCC(F)(F)CC2)n1]
or
a solid form of Compound A, including crystalline anhydrous forms, salt, solvate, or co-crystal thereof.

2. The salt, hydrate, solvate, or co-crystal of claim 1, selected from hydrochloride salt (Compound A-HCl), mesylate salt (Compound A-MsA), tosylate salt (Compound A-TsA), sulfate salt (Compound A-sulfate), variable hydrate (Compound A-variable hydrate), tetrahydrofuran solvate (Compound A-THF), ethanol solvate (Compound A-ethanol), 1-propanol solvate (Compound A-1-propanol), isopropyl alcohol solvate (Compound A-IPA), methanol solvate (Compound A-methanol), isopropyl acetate solvate (Compound A-IPAc), acetone solvate (Compound A-acetone), cyclopentyl methyl ether solvate (Compound A-CPME), dioxane solvate (Compound A-dioxane), ethyl acetate solvate (Compound A-EtOAc), acetonitrile solvate (Compound A-MeCN), methyl tert-butyl ether solvate (Compound A-MTBE), toluene solvate (Compound A-toluene), dodecyl sulfate (Compound A-dodecyl sulfate), dimethyl formamide (DMF) solvate hydrate (Compound A-DMF-hydrate), dimethylacetamide (DMAC) solvate (Compound A-DMAC), monobesylate hydrate (Compound A-besylate-hydrate), caffeine co-crystal (Compound A-caffeine), citric acid co-crystal (Compound A-citric acid), saccharin co-crystal (Compound A-saccharin), L-tartaric acid co-crystal (Compound A-L-tartaric acid), or urea co-crystal (Compound A-urea); or the solid form thereof.

3. The solid form of the Compound A-HCl of claim 2.

4. The solid form of the Compound A-HCl of claim 3, which is a crystalline Form 1, characterized by solid state ¹⁹F NMR peaks at −91 and −103±0.5 ppm.

5. The crystalline Form 1 of the Compound A-HCl of claim 4, further characterized by XRPD pattern peaks at 7.5, 16.9, and 20.2±0.2° 2θ using Cu Kα radiation.

6. The crystalline Form 1 of the Compound A-HCl of claim 5, further characterized by XRPD pattern peaks at 12.8, 18.2, 22.7, 23.6, 24.8 and 26.1±0.2° 2θ using Cu Kα radiation.

7. The crystalline Form 1 of the Compound A-HCl of claim 6, further characterized by XRPD pattern peaks at 10.9, 14.5, 15.7, 15.9, 19.8, 20.6, 21.6, 23.2, 26.1 and 26.8±0.2° 2θ using Cu Kα radiation.

8. The crystalline Form 1 of the Compound A-HCl of any one of claims 4 to 7, having an XRPD pattern substantially as shown in FIG. 1.

9. The crystalline Form 1 of the Compound A-HCl of any one of claims 4 to 8, having an endothermic transition at 268.5° C. to 274.5° C., as measured by Differential Scanning Calorimetry.

10. The crystalline Form 1 of the Compound A-HCl of claim 9, wherein the endothermic transition is at 271.5° C.±3° C.

11. The crystalline Form 1 of the Compound A-HCl of claim 10, having a Thermogravimetric Analysis (TGA) substantially as shown in FIG. 2.

12. The crystalline Form 1 of the Compound A-HCl of any one of claims 4 to 11, having a single crystal structure substantially as shown in FIG. 5.

13. The hydrochloride salt of Compound A according to claim 2, having the structure:

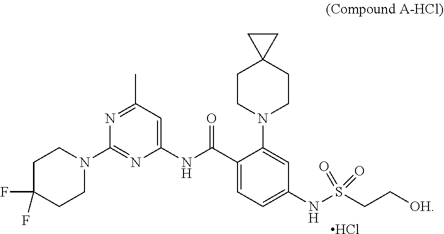 [SMILES: Cc1cc(NC(=O)c2ccc(NS(=O)(=O)CCO)cc2N2CCC3(CC2)CC3)nc(N2CCC(F)(F)CC2)n1 ; Cl]

14. A pharmaceutical composition comprising the solid form of the Compound A-HCl of any one of claims 2 to 12, or the HCl salt of Compound A according to claim 13, and a pharmaceutically acceptable excipient.

15. A method of treating a subject suffering from a disease mediated by KIF18A inhibition, comprising administering to a subject in need thereof a pharmaceutically effective amount of the pharmaceutical composition of claim 14.

16. The method of claim 15, wherein the disease mediated by KIF18A inhibition is cancer, selected from ovarian cancer, breast cancer, lung cancer, or endometrial cancer.

17. The method of claim 15, wherein the subject has relapsed or is refractory to at least one line of systemic chemotherapy.

18. The method of claim 16, wherein the cancer comprises cells that are positive for an inactivated TP53 gene and/or positive for at least one of an inactivated Rb gene, (ii) an amplified CCNE1 gene or overexpressed CCNE1 gene product, (iii) an inactivated BRCA gene or (iv) a combination thereof.

19. A method for preparing the Compound A-HCl salt or the solid form thereof of claim 2, the method comprising: combining hydrochloric acid, Compound A, and a suitable solvent to form the Compound A-HCl or the solid form thereof.

20. The method of claim 19 wherein the suitable solvent is selected from acetonitrile/water, acetonitrile/1,4-dioxane, tetrahydrofuran/water, N-Methyl-2-pyrrolidone/ethanol or acetone/water.


SEQUENCE LISTING (13 sequences)

SEQ ID NO: 1 - DNA, 2512 bp, Homo sapiens
  source 1..2512: mol_type = genomic DNA
ctcaaaagtctagagccaccgtccagggagcaggtagctgctgggctccggggacactttgcgttcgggctgggagcgtgctttccacgacggtgacacgcttccctggattggcagccagactgccttccgggtcactgccatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggggcccacttcaccgtactaaccagggaagctgtccctcactgttgaattttctctaacttcaaggcccatatctgtgaaatgctggcatttgcacctacctcacagagtgcattgtgagggttaatgaaataatgtacatctggccttgaaaccaccttttattacatggggtctagaacttgacccccttgagggtgcttgttccctctccctgttggtcggtgggttggtagtttctacagttgggcagctggttaggtagagggagttgtcaagtctctgctggcccagccaaaccctgtctgacaacctcttggtgaaccttagtacctaaaaggaaatctcaccccatcccacaccctggaggatttcatctcttgtatatgatgatctggatccaccaagacttgttttatgctcagggtcaatttcttttttcttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtccagctggaagggtcaacatcttttacattctgcaagcacatctgcattttcaccccacccttcccctccttctccctttttatatcccatttttatatcgatctcttattttacaataaaactttgctgcca

SEQ ID NO: 2 - protein, 393 aa, Homo sapiens [sequence omitted]

SEQ ID NO: 3 - DNA, 4768 bp, Homo sapiens
  source 1..4768: mol_type = genomic DNA
agttgccgggcgggggagggcgcgtccggtttttctcaggggacgttgaaattatttttgtaacgggagtcgggagaggacggggcgtgccccgacgtgcgcgcgcgtcgtcctccccggcgctcctccacagctcgctggctcccgccgcggaaaggcgtcatgccgcccaaaaccccccgaaaaacggccgccaccgccgccgctgccgccgcggaacccccggcaccgccgccgccgccccctcctgaggaggacccagagcaggacagcggcccggaggacctgcctctcgtcaggcttgagtttgaagaaacagaagaacctgattttactgcattatgtcagaaattaaagataccagatcatgtcagagagagagcttggttaacttgggagaaagtttcatctgtggatggagtattgggaggttatattcaaaagaaaaaggaactgtggggaatctgtatctttattgcagcagttgacctagatgagatgtcgttcacttttactgagctacagaaaaacatagaaatcagtgtccataaattctttaacttactaaaagaaattgataccagtaccaaagttgataatgctatgtcaagactgttgaagaagtatgatgtattgtttgcactcttcagcaaattggaaaggacatgtgaacttatatatttgacacaacccagcagttcgatatctactgaaataaattctgcattggtgctaaaagtttcttggatcacatttttattagctaaaggggaagtattacaaatggaagatgatctggtgatttcatttcagttaatgctatgtgtccttgactattttattaaactctcacctcccatgttgctcaaagaaccatataaaacagctgttatacccattaatggttcacctcgaacacccaggcgaggtcagaacaggagtgcacggatagcaaaacaactagaaaatgatacaagaattattgaagttctctgtaaagaacatgaatgtaatatagatgaggtgaaaaatgtttatttcaaaaattttataccttttatgaattctcttggacttgtaacatctaatggacttccagaggttgaaaatctttctaaacgatacgaagaaatttatcttaaaaataaagatctagatgcaagattatttttggatcatgataaaactcttcagactgattctatagacagttttgaaacacagagaacaccacgaaaaagtaaccttgatgaagaggtgaatgtaattcctccacacactccagttaggactgttatgaacactatccaacaattaatgatgattttaaattcagcaagtgatcaaccttcagaaaatctgatttcctattttaacaactgcacagtgaatccaaaagaaagtatactgaaaagagtgaaggatataggatacatctttaaagagaaatttgctaaagctgtgggacagggttgtgtcgaaattggatcacagcgatacaaacttggagttcgcttgtattaccgagtaatggaatccatgcttaaatcagaagaagaacgattatccattcaaaattttagcaaacttctgaatgacaacatttttcatatgtctttattggcgtgcgctcttgaggttgtaatggccacatatagcagaagtacatctcagaatcttgattctggaacagatttgtctttcccatggattctgaatgtgcttaatttaaaagcctttgatttttacaaagtgatcgaaagttttatcaaagcagaaggcaacttgacaagagaaatgataaaacatttagaacgatgtgaacatcgaatcatggaatcccttgcatggctctcagattcacctttatttgatcttattaaacaatcaaaggaccgagaaggaccaactgatcaccttgaatctgcttgtcctcttaatcttcctctccagaataatcacactgcagcagatatgtatctttctcctgtaagatctccaaagaaaaaaggttcaactacgcgtgtaaattctactgcaaatgcagagacacaagcaacctcagccttccagacccagaagccattgaaatctacctctctttcactgttttataaaaaagtgtatcggctagcctatctccggctaaatacactttgtgaacgccttctgtctgagcacccagaattagaacatatcatctggacccttttccagcacaccctgcagaatgagtatgaactcatgagagacaggcatttggaccaaattatgatgtgttccatgtatggcatatgcaaagtgaagaatatagaccttaaattcaaaatcattgtaacagcatacaaggatcttcctcatgctgttcaggagacattcaaacgtgttttgatcaaagaagaggagtatgattctattatagtattctataactcggtcttcatgcagagactgaaaacaaatattttgcagtatgcttccaccaggccccctaccttgtcaccaatacctcacattcctcgaagcccttacaagtttcctagttcacccttacggattcctggagggaacatctatatttcacccctgaagagtccatataaaatttcagaaggtctgccaacaccaacaaaaatgactccaagatcaagaatcttagtatcaattggtgaatcattcgggacttctgagaagttccagaaaataaatcagatggtatgtaacagcgaccgtgtgctcaaaagaagtgctgaaggaagcaaccctcctaaaccactgaaaaaactacgctttgatattgaaggatcagatgaagcagatggaagtaaacatctcccaggagagtccaaatttcagcagaaactggcagaaatgacttctactcgaacacgaatgcaaaagcagaaaatgaatgatagcatggatacctcaaacaaggaagagaaatgaggatctcaggaccttggtggacactgtgtacacctctggattcattgtctctcacagatgtgactgtataactttcccaggttctgtttatggccacatttaatatcttcagctctttttgtggatataaaatgtgcagatgcaattgtttgggtgattcctaagccacttgaaatgttagtcattgttatttatacaagattgaaaatcttgtgtaaatcctgccatttaaaaagttgtagcagattgtttcctcttccaaagtaaaattgctgtgctttatggatagtaagaatggccctagagtgggagtcctgataacccaggcctgtctgactactttgccttcttttgtagcatataggtgatgtttgctcttgtttttattaatttatatgtatatttttttaatttaacatgaacacccttagaaaatgtgtcctatctatcttccaaatgcaatttgattgactgcccattcaccaaaattatcctgaactcttctgcaaaaatggatattattagaaattagaaaaaaattactaattttacacattagattttattttactattggaatctgatatactgtgtgcttgttttataaaattttgcttttaattaaataaaagctggaagcaaagtataaccatatgatactatcatactactgaaacagatttcatacctcagaatgtaaaagaacttactgattattttcttcatccaacttatgtttttaaatgaggattattgatagtactcttggtttttataccattcagatcactgaatttataaagtacccatctagtacttgaaaaagtaaagtgttctgccagatcttaggtatagaggaccctaacacagtatatcccaagtgcactttctaatgtttctgggtcctgaagaattaagatacaaattaattttactccataaacagactgttaattataggagccttaatttttttttcatagagatttgtctaattgcatctcaaaattattctgccctccttaatttgggaaggtttgtgttttctctggaatggtacatgtcttccatgtatcttttgaactggcaattgtctatttatcttttatttttttaagtcagtatggtctaacactggcatgttcaaagccacattatttctagtccaaaattacaagtaatcaagggtcattatgggttaggcattaatgtttctatctgattttgtgcaaaagcttcaaattaaaacagctgcattagaaaaagaggcgcttctcccctcccctacacctaaaggtgtatttaaactatcttgtgtgattaacttatttagagatgctgtaacttaaaataggggatatttaaggtagcttcagctagcttttaggaaaatcactttgtctaactcagaattatttttaaaaagaaatctggtcttgttagaaaacaaaattttattttgtgctcatttaagtttcaaacttactattttgacagttattttgataacaatgacactagaaaacttgactccatttcatcattgtttctgcatgaatatcatacaaatcagttagtttttaggtcaagggcttactatttctgggtcttttgctactaagttcacattagaattagtgccagaattttaggaacttcagagatcgtgtattgagatttcttaaataatgcttcagatattattgctttattgcttttttgtattggttaaaactgtacatttaaaattgctatgttactattttctacaattaatagtttgtctattttaaaataaattagttgttaagagtcttaa

SEQ ID NO: 4 - protein, 928 aa, Homo sapiens
MPPKTPRKTAATAAAAAAEPPAPPPPPPPEEDPEQDSGPEDLPLVRLEFEETEEPDFTALCQKLKIPDHVRERAWLTWEKVSSVDGVLGGYIQKKKELWGICIFIAAVDLDEMSFTFTELQKNIEISVHKFFNLLKEIDTSTKVDNAMSRLLKKYDVLFALFSKLERTCELIYLTQPSSSISTEINSALVLKVSWITFLLAKGEVLQMEDDLVISFQLMLCVLDYFIKLSPPMLLKEPYKTAVIPINGSPRTPRRGQNRSARIAKQLENDTRIIEVLCKEHECNIDEVKNVYFKNFIPFMNSLGLVTSNGLPEVENLSKRYEEIYLKNKDLDARLFLDHDKTLQTDSIDSFETQRTPRKSNLDEEVNVIPPHTPVRTVMNTIQQLMMILNSASDQPSENLISYFNNCTVNPKESILKRVKDIGYIFKEKFAKAVGQGCVEIGSQRYKLGVRLYYRVMESMLKSEEERLSIQNFSKLLNDNIFHMSLLACALEVVMATYSRSTSQNLDSGTDLSFPWILNVLNLKAFDFYKVIESFIKAEGNLTREMIKHLERCEHRIMESLAWLSDSPLFDLIKQSKDREGPTDHLESACPLNLPLQNNHTAADMYLSPVRSPKKKGSTTRVNSTANAETQATSAFQTQKPLKSTSLSLFYKKVYRLAYLRLNTLCERLLSEHPELEHIIWTLFQHTLQNEYELMRDRHLDQIMMCSMYGICKVKNIDLKFKIIVTAYKDLPHAVQETFKRVLIKEEEYDSIIVFYNSVFMQRLKTNILQYASTRPPTLSPIPHIPRSPYKFPSSPLRIPGGNIYISPLKSPYKISEGLPTPTKMTPRSRILVSIGESFGTSEKFQKINQMVCNSDRVLKRSAEGSNPPKPLKKLRFDIEGSDEADGSKHLPGESKFQQKLAEMTSTRTRMQKQKMNDSMDTSNKEEK

SEQ ID NO: 5 - DNA, 1954 bp, Homo sapiens
  source 1..1954: mol_type = genomic DNA
aggagcagccggcgcggccgccagcgcggtgtagggggcaggcgcggatcccgccaccgccgcgcgctcggcccgccgactcccggcgccgccgccgccactgccgtcgccgccgccgcctgccgggactggagcgcgccgtccgccgcggacaagaccctggcctcaggccggagcagccccatcatgccgagggagcgcagggagcgggatgcgaaggagcgggacaccatgaaggaggacggcggcgcggagttctcggctcgctccaggaagaggaaggcaaacgtgaccgtttttttgcaggatccagatgaagaaatggccaaaatcgacaggacggcgagggaccagtgtgggagccagccttgggacaataatgcagtctgtgcagacccctgctccctgatccccacacctgacaaagaagatgatgaccgggtttacccaaactcaacgtgcaagcctcggattattgcaccatccagaggctccccgctgcctgtactgagctgggcaaatagagaggaagtctggaaaatcatgttaaacaaggaaaagacatacttaagggatcagcactttcttgagcaacaccctcttctgcagccaaaaatgcgagcaattcttctggattggttaatggaggtgtgtgaagtctataaacttcacagggagaccttttacttggcacaagatttctttgaccggtatatggcgacacaagaaaatgttgtaaaaactcttttacagcttattgggatttcatctttatttattgcagccaaacttgaggaaatctatcctccaaagttgcaccagtttgcgtatgtgacagatggagcttgttcaggagatgaaattctcaccatggaattaatgattatgaaggcccttaagtggcgtttaagtcccctgactattgtgtcctggctgaatgtatacatgcaggttgcatatctaaatgacttacatgaagtgctactgccgcagtatccccagcaaatctttatacagattgcagagctgttggatctctgtgtcctggatgttgactgccttgaatttccttatggtatacttgctgcttcggccttgtatcatttctcgtcatctgaattgatgcaaaaggtttcagggtatcagtggtgcgacatagagaactgtgtcaagtggatggttccatttgccatggttataagggagacggggagctcaaaactgaagcacttcaggggcgtcgctgatgaagatgcacacaacatacagacccacagagacagcttggatttgctggacaaagcccgagcaaagaaagccatgttgtctgaacaaaatagggcttctcctctccccagtgggctcctcaccccgccacagagcggtaagaagcagagcagcgggccggaaatggcgtgaccaccccatccttctccaccaaagacagttgcgcgcctgctccacgttctcttctgtctgttgcagcggaggcgtgcgtttgcttttacagatatctgaatggaagagtgtttcttccacaacagaagtatttctgtggatggcatcaaacagggcaaagtgttttttattgaatgcttataggttttttttaaataagtgggtcaagtacaccagccacctccagacaccagtgcgtgctcccgatgctgctatggaaggtgctacttgacctaagggactcccacaacaacaaaagcttgaagctgtggagggccacggtggcgtggctctcctcgcaggtgttctgggctccgttgtaccaagtggagcaggtggttgcgggcaagcgttgtgcagagcccatagccagctgggcagggggctgccctctccacattatcagttgacagtgtacaatgcctttgatgaactgttttgtaagtgctgctatatctatccattttttaataaagataatactgtttttgagaca

SEQ ID NO: 6 - protein, 410 aa, Homo sapiens
MPRERRERDAKERDTMKEDGGAEFSARSRKRKANVTVFLQDPDEEMAKIDRTARDQCGSQPWDNNAVCADPCSLIPTPDKEDDDRVYPNSTCKPRIIAPSRGSPLPVLSWANREEVWKIMLNKEKTYLRDQHFLEQHPLLQPKMRAILLDWLMEVCEVYKLHRETFYLAQDFFDRYMATQENVVKTLLQLIGISSLFIAAKLEEIYPPKLHQFAYVTDGACSGDEILTMELMIMKALKWRLSPLTIVSWLNVYMQVAYLNDLHEVLLPQYPQQIFIQIAELLDLCVLDVDCLEFPYGILAASALYHFSSSELMQKVSGYQWCDIENCVKWMVPFAMVIRETGSSKLKHFRGVADEDAHNIQTHRDSLDLLDKARAKKAMLSEQNRASPLPSGLLTPPQSGKKQSSGPEMA

SEQ ID NO: 7 - DNA, 7088 bp, Homo sapiens
  source 1..7088: mol_type = genomic DNA
gctgagacttcctggacgggggacaggctgtggggtttctcagataactgggcccctgcgctcaggaggccttcaccctctgctctgggtaaagttcattggaacagaaagaaatggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtagttctgtttcaaacttgcatgtggagccatgtggcacaaatactcatgccagctcattacagcatgagaacagcagtttattactcactaaagacagaatgaatgtagaaaaggctgaattctgtaataaaagcaaacagcctggcttagcaaggagccaacataacagatgggctggaagtaaggaaacatgtaatgataggcggactcccagcacagaaaaaaaggtagatctgaatgctgatcccctgtgtgagagaaaagaatggaataagcagaaactgccatgctcagagaatcctagagatactgaagatgttccttggataacactaaatagcagcattcagaaagttaatgagtggttttccagaagtgatgaactgttaggttctgatgactcacatgatggggagtctgaatcaaatgccaaagtagctgatgtattggacgttctaaatgaggtagatgaatattctggttcttcagagaaaatagacttactggccagtgatcctcatgaggctttaatatgtaaaagtgaaagagttcactccaaatcagtagagagtaatattgaagacaaaatatttgggaaaacctatcggaagaaggcaagcctccccaacttaagccatgtaactgaaaatctaattataggagcatttgttactgagccacagataatacaagagcgtcccctcacaaataaattaaagcgtaaaaggagacctacatcaggccttcatcctgaggattttatcaagaaagcagatttggcagttcaaaagactcctgaaatgataaatcagggaactaaccaaacggagcagaatggtcaagtgatgaatattactaatagtggtcatgagaataaaacaaaaggtgattctattcagaatgagaaaaatcctaacccaatagaatcactcgaaaaagaatctgctttcaaaacgaaagctgaacctataagcagcagtataagcaatatggaactcgaattaaatatccacaattcaaaagcacctaaaaagaataggctgaggaggaagtcttctaccaggcatattcatgcgcttgaactagtagtcagtagaaatctaagcccacctaattgtactgaattgcaaattgatagttgttctagcagtgaagagataaagaaaaaaaagtacaaccaaatgccagtcaggcacagcagaaacctacaactcatggaaggtaaagaacctgcaactggagccaagaagagtaacaagccaaatgaacagacaagtaaaagacatgacagcgatactttcccagagctgaagttaacaaatgcacctggttcttttactaagtgttcaaataccagtgaacttaaagaatttgtcaatcctagccttccaagagaagaaaaagaagagaaactagaaacagttaaagtgtctaataatgctgaagaccccaaagatctcatgttaagtggagaaagggttttgcaaactgaaagatctgtagagagtagcagtatttcattggtacctggtactgattatggcactcaggaaagtatctcgttactggaagttagcactctagggaaggcaaaaacagaaccaaataaatgtgtgagtcagtgtgcagcatttgaaaaccccaagggactaattcatggttgttccaaagataatagaaatgacacagaaggctttaagtatccattgggacatgaagttaaccacagtcgggaaacaagcatagaaatggaagaaagtgaacttgatgctcagtatttgcagaatacattcaaggtttcaaagcgccagtcatttgctccgttttcaaatccaggaaatgcagaagaggaatgtgcaacattctctgcccactctgggtccttaaagaaacaaagtccaaaagtcacttttgaatgtgaacaaaaggaagaaaatcaaggaaagaatgagtctaatatcaagcctgtacagacagttaatatcactgcaggctttcctgtggttggtcagaaagataagccagttgataatgccaaatgtagtatcaaaggaggctctaggttttgtctatcatctcagttcagaggcaacgaaactggactcattactccaaataaacatggacttttacaaaacccatatcgtataccaccactttttcccatcaagtcatttgttaaaactaaatgtaagaaaaatctgctagaggaaaactttgaggaacattcaatgtcacctgaaagagaaatgggaaatgagaacattccaagtacagtgagcacaattagccgtaataacattagagaaaatgtttttaaagaagccagctcaagcaatattaatgaagtaggttccagtactaatgaagtgggctccagtattaatgaaataggttccagtgatgaaaacattcaagcagaactaggtagaaacagagggccaaaattgaatgctatgcttagattaggggttttgcaacctgaggtctataaacaaagtcttcctggaagtaattgtaagcatcctgaaataaaaaagcaagaatatgaagaagtagttcagactgttaatacagatttctctccatatctgatttcagataacttagaacagcctatgggaagtagtcatgcatctcaggtttgttctgagacacctgatgacctgttagatgatggtgaaataaaggaagatactagttttgctgaaaatgacattaaggaaagttctgctgtttttagcaaaagcgtccagaaaggagagcttagcaggagtcctagccctttcacccatacacatttggctcagggttaccgaagaggggccaagaaattagagtcctcagaagagaacttatctagtgaggatgaagagcttccctgcttccaacacttgttatttggtaaagtaaacaatataccttctcagtctactaggcatagcaccgttgctaccgagtgtctgtctaagaacacagaggagaatttattatcattgaagaatagcttaaatgactgcagtaaccaggtaatattggcaaaggcatctcaggaacatcaccttagtgaggaaacaaaatgttctgctagcttgttttcttcacagtgcagtgaattggaagacttgactgcaaatacaaacacccaggatcctttcttgattggttcttccaaacaaatgaggcatcagtctgaaagccagggagttggtctgagtgacaaggaattggtttcagatgatgaagaaagaggaacgggcttggaagaaaataatcaagaagagcaaagcatggattcaaacttaggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagcagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactgactgcagccagccacaggtacagagccacaggaccccaagaatgagcttacaaagtggcctttccaggccctgggagctcctctcactcttcagtccttctactgtcctggctactaaatattttatgtacatcagcctgaaaaggacttctggctatgcaagggtcccttaaagattttctgcttgaagtctcccttggaaatctgccatgagcacaaaattatggtaatttttcacctgagaagattttaaaaccatttaaacgccaccaattgagcaagatgctgattcattatttatcagccctattctttctattcaggctgttgttggcttagggctggaagcacagagtggcttggcctcaagagaatagctggtttccctaagtttacttctctaaaaccctgtgttcacaaaggcagagagtcagacccttcaatggaaggagagtgcttgggatcgattatgtgacttaaagtcagaatagtccttgggcagttctcaaatgttggagtggaacattggggaggaaattctgaggcaggtattagaaatgaaaaggaaacttgaaacctgggcatggtggctcacgcctgtaatcccagcactttgggaggccaaggtgggcagatcactggaggtcaggagttcgaaaccagcctggccaacatggtgaaaccccatctctactaaaaatacagaaattagccggtcatggtggtggacacctgtaatcccagctactcaggtggctaaggcaggagaatcacttcagcccgggaggtggaggttgcagtgagccaagatcataccacggcactccagcctgggtgacagtgagactgtggctcaaaaaaaaaaaaaaaaaaaggaaaatgaaactagaagagatttctaaaagtctgagatatatttgctagatttctaaagaatgtgttctaaaacagcagaagattttcaagaaccggtttccaaagacagtcttctaattcctcattagtaataagtaaaatgtttattgttgtagctctggtatataatccattcctcttaaaatataagacctctggcatgaatatttcatatctataaaatgacagatcccaccaggaaggaagctgttgctttctttgaggtgatttttttcctttgctccctgttgctgaaaccatacagcttcataaataattttgcttgctgaaggaagaaaaagtgtttttcataaacccattatccaggactgtttatagctgttggaaggactaggtcttccctagcccccccagtgtgcaagggcagtgaagacttgattgtacaaaatacgttttgtaaatgttgtgctgttaacactgcaaataaacttggtagcaaacacttcca

SEQ ID NO: 8 - protein, 1863 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 9 - DNA, 11954 bp, Homo sapiens
  source 1..11954: mol_type = genomic DNA
agaggcggagccgctgtggcactgctgcgcctctgctgcgcctcgggtgtcttttgcggcggtgggtcgccgccgggagaagcgtgaggggacagatttgtgaccggcgcggtttttgtcagcttactccggccaaaaaagaactgcacctctggagcggacttatttaccaagcattggaggaatatcgtaggtaaaaatgcctattggatccaaagagaggccaacattttttgaaatttttaagacacgctgcaacaaagcagatttaggaccaataagtcttaattggtttgaagaactttcttcagaagctccaccctataattctgaacctgcagaagaatctgaacataaaaacaacaattacgaaccaaacctatttaaaactccacaaaggaaaccatcttataatcagctggcttcaactccaataatattcaaagagcaagggctgactctgccgctgtaccaatctcctgtaaaagaattagataaattcaaattagacttaggaaggaatgttcccaatagtagacataaaagtcttcgcacagtgaaaactaaaatggatcaagcagatgatgtttcctgtccacttctaaattcttgtcttagtgaaagtcctgttgttctacaatgtacacatgtaacaccacaaagagataagtcagtggtatgtgggagtttgtttcatacaccaaagtttgtgaagggtcgtcagacaccaaaacatatttctgaaagtctaggagctgaggtggatcctgatatgtcttggtcaagttctttagctacaccacccacccttagttctactgtgctcatagtcagaaatgaagaagcatctgaaactgtatttcctcatgatactactgctaatgtgaaaagctatttttccaatcatgatgaaagtctgaagaaaaatgatagatttatcgcttctgtgacagacagtgaaaacacaaatcaaagagaagctgcaagtcatggatttggaaaaacatcagggaattcatttaaagtaaatagctgcaaagaccacattggaaagtcaatgccaaatgtcctagaagatgaagtatatgaaacagttgtagatacctctgaagaagatagtttttcattatgtttttctaaatgtagaacaaaaaatctacaaaaagtaagaactagcaagactaggaaaaaaattttccatgaagcaaacgctgatgaatgtgaaaaatctaaaaaccaagtgaaagaaaaatactcatttgtatctgaagtggaaccaaatgatactgatccattagattcaaatgtagcaaatcagaagccctttgagagtggaagtgacaaaatctccaaggaagttgtaccgtctttggcctgtgaatggtctcaactaaccctttcaggtctaaatggagcccagatggagaaaatacccctattgcatatttcttcatgtgaccaaaatatttcagaaaaagacctattagacacagagaacaaaagaaagaaagattttcttacttcagagaattctttgccacgtatttctagcctaccaaaatcagagaagccattaaatgaggaaacagtggtaaataagagagatgaagagcagcatcttgaatctcatacagactgcattcttgcagtaaagcaggcaatatctggaacttctccagtggcttcttcatttcagggtatcaaaaagtctatattcagaataagagaatcacctaaagagactttcaatgcaagtttttcaggtcatatgactgatccaaactttaaaaaagaaactgaagcctctgaaagtggactggaaatacatactgtttgctcacagaaggaggactccttatgtccaaatttaattgataatggaagctggccagccaccaccacacagaattctgtagctttgaagaatgcaggtttaatatccactttgaaaaagaaaacaaataagtttatttatgctatacatgatgaaacatcttataaaggaaaaaaaataccgaaagaccaaaaatcagaactaattaactgttcagcccagtttgaagcaaatgcttttgaagcaccacttacatttgcaaatgctgattcaggtttattgcattcttctgtgaaaagaagctgttcacagaatgattctgaagaaccaactttgtccttaactagctcttttgggacaattctgaggaaatgttctagaaatgaaacatgttctaataatacagtaatctctcaggatcttgattataaagaagcaaaatgtaataaggaaaaactacagttatttattaccccagaagctgattctctgtcatgcctgcaggaaggacagtgtgaaaatgatccaaaaagcaaaaaagtttcagatataaaagaagaggtcttggctgcagcatgtcacccagtacaacattcaaaagtggaatacagtgatactgactttcaatcccagaaaagtcttttatatgatcatgaaaatgccagcactcttattttaactcctacttccaaggatgttctgtcaaacctagtcatgatttctagaggcaaagaatcatacaaaatgtcagacaagctcaaaggtaacaattatgaatctgatgttgaattaaccaaaaatattcccatggaaaagaatcaagatgtatgtgctttaaatgaaaattataaaaacgttgagctgttgccacctgaaaaatacatgagagtagcatcaccttcaagaaaggtacaattcaaccaaaacacaaatctaagagtaatccaaaaaaatcaagaagaaactacttcaatttcaaaaataactgtcaatccagactctgaagaacttttctcagacaatgagaataattttgtcttccaagtagctaatgaaaggaataatcttgctttaggaaatactaaggaacttcatgaaacagacttgacttgtgtaaacgaacccattttcaagaactctaccatggttttatatggagacacaggtgataaacaagcaacccaagtgtcaattaaaaaagatttggtttatgttcttgcagaggagaacaaaaatagtgtaaagcagcatataaaaatgactctaggtcaagatttaaaatcggacatctccttgaatatagataaaataccagaaaaaaataatgattacatgaacaaatgggcaggactcttaggtccaatttcaaatcacagttttggaggtagcttcagaacagcttcaaataaggaaatcaagctctctgaacataacattaagaagagcaaaatgttcttcaaagatattgaagaacaatatcctactagtttagcttgtgttgaaattgtaaataccttggcattagataatcaaaagaaactgagcaagcctcagtcaattaatactgtatctgcacatttacagagtagtgtagttgtttctgattgtaaaaatagtcatataacccctcagatgttattttccaagcaggattttaattcaaaccataatttaacacctagccaaaaggcagaaattacagaactttctactatattagaagaatcaggaagtcagtttgaatttactcagtttagaaaaccaagctacatattgcagaagagtacatttgaagtgcctgaaaaccagatgactatcttaaagaccacttctgaggaatgcagagatgctgatcttcatgtcataatgaatgccccatcgattggtcaggtagacagcagcaagcaatttgaaggtacagttgaaattaaacggaagtttgctggcctgttgaaaaatgactgtaacaaaagtgcttctggttatttaacagatgaaaatgaagtggggtttaggggcttttattctgctcatggcacaaaactgaatgtttctactgaagctctgcaaaaagctgtgaaactgtttagtgatattgagaatattagtgaggaaacttctgcagaggtacatccaataagtttatcttcaagtaaatgtcatgattctgttgtttcaatgtttaagatagaaaatcataatgataaaactgtaagtgaaaaaaataataaatgccaactgatattacaaaataatattgaaatgactactggcacttttgttgaagaaattactgaaaattacaagagaaatactgaaaatgaagataacaaatatactgctgccagtagaaattctcataacttagaatttgatggcagtgattcaagtaaaaatgatactgtttgtattcataaagatgaaacggacttgctatttactgatcagcacaacatatgtcttaaattatctggccagtttatgaaggagggaaacactcagattaaagaagatttgtcagatttaacttttttggaagttgcgaaagctcaagaagcatgtcatggtaatacttcaaataaagaacagttaactgctactaaaacggagcaaaatataaaagattttgagacttctgatacattttttcagactgcaagtgggaaaaatattagtgtcgccaaagagtcatttaataaaattgtaaatttctttgatcagaaaccagaagaattgcataacttttccttaaattctgaattacattctgacataagaaagaacaaaatggacattctaagttatgaggaaacagacatagttaaacacaaaatactgaaagaaagtgtcccagttggtactggaaatcaactagtgaccttccagggacaacccgaacgtgatgaaaagatcaaagaacctactctattgggttttcatacagctagcgggaaaaaagttaaaattgcaaaggaatctttggacaaagtgaaaaacctttttgatgaaaaagagcaaggtactagtgaaatcaccagttttagccatcaatgggcaaagaccctaaagtacagagaggcctgtaaagaccttgaattagcatgtgagaccattgagatcacagctgccccaaagtgtaaagaaatgcagaattctctcaataatgataaaaaccttgtttctattgagactgtggtgccacctaagctcttaagtgataatttatgtagacaaactgaaaatctcaaaacatcaaaaagtatctttttgaaagttaaagtacatgaaaatgtagaaaaagaaacagcaaaaagtcctgcaacttgttacacaaatcagtccccttattcagtcattgaaaattcagccttagctttttacacaagttgtagtagaaaaacttctgtgagtcagacttcattacttgaagcaaaaaaatggcttagagaaggaatatttgatggtcaaccagaaagaataaatactgcagattatgtaggaaattatttgtatgaaaataattcaaacagtactatagctgaaaatgacaaaaatcatctctccgaaaaacaagatacttatttaagtaacagtagcatgtctaacagctattcctaccattctgatgaggtatataatgattcaggatatctctcaaaaaataaacttgattctggtattgagccagtattgaagaatgttgaagatcaaaaaaacactagtttttccaaagtaatatccaatgtaaaagatgcaaatgcatacccacaaactgtaaatgaagatatttgcgttgaggaacttgtgactagctcttcaccctgcaaaaataaaaatgcagccattaaattgtccatatctaatagtaataattttgaggtagggccacctgcatttaggatagccagtggtaaaatcgtttgtgtttcacatgaaacaattaaaaaagtgaaagacatatttacagacagtttcagtaaagtaattaaggaaaacaacgagaataaatcaaaaatttgccaaacgaaaattatggcaggttgttacgaggcattggatgattcagaggatattcttcataactctctagataatgatgaatgtagcacgcattcacataaggtttttgctgacattcagagtgaagaaattttacaacataaccaaaatatgtctggattggagaaagtttctaaaatatcaccttgtgatgttagtttggaaacttcagatatatgtaaatgtagtatagggaagcttcataagtcagtctcatctgcaaatacttgtgggatttttagcacagcaagtggaaaatctgtccaggtatcagatgcttcattacaaaacgcaagacaagtgttttctgaaatagaagatagtaccaagcaagtcttttccaaagtattgtttaaaagtaacgaacattcagaccagctcacaagagaagaaaatactgctatacgtactccagaacatttaatatcccaaaaaggcttttcatataatgtggtaaattcatctgctttctctggatttagtacagcaagtggaaagcaagtttccattttagaaagttccttacacaaagttaagggagtgttagaggaatttgatttaatcagaactgagcatagtcttcactattcacctacgtctagacaaaatgtatcaaaaatacttcctcgtgttgataagagaaacccagagcactgtgtaaactcagaaatggaaaaaacctgcagtaaagaatttaaattatcaaataacttaaatgttgaaggtggttcttcagaaaataatcactctattaaagtttctccatatctctctcaatttcaacaagacaaacaacagttggtattaggaaccaaagtgtcacttgttgagaacattcatgttttgggaaaagaacaggcttcacctaaaaacgtaaaaatggaaattggtaaaactgaaactttttctgatgttcctgtgaaaacaaatatagaagtttgttctacttactccaaagattcagaaaactactttgaaacagaagcagtagaaattgctaaagcttttatggaagatgatgaactgacagattctaaactgccaagtcatgccacacattctctttttacatgtcccgaaaatgaggaaatggttttgtcaaattcaagaattggaaaaagaagaggagagccccttatcttagtgggagaaccctcaatcaaaagaaacttattaaatgaatttgacaggataatagaaaatcaagaaaaatccttaaaggcttcaaaaagcactccagatggcacaataaaagatcgaagattgtttatgcatcatgtttctttagagccgattacctgtgtaccctttcgcacaactaaggaacgtcaagagatacagaatccaaattttaccgcacctggtcaagaatttctgtctaaatctcatttgtatgaacatctgactttggaaaaatcttcaagcaatttagcagtttcaggacatccattttatcaagtttctgctacaagaaatgaaaaaatgagacacttgattactacaggcagaccaaccaaagtctttgttccaccttttaaaactaaatcacattttcacagagttgaacagtgtgttaggaatattaacttggaggaaaacagacaaaagcaaaacattgatggacatggctctgatgatagtaaaaataagattaatgacaatgagattcatcagtttaacaaaaacaactccaatcaagcagtagctgtaactttcacaaagtgtgaagaagaacctttagatttaattacaagtcttcagaatgccagagatatacaggatatgcgaattaagaagaaacaaaggcaacgcgtctttccacagccaggcagtctgtatcttgcaaaaacatccactctgcctcgaatctctctgaaagcagcagtaggaggccaagttccctctgcgtgttctcataaacagctgtatacgtatggcgtttctaaacattgcataaaaattaacagcaaaaatgcagagtcttttcagtttcacactgaagattattttggtaaggaaagtttatggactggaaaaggaatacagttggctgatggtggatggctcataccctccaatgatggaaaggctggaaaagaagaattttatagggctctgtgtgacactccaggtgtggatccaaagcttatttctagaatttgggtttataatcactatagatggatcatatggaaactggcagctatggaatgtgcctttcctaaggaatttgctaatagatgcctaagcccagaaagggtgcttcttcaactaaaatacagatatgatacggaaattgatagaagcagaagatcggctataaaaaagataatggaaagggatgacacagctgcaaaaacacttgttctctgtgtttctgacataatttcattgagcgcaaatatatctgaaacttctagcaataaaactagtagtgcagatacccaaaaagtggccattattgaacttacagatgggtggtatgctgttaaggcccagttagatcctcccctcttagctgtcttaaagaatggcagactgacagttggtcagaagattattcttcatggagcagaactggtgggctctcctgatgcctgtacacctcttgaagccccagaatctcttatgttaaagatttctgctaacagtactcggcctgctcgctggtataccaaacttggattctttcctgaccctagaccttttcctctgcccttatcatcgcttttcagtgatggaggaaatgttggttgtgttgatgtaattattcaaagagcataccctatacagtggatggagaagacatcatctggattatacatatttcgcaatgaaagagaggaagaaaaggaagcagcaaaatatgtggaggcccaacaaaagagactagaagccttattcactaaaattcaggaggaatttgaagaacatgaagaaaacacaacaaaaccatatttaccatcacgtgcactaacaagacagcaagttcgtgctttgcaagatggtgcagagctttatgaagcagtgaagaatgcagcagacccagcttaccttgagggttatttcagtgaagagcagttaagagccttgaataatcacaggcaaatgttgaatgataagaaacaagctcagatccagttggaaattaggaaggccatggaatctgctgaacaaaaggaacaaggtttatcaagggatgtcacaaccgtgtggaagttgcgtattgtaagctattcaaaaaaagaaaaagattcagttatactgagtatttggcgtccatcatcagatttatattctctgttaacagaaggaaagagatacagaatttatcatcttgcaacttcaaaatctaaaagtaaatctgaaagagctaacatacagttagcagcgacaaaaaaaactcagtatcaacaactaccggtttcagatgaaattttatttcagatttaccagccacgggagccccttcacttcagcaaatttttagatccagactttcagccatcttgttctgaggtggacctaataggatttgtcgtttctgttgtgaaaaaaacaggacttgcccctttcgtctatttgtcagacgaatgttacaatttactggcaataaagttttggatagaccttaatgaggacattattaagcctcatatgttaattgctgcaagcaacctccagtggcgaccagaatccaaatcaggccttcttactttatttgctggagatttttctgtgttttctgctagtccaaaagagggccactttcaagagacattcaacaaaatgaaaaatactgttgagaatattgacatactttgcaatgaagcagaaaacaagcttatgcatatactgcatgcaaatgatcccaagtggtccaccccaactaaagactgtacttcagggccgtacactgctcaaatcattcctggtacaggaaacaagcttctgatgtcttctcctaattgtgagatatattatcaaagtcctttatcactttgtatggccaaaaggaagtctgtttccacacctgtctcagcccagatgacttcaaagtcttgtaaaggggagaaagagattgatgaccaaaagaactgcaaaaagagaagagccttggatttcttgagtagactgcctttacctccacctgttagtcccatttgtacatttgtttctccggctgcacagaaggcatttcagccaccaaggagttgtggcaccaaatacgaaacacccataaagaaaaaagaactgaattctcctcagatgactccatttaaaaaattcaatgaaatttctcttttggaaagtaattcaatagctgacgaagaacttgcattgataaatacccaagctcttttgtctggttcaacaggagaaaaacaatttatatctgtcagtgaatccactaggactgctcccaccagttcagaagattatctcagactgaaacgacgttgtactacatctctgatcaaagaacaggagagttcccaggccagtacggaagaatgtgagaaaaataagcaggacacaattacaactaaaaaatatatctaagcatttgcaaaggcgacaataaattattgacgcttaacctttccagtttataagactggaatataatttcaaaccacacattagtacttatgttgcacaatgagaaaagaaattagtttcaaatttacctcagcgtttgtgtatcgggcaaaaatcgttttgcccgattccgtattggtatacttttgcttcagttgcatatcttaaaactaaatgtaatttattaactaatcaagaaaaacatctttggctgagctcggtggctcatgcctgtaatcccaacactttgagaagctgaggtgggaggagtgcttgaggccaggagttcaagaccagcctgggcaacatagggagacccccatctttacaaagaaaaaaaaaaggggaaaagaaaatcttttaaatctttggatttgatcactacaagtattattttacaagtgaaataaacataccattttcttttagattgtgtcattaaatggaatgaggtctcttagtacagttattttgatgcagataattccttttagtttagctactattttaggggattttttttagaggtaactcactatgaaatagttctccttaatgcaaatatgttggttctgctatagttccatcctgttcaaaagtcaggatgaatatgaagagtggtgtttccttttgagcaattcttcatccttaagtcagcatgattataagaaaaatagaaccctcagtgtaactctaattcctttttactattccagtgtgatctctgaaattaaattacttcaactaaaaattcaaatactttaaatcagaagatttcatagttaatttattttttttttcaacaaaatggtcatccaaactcaaacttgagaaaatatcttgctttcaaattggcactgattctgcctgctttatttttagcgctatcacaggacccagagcctatgcccttttaaacttaccacaaaagcagaagattaattcaatttaagatgatactctcatttgttacgtcctttttttttttttttggagatggagtcttgctttgtcgcccatgctggagtgcagtggcatgatcctggctcactgcagcctccacttcccgggttcacgtaattctcccacctcaagcctccctagtagctgggattacagggacgcaccaccatgcccagctaatttttgcatttttagtagagactgggttttaccatgttggccaagctggtctcaaactcctgatgtcaggtgatccatctgcctcagcctcccaaagtgctgggattataggcgtgagccactgtgcccggccaatatttgttactttcttaggtttaatagagaaaagggataaaacatttctaactgggagttaattgcatggagaaggtcttaaatcagatgttttaatgccttaaatgtctgtataatatcatgttttcaaatctaattataaatacgtttaaagccaagaataaatcttttaaaaaattga

SEQ ID NO: 10 - protein, 3418 aa, Homo sapiens [sequence omitted]

SEQ ID NO: 11 - protein, 898 aa, Homo sapiens
MSVTEEDLCHHMKVVVRVRPENTKEKAAGFHKVVHVVDKHILVFDPKQEEVSFFHGKKTTNQNVIKKQNKDLKFVFDAVFDETSTQSEVFEHTTKPILRSFLNGYNCTVLAYGATGAGKTHTMLGSADEPGVMYLTMLHLYKCMDEIKEEKICSTAVSYLEVYNEQIRDLLVNSGPLAVREDTQKGVVVHGLTLHQPKSSEEILHLLDNGNKNRTQHPTDMNATSSRSHAVFQIYLRQQDKTASINQNVRIAKMSLIDLAGSERASTSGAKGTRFVEGTNINRSLLALGNVINALADSKRKNQHIPYRNSKLTRLLKDSLGGNCQTIMIAAVSPSSVFYDDTYNTLKYANRAKDIKSSLKSNVLNVNNHITQYVKICNEQKAEILLLKEKLKAYEEQKAFTNENDQAKLMISNPQEKEIERFQEILNCLFQNREEIRQEYLKLEMLLKENELKSFYQQQCHKQIEMMCSEDKVEKATGKRDHRLAMLKTRRSYLEKRREEELKQFDENTNWLHRVEKEMGLLSQNGHIPKELKKDLHCHHLHLQNKDLKAQIRHMMDLACLQEQQHRQTEAVLNALLPTLRKQYCTLKEAGLSNAAFESDFKEIEHLVERKKVVVWADQTAEQPKQNDLPGISVLMTFPQLGPVQPIPCCSSSGGTNLVKIPTEKRTRRKLMPSPLKGQHTLKSPPSQSVQLNDSLSKELQPIVYTPEDCRKAFQNPSTVTLMKPSSFTTSFQAISSNINSDNCLKMLCEVAIPHNRRKECGQEDLDSTFTICEDIKSSKCKLPEQESLPNDNKDILQRLDPSSFSTKHSMPVPSMVPSYMAMTTAAKRKRKLTSSTSNSSLTADVNSGFAKRVRQDNSSEKHLQENKPTMEHKRNICKINPSMVRKFGRNISKGNLR

SEQ ID NO: 12 - DNA, 87562 bp, Homo sapiens
  source 1..87562: mol_type = genomic DNA
cactttaaaatctacatctttaatatatcaagtagcaagggacaatattttatttgtgatgactaaataatatttacaatgattttatgggcacatatatttacacttgtacattaaagtatttttgaaaaggaaagtcatatatatgattaaaataatgtatcaaaacataatagagaaccaaatacatcttgggccataattttaccacttctaaattcttatttttaaaaaataataaagataatttaactcactaccatttactaaaatgtcctagtcaacttgttgtggcatagtaaaactaagctgccaccatggtaacttacactcagggattttaaaaggcaacaaatatttaaaaaacttaaaagacaaccttttcttttgaaattttatttttttagaacacaaaagaagaaaacaaagagtttcatttttctgcttgctgaaagtacttgggtaaacttagctttaagatgggtcttctttcaaagattttaaatatatttttgaaagggtattgataaactttgaaaagcagatttgatcaacttcattttgcttggttttgaagtgatttatcttagatttccttttgaaatatttcttccaaattttctaaccatgcttggatttattttacagatgtttcttttatgttctagagaagaaataaaaagatgcataaatatggcataaatatcattcaaaaagttttcatcgttcaacttataaaaattatgaccatagaaagaaaaatttactcttgaaattcatagttgttagttaacataatttaaaatataacaacaacaaagagacattaatacctttggtttaaaagctgttcaaattcatattgcaaactaaagtttcagtgagtgcgtgcacatacacagacatcgggtgaagtcgctgaattaaatactatgttcattttctttctttctgactaataaaagaccatttctacaagcatttcttcattttctttgttgtatcaccattttttctcattccttggattatatttccccagggatttaaacttccatatttttccaattttaaaattatcatttgcatctagtacttcctaaagggctgatcttaatcctatagctcttttttcttttactgccacctctcaagtatgttctatttatgcccattcttctggttcttcattagctttcaccgtcattttctactcaacaaagtgttgttacaacagggtgccattgacttttttcaggattagtttaattgcttatccactttgttggccatattaggttttagttttagagggacatattcccttaacactctctgttatttctctaactgctaagttaacaggagttgcctttttccctactgatggtcctatgtttctttatttgtacattgttagttagaaaactggtttaatacatttttcacttgaacattataatgtatataaaatatctttccttacaaactaatacctctttggcaagcattgtggtttattatcatctcccacttttttttccccatagtagatatttttactcacatctctttatacatgaaatatttgatgaatttgtatcttcagataataagcatattagaaaaagccaagttctatgatttgagttttttttttttttgagacggagttttgctcagtcgcccaggctggagtgcagtggtgcgatctcggctcactgcaagctccgcctcccaggttcacgccattctcctgcctcagcttcccgagtagctgggactacagccgcctgccaccatgcccggctaattttttaaaatttttttggtagagacggggtttcaccatgttagccaggatggtctcgatctcctgacctcgtgatccacccgcctcagcctcccaaagtgctgggattacaggcgtgagccacggcgcccggccaatttgagagtttttataaatttattatttggctgtagaattatgattcttgaagaactttttaagaagatcagtggggcagtacacacctaaactaactacagatacacatttttaagcattattaaatacaacatgtcaaaccaaatgatcaaggtaaattttgtactttactaccttactgtctgatgtttcaatttgctgtctgatgcttcaatttccattgaacttaaggtaaacactggctactacaggaagcaggcatttaccaatatagtttgacaaagttactgaggaattttatattactctaatcaaagggcatcgacagaaaaaaactaacaatagctttcatgttatccttagaaaaattatatcatgagacacctttaaattgcatttgctataattatcagttcctatagagaacaatcaaactgtgtcttactgctctctgtattcatcacaggacatggtaaatgcctagtgaataaatagtttctgaatgaagtcttaggaactgtagtatcagagacagaggtctagatgttggggcctgaaaactggctggcatgatacaccactagtttaactgaagtggttttgttaccataaactcatgtcactcccatatgcggttgatggaatgtagtatctttcatactgatgggaaagatccatcagtatcttttccaacttgccaatgggagttacaatatgattgtaactagatacagactggaatctgtgtagtagttacacattgagttagaggccaaattattcaaaatattacgctaagagaaaaatttaaagtgggtatagtttatctgcaaaatacacttaattagtaacattaagtaatacatttagccaattatttcaattattatgagacagtagggataactaaaagggtgtagaaagtagagttttaagatctggttcttgagttttggctctaccaatataatatctgtgtgatcttgggtaaatgcttttgtgttttaaagtttccttatctataatatgtggataatcatacttgttctaccacatagtaaaggtgatgaataaagggagattcttatgaaaacacaataaaaaacgtaaagtactatatgtgtgttacagagtcataccattaaatatcaaattaaaagctgagacatacccattgttggtttgttttcttgtaagtgcttctcacttgaattatcttgtcgaacacgtttggcaaatccagaatttacgtctgcagttaacgaactgtttgatgtagaacttgagaggaaaagtttttaatttagttaagcattttttttatacctcaaagttctaatacatattgtacatgcgacaatgattaaagaaataaaacacaaaaatattaagttgtgaaatctcacagaagtggaggtactaaatacatttttggcccacagagaaatttagctgtgtgtgattttaaattggatattttatatttttattcaaattgaatgttatgattgtaggtgtatgttacaattctgtagcagaatattccagggtgctttgctggcagataagtaagcactattattcaaaatgtagagatcagtagcacatagtcacaagaggttaaaaaaaaaaaaaaaaaaaactaagctcatgttacaaatttggaccaagaatcctaatgtgaaaggtataaacaagtgagattgtttgaaaaaccttgttttaaaatgaaagcagggtaacaataaaaatctctaattattacaatcacagaaactgaaataaaaagaatgacctgttttcatttgctctagcttcaaaaatttaatcagtcttacactgctgatggttcaactttcttactcaatttgagttgccatattagtaaacacctattatggccttgtgttacatgatatacaagtaaacaaggcacaaaaatccctgtcctaaaaacttatagaatagacatacagtggagatgagaaaaaaataagataaatacatgaaataatataaggtaaagtaaaaatatagtgccaaggtttccataatacaaggagctatagattcttgagtgtagagtaagaaagaataatggaaattttgtggatgcaatgaatttgatctgggtcctgaaaaacagcaatccttcctagtcctaaagaggatcaatttttaacaggtaggattggggggggttgggtggaaaaagaacatccttccacgctgattcaaacaacctaagcttagggaaattctggctaataaagatactatatagtaatcccccttatcgacgggggggatatcttctaagatcgccagtggatgcctgcaaccgaggatagtatcaaaccggatatatactatgtattctcctacacatatctatgataaagtttaaattataaattaggcacagtaagagattaacaataataataaaatagagcaattataattataattattataaggttatgtgaatgtggtctcgctctctattaaaatatcttattgttctacaatcgcctgtttttgaactgcggttgaccatgggtaactgaaaccacagaaagcaaaacttgaataaggggtgactactcagtggaagtaatgaaaatgacacagtttaagaagataaaatcacaaatttgtatgcacaaatattatatcaactacagtagtccccacttcttaaatggtgataaattactatgattctgttttgcatattaaacacacttttgtatatatcagctgtccaaggcagaaattcttttttgatcatttggtgattttattttttcaaatataaagatcaatattattagttacttttgataaaacattgttttttcttaaatttatcccaaagttgtctttattatttcatagtacgattatgcattatggctagttccaggaccctactttttactcatagcatgttctttcttgaaaattaggcttttgaataaagccatttgattagaactcatatttctagaaatcaaatccaaccctaataaattgcgtatttatataaaagtaaaatatacccaaacatgtttacgatttagttaaatagtccactgatgaaatgtttaacatttatgttgtaggtcataaactacaaagacttaaaactgatgagcgcaagttcaaattatgttaaaggctatactgaaaaatccaaagacacttccagttgtgcacaaaacatattcccataactataaataaagttaagaattaaccttttgctcccttatataggtagaatgtttctactcaacataatacctcttctactattttatctgaaataatttctagttgaaataattagacatctttcaaaacaaaataaaagtgtatggcaaatatgaaactgctttggggtgctcagaagattgtggacaaacaaaaaaccaatcaaccaaacaaatacaaaacaaacaatccaaaactctgattaatataattgagaagtaaaagctaacattatgtatgcattatatttaaatatttgataggctaatcaatttagtaaaatagtgtgctagagaaatattaaataaattgttaagattaaaaatctaatatactcaattatttaaacaaatctgttgggtaggttttggaatatccagtgctttcaaactctaatgccattttatgcaaaataaatgaaatacatatatagagagcttccccttctttattttaaaaactatattagaatatatacacaactaaatatgtatataaagctgctaaatggaaaataccacagtttattactctgaagcgcatgtaactttaattggggaaaaagaagattaaaaaaagtaatcatgtacatttctagcattaatctcttttaaccttagttaatggcaggatctaattactggtaccagagggaatgtaatagaacaattaataggctataagaaatcaagttactttctttacatgtcctttaatattatttcacaatattagttgttcatgtggaagccctttaacttatctaaaatcactccaaatacacaattcacatacctggacatttcctgaaaaactatttgattttataatagctaagttcatatttaaaaattattcttatctataaacatttgtgacatcttagtaaacacaacatactaccagtgaaggacaagaaaactcactaaaattaattaatggaacaaatgacaaaactgacactatgcaaattagtgataaaagtagacgaaacagatgattgtggttagctaggctcactcataaagtgacagacttctgtatataaaccagaataattgtacaaatcaaatttgtaccttagcaaatataaaattttaaatgtcatagatgagacatttaggaaaaatccataaattttaaatttacacattccaagtctctgttatactttgtttaggagggacaaggacagatattaaattcacttatgagaagatactctatgtagcacaaataagtaggctacttgtaaattcgcttcctgttacttaaatgataacaggctcctggtggaaaatacatttaaaaagagtcattttaaggtcttctataaacttttctccccataatcacatagcaaagaaaaagaggtagaggaagtcagatccattttatatttattgagtatatttgtcattgcattcaatttataggatataaagaatgcatcatgaacttctagtcatataactcaatatatttaagatttttccttatctttagtttttagttgttttactactatatacctaggtatgtttctctttatattaacccggcttttggtttgcagtgctactcaaatccatggtttaatgtcttttgttagttttgaataagtattagccatcatctcaaatattacttttccctattctttctctcactcccttctgagacacaaaatacatattttctagacttttaaaaattgtgtcccatgtctctttcacttcttcccatatttttatcatactgcacatcagtttggatattttcctaccagtctatctttcagaaaaaattccaaaacctgaacaaatgaataataagtaacaaaatcaaagtcataataaagtctcccagcaaagaaaagcttggaatttgatggcttcactgctgatttttaccaaacatttaaaaaagaaccaataccaatcttactcaaactattctgaaaaacagaggcggaaggaatacttccaaaatcattatatgaggccagtattactctgataccaaaaccagacaaggacacatcaaaacaagaaaactccaggccaatatccctgatgaacacatgcacaaatccttaacaaaatactagcaaactgaatccaataacaaacctgaaagatcattcatcatggccaagtgggatttatcccagggatgcaagatggttcaacatatgcaaattaaccaatgtgatacattgtatcaatagaatgaagaacaaaaaccatatgatcatttcaatagatgctgaaaaagtatttgataaaattcaacatccctttatgataaaaactctcaccaagcggacttaatagacgtctacagaactctccacccacaatcaacagaatatacattcttttcagcaccacactgcacttattccaaaattgaccacatagttggaagtaaagcactcctcagcaaatataaaagaacagaaattagaacaatctgtctctcagaatacagtgcaatcaaactagaactcaggattaagacactcactcaaaaccactcaactacatggaaactgaacaacctgctcctgaatgactactgggtacataacgaaatgaaggcagaaataaagacgttctttgaaaccaacgagaacaaagacatgacataccagaatctctgggacacatttaaagcagtgtgtagagggaaatttatagcagtaaatgcccacaagagaaagcaggaaagatctaaaattgacaccctaacatcacaattaaaagaactagagaagcaagagcaaacacattaaaaagccagcagaaggcaagaaataactaagatcagagcagaactgaaggagatagagacgcaaaaaacccttcaaaaaatcaatgaatccaggagctggttttttgaaaagatcaacaaaattgatagactgctagcaagactaagaaagaggaaaagagagaagaatcaaatagacacaataaaaaaagataaaggggatatcaccaccgatcccacagaaatacaaactaccatcagagaatactacaaacacctctatgcaaataaactagaaaatctagaagaaatggataaattcctggacacatacaccctcccaagactaaaccaggacgaagttgaatctctgaatagaccaataataggctctgaaattgaggcaataattaatagcataccaaccgaaaaaagtccaggaccgggcggattcacagctgaattctaccagaggtacaaagaggagctggtaccattccttctgaaactatttcaatctatagaaaaagagggaatcctccctaactcattttatgagaccagcatcatcctgataccaaagcctggcagagacacaacaaaaaaagagaattttagaccaatatccctgaagaacattgatgcaaaaatcctcaataaactactggcaagcccaatccagcagcacatcaaaaagcttatccaccatgagcaagtgggcttcatccctgggatgcaaggctggttcaatatacgcaaatcaataaatgtaatccagcatatacacagaaccaaagacaaaaaccacatgattatctcaatagatgcagaaaaggcctttgacaaaattcaacagcccttcatgctaaaaactctcaataaattaggtattgatgggacgtatctcaaaataataagagctatctatgacaaacccacagccaatatcatactgaatgtgcaaaaactggaagcattccctttgaaaactggcacaagacagggatgccctctctcaccactcctactcaatatagtgtgggaagttctggccagggcaatgaggcaagagaaggaaataaaaggtattcaattaggaaaagaagaagtcaaattgtccctgtttgcagatgacatgattgtatatctagaaaaccccatcgtctcagcccaaaatctccttaagctgataagcaacttcagcaaagtctcaggatacaaaatcaatatacaaaaatcacaagcattcttatacaccaataacagacaaacagagagacaaatcatgagtgaactcccattcacaactgcttcaaagagaataaaatacctaggaatccaacttacaagggatgtgaaggacctcttcaaggagaactacaaaccactgctcaatgaaataaaagaggacacaaacaaatggaagaacattccatgctcatggataggaagaatcaatatcatgaaaatgtccatactgcccaaggtaatttatagattcagtgccatccccatcaagctaccaatgactttcttcacagaattggaaaagactactttaaagttcatatggaaccagaaaacagcccgcattgccaagacaatcctaaggcaaaagaacaaaactggaggcatcacgctacctgacttcaaactatactacaaggctacagtaaccaaaacagcctggtactggtaccaaaacagagatatagaccaatggaacagaacagagccctcagaaataataccatacatctacaaccatctgatctttgacaaatctgacaaaaacaagaaatggggaaaggattccccatttaataaatggtgctgggaaaactggctagccatatgtagaaagctgaaactggatcccttccttacaccttatacaaaaattaattcaagatggattaaagacttaaatgttagagctaaaaccataaaaaccccagaagaaaacctaggcaataccattcaggacataggcatgggtaaggacttcatgtctaaaacaccaaaagcaatggcaacaaaagccaaaattgacaaatgggatctaattaaactaaagagcttctgcacagcaaaagaaactaccatcagagtgaacaggcaacctacagaatgggagaaaattttcgcaacctactcatctgacaaagggctaatatccagaatctacaatgaactcaaacaaatttacaagaaaaaaacaaacaaccccatcaaaaagtgggcgaaggacatgaacagacacttctcaaaagaagacatttatgcagccaaaaaacacatgaaaaaatgctcatgatcactggccatcagagaaatgcaaatcaaaaccacaatgagataccatctcacaccagttagaatggcaatcattaaaaagtcaggaaacaacaggtgctggagaggatgtggagaaataggaacacttttacactgttggtgggactgtaaactagttcaaccattgtggaagacagtgtggcgatttctcaaggatctagaactagaaataccatttgacccagccatgtcgttactgggtatatacccaaaggattataaatcatgctgctataaagacacatgcacacgtatgtttattgcggcactattcacaatagcaaagacttgtacccaatccaaatgtccatcaatgatagactggattaagaagatgtggcacatatacaccatggaagactatgcagccataaaaaaggatgagttcatgtcctttgtagggacaggatgaagctgaaaaccatcattctcagcaaactatcgcaaggacaaaaaaccaaacaccgcatgttctcactcataggtgggaactgaacaatgagaacacatggacacaggaaggtgaacatcacacaccggggcctgttgtggggtagggggagcgcagagggaaagcattaggagatataccaaatgtaaataacgagttaatgggtgcagcacaccaacatggcacatgtatacatatgtaacaaacctgcacgttatgcacatgtaccgtggaacttaaagcataaaaaaaaaaaacaactctcaaaaaactgggtatagaaggaacatatctcaacacgataaaagccatacacgacagacccacagctggtactatcctgaatgcagaaaatctgaaattcttttctctaagttgtggaacatgacaaggatgcccactttcaccagtatttattatttaacatattactggaagtcctaagtagagcaatcagacaagagaaagaaaagaaagaaagaacatccaaattggaaaggaagaagtgaaattatccttgtttgcagatgacatgatcttatcatatatttggaaaaacgtagagtccacaaaaaactatgagaacctagaaacaaattcagtagagttccagaatacaaaatcaatatacaaaaatcagtagcatatctatatacaaacactgaaataatttgaaaaaaaaagtaatcccctttacaaaagctaaaaataaaataagtaggagttaaccaaagaagtaaaagagctccataatgaaaactataaaacactgatgtaagaaaagcagatacaaaaaaatagaaagatattccatgttcatggattggaagaatcaatattgttaaaatgtccacactactcacagtggtctacagaatcaatgcaatccctatcaaaataccaatgacactcttcatagaaatagaaaaaacaatcctaaaatttatatggaatcacaaaagacccagaatagccaaagctatcctgagcaaaaagatcaaaactggaggaatcacattacctgacttcaaattacactacagagctatagtaaccaaaaaagcacgggactggcataaaaccagacacacaaaccagtggaacagaatagagagccccaaaataaatctacatatttacaataaacttatttttgacaaagctgccaagaacatacattgggaaaaaagatagtctcttcaataaacagtgctggaaaactgtttatccatatgcagaagaatgaagctagatccctatctcttgcaatatacagaaatgaaatcaaaatggataaaaatttaaatttaagacctcaaactatgaaactgctaaaagaaaacattggggaaactctccaggacactggactggacaaagatttcttgagtaataccctacacagggaaccaaagcaaaaatggataaatgggatcacatcaagttaaaaagcttctgtaaagcaaaggaaacaatcaataaagtgaagagacaacccacagaattacagaaaatacctgcaaactatctatctgacaagggattaataaccataatacataaggagctcaaacaactctaggaaaaaaatctaatgatctgatttttaaaatgggcaaaagaatagacatttctcaaaagaagacataaatggcaaagaagcttatgaaaaggtactcaacatcactgattatcagaaaaatgtaaatcaaaactacaatgagatatcatctcaccccagttaaaatggctttttttccaaaagacagtcaacaacaaacactggcaaggatatggagaaaagggaacacttgtatactgttcatggggatgtaaattagtacaaccactataaagaaaagtttgaagagtcctcaaaaaactaaaaatagtgctaccatacgatccagcaatgcccctactagatacatgccaaaatgaaaggaaatcagtatactgaagagatatctgcactaccatgtttattgcaacactactcacaatagccaagatttggaagcaacctaagtgccaatcaacagatgaatagaaaaagaaaatatagtacctatacacaatagagtaccattcagccatgaaacaagaatgagaccctgtcatttgcaacagcatggatggaactgatggtaattatgttaagtgaaataagccaggtacagaaagataagcttggcatgttctcacttattttggggagctaaaaaataaattgaactcatggagatagagagtagaatgatggttaccagaggctgggaagggtttttcgggagattggggaggatggttaatggggggatgcttaatctaaatataacgaataagatctagtatttgatagcacaacagggtaactacagtcaacaataatgtattgtacatttaaaaaataactaagagtataattagattgtttataacacaaaggataaatggatgaatactccatttacactgatgtgattattacacattgtatgcttgtatcaaaatatcccatgcaccccctaatatatacacctactacgtacccacaaaaataaaaagtaaaaaacaaatccatctttcagttcactaatactctcttctgctgtatgtaatctgctgaattgatttattgagttcttcatctcagtaaattctatattttcagttctagaatatccacttgattaattttaatagaatctggttctttggggaagtatctatttgttgaaattattacagttaaaatttttaaattcctatctgataattccaatatctgcctcacgtatgaatcactgctcccatgatgtttttttctcttaatttttagtcatttggtcctgtctcctggcatactttgtaatttttgtttaagtgccaaacattgatgcagagcaaagataaatcaccttgatccaatcaagaattaagttggtctaaggtctgtttcagactttcaagaactggcctatttctaggttgccctactcctagtgaaacacccttcagggatctcaaataaaatcctaatgtgtttaacagggtcttttcttcatggtggtccctgaactccaatttttctgtgaaagcctcataagaacatcaaaactctgcttaatactttagcctcttagcagctactttcagctaggcatcttagcctctcattccacaaagctgagaacttggcgaataccttgaggacaaaacctgtacagaataattttgtttccctagctcagagtaagacagctgatagctctaagtttccattttctgttcggcctctatgccttatgccataaattggcaaatgctaaataaaaaacagaggcagagaaagtctagctgtctatagcatgtttctcttttctctgggatctcaggctctccatcctggctatcatggttgttctccattgccttcaagcagctgttggttctattttatgcactttttacaactgttcttgataggtagcttggtctgatacaagctacactgtcttaggcagaaataaaagtctcttcactcattatattttagtcatatatcttaatccattaaaataacataattctatgatggaagaaatttggtaatgtttaaaaaggataatgaagtaagtagaaataacgaaataagtaaaaattgtcaccccagaaaagttaaaattaagtagtttttgacagaaaacttttcaatttatttttaagtctgtaatatgaaatattttataaaatatcaagtgaaatacttgagtttttcaagatctccctttgataatataaatccagtaccccattcagagaaatttatcctaagtatgcttaaacatgaattgagagtctaattataaaaaataaaaaagcaatagcatataatatataaagattatcttatataactacagaaccaaaccagtttatgtttaatcataccttgttaatttccgtttccttttggcagcagtagtcattgccatgtaggatggcaccatgcttggtacaggcatagaatgcttagttgagaatgaagaaggatcaagcctgtatattaatagtgacaaataaaaaataataattttgatttgaactatgaagagaaaaggaagcaaatgtgtccataattagaaaaccatttggtattaatatataaagaaaaacattctgctatactgtaaagattcccttgcttgtcctggggtattcttttctctttataacacaggtaaagttgctatcaagagatttctgcattcccagtggcatagaatagtgaaggtatttttctttccatatttagattgggtatgattatgcagataaagtgagaaggatatgtttgcttggatgacacacattcaaccatactttaggggtatgaccatttcagagtaagttgaggtttgctaaacatcaatagaaagtgtgaaatttccttcagtgaaaatattaatgaataataaacatgataatcctccaacaatagcttgtataatccaccttcagagtaagaagaacaaactaaacatatcttaaggtctctcctagactaacgacggtcaactgacttaagatatcaaatttaagactttaaaatattttgttagtgaattaatattcagagagaaagataattaaaatttaatgaaaataaaacagactgttttataatcaaaatggcacacttggtttatgaaataaacctcaactaatagttgaaagtctatgttaaaaaaaaagaattggcaaatattatttttttgtaccgttgtaaaatgtctttgttatcatttggtagtgattcttgttcgggtaatttacacttcgagctcttgatgtcttcacatatagtaaatgtagagtccaagtcctcctgtccacattcttttcttctattatgagggatagctacttcacacaacattttcagacaattatcactgtttatgtttgagctgatagcctgaaaacttgtagtaaatgatgatggtttcattaaggttactgtagacggattttgaaaagcttttctacagtcttctggtgtatatacaataggctgaagttctttgctaagagaatcattgagctgcacactttgagatggtggagactttagagtatgctgtcctttcaagggagatggcattagttttctccgagttcttttttctgtaggaatcttaaccagattagttccacctgaagatgagcctattcaaaaaaataaaaaaagacactcgataatgtttcctagttttttaaataacccccattacttttaaaactaagaaaccctaataataaggtatttttatgccaattgataaaaatctgaaaaatataagttggaaaggaaaatatcacggtttactgctaaaaactttaaataaagataaactcattataaagaaattctataacaaaagaatatattgctaatatcaatattaatttataggtatagctttcttgagatattgggtaattaggaaacaaattatcaatcaattccaataaaattcacaatttctagctttcatgtttgatttgggtttaagatgaaaacagtattaaacttttataggcacagaggttaacacttgttccattgtaaaatattaccagtttattacttttgttttgccttgttttttagatttgcgggtacacgtacaggtttgttatatgagtatactgggtgatgctagggtttgggtgtcagtgaccagtttattactaattgagtggaagtctagtctaaatgaacaagaatggcaataaccagtaaaattctgggtgaatgaaagcattgtttctgttacagtgtcaggtatacagagttgaccctcagtagctgcagattctgcatctgtggattcaaccaaccagagactgaaaatattcacaaaacaaacaaacaaataaaccaaaacaattaaaaaaatatacaatataacaaccacttaatggcatttgtattgtattaggtattataagtaatctagagatgatttaaagtacacagaaggatacatgtaggctatatactatgccattttatgtaagggacttgagtatccatggattttggtatctgggagggttcctggaatcaacttgcctgtccccaggatattgagagaaaactgtagtcaattaagcattctagttaactgatttgtctcctgtatcatacataactcataggtttgtaaaaacaagatttaatactaaagataaatgaaataattcagagagttcagagtactttcaccctcatcactctggagaagtcagtctcccaaatgtcaagaggtcactaagtctgatgatctttctcagccttatctgataattcctgtagcttttcaaaattatatctgtactattctaacatctgtgttattctcttcttccatctagtaaatgtacacatttccctaaggagctgtacttgaccctctagttgccttgttctacacagtttctcatatttctaattatcctcttaccattaccttctctgtcacctgggttccaaattactagatttttttttctaccttttcccttgcattttccaatgtataccaattctgtcttttttatgcttatagttaaaacataattctgttcatgtcatccttctgattaaagactttttagtaatctttaaatagaaacaaaatatgctgatatttttagttgggtaattcttggtccttggtccaaaattaagaaacaaattgtttcattagaatttattaccacaatactagatttagtatttaatgtttaacatttaaaaacacaaacatctggtaaacaccttttattaatgccccaaataatgacatatgtaacaatacacttgatcaacttcagtgtgattatcttatatttttcaattaacttttttctactctacctacttcattctggtgaacttcatggtgtctgacatgaaatacctttgcttggagtgtacggtcaatggaagaaactattacgtgtatttccattcacgttgtctaatcatgaagtaaaattcccagttgatatttactttactgtataatactaacttaaagaacattgaaagcagaataagataagtgttatgcatttcaataaagcaattcaaactttcaaccaagttagaaaatacacttaaacaatcagcaacagtacaagcactattcttattatattagttgacaatatgctctgctttggctaggttgacaattaccctctaggaaataaaattcaaaaacagaagaaaaatatgatccctgttctactgaggaattttacttttctgagaactagataaatgagatccagatcttaaaataattttccactccctttaacatgacatttgtcaaattttccattaccagatatttttatactaatgctgcagagtaaagacagttacttttctccactgtgctgctaccaccaatttggtggctattaatagctggcagattaacttcttttcccatgagaggcagcaggggtataggggatagaaaactacatggagggtagaggactgaattgggagtcaggagacttggattctatttctggctccaccaacagctcaccaagtgaccttgggcaactcacttaagcagtgtgggcattaatatcccctagaaaaaagcggatattacctttttatatatgaataggattatatgataagggttcaaagaaaggctctagaaatgcaaatatgtttcaactgttaatattacaacttttacaaggagagttgtaatttactttccaatcatcaaaaacttactgttatctctaaaacaatatatttcaaacagtgcttcactttcagtttatcagatatccagtttatcaaggctgatgattaatataaaccacaggtattattgaaacaggtatatatgttctttgcaaatgcagaccttataataacattttaaactttaaaatacaatggaacatgaaatctcaagggacaagaattatagagtcattattttttaaaatacatacttagcaggtataatctctgggagtggcaactgggcaaattcccatcatggtggaccacattttccctactgctagtaaagagtgataaaaatcacagtcttctggtagaacactgaagaaatatccacgtgtcctctgggataatctaagcatacacccgacatcaaatttagtatgatcagactgactaacaatttgtcagtaaaaggaatagtggtgttacagtgatatatctttagctataatcctgctctcctggcagagtataagctgaaggaatagcatggagatgtggtagttaatgtggatttacatacaattgttttactgggcctagaagaagagatgctacagacacacaacatgtatatgatataaatctaaagagttcttttcattctctcaattattaagttcagataaaataaaagggcagaaggcagatggtcaattcagaactacttaggctgattagttgcttagaaagcttcaggagctaaccagagcaaaaacaaaatttaaaggtttaaataaaaatttaagtcaatcttctgtaactataaacataaaaggaaaatgacccaacttaatgaaaactaaaagcataaacaactaaattttactctgaatacaatttttataatatataaatacaattacataacaaataattattatcctttagctaagttaggtaataaagaactttatgtgggtatggcaagtggagagtgttgagttgggggatcagaaagttcaataggctataagaactgttggatatgttaggtctatgtctataaaagagatctgctgtacttttccattagaggaaaagggtgtacagtaaatttccagaagagcaggcatgggacagttgtagtttcccctgattcagcttgggaaaacactatctaacaatattccataattcttgtgcttatagagtagaagtgataagaaggaacttggaggcagattataattccttcagaaaggccccataaaatgagaaaatgaatcacaaagttgcaggctgtgccttgcagaacaagagaccatatagatgatgatagcccataaaataccctagcaacgtgggtgctacacagaagggaacaggaaattaaaaatgcattgttataaggcacgctcttgcaaactggaatagggaagactggcagtcacttgggaagacatatctctagggtctatctaaggaactagtccagtgtatcatatacttaaatataagaggtagtgggtagcaatgtatgttatggtttctgagtaattttgctatttccacaacatctcttgagactttaactcctcaacatattgtaagtgtgctccatttctattaccttgaatgattatgacccatgggatgtaagtgaataggactttttgaaatttatgcccaaataataacaggaagcataatgtcaaaaaatttaaaagaaaaaaatcactactactattttgaatccaggatcaacatatgttttatttatgaaaaaaagttgtagtcagctatggtacaaaagcaacaatctaaaaggagtacacctgcataaaaagaattcactgttcatcccacccaatatttattaagtacctaccataccatgctaggtacagtggtaaacaagacagttaagaatttacatttttgaggggagaaggcagatgacaataaacaaataaaataaattaggatgataaattaattgtaaagacaataaaacagagaaatggaatagacagtgatgggagataggatgttttaactatgatggctagaataacaattctaaggatggaatatgtgatctgagacttgaatggctaaggaggcagttctatgaagatctgggggaaaaggcagaaaaaatagcatgtgcagagatccaacggcaacaatgaatttgttgagctcgaggaaaagaggctagtttgattgaagtgctgtgagttagagggacaggcagaataagaggctagagagatcaaaaggggcaacttatgttaggatttatggacaagacaagggtttttatttattccaagtgtaaagggagatctgtggagggtttcaaatatggatataatgtgatctgattgatgtttaggaaaaagagtgatttgcttgctgtgtcaagaatggtcaagagagaaaacagaaaaatcggtaacaaggctattgctatagcttaagcaagaaatgacagcttgggtaagagtggtaggagcagtgatggagagatgtgagaagggtattataatatctatatatgtaattagctgccttataataaagagttcacagaaagtagtatatattaattctacataagtaaattaagagaaattgacaatgctccataaaaccatgaaccatttatgcatgtcattccctcaactttggcattcttttttttttgacatccccttccctctactcccactcatcaagcttgggatcctgtcctctattcaggagttctgatccatgtaactgtaggagtcctggaatctgcatttttccctttctagtttggacactcatagagacttgaggcagtagaaactctttggggtttgaggctgtgatttcttattcagagacgatgagtgttattattatgctgaggtaggggaaaggaaagaaaaggagtaaggataagtaggaaacagtcctagaacaaaagtagcagatagagacagtattgaaaatgaagttcacctcctaggaagttgtaccaaaggcaattaatgggtaagatttaagttggtagaatgatgttaaaaacagcatctctttttaggttaggcaccctactgtctctgcatattcatctctggacactcccaaacaattaaaacaggtttatagacagttccaggcatagacatactatgttttcagatacaaggtctaattgctcctcttataatattttgttagtatagcattcagttctttaaaaaggagccaatgcattttgatccatgtttaaggaagcttgaaaatgcaaaatgcaaacttgtaatgaatgagttttgataaaagaatatacatattagaatcttttctttttgatagggaaggaatttagaaagacactggctgcttgaagaatttctcactagaaacagtacttggttggcacacttgagaaatacccatagtacaaggaggcatttgaaggttaactgcaaaagacaattttaaccaaagtgataacctgaatagaggcatctagcgcccactcagattccctttaaaatgatcacatcattaaaaaaagggtaaacttgacctaatgttggtaatgggaaaacattatcatctgagtgccagaacattagaggactttctaaagagtattgtatagctcagactcttaagggaaggcatggaggagaatgacttatagtactccctatcctttacccttcagtgctatcccaaggagttaagtaaaagacactggcagatatcagtaaagggccttaaattagaaccagggatgatggggtaaggtgggagttggggagaatgagtgagacacagctatgcaatatagtaaaatggcaacatctgaacactaaaagtagcgctaagaggctctaaaattcagtcctggctcagttgtttcagtgcagaaaaaaaatattaaaaagtacccgatattatcaggcttgttcccatcactgttggctatatcacacatggatgagatactcttgggcaaaggggcagatttctagctgagactacctgtggctctggttccttccctacattccctgggctgttggatcagtggacaggaatatattttttccccttaattttacaggaaaaaacataattataataagcaaacagaaaactcactggacctataaaaagaaaatgtaaagaaatgacagaactggcatacaaactcagaccttttgctaatccagatgccaagttcttcaccaccacacaatgctgagcccctaccacagaagttaattaaatcacttaggcaatgactaaataactaaatgaacatgaacatccacaattacactgatatttaaaactatattttatagattatttgatgatataaaaaaatgttcattaatgaggtagtatacagtatgatcccaattttgacaaaacaaaaacaaaaacaaaaacaaaaaccaaatcaaagcgcacaaacctcctgagcaaaaatggggggaaaagtaatagaaaacagagaaggaaatataatccaattcatcaaaatgttaacacaggtttatagctcactgggaaaataatgaattattttattttcctttttatacatttgattattttatataataaatataatcctgctctgtaaaataataaatgaccaaaaaaaaaaagggggaataatgtttggggtggtgggtaggaagccaataaaaaatcagctctttcaaacctctgaagatctcaggaactgaaggcatgaggttgtctctgaaagcacagggtggagttaaaacaggagaattcttggaagtctgtataaggaagttggattcccagatctcccttaccccattaccatcacctacattgctaggtaactgctcctttgttaccctattaaaagacatgaagttttactctctggataggctgtagactggggccaacatgcacagctgacactaggtgcattaaataatttcttaataataaatatatgctgatttttctcccacttctctgtaatactggtagccaggcccatataccccctaaatggaaaatcaaaaagttctttgtaaatgaaacataaaaataaaaaagatggacatctgtaggttcccccagattgactagttttctggagataactataaatcagagctttgtttaataagtccaaccctagttttgtaatagttactcattaatagtttagcaaacatttttaaaatatttatatgatgctttaaggcatcctataaaaaactgaaagtatgcgtttcaggataaagtggcgagtgttctattcatctgttcctgcattatggctagagattcccgcttcaaaaagtttgtttactgtaagtccaaagacagacttaaaaaggaagttagacaaaaacattttgttgagactgccaagttttagaagcctctcaaaagacttttaagtattaaaaccacttttaaaattaactgtaaatgcatgcactgaaatcatggattagatggagaagaaatacaaatattttcatcaataacccaggttctaaaacaatgtaacatggattacaacagaagtatattaacattagtattatagagtattaaatttgctattaataaaaccacagctaaaaataaaaacaaaaacagtgaaatcataatgaaaacataattaatttgcatgacaaactacatttgctaagtggttataaaaattataaataaatatttggtgagtggttaagtggttaaaaaagtaaacaaagtttggatattcatctgccattcactatctctcaaatgtgtaggattttaagacatataattgatgaatggtagtgtaataaaagcaataaattgggcattttgattttgacaataaaacataggataatctgatgaagtacataaaaaagacttgaaaaaagtttgataaattgactgcctatacctcaagagaaacatctacaaatgtatttattggtggtattgctctttaagctacttaagtctacctataaggacaatcaatctacttcagtatttgcatctttcaaatatttcaattctccaggaagtctatctcacagatttggctgaggactgtgaaacgtttccagtaaaaagcagaaaaaaaggaaaaaaaaaaagagatgcttcatgtctgtttccctaaaattagttttaagaaatgtcagtatattcttacttctaataaatgttttaaataatattttatttaattataaaatatttccttaagtaggctctaaatgttacacatattaacatatgtataaatataatgtgaaattgcatactttatgtatatattatataacatacacatgtcacttcttacctgatcctgctaaaatacattagcaaaaccagagaagaaataaggtggcggtgttgtaagtgagctaattcttcatctctcatgattagaagttataacatctaaacactaatgaatcaagaaacagcagtatagcatactgtttagaaatattgtaggttgcaacctactcatctgacaaagggctaatatccagaatctacaatgaactcaaacaaatttacaagaaaaaaacaaccccatcaaaaagtgggtgaaggacatgaacagacacttctcaaaagaagacatttatgcagccaaaaaacacatgaaaaaatgctcatcatcactggccatcagagaaatgcaaatcaaaaccacaatgagataccatctcacaccagttagaatggcaatcattaaaaagtcaggaaacaacaggtgctggagaggatgtagagaaataggaacacttttacactgttggtgggactgtcaactagttcaaccattgtggaagtcagtgtggcgattcctcagggatctagaactagaagtaccatttgacccagccatcccattactgggtatatatacccaaaggactataaatcatgctgctataaagacacatgcacacgtatgtttattgcggcactattcacaatagcaaagacttggaaccaacccaaatgtccaacaatgatagactggattaagaaaatgtggcacatatacaccatggaagactatgcagccataaaaaatgatgagttcatgtcctttgtagggacatggatgaaattggaaatcatcattctcagtaaactattgcaagaacaaaaaaccaaacaccgcatattctcactcataggtgggaattgaacaatgagatcacatggacacaggaaggggaacatcacactctggggactgttgtggggtagggggaggggggagggatagcattgggagatatacctaatgctagatgacgagttagtgggtgcagcgcaccagcatggcacatgtatacatatgtaactaacctgcacaatgtgcacatgtaccctaaaacttaaagtataataataaaagaaaaaaaaaaagaaaaaaaatattgaaattaaatgaaaaaaaaaaaaagaaatattgtaaatactgtctggctgtggtggctcatgcctgtaatcccagcactttgggaggctgagataggtggatcaccggaggtcaggagtttaagaccagcctggtaaatgtggtgaaaccccttctctactaaaaatacaaaaaaattagccgggcctggtggtgggcgcctgtaatcccagctatttgggaggctgagggaggagaatcgcttgaacctgggatgcagaggttgcagtgagcctaggtcatgccattgcagtccagcctgggcaacaagagcaacacttcgtctcagaaaaaaaaaaaaaaaaaaaaaagagaaagaaatattgtaaatattgtaagtttgttatatataatcatccagcatatattccaatagaaaacagaaaaatccccagtaattttatatcttcctaaggatagttttaagaaatgttggaaatgtccttaaaacagttgtcaatgacaagtcaaatccaaataatttgaaaccaaatgcttggttttagtccatcgctacaagaaaaaaggaataaacttacacacaaatgtttcctaatagcaaaaactgaaaggctcaagcaagataaggccttacgtactttaactatgacatttctatgtcatgggaattatatagtttagcaatctatatctatttttcactgaatctctactttaaaagagtggtcctaactactggccaccaggaaagttacaatggggatcatcttatacaaacaaccagaatttagtactagatggttttagtgtttatttaaacttcattattttataatttgttgctcacagcaagaaaatgacattctcaaagtcatgatagcaattatatttgttgttgccagagttggagtcatgtttcttattttccaggtagaagtcaggtaaaaagtaatgcaagtaatacagagtaataacatatataataaacataatggagacttaaaaaaataaaactttcttcacagactcaaaaatatgaataggaggcaacatactacttcattgctcccaaaatgaaatgctatttcctattattactgaaaagagaaagtaggcaaataaaatatgaatatcttcataacttaaaattgggttacataaaaatcaaaaccaaccatgaataagagagaaaaaaatacatgtaacacatttggcaaaggatttagagctcgtgtatgtgtatgtatgtatgtctatgtatacatgtgtatataggttggctggtctgtctctgagagaaataattcttacaaaacaaaatggataagagcaaaataaggcaaaggaagaggcagttaattcagaacaaactataactagacaaaaagcacatgaaaaaatgttcaccttcaccttcattacccacttacaatggtagactacacttataatagttctgggggtggggaatctgacaatgcatatctaatgcctttaaagtatacatactgtttgattaagctattcttcttctaagggcttagcctaaggaaataattaggcaagttgcaaaataaatacacatgcacagacatttaatggggtgtcgcttgtaataataaagtgaaaacaaactaaatgttaattagcaggagattaggcaaatataatatgcagctattaaaaatgacttaaattcatattcgttgatagcaaaatgtgtctacagcaagtagttcagtgaaaaagaggctggttatcaactatatatatataatagattccactgttcttgttttaaaaaagcaaaccacaaatgtatgcttagaaaaaaagttcagtaggctattaaaactaaatactaatataagttaataaagtatatcttgtttcttttggcttcacttttttccaaattaaaataaacaaatatttattgctaagacagtaacaaggttaaaacactagtgaaaggaataaaaaacttaaaagccatgggaatacagatgaatatatgcaggcaaaggaaagatgatccattaaaaggaagatttaataaagaaggaagtatctgaattggactctcattgtggagaaagatttaggccaagaagtgggatggatgtagagaaaacaagcatttgaaattgatggaaaagcttggaaattgcagaggccagaaacagaaatcatgataacagaaggttgggatacaaggtgctggacaaaagtatgcctatagcttgacactggacttggaaagcctttgaagactcttctgagaggtctacactttattaggaagatcactttgttggcagtgtgaagtgtagactgcagtggaaagagacaagagaaattaaaactagtttagatgtcactatattattcatttcttatgccaaattttctaaaaatattttaaaataaattaagaaacatctttttctctctttgttttaaataaatctctaaaacataattgctaaatggcaattcatgtggtttcttaagtagagggggctttatatttatttttcagcttttggaaatatttcacagatggttcctttacataaaataaagagactagataatttatcactaatgggtttaaaatccctctcttaaaaatgggcagggcttttggggtcatcttttgatgtgccatatcatgtgttctgaatgattttacccagaggaagtaatttatcaaaagtttaagactgtaaacgttaagagatttatagagctgtatgttattgtaaaggaagaatgcggtatttagcaaacatgagtcataggctgcaaagtagggttcatgacagggagacattgatgaatgggattagatctaattgaggtttggtacaaaacgatggcaaggattcttcctttgcaagcaagcactcctgttaatatgtacgtgaggagaaattctgggaaactgtgctaaatcaaaattagttggcaaagttctaaagtgaaatcaaatattaatgtatacatttcaataaaaacttctaggctacaaatgattgatacaattagtgtactcatcataaatctggattctttttaacagccttatgactcatggaattcatagttgcaaatgatacaggtaaatgaattttaactatttataagatacgtaaacttatcttataagataaagtaccattaaattaaccaaatataagataaggtaccactaaattaaccaaactaataaacattttataatacacttatgagacactctcatggttagggatgtataatagttccctaatacatagggaactattgtcctaatacatagggaactattatagtcctttaaattaatccagcactttgaataataaatcaacccataaccttttgatattttttctgtgggaaatagcagatatcatgtttctctctcagtttcattttctcagtatttacacttaccaaagatggaaaagaagggacaatacaaagaaccttttaattcacagtaatatttgcagctatagatgaataactaaatactgtcttatgtacatggggatgataagtcaaacaaaatgtatttagggatacaagcatgatttccattgtttcatgattaaattttgttgccaaatggaaaaaatatggaagatttggtttattaacattttcctgctttatatttgttatatgagaaattagtatagagatttcctatctttcaaatgaaatttccttgtattcaagtaattttaggggaaacttaaacttatgtgtgaactctatattcaatcagaatattggtactatgttaagagactggttctcctagtatcttttattgtactttaagataaaatcactgacattgggtgcaaaattttaaaaaaggaaaggttacagacagaagtattaccaaatgtgagtttccagatggataatgaggaaatcttgacttttcatttaatgtcagaaattttatcagttccataaataaattttgctgtcagcacccccgtcttgcactacacttagtgagtgtgctatctattttccagaccccaaaataggtacatattttactcttagtcaatacacttgtagtttgaaggtaaagatttcacgtcaaataaatatctattcaaaaatcaagtctatttattagaaaataattaaaattcataaaatttacaaaattagtttcttccaaaataagaatttaggcttattaatttaagaaaagtaaaaagtagtctatgtcacaaaaatacaaaatattgtatctagggatctattcagtcaaatttataaaacacttacgaagaaaactctgaaactttgctgaggaacatacaaaaagatatgcatacagggagacaaactattctagagtacgaagatttgatattggaaatatgtgaagtcaatttatgaattaaaaaaatttcatttccttgaaactggaaattattctgatattcatttcctagacaaagcaaagaatatttttcagacaaagaataatgaagaagggctagcatactatataaaaaaaactataaagtatcacatattaaaataatagctactggccctaagacagacaaacaggtgaaacaatagagtagaaattgagtaaacagaattatatatgactacttactgtacaaaaaacacagtggctgttaaggatgcattgccaatatatctatataagaatgctagtctccttggaaaaaataaatttagattcttgcctatcatttaacaccaacataaattaggagaaagtggaaacaaaaatttaaaagcttatgaatggaaaggccttttgaggaaagatcccgaaggcagtaagagagagaaaaaagctattttactacataaatgttttaactttcagcattaaaaaggaaacctcaaacaaaagcaagaataagtggcaaatcttgaagggtatagttatactatgtatattacaggaaaaagttaatatcccttagtgatttcatcctgtcctatggctttaaatatcatctaaatgttaacaactctcgaatttatgttttagcttagacttctctcctgaacttcagattcttaactgcctacatgatgtctccacttggatgtatcttagacaaatttaacatgcctagaactgaactcctaattttctacaacctccaccctgtatacctgctccatctgaagtcttccttatctctctgaaggtgactcaattcttttagatgctaagttccaaaattttagattcagtcttaagcatttctttctctcctattccaaatctaattttcttggaaatcttgctggctctaccttcaaaacattttagaatctgaccacttctcaccagctccaatgctattatcctggtctgaataacctccatcttttgcctgggttattctagtgcctcctcatgtttcagcccttgccatagtataatctattctcaccacagcagtcagagagatcttttaagagcacaattgaggtatgttacatttctgctcagaccatgtcatggctccttgttttattcagagtaaaagactaattaagtcctgatagtaacctacaaggccctatgtgatctgccttccttccctttatctctctgatctcctcactactttcattctctttttctttgtaccagttatatatcctcaccagcctttgaatatgtcaggcaaattcctatgttagagattttgcactgtgattgtgtccctctacctaaaaaagtttcttctcccagatattcatatagctagtcctgtgaagtccttcaagttttatcagtgagatcctccctgaccatgctactgaaaatagcaatctctctcctccctggccacactcctgaactccctttatttctttcttatagcatgtagtacctcctagcattctgtttaatttatttactatattattgtctttctctactagaagtgagctccttgagggtggtggagtttgtttcatactttgatccctatcatcagacaagtgcctgaaatactgctgtaagttaaacactgttgaatgaatgaaaaaaaacctacacttaagatcttgtaaaaaagtaacgattgttactaaagatgactatatcagaaaaatgtcaaaggatggctgggcaattcacagaaaaaagactcttcaattaaaaaaaacacactcaactatattagtaatgaatatggaaaaaagatagtatttatgactattggcaaagtatcaacaagcattgccattactttcaatgatgaaaaccacaattacttctgcactaacctaaaaattagcaaagacaaaagtgggagagagtgtccaagattaaaaaagactgcataggcatgaaccttgtttgtatcttaattcaaacaaatcatagaaacaaataatttatgagacaatgggggaaatttgcacacattagatatttgatgacatttttcttttatacacacttgatgtaaatggaagaaatgatgtgctgtctgagatctacttttttttgttttgttttttgaaataaaactttatttattaacactgaaatttgaatgttttataattatcaggtgtcacaaaatatttttctaattttttccagtcatatacaaatgtgaaaaaaaaaatcttggcacatgggacataaaaaacagatagtgaatcagctttggcctgcaagctacaactaaatgaatcctaatctttttttaattttattatttatttattttttaattatactttaagttttagggtacatgtgcacaacgtgcaggttagttacatatgtatacatgtgccatgttggtgtgctgcacccattaacttgtcatttaacattaggtatacctcctaatgctatccctcccccctttcccccaccccataacaggccccagtgtatgatgttccccttcctgtgtccatgtgttctccgagatctactttaaaataatctagtgtaagagctggggtggaggaaagcagatgggttttcaagactagacatgagctgataagttttaaagttaggtgttgggcacatggtactcatagtattctttgtgcttatatacatgtttgtaattgatttactaaaggtagaaattaaaaatattttaacatcaaaagctggtgaggctaaaaaaaaaaaaaaagctccttcatttatactgcttgatgggggttaaatcacttgaaggaggagagttatttggcaatatatatttcaaattttgcataatgggcatttatcttgagccagaaattttacttctaagaatttattgtagggatagtcagatatgagcacaaagataaatatacatatttgcttactaaaacattattggcaataatagtgaaaatttattaaacaacccaaatgcccaacaatatagactatattcattatagttaatctcatcagattttttttttttttttgagacagagtcttgctttgttgcctaggctgaagtgcaatggtgcaatctcggctcactgcagcctcaacttcccaagctgaagtgttcctcccagttcagcctcccaagtagctaggattacaggcatgggccacaatgccttgccaagttttgtattttttgtagagacagggtttcgccctgttgctcaggctggtcttgaactcctgactcaagcgatcggcctgcgtcagtctcccaaaatactgggattgcaggcgtaagccactgtccctggcccagattcttaaaagcactgcatgtgtgtgtatttaagtaaaaagaatatagttaatctagtaaatacttttaaaggtccctaaggcacttgaactcacttataaacaccgttcctattattcctccttgacactattagtcatgtatacaacatacataattcatgaaatggaaaatacataataaataaatagttgatctctaaaatcatttgaatgaaatagtaaagtaacgaaccaagaacaaaacattataaaaacaaaactcaccttctaatatgtgttcaagagaaaacacgttaattataaaatgtaacatggcttcaagtctttccttcaaaaatatcatgagaaggaattaagagtcaagtaaattcttttgttttattgttgacaagtatagtctgtgcaattatactgtatgtgaaatatagatttttaaataaggaataatgagcatcatatagcaggtggtcagtatttctcaatcttggtgttaaaatcctatttcctttctaacatcttgagtacattataaaaaatgaatcatgcccgaaaggtatgagagaatctgaaaaaaacttaagttttgctttccagagatcacaactaggtcaggtctaatgctttattaaaagaaaaagaatacatatgtgtatatacaatatatatgttttaagataatctatccaacttccgtattttcttaatgtaaaggtataatatagggaacttctgctaataataaagatggagataactcagaccaacctttcttctgaaggcaactaaaagtctggacaaaatataaaaaacatctgtttgaaggcactggattattaataagatggtgaaaaattacaggtgagtatcaggaggaagctggaggctcaggaaagtaatttctgcaatttcagctaatttccccttgaggttgtttgcttatgctggaagagctggcagaaaagcatagcaacagtgctaagagctttgggggaatagggtgctggaactggaatttaggatctttgtaaatcttccttgctttgagttgggactctgaacagctatactgtagagggcaaatcagaaataggcaggacctcaggaaactacagtttagtatcaaattacctcagtctctgaaataggactgaggtgattctgaattgctaatgctacctagaatatgaaagaaggaactgtaaatattctctgagatatttaatatcatgatcagatgcatcaaaatattctgcaaataattttgcaaatacctgactggcacacaaaaaatttgcatgcatatgaggaattaagaccacatgcaagaaaatcagtctaaacagatccagtggttacagactttggaattctcagacacagactttaaattatagttgtctctcagtatctgtgagggataggttgcatgaccatactaaaattcatggatgctcatatcctttatatataaaataatacagcatttgcacacaacctatacaatcctcccatatgctttaataatctctagattacttacaatacctaatactatatatatatactatgtaaatagttatgatgcattgttcagggaataatgaccaaaaaaagtctatatgttcagtacagatgaaaccatccatgttttttttttttttgattagttggttgaatccatggatgcagaacccatggaatatggatggctgactataattataccctttgtgcttaataacaatttttgcaaagatatgaaactataaaaaagaataaaatggaaattccagaattggaaatatagcgactgaaattaaaaattttatggatttaaggatcatttgataaagctgaaaaaagaaagtaaactgaagaatgtgagaaacaaaattatccaggaagagaaaaaaacattaaaagcaaaagagtaaagggcatagacaatacaatgggaaagtgccatgcaatataattggagtcccagaagtagaggagagagcaaaagtgacaaaagcaataggtgaggagataatggttaagaattttccaaaacaaacaaaagataatgcagaaacaaattcagaaagtcaggaacccacttaagagaagtaaaaataaagtcaactaggcataacataataaaactattacaagccaaagactgaggaaatatttcaataagccagaagacagattgtattcagaggggcaacacttaacataaaatctgacttcaacagactattattctctgtgaataaggtcttacatattagcccttcactcccaactatttgtctatttgtcttaggtgagctaaatgtgtgctgggacacatttgagccaaatgtcccagcacacatttagctcacataagaaaaatggactctagttgggagtgaggggctaataaacaccagatcccaagaaaattccccataatggaaaatataggcagatttgactatataaaagttaaaagtccaataaattgatcaattattgctcaacatatatataaaaaaaagcagcaccaaatgaacaaagagttattagatacgaagtgccaagtaaaaaaccaagcaaacaaaaagcccctcaaacccataacaaataaacaaaaatttccactagtgtgaatgagcaaaggatacctgacaggggatgaattggcctcatcgataatctgagagttacacattttaaaagactatgcagctgggcgcggtggctcacgcctgtaatcccagcactttgggaggccgaggtggatggatcacgaggtcaggagattgagaccatcctggctaacatggtgaaactctgtctctactaaaaatgcaaaaaattagccaggcatgatggcgggcacctgtagtcccagcttctttggaggctgaggcaggagaatggtgtgaacccaggagacggagcttgcagtgagcagagatcgcgccactgcactccagcctgggtgacagagcgagactcggtctcaaaacaaaacaaaacaaaaaaaacaaaacaaaaaagactatgcattattattgctgttgttactatttttggtttaggtaaagatgaaacagttactctcatgggtactaagtacagaaaaaaattatgaaaagcagtttaacatgatgcattaaaaaaaaaatgatatgtcctttaacctggcaaatcagcttctaggattctgctatgcacgcacaaatgtgaatacacagatgtgttctctgatgtactgttcactaatagaaaaaaggttaattaaattatggtacatctattctctggattctcaaaaacaataaaaaaatgaggtagattcttcatgtatgcaccctaaaaatgttcataataaacaaataaaaccaataaacataataaactaaacaaaaaagttgcaggtcactatgtgtaatgtgctagcattcacgtgaaacctaacaaaaagctttatgtaatacatgtgtattacacacatactgcaaagataaatatttaacagcatttaatctctgagatagggcataaaattgaggttctgagaagagttgaaggtggcagtctacttcttttcttctacattgtctgaatttttttcttaactaagattatatgttagctttgcaattaaaatgtctcacacatacttttatgagagaaagagaaatatcaagtgatttgtcaatagaaaaatagtaaactctacactgttagttgatcttaagaacagattattatcataaaatatgatatgattaattataatttcctgcccttaatagaggttacattttaaagaaggatgggaagcaaaattgttccatcaaaaatagataaaatgggttaggcacagcagctcatgcctataatctcagcaatttgggaggccaggggagacaactgcttgagcccaggagtttgaggtcagcctgggtaacagtgatactttgtttctacaggaaaaaaaaaaaaaaaaaaaaaaaaagccaggagtggtggtgcatgcctgtaattcctagctacttgtggggctgaagtggaaggattgctagagcccaggaggtggaggctgcagcaagctttgattgcaccactggactccaggctgggcaacagggtgagaccctgtcaccaaaaaaaaaaaaaaaaaaaaaaaaaaaaaaaagaaaagagttgtaaaatgaattttactgctagagttatatagaacatcaatgcatacttgttctagatctttggaagatactggttaattgaaatgactataaagctaattttcccattaattttcataaaatagacaaacctattaaaattaactgttctatagattgatatacaaaggttctctttagaaataatgttactatttacttaaaacgaaagttatacttacaacaaggaataggctgaactggtccaagttgtggaaaggtcataagaacagaaatccctggtagatcgttttgctttggttgttcggcagtttggtcagcccaaactaccacttttttcctctctaccaaatgttcgatctctttgaagtcagattcaaaagcagcatttgacaggccggcttcttttaatgtgcaatattgttttcttagggttggaagtaaagcattcaatactctatatacagaagatgagaagaaaggagagataaatatgacattttaaacacattattttagttcttttatgtaacacccaaagcattagcatatcattaaaataatttcccatgtatgaactgagttctagtctcagattgtcttcacttgcttccatatgaccttggacaaggcaaaaactgaagtttttcctctgtaatgtgagggacagataaatagccactacttataataggagattgctttgagggctaaatgagttaagtatatttgtttatttattttttagagacagggtctcactctgtggcccaggctggagtgtagtggcatgatcatctcattgcagcctcgaactcctggcctcacttgatcctcccaccttaggctcccaagttgctgggaccacagatgtgtgccactacactgactaatttttttattttttggtagagatggggtcttgctatgttgtccaggttggtctcaaacacttagcctcaagagatcttcttgcctccacctcccaaagtgctgggattacagggatgagccactgtccctagctaaatgaattaaatgtacataaaagctttttgcaaaacataatgtgatttccaaactacagatgttgttactgctactaattatcataaaattgaaatgaaaaattaatatactatgtgaaatgatgagacatattttaggtatacaacattaaaacacacacacacacacaaacacataacaaaataacaaaacctacctctcctttaagttaaatgacctaaagaaatggcttttggaatggcacttaaagggattttaaggagaaaagttgtggataaggatggggagtataaataatcgttagggcactggattcacggctttaactcaaaatctcccttattagcaacaattacagcagcaccgaatgtaattttttgtgtatcagacctctgtgtaagtgttcattgaagaaatgtcacagctaaacagttataaatttatacactcaatcagtggttcccaaattttctataaagaaatatgtctaacaacatgaatgatcttgaaatgattcagtcctggaatcctgagtccatcaatagatgatgttccagttgactcataacatcgaggagaagagaaccattagaagtatggatagggtagtgggaaaagactaatcgggcattcactttcatacacaaagaaataccaccttttaacctacaagtcttaaatatttcaatagcagcacaagaaattgtgacctttttccttttggtgggggtggctggagggtgctgcatttaagtcaagtaggtaacaagtacaagaaaaagttattaatcaaagtcatttaaacaattacttatttagcatctacactaatcaggtactgtgcaggaaagttatcatagtaggtctgacactgctaagaaaggcttggttataaggttgatacctggctggtattttaaaatatggattttaggagggttcccagcatttccagaactcatatgacaagagtggctcactgtgcttaaactgtttgcacaaacaatgtggtttatgctaaaacaccttctctccttttgaaaagtctggaattttgatacatcctggcagaaagtgcctatgcaaccagctcccaataaaaacctgggacatggaactcaaaagagcttccaaagcagagaacatttcacatgtgctgccacaactttttgctggaggaattgtgcacattctgtgtgattccactggaggaagactcttgaaactctgtgtcagggttcctgcagactttgcgccctgtaccttttccctttgccgaatgtgctttgtagcctcttgctgaaataaagcacagctaagaatacaactatctgatgagtcctgtgagtcctcctaatgaatcactgaacctgaaagtagtcctaggcacctataacacaggaattatatcatgttatttatttttatgtttaatcttttcaaaaactttatatgaaatgtattataatctattcataacctttatatgaactgtattataatctatttttatataagaaaactatagcttagcaaagctaaataatcagcgtaaggttatattgctcatataagctagaattcaagattgattccaaaactcatcctctttcttctatgctacatttcctctgtgatcctctggttcaccagttttctaaattgttgagttagtctttttccccccattttaaggtaaatgtttatcttaagatagtatattcatctggggtctgatatagcaaatgtaatatggttaaaagaaattattgtttcttgatgacttcagctcttgtaagtggtttgagtgttagtaaatgaatattcaatatctgctctaggaatagacacagaaagtataaagagattagtctgaacttccatccacataagagtaactccctgaagtttttcatttttcttttttaaaaaaagctatactaatatgatttcctctaaatcaagataattatcagttatttgagcataccagtagaacatgaatagattcttagagtagcagaaagcacttagagaaataaatatcccatttgcatggaacatcaaagtgatgcctaaggagttgactgacgtatccaatgtgacaaaaaaaagattaagccatactttttattattatgttttaaaaactaaatgactaatgttactactgagctagaagagagaacataaaaggtctgttatagcatcacataaaaatcaggacacctgatatttagcaacttcactgtaatgacagcaaaaaattctggaattatgaataattaacaattgcctacttaaaaattcaagtgatgttaacagcaattgcttctaagtggagaaatggatggctagaagatagaggtagaaggcagatttcttttcaatgtgtgtaggtacttatgaaattttgtattacatagcatatattaagtaaaagtaacatacttttaaagaaaaacactttcaaatttataaaccaatgtaaaaactttgttggaaggaaataactctcttaatgttttctggttcagacagacacttaaaaataatgtatcctgtaacacataaaactcaactttctggcagtggaaaattgaacttctgtgcttcagatatagtgttaaaattggaaaataggtaaatgtatgtactcacgcttcagtctgcctgtgttgctgttcctgaagacaagctagatccatcatatgtctaatttgtgctttcaaatctttgttctggaggtgcaaatggtgacaatgaagatctttcttgagttcctagggcaaacaatacaaaatgtacttcagatcactctaagaatattgaaattaaatcagtttaataattttatattgttgccaatagttttaattctttttagatattaaataatatgtttagaaacaatttgaaaaataagagttctggaaaaggtggtctataatttacaagcaagataagttttcatgtttcctcagaaactgaatgttaggaattcagaagaaattttactatgatgacagcttcttggtctatccaacttgttcacaatgttgtaaaaatacataaatatgaccagtttttgcttaaatgtcacttcctcagaaaggctttccttgagcattgtatttggatagacactctccacccctcaatattgtcatagcatttactctattgatgagtaacaatttcattgatgtattcatttatttataatttctctgttcacaccagaacatatagtttaccaccaggacaggcaccgagtcaggttagttcagtgttaaagccccagtgtacaaagtcttgagttgtgtgcaataaaaatttctgtttttgcatgaaacaaaagatcatatggttgatatcacctttggttggatgacatggttagagcactggcctatgagtcatgtgattttagagatgggcttataaggccttgaaacaaatgctaccttaagttgaatatacagcctttatggagatcagtttactggactaaaatttaagagctggatcagatgatctcttcagtcctttccagttagaaaattaaatgatcctaatagtgtgaacatgtttgccaaatattacgtctgttctcctttaaaagaaaaatatcctttatatacatcctctcataatctcctttatcttcttgaatctttcttcttttaaagagacaagacttgagacccactcttgatattagctccatcctaggcaaaagtgtgaaaatgtttttttgcaggctccattttcctaagcctcatctgtctagggccaggtgtcctgtaatctaccacctaaagttacttgtctttccttaaacctactaaaccttatctacagaatatatcagcgaagggattacaccaaagtataaatggtgataatctagtaacattctcagaaattttattggaaaaatttcaaacaatacaatgaaaaaaatttaaattctttggacattttgaatggctaagacaggaaccccatatgcaggggagctatgcctggaaactggaaacttcctcctagggacaattctcataatccatcttagctttagcgagtggaaacaatgcaaagtattcttcagattcttttaaagaatattaagattaaatcaattttaataaaataattttaataaatcaaatttaataaaactcctttccaatctgcagttgaccaataattttctaaaatgtcaaagttatttttctcctttttagaaagtacttccaatgccagttattgagactatgacagagaacaatgtcactcatcctatttatatattaaagttaggagttatttatatatattaaagttaggatatattaaatttaggagttatttatataattcattatacttcaagaaatatagtatatttatatattaaagttaggagttatttatataattcattatacttcaagaaatatagcaaatgttttagaaaaaaatttatctttatggaggcaggacgatacggctagagtcatgtgattttagagacgggcttagccttgaaacaaatgtcaccttaagccaaatatatgccctttatggagatcagtttacttgactaaaatttaagagctggatcagatgatctcttcagttctttctattactagttagaaaattggatgatcataatagtgtgaacatgtttgccacatattgcatcttttttcctttaaaaaaatctctccttatagcattaggagaaatacctaatgtaaatgatgaattgatgggtgcagcaaaccaatatggtacatgtatacctatgtaacaaacctacatgttgtgctcatgtaccctaaaacttaaagtataaagaaaaaacaatccctcctttatatacatcctctgataatcttattcctttatcttcttgagtcattcttttaaagagacaagacttgattgtataattgtattagtaataatgggtctttggctagcctgttaactttggaaaatcaagtttacaatttaagtctttcttgacatactaatgactaaatctgattggaagaacatggcttattttgagaactataagtaggccagtaatggctaaagtatagagtataatgaaaggtgatatacaagccttgagaagtattcaggtgccagaccatgctaggcttggtaaatcacaatggagaatttattctagggagaaggcagtgaagaacaacaagatcagttttgcgttttaaaaacttgctttggttgcagtggagagaatgaattgcagtagaacatgaacgaaagcagaaggaccaaacgtagcagtccaataatggggcataggagtctgcattaggatagcagcagaggggcataaagagaagtggatagattcagaatattgaagagtaaataatgcaggagagtacagagaaaacagcaccaatttgaaagtcagaagttctgggtatctgtccagagttgtgtgaccttggataagtccctttatcgtaaagttatgtctcaatttgcttatgtaaaaaataatggcttataactagataattttctcagctattttttcagctataaatatctgcacaaaaaagaattgagggttagatttgggtgatggcaatgaaaatggttttaattttatttgctcaagttatttgtgataaaatgcataaaaatcaaagatacaaaaatcaattccaggttcacattcaggtgtacaaaagttcttcgtaagagaaatctagaagacagattgctagaaaaattagatttgaccatattgaaaaagttttaatttattaaaaatccagacaatattgattaacttcataaacactactaagtagacaaaaaagcaaacacaaaatagcgataataatgatatcagaaacaataccataccatttttaaaattggaagcttacggtaatgtgggatagaaaaggcaaactagtaaatttctacaatcgacttttttgcctgcttaatacttcataagttcttagtacttctgtagttggaatgctgattacatggacaaacatataatacaagcataatgtaaattaagttaggatcttgagaggtgaaaatactctagcaatatcatttaaaacttatatacacaaacatatttaaaatttttaatgaatatacttaatgccactgaactgtatatgtacacatggttaagatggtaaattttattttatattttactacacacaaaaaattgtttcaagactcttcttgagaaaatttttgccaactataaggggtaaataatctatgagaatatcacagccataagtaaacaaacaaattaaggaagcttcagtagttaattttaaatgaggtcaaagtttaatactaaccaaaagacttagttttatgtagtttagaaaatgataatcttgagaatatgactgctttagatatacagtctccaaataaactattaaataataagtgaaataaactgaaaagtactgtcaacattccacacctgctaccaatccaagagaatcaactcttgtgtattaaatgagaaacactcatttggccagggttatccagctgttttctttgaagtgattttatcaacaacataaccatatttcagtattcctacaggcactgtgatagagcctgttgcattctatgatgagcctctatggctttatatggaatttcagaagttatatcctctgatcctagctaacttatattatagtttattaatgaatcttgtctttttttgcttactccttttgagtcattatatcatcttaagatggacacaacgtttaaaaaaaacgtgctatagtttccttctgaagtagatattcttaatcttgtgtcaatggaagggctttaggaaacccaatgacttaggaaatatgcaaaattgtataaatttgacctccccaatctcccagctggaagaaaatctacacagctttgaacagattctcaaaggcatctatgacccccaaaaaggtcagaatcattgttctggaagagaaattatattatattatattatattatattatattatattatattatattatatgatgcagttaactctgttgttaactatgtacacttttctttgtacaacttgcttggatattgatgtctatgctattgttgcttagaatagtattaaactattatttatcatccattattatttgttaagtgttgctaggttttgaatgtagcactccacttaagctcataatatcttaaatacatttacagatgacaaattaagacatcaatgttaaacacactgtccaaagtcatataattattgttagagttgttattcaaaacaactactatatgccagaattaccaaacactccaactccaaagtctatccttttcccaatgtttacattatttttgtggggaatattttaaagtgccttacaaactaagttatattattgcaaaatattactagaatgtgctgtattctatgtttaatgtcctaatctacttaactaactgactggtatgcttttatcaagatattttgaggtgcagattaaattgttggctcaataaatatccacaataaggaaatactgtctttggtttccttcttaggaaacaggatatctgctgcttgctcctttcactactttgaactgtcatcattaactactactgctagtcttttctctccagtcatgtctgagcttccatattaacagaataatcatttacagccattgctagcccatcagagaatccattccccagtcataatgcattgactagtatctttgaattataaaaaaattagtacttttgaacatttaaagatctctggcatttgttggtaaatatttcatattttccttcacctgtattttctgtctggtgcctttctaggaattcaaagattttagaagtttccatatcaattagctgatacagagttatagaatttcagaagagcatcaccaaagtagagactgtagatattttttcttttagaaaaagactgaaaagaagttgttcttttaaactattaagtatttgggtgaaaattagttatttcatagcagttgctcctagttgcttagcccacatttactttcttcttctttcttattaagagatctccaatcccattactgggtatatactcaaaggattataaattattctactataaagacacatgcacacatatgtttattgcagtgctatttacaatagcaaagacttgaaaccaacccaaatgcccatcaatgataggctggataaagaaaaggtatattgcacatatataccatgcaatactatgcagccataaaaaagaatgagttcacgtcctttgcagggacatggatgaagctggaagccatcattctcagcaaactaacacaggaacagaaaactaaacactgcacgttctcactcacaagtgggagttgaacaatgagaacacatggacacagggaggggaacatcacatgccagaggctgtcaggggtagggggcaaggggagggagagcattaggacaaatacctaatgcatgccgggcttaaaacatagatgacgagttgataggtgcagcaaaccaccacagcacatctatacctatgtaacaaacctgcacattctgcatatgtatcccagaacttaaagttaaaaaaaaaaaaaagggagctccaatactgttcagagtaattatgcaaaaaaaaaaaaagacaatcttttcagagtgtcttctatctacagatagccatgtgacatatttctagccaatacattagtctgttaggaatttttggaaacatctttgctctcctgatatagacatccctcctcctttctttcctcttcttctggcttaggtgtgtaaacttgagactagatatgaaacagtaatattacaatcatgaaacgacaagcacgataatgacattttaaaggtactgcattagaaatgaactgcatacttccaagacttattgttaagagaacaaaagacagcccacactagttaaggcactaaagttagactactgcaaccttgtagcagaacataattctaatatgatactgtcattacccatcactacacttcaaaaataaaaccttttgccattctaaggtggaatatcttcagttacacacacacaccccctttcaaatcaactttctttaaccataacacatttaatgttgacctcatttaaactatttttgcataaaatatcaagatttcaaacaaattacctacataatgaatcctcaagtgcaggttgaaatagaattgtttattttaacctaaaagctaccttataaaaggatgcaatcacttagaaataacatttaaaagtaaattaaatctcagagttatgaggcaataatagttgtcttatgaaagacatttactcaataagaaaaagaacacattttaggagctcagttcctacaaattaaatctgaacatacagagatatttgtacctttggaatatgaccgttttgacttaagagtcccatttctttttcgacacgatggagccaattagtattctcatcaaattgcttcaattcctcctccctccttttctccaggtaggagcgacgagttttcaacattgcaagtctatgatctcgttttccagtggcctgaaacacgattcatttaacagtaaatctaatttttatgatatttgatgtacatgatattagtaaactagtatattttcttatgtgtactattgtagtaaattaagttaggctacatttttgtatttaaagctatttcccccttgaaatctcagaatactactgcttgcaagaaaataggaaattgagctgtgagtcatttttataaaattacaggttgagagtttatggctttaagaaatacaacgaccaaatcaaagactataactttttttttttttggcgttaaagtacagtcccttcagtatgtttcatttgaaaagttcatacaaggtcagatacattccatgatctccttactcaaaatcaaagcagatgcatatattgctcttgtctttgatgtgaagaaataatgtgttctattgttctgtgtttgcctataaggcctcaaattggctgatataaatcacagaaaaaagagcttatatcagagtgcttatcagtatctgatcttgcctgtagcttggttcaggagccataatgagtattcaaaatatttttggtgttttacctaggcacaaattgaaattaaaattagtctttgatttcttaccctaagggtcttgcacactatcatgaaaacaatgggccagaacatcttgacgtctctggggtatttgcccacagacactaaagtcaataattatcagataggatgggtagacctgaaacagtggatccccaaaggttaaaataagcatttgcagctggttctggaagcaggaaatcaaaaaaaagaagagcagaaactagatttgaatttggtcgaagaactccccctctttgggagataaatatctcttaattttgacaaaagatgtactattgagacttgggcatataatagaaaaatatggttatcttcatctggatagaggatatgagcttataaatgatttaaggtcaggcaaaggtggctatcatgaagtcataaccaaatcttagatattcataaaattttacttaccttcccctcaagggaaatacaagagtcagagagaagcagattccatttcaggctagagtcatagataagggaaaaatagttgatagccaccgtcattctcactaaggttcctgactcttttttatggcttctctggttaattataatatcctagtctctgtctgtcacagtaagacagtaacaaaaacaaaacaaatgtctcaaagaaacaaaaacctggatagtcaagaagagaaaacaaaacaaaatgtaacccatcaaactgaccacagcgctaggatgagaattgtgacagaatgctacaatgcaggctgggatggtggctcatgccagtaattctagcactttgggaggctgaggcaggaggactgcttgaggccagaagtgcaagaccatcctggacaacatagagagaccgtgtctccacacaaaaattaaaaaataaatttaggctggtttcgtggcatgtgcctgtagttccagctacttggtggggtggaggggctgatgtgggagcatcacttgagtccgggaggttgaggctgcagtgcagtgagccatgactgcatgattgtgccactgcactctagcctaggcaacaaagtgagaccctgacagcaaaaactaaaaaaagaatgctatcatgcaaaggttacccaagcatggtacagggacccctaaggattcctgagatcctttcagagagttcacaaggttaaaactatcaaaaccatttttataatatcaggaaaatgttattagtcttttaaaattcttgtttgctcatacagttagttgcacagtagttttctagaggctaaatgatgtgtaacatcacaacagaataacaattcagcagttatgagaatagagctgatatgaaaagccaggcattaaagaaatttgcaaacatgtaaaacaacgccactcttctattttcactatttttgagaaaataaaattatttatgtaaccacacataatttttattttaattattatttaacatttaattatttataatttattgctatttattatattttattaatataatttttattatttacaattaagctattatttaatattatttattttaaaatgaatcaatgcataaatatatttttataaatctgttttaatttctaatatagaaatattggtagagggaatacatataaacaaaagctctttgaagtcctaaataatttttaaaagcagaaaggggtcctgaaacttgaagtttagagccactgctataacagaaggtgggaggtgtgacctattgacttttctctgctattcagcagatgtaaaaatttaggcaaacagattttagtgggtcttcagttacttcgtctgaaaaatgaagatgaaaatattgtacctaactgaaggcaggcagctaattaaatcatctttattttgctttagcatttacagtcgtagggactcttagaaaccagggcatgtgggtctggtggcagcaatatttaatatcagaaaatcctaaatgaaacctcaaaaagtccatttatgactttttacttctaatgtccatcaaatgtatcctctgtgatggttctcagaggacattttccatcagaatactaccaccacccattgctctaaaatggcaagtaagtgtttgggaatacattctaaaatatgcatgttaaaccctgacattagagtgaaatgtttcccattaagttttaaaaaagctttacatatttcattaacaaaatcccctgcacaccacaaaagtcttgcgagagaacaaacataattctacaattttagttgttactactgctttgtgtaaaataattgctaatatggtaaattcatatttttacattaaaaacaatcttacagattatttttatggggtcatgaaattacccaggagcttccctttaatagtaatttcaaaatccatactccagaactatttatagctgaatcgatgttatgagattctttcagcacttctgataacaaacctctattggagagaatccaaaacataaaattccttgccttagaaaagcagtttactcatactacagtttgacacttctgtgtaaaaacaaaacgaatcaacctctagatgtcttcctatgtttttcagtgggttaactacacccaataactcaagagtttttatctccactgcttacctagtctcataagaatagctatgataattgggaacctgaggattgaaatgaccaaaagtaagagatcagaaaaggcaattgggttgaaaaaagtctaagctactgtgcagaaaaataaggatgaaaacagaaaaaaaaaaaacaaaaccataaaaccagtcaaacccattctattactctatgtcttaatctttctagataaccacattcaaaattcaatggaggaattaagacagtaaaataacaattatctaaattactttgggattacatagtccaagctcattaattatactaaataataatattcaagtgtttaaaaaatggaactcaaagttttgataatgctactcccttgcttgaaagactgcattttggctacaaaaatagattttagactcctagcaaccaacaatttcacaatctataccaatatcctaccttcccaatctattgttaataattcctaacatggcaaatatgtttaacatcatgtgccaactccagtccattcattgtggttctggctactttcaaattctgagagaaggtatggccacagatcagtgaaatttgctgtgggtaagggagtgggaattggcacgaagttttgagtatagttatgcatttctacacgtattgtccaacaaatgtgacctgctctggtcaaactcagcagagcaagtcacatttgttggacaatatgtatagaaatgcaaggcccagtacaaatctcatctttccaccaggaagccttcttactaaaaataggaagtcactgcttacttttctgaatagctatagcattcacggtacttagaatatagcagagtgattaagagtgcaggctctgaagccagacagctggggtttaatattggttcagccacattaattaggtaagtaactccagacaacctacttgagttctcagtgcctctgttcctcacaaatggtgataatattagatctatctcataggcttgttataaatactaaattaattaatccatgcaaaaaactttgaaaaatgtctggcaaatagcgttcaatatcagctgtttttattattactatttatatgacaagcatatagtgccttatcatttttattatttttattgctccaactagtatgtaaataattgaaggacaaacacttctctttctttacctccattgtgtcaagcacaagggttttacatgtaaacaagtgttcaataagagtttcgttaaagttgagatgttaagtcaatatcagcttcctatggctgcatttcttgccttttgtcaaaaagtaaatcttagataactgcacattcaaataattgacacttatataatctacacaaaactgagaagaattattacccactgaagattactatgtaatataagtgctaataattgaaataaaccaggcatggtggtggacacctgtagtctcagctacttgggagtctgagctacttgggaggtttgtgtgagttgtggagtgagaccttatctccaaaaataaaataaaataaacataaaaaataaagaaaagaagaaaaacatcttaaaaatttgaaataaaatatataattcttatataatatggtactattccagaattctgttcagcaaccatatgaaagaagttcacaacaacaaattgataaattaatccaatgagatagttatttggaagtcattacatttatcaaagaatattggcaacaatttgaagttcatgaaagtgaaagtacaaatatagcagcaagatataaattcgaagaaataaattgtttcctttctgccttaaaagaatgcaaggaacatcttaaatgcatctcaaaacacaaaattgaaaagaaaatttcattaaggcataaaaagcacacactcaataaatatattactaattttgataatatagtttaatagctactgacaaactggtatcaatgatacattactgaatgctttatgattaatttccggtacagcgtatctttcccatttcataggctaacaaaaatctaaaatgttagctttgaaaagcactctttaaaatctgacagcttctggacattaagaaatgcaaaatttttctatataattattacttctgtgaaagtaggaaaagaagaaaacatgtttggggtggtagtttgagaggaatgggttgttacttataagataaagctttttaataagtagtatttgaacatcatatgtctatgtaattaaattacataaaatacataaaaatacaaataaactcatcaccaatgaatatcgaaactggggagaattgtcagtattttcaaaatatttatttttctgatctttggtgctccgtagtttcacacgtttaggtatatatttatcttatactgcttagaactcatgcttcttgaatttacatcttattagtcctagataattctcaaccacaaatttttgcatattgctctttttcactaaatatctccttctgaaatgcctattagatgcatgtatgacttttcttcttgttacatgaagtctatcctttatacaacatcttgtccacatgtgttatctttatcctgcatttggtgtaattttctcaaatctgtctcccagctcatgtaaaaaagcataaacataagagcacaggctctagatctggcatgctgcagatcaaatcagaactcagcacctaggagctgtgtcaccttggacaaaatgacttgtattttggtttcctcattgattattaggataatattactaaatatttcatggggttttctgagaattaaaaagagtcaatatatgtaaagtgctttaacattttctgatattgtatcaataaggtgagtttttaattttaatgaataaatttttaatttctggaagtttcatgtgttttttacctcatatttgaaagttctttttttgtggtgtctttatctttgattcctttatctttgttcattcttatattttctattagattgctctagtatctaaagatttgggagttctgtcatatttgctaagagtactgaatcttgtctagagtgaattatttctccatctgttttgtaattttgaaagttgaatttattagtggctagagttgagaatctgtttctcttaagaggctgatttttgcattgcttctgccaggttttctagtggtatcattggtttgataatgtgcattgtaattgaccaactttaggggttttctatacaaaatacatgggttgtaacaatttaaacctcaatcattgtgaaaagcaagcctataatggtagtgttcaaatgacttttttttcttaccccaggcatggttacttggggtaagaaaagcaaccatccttgccaacagtgatggttggcaagaattttttttttcctggtatacccctagtataaaggatataggccactgaaatttcaggctttagataggagctctcatttctaactcaccgtctcctttgggtctcttatctaagtagtgatgtaatcaaagttagaagagtgggagtggtctgtgtttggaagggagagggcagtatattattgtattaaaaaaaatagtaaaactgattaaatgtttggtctgcttttcattaggcccctgccctggcacatctaacaatttcagtgataaaattcttctctgacaacaaaaatctcctgttggtctaagttgtaagctactgcctgcatccaggatagactgcttctattgctctacctttagtaactggctaacctgagtgggtaaacttcgtgttaaaagttccataaaatgcccagaaaaacaacagttgtaactgagattctgttgtttttgtttatttcctttttgttactgacatccaggaattaaaatttctttcttgtgcacttaactatgtattttaaaaaatgtttgctacattttctccaacatttccagatgtctgtaaagaatgggtgagtaagtagccttgactataaaatttccagagttagaaccaccgccaccaggccgggcacagtggctcagcctgtaatcccagcactttgggaggctgaggcggatggatcacctgaagtcaggagttcaagactagcatggccaacattgtgaaacatcatctctactaaaaatacaaaaattagctgggcgtagtggtgcacgcttgtaataccagctagctgggaggctgaggcaggagaattgcttgaatccggaaggcagaggttgcagtgagctgaggttgtgccactgcactccagcttgggcaacagaagaagagactccttctgaaaaaaaaaaaaaaaaaaaagcccccatgtttttatactttctaaaatttaattataaaattgttaattaccttttctactttgtcttcagaacacatcatttctatttgtttatggcactgttgttggtagaatgatttaagttcattttctttaagtaacatttccaacttcagatattcttgtctaatttcttctcgattctggaacaagcagttcaggatttcttgaaacctaccaaaattaaaacacattacagaatctcactaattttgtagcaaatcatacaaagtttacttaagagaaatgcataaacaaaggaatatatgtaattcacagaactacactcaatgataaacagcaaataggtaaaaatgactatttcctattataaaaaatgtttagaaaaagttattaactttacttatactacattaggtttctttctcacccaaggaagatcatgttgctgaaagatatgggcatcataaggtgtctggtattgctaacatactgcattttgtctatgcttacatctatatgtaatcctattgcttagcaaaaataaaataatttttgtttgttggaaaaggatgataacctgggcatgcactatccaaattcacaaggccctattagcaacacagcattatgtgttgttacggtgttggctcaccgtgaaggaataagcagtaggaggcataatcatatggtgcctccatggccccctagggaaagtgacagagccagtttttagcttcagaatttgttatagtgaaaacagaccatacggttaggtaccccctacagcctggctaagaatcaactgacacttccaggacttaaaacatccaagtttataatgtggtaaacctagaaaagctgctgcagtgtgcttcctcctaagggcaatgtaggtctagaatatcaaagatgaggagtttgattcatatttccaaatggccacagacttcaagctgattaaattttatgaaaactctggatggaacctgcaatcctgtatccttctgggtcagtagaacactggtcacagtaatattttaattttttaaaatatattcatggttttaagtcatagatcagctctgagctacagtaacagtataaacattcataacagtgaagactttaaaataaagtaaaatcaagtacatgcatttaaaaaaaatgttctaaactggccagaattttagatatactcataaggatcagcggtaaaaacactgccaatttacgtcagtagctcttaatgaaactctattgggaatgaatataagccacaaaacactgactttactgcagttttgtcaaactgataaatatttcataggaaatatgtacagacactgtgatttcattgcattatattacaccaacggtaatgccagtaaatgaattgataagttatctttaggttatacttaaagagataatttcttagtttttctataactttttattcaaggacaaagttaatatttgtcaattttgtgatctagacttattttaggtggtggtgttcactgattaggatgaaaggaagggaattggtatcaaaggtttctagagagctccttaacaacaccaaaaaaagctatcgttttaaaactgaaagagttctaagcaaacattcaagttaaaagtatctgcaccaatatttttaggtaatacatgagtattaaactccacaattttcttggtatagtcataagtgaagttttataattactctttataaaagaacaattgtgttttctttttatgatttccttttgagaattacctttccaaatagctctgatagacagaaaatactgcagattcttccttttatctgtttaaacttcataaaaatgttatttaagttttataacaaaagacactttgaaaattcatggcattaaaaaagtcccatgtaacagtatgatggataaaataaaagatatttcatacatttatttcaaaaggcagtaatttttaggggaaataacaaaagttatttggtacataatttttatcattaacttcttcataaatacaagtaagttacttaacatattgccagaattaaaatcaaagcttttattgttggtcaaaagtcaggttgaaatatgaaaaacatccaaataaatacttatttcatagctcaggcccttccctcctgagaggagggccttaccagaaagaaaggttcaattatttaaagccatcaactccagaaaagtttactaatctgtaaggttttaattggtatagtagacttggttaagcagtgctgagcaattttatggaatcagttactctaaatttgagaagctggagaattctaatagatatccaattacatgaggaccgctcccacatttaactaacaaaatgaaataacaacaactaataatactatatcagagttaaaagttttctttgatgagatgaaaaaccagaaatcatttttacttatttcttaagaaatactgtaaaatattttatgtaaaacttcaacacacaaatgcaattttaaaaaattatagctgaatcaggtctgaaagccacttttgactataatcttgttttccttatagatactgagacactttctactaattctagagctccaaaacagtatgaataaccattggtttaagttgtagttggtttctcatactaataacagataactggctctgcagaatgtcaaataagtttacctttactcaattttaatgaaagttgaacagattcctttactacaggttttccctttaataagctgatgtactttggaagtttcctagattccttatggagtaatttccaaacttgtttatacaggaattccttattgtcctcctagaagtactcaagattaacatcttgatccagtgtctcaatgggtcaatttggaaaactgtcttagctgcttgaggaaaaagtacttttgcatactgtattactaactgctttctttttaaaaatttacctgctaagaagcttagtagaaacctctcaaaattctgtattggtttatatcactgacagatgttcattcaaataattacccattcaggctcaataagtagattagttaagctatagagagttaataaaatgaagaatagagtagatttacatgaaatatacaaataatgattatgtcagaaaagaacaatatgagcgccactaaaattaaactaagtctacagggtgacataactggctttgtctccactatcttatacctaagttctggttctcttcaatatatgtaaaataaaaactaaaattacttgatctacaaagtgtgaaatataaagagaaatcactgacttcttgaattcagttgagaatatatatacaggcataccttgttttattgtgctttgcagacactgttttttttttttttttttaaacaaactgaaggtttgtggcaatcttgctgtgagcaagtctataagcatcatttttcctacagcatgagcattcagtgtcacattttggcaattcttgaaatattgtaaactttttcattattacatatctgtcatggtgatttgtgaccaatgatctttgatgttaccattgtaattgctttagagtgtcacaaattgtgcccatataagacagtaaacttaatcaagtgttgtgtgtgctctgacggctccactgactggctgtttccctacctctcctcagccctccctattccttgaaacacgttgaaatcgggcaaattaataaccctaaaatggcctccaagtgttcaagtgaaagagttagttgcatgtctatcattttaaatcaaaagctggaaattactaagcttctaaggaaggcttgtagaaagccaagataggcgaaaagctaggtctcttgcacaaaacatttagccaagttgtgaatgcaaaggaaaaattcttgaaggaaattaaaagtgctactccagtgaacacacacactaagaaagtgaaacagccttattgctgatatggaaaaagtttcaatggtctggatagattaaactagctatcacatttctttaagtcaaagccttatccagagcaagccttctaaatctgttcaattctacaaaggctgagagaggtgaggaagttgcagaggaaaattctgaaactagcaggtgttggttcataaagtttaaaagtgtaagaaaaaaagccttctctagaacataaaagtctaggtgaagcagcaagtgctgatgtagaagctgcagcaagttatcgagaaaatttaggtaagatcactgatgaaggtggctacactaaacaacagattttcaagatagacaaatcagcttttaattggaacaatatgccatctaggaccttcatatctagagagaagtaaatacctactttcaaagctttaaagaacaggctgactctcttgtcaggggcttttgcagctggtgactttaagttgaagtcgatgttcatttaccatttacaaacatcctagggcccttatgaaatatgctaaatttaactctgcttgtgctctacaaatagaacaacaaagtctagaggacagcacatccgtttacttaataattttaagcccactgttaagacctattgctcagaaaaaaagattcctttcaaaatactaatgctcactgacaatacacctggtcactcaggagttctcatggagatggacgagaagattaatgttgttttcatgtctgctaactcaacacccagtctgcagctcatggatcatgcaattttgacttttgtgtcttcttatttaggaaatacattttgtagggctatagctgctatagatactttctttcatggatcatgcttctctgacggatctaggcaaaataaattgaaaagcatctgaaaagaatttgccattgtagatgccattaagaacatttgtgatatgtgtaaggaggtcaaaatatcaacattaataggaatttggaggaagtagattccaatcctcatggataactatgaggggttcaagatttcagtgaagaaactaactgcagatgtggtggacatgaaagagaactagaattagaagtggagcctgaagacaggactgaattgctgcaatctcatgattaaacttgaagaaatgaagagctgcttctatggataaccaaagaaaatggtttcttgagaaagtttaccacctactccctataaagatgctatctacgttgaaataacaaaggatttagaatatgacataaatatagctgatagagcagtggcaaggtttgaaaagattgtctcaaatttttaaagaagttctaccgtgggtaaaatgctatcaaagagcatcacacactacagataaatctttcatgaaaggacgagtcaatctatgcagtaaacttcattgttgccttattttcagaaattgtcacagccactccaacttgcagcaaccattactcttactagtcagatgccataaacattgagggaagacctccaccagcaaaaagattatgacttgctgaaggttcagattatcattagcatgttttagcaataaagtatgtctaaaattaaagcaaggcattatcttttcagatataatgctattgcacacttaatagactatagtgtagtgtaaacataatttttatatgcaccggaaaaccaaaacatttgtgtgactcattttattatgatatttcatttattgcagtggtctggagaactaaatctgcaatatctctgagtcaagcctgttatgtatgtatgtatgtatgtgtgtatatataaaattataaacatacatatctatgtacatataaaagacataacaaatgataattaacataaaatacttaacaaaattttaaaacaataaattcctcaaaattagatcttaatgtaatgtttacctttcgatttctttttcctgagggtttgaaatcattaactttgcttggtcattttcattagtgaaggctttctgttcttcataggcttttagtttttcttttaacaataaaatctagtagagagaaatcactagttaaaatacaaaagaagtcatttacatggccacagccaaatttagctgtaaaaaataactgaattaaccagattttaattttaatagaaaaatatgttaaatttttggacaattaagattataaaattctataaatgaagaactgcgcaagactagcataaaaatataaacagacatacctctgccttctgctcattacagatctttacatattgagttatatgattattgacattaagaacattgctcttcaactgttgaaagatagaaattatgattgtttatagagagataataatcacagagataaatacatgaataacatgacattgcattttcattgagttccatgaattcagaatttgtgataattatgctcacatttaaaatctatgacaagaggatctgctgagcacattaataatgtaaatgaaattataaagaaattactttattgagaaaaggtatttataagcatgtaattgcaatataataaaattccctactttcttactcctttgacttctaagtaatgcagagatagtttttcatgctaccctttcaggaattcagttggttttcactgagttcttactatattttaacatttttatcatagtatgggccccttgacatttatggtatagtagagaggatattaaacatataaatccaaatacttgatattgagaccttgcctacttctcacttaacctgacttagaattcgaactgtttgatgtttgggttactcatctgtaaagtggagctaagaattcattcctcaaggggctagtgtaagggctacatgaaaatcatgtgttacaaacctttctgtacacccatatagtatgaccttttacaattaattatataatgagcctgggaacatttttcttaagatgacaataacatgctcatcttcagacacttaaggagagtggcagaggggaaagggagaagaggaggagtcatcacaacagcttaactctaggttttttcaaccccgatatattataatttaaacatacatattttgatatacatgtttttaagtacaacacatctttggcaaaaatactaatgtaaccaattattattattatatttctggagaaatacagagattggtacatcaacatcatgaatgtggactataaattatgtagaaaagactttttaaaaaataagctttatacctattttacagatgaaataataggcttggaaagcttaactaacatatccaagattttcaggtgaaagttttggttctcagctaaagcagagcatcatcctgggctgcaacaaatgtgttcaagttttgagtgattactccaagatgccatccaaatacaatacgccatgacatcaaagaggttagggagaaagccactcaagtaccatagtacccaaaagatcgaataatagtatctattatatatatatatatataatcaagatgctttatggtttaaccgtctaaattaatgttcgactaagcaggttagcaaagacccttctaaccctcagctgcctaactcattatgtagaaacataacctgaattaatactttcattacaattataaaaatcatatgcagacaataccttcacaacaaaggcagagtaaacagacttacagaagatttaatgtcctttgcccggttagcatacttaagagtgttatatgtgtcatcgtagaatacagaggaaggactaacagcagctatcattatagtttgacagtttcctccaagagaatcctttaacaagcgagtaagcttactatttctgtaagggatatgctgattctttctctgaaagcacaaaaaagagatcttatgtcattttccacatttaaatgcaaacctgctaccactttagcacttcactgtggggggaggattacctaggtgccgaggcaagagactgaaggtataaactgtttcagtataatgaaaaaaatagctagaataagaatagtcataatacaacttagatatagagatgatcatggacaattatcaatcactattataaacattattaatcattagcttttaatattactctttgttgcattacgaatataacctaggaataaccggcgggtatggggtcagctgctgaagggacattgtgagaagtgatctagaaggcaagaggtgagccttctgttatgccggcacaaacgccgcttgagggctccttggtcaagtggtaatgccagtgtctgggaagacacccattacttagaagaccgcgaaagggagtctcctttccttggaggagtcagggaacactctgctccaccagcttcctgtggaaggctggatattatccaggcctgcccgcagtcatccggaggcctaaacccctccctgtggtgctgtgcttcaatggtcatgctccttgtccactttcatgttcctcctgtactcctggttcctctttgaagttcacagtagacagcaacagaagaaatagtgaaagtcttaaagtctttgatctttcttataagtgcacagaagaaaacacggatgtatgcgccatctctctctctgttatcggctacctaagagggaagggtcccctgtcctatgatcacgtgacttgcctcaccttgtcaatcacttagaagattcaccctccttaccctgcccccttgtcttgtatgcaataaatatcagcgcgcccagctgttcggggccactaccggtctccgtgtcttgatggtagtggtcccctggggcccagctgttttctctttatctctttgtcttgtgtctttatttattacaatctctcgtctccgcacacggggaggacacctgccaagtcccgtagggcttgaccctacacttcactataagaacttaagcgctacccaattttatccagacttataattggtgattgaggtgatgacaaagaatcttacaggattattcagtgaatatatgaaacaaatatcatatttaaaaatatctgcatctgagtaagtatgaactttagttcaaaataaaataatacattaatattggttcattagttgtgatatatgtaccatattaatataaaaaattaacaatagggaaaaccaggtgtggagcatatttaacaaaatctgtatcatcttagtaacttttctgtaaatctaaaactatttgaaaatgtctatttaaaaatatttccaatcattgaaaaaaattaaaacacaaaagtaaacagatgtcctatacaaatatactagaggaataataactgcatggcttagagctacttttctccttaaataaccatctaatatcgatatgaaataaattataattacaatgttacagacatttctcctgaaatcactatagtgttctaaaagaacctaatacttcaagattttcttttacctaaaaagaaaaaagtaaaagtaacaaataaattttgttaagttttgtttctgattttgaagtaacaaataaattttccagaaagtagaatgtttatataagaacagattctttacaatgttaagctaaaagcaacctaatgttatccaatgtatacctttcatattgcaaaacaggaaattgaagctaagcataaagtgaaatgatttgactagggtcacaaagctagtagttcaaagagccagaaataggtcctagttcccccaattctcagtagcaatgtgcttccaccatagcagtctttcccaaacagtgtgaaatatgcactgctggtgctattttaagtgattttacatgtacaagacatatattaaacattgactcacaaatgactcaataggagtcattattaaaaaattatagattagttcatatggcaaaaatcttatgaatggcttaagtttacaaaatccgtctctgtgtcatgctgtctgcttacaagtttgagattttaactcagaatataaaagacatcttgcttttgcaatcttcatgtgatttcagtaatttgcctttttttaaaaaaaaaacctgctccaattattttaatatacttattctaaatcaaaggtaacattttgaacagtttaacaagccaaagcttttttaaaaaaattaaatttaattttttaattatattttaagttctgggatacatgtgcagaacgtgcaggtttcttacataggtatacatgtgccatggtggtttgctgcacccatcaacctgtaatctacattaggtatttctcctaatgctatccctcccctagcccccacaccccctgacaggccctgcctggcgtgtaatgttcccctccctgtgtccatgtgttctcattgttcaactcccacttatgagtgagaacctgctatgcagtgtttggttttctgttcctgtgttagtttgctgaaaatgatggtttctagcttaatccatgtgcctgtaaaggacatgaactcatcctttttcatggctgcatagtattgcatggtgtatatgtgccacattttctttatctagtctatcattgatgggcatttgggttggtttcaagtctttgctactgtgaatagtgctgcaataaacataggtgtgcatgtgtctttctagtaatgatttataattctttgggcgtatatacccagtaatgggatggctgggtcaaatggtatttctggttctagatccttgaggaatcgccacactgtcttccacaatggttcaactaatttacactcccaccaacagtgtaaaagtgttcctatttctccacatcctctccagcatctgttgtttcctgactttttaatgatcgccattctaactggcatgagatggtatctcattgtggttttgatttgcatttttctaatgactaatgatgagcttttcgaacagtttaacaagctaaagctttcgagtatggtttaacttaaattgccatattataatttttttgaaaggatgtcatttttacaaatatctatgttgtgaaatacactgtgttctaggtaagcacttctggcatatggtatatgtaaactatatttaaatatgcccagaagcatctagtaaacattaccttattaggtgatttcatgacattttataacttaaaccccccttaaaaaaaaatcaacactctttaatacttaaccctattttataatatataaaatactaaacaacagaatttagtttttaaaaatatggttagtttgtcttcatgccaatttaattaacatgtttgctgtgtagtgatcactttaaaaaccaagttcaaaataagtgatgagaatgtaccataaaatattaactatcaatctggtattcattactttctatttaaccaattctcattataaaactggaaattaaaaaagaaataaaaatcagtttaatacactaggttttctgaggtctttgatctatatattaacattactgatatacccttgaaatgtgatatatatcagacctttaaaacaaaatcatattaaatagtatctttcttatctatttcaatatttcaaactatttaacaaataaacatataatgcctacctttgaatctgctaaggcattgatgacattcccaagagctaaaagtgatctattaatatttgtgccttctacaaatcgggtccccttagcaccggaagtacttgctcgctcagatcctgccaggtcaatgagtgacatcttggcaatacggacattttgattgatacttgctgttttgtcttgttgtcgcaagtaaatctttttgaaattacaaaatagaaaaaaatgaggataagatttaacaaaattaaaagggaaatatatactttaatagagcacaaaatcaaaattattttcattttaaaaaacaactgaacaaggtataatctaattgatatttaagagcactataagtaatcactatagctgagaaaaatataaaaatgaaaagactttctatatgtgtaccagaggaaaaataactcttgacttgatagaagatcaaaacttccatttctattctggtaccccttgaaccaggacatggaattatcagaggtgatggttagaaacacaggtcctatagaaataacgttcctaagaaaaaaccaaagctactcgttagaatgccttgtatgtatctagagattgcccctcaagggaagctgaattcgaatcttgatccaaactgaagagtaaatttattctaatcccccaaacctgtatatgtcctcataattaaaatatcatatataaaaaagatagtacagtcttgcatcacttaacaacacagctactttctgagaaatgcactgttaggtgatttcctcattgtgaaaatatcataaatcaaacatgagagaaaatgataaaatccacagacttggtaaacagaatgtagcagactgcttttggggtaataagtaggagcatactctaaaataacaataaaaagtaaatacataaaccagtaacacagtcatttattaccaagtctcatgtactgtacataattgtatgtactatattttcatatgactggcagtgcaggttcgtttacaccaacaacaccacaaacatttgagtaatgtgttacgccacgacattatgatgagtatgacatcactagatgaataggaacttttcagctctgttgtaatcttatgggaccactgttgtatatgtggactgttgttgactgaaatgtcgttatgcggcacatgactatacagtaaagtctacttgtcattttcaactgcctagtaaagcataatttattcaaatattttaagctactctgtaaggattatctgaactacattaggtggcaaacaaaagatgaggaaagtaagatattacttaattataaacccatttattaaattgttttataaagcaggatctaagatatttcagagacatctcagtttcacagagaaacatttctctcaagactattaataaccgaagagttagtggtaagaacaaaaaagattaatgaaatttttgttgaccagggactatatgaatggtttgattgccttcaatatcaattctttcaactataacaatttctttcttataagatgtatctcagatggcataatactaaaagaagctttatattatcatttgggtattaaaaggactagaaactataagaaataatagctattaacctatgctttagctctttctggaattgtattcctaagacttactgtgccaactgaatcatacttccttaaggaatgcgactatttacatggtccttatggtgaaatcttttaaagatgaatcagtatgttcccaaattattagttctacaagtctagatttatagagataagttttactgtgaatataagattatttattggaaagattaaatattttcagcaaaaacaaaacttttattactacaactttatcctcctgtatgtttacaaaggtagaaattttgtatttttgttttaagcatctcaattataaatactcttggtgacagaaacttttcaaagctataaaggaaagtactgacaagtgaagtctacagacagatttttttcacatgtatgaaaatgatgattcatgaatttagcttcatttattttaaatccaattttaagagtgatagtctagtggcctcttacttggaaaacagcatgagaacgagaagatgtggcattcatatcagtgggatgttgtgtcctgtttttgtttccattatccaataaatgtaaaatttcttctgaggatttgggctggagaagtttaagtagaagcaaaatttaaaaatcagcaatatatctttaaatttttcaataaatgttcaaaaaacaaaaagatttaaaggtaaaagaaaatgttttctataaattcaatcctattaaatactaactttgtctcatctgcttagtattgtgtgtgtttatatgaagaataaaagataaaatctagtttctcaggagtggagtctattgagtagaataatatgacattaaatgttcaagcagatttttgaaagaaaagctcactcattgggaggctgaggcaggcagatcttgagccccagagtttgagaccagcctgaataacatggtgaactcccgctttctaccaaaaatacaaaaattagccagtctcatagcccggtcacaaaataaataaataaataaataaataaatacatacatacatacatacatacatacatacatacgtagattaaaatttaaaaataaaatttaaaaaatgtttttaaaaagttcactctattttaaaagaaaacaaaataagactaaaaatataccctaatcatttaaattctgtacctcattagttttttttctcaaacaggatggaaagtaagtgcatggctggtgaaaatagcttaatagtcacatttgctattctaacagggaaaaagatgtttatatacatacctggtgtaaagtaagtccatgaacgaccacccctttttgggtatcttcccggacagcaagtggccctgaatttactaagagatcacgaatctgttcattatataccttaaaataaaaagaactgctttagcattgatgtaaaaaaaaaaaatggtgattacatcacacatacactgctaatttaaagttattagaattttatcctaaagacaagcagcacctcacttttattcacctcataccttcgatctcatgttgccattaattttctatcattccagaaaattgaagagtttgattacctaggtttgaaattgaaacttaaagttggagtcatcccctcactcataattcctgccttgatgactatggtgaaacagagataagtaagatagctgaagtgcattttcttcttttctttttttttgagacggagtctcgctgtctcccaggctggagtacagtggcgcaatctcggctcactgcaagctccgcctcccgggttctcaccattctgctgcctcagcctccccagtagctgggactactggcgcctgccaccacgcctggctaattttttgtatttttagtggagacagggtttcaccatgttagccaggatggtcttgatcttctgacctcgtgatccgcccacctcggcctcccaaagtgctgggattacaggcgtgagccaccgcgcccggcccattttcttattttcaggaaatgtatggaacacacggtactgggaaactctgggaaatgcaggttgtatggagatggcatggttcttactgtggaagatatctattatttcacaatgtccaagtagtccaaacttttgaaattttagagcttaaatgttacagaaaacccataccaaggacaggtggtatttgtttcggttatccttcaattacattaattttgacatataattactaataataaatttcaaaaggagacccatttgaatgatgagggaactaagactctgagaggttaaatgatttgtccatggtcataaaggtattagcagagtaggaactggagcctagacagcttcactctactgcatcatactgaatacatatctgtagtttatatctctggccattttttaaaaagtaatttttataagtaacacacatttctatgtagtactagtgacccgattttatgaatttattaaaggtataaagtttattacttgctaaccaaatgacaattacaactattactgacaagatttaagaaaaaaaataacaaaatttagtctctaaaaaattattttgtgttatttccttctgtgttctaataagtagctcatagtgataattcattgctggtacaattctctacaatgaatgataggcattagaatgtaataaacccttaaaagtagtaaaatgataatatgaagaaatatttttaaatcatatctttttgatgagcagcagtcactttactattgcttccccttacccacttcaatcataacaaatcaaaaattagaataattgttctcaagctggtctatgtatgacactcaccaaggagcatttaaaaaacacagatgcctcagccacaccccagacctactaaattaaaatatcccaagtgtgaggcctaggcctatttttaaaaagttctataggcaattctggtgcacagcttgagtagaaaaccatattatacactgccttatcattaataatatgaaagtagattaaaagaaaaaataaaaattgtgaaaataaccaaacttcaaaattctcccagttataaaaaggatctaaggaaaaaataatgaatgctggcaccctcttttcccaattgagttcagaacaattcttttcatagttaaccaaccaaagaaaatgagttttacttcttttttatctataagttctaatgaagaaatttttagcaacatatatgattttaatcaatctttggaagaaattctcagtagggaccaaactacaagatgttaataagagagaaattttatggcaccagaaataaataggcaatgaaatcagggtttttatgtttctgcactggtttctgaaacactatttgaatgcaaagttagagtgtctgccacttagttaccatttgttgactattaagagggtagaaggcctcaaagaatgatggggtatttcaaaaggacacaagagacaacatgaagaagtctcaactagccaaatcttacacaatttgaacatacaaaaaatgattacagaaagggattataatctattcaataaagtaaaaatgcaggaggctgatataataaggggaaaactcttccttatagtagaaagtcaataaagaaatgtacaagaaatgatggaatttaaaaatcaccatcatctaccatcataatagtaattgattcagtcaccaatcatcagtggatactaataacaatagataaaagtttgaggagtaaggaatatttacattgtctacatacatagtacttactaattataaatggtaaaacgtatcttcaaagtggagaaactctacagaccacagcataataaaactgatcaaagttaacattcccagtatggggactaatcaacggcttaagccttttttgatgcagtaagaagtgaacatcactttgcttgtgttactgtcagtgcataatctgaatctaatcatgataaatccaaactaaggatttttatggcagtgttataaaagacaaaaaattacagattaaggagactaagatataacaactgaatacaattttcttttacaataaagggtattaatgaaataactagcaaaatctcactaagatctacagataattatattgtatcattgctaatttcctgattttggtacctatttagcagttatttttaaaaagtcctgtttttacaagtctgcaacctagctcagaaagaaagaaatgtatacacacacatagaaagcaaatgcagtaaaatgctaacatatgggggatctgtatgaagggtatctggaagttctttgtactattgttgcaacttctctataagtctgaaattttgccaaagtaaaaaaaaaaaaaattaacagaaaaagatgcataacaaatattcaagacaaggaattttaatgattttatatattcaccggaaaactcttatataataaaatcatttgtatgaatcatgtcaatgatttcccaaaactattgattaatctaaattcccaacttacctccagatatgaaactgcagtactacatattttctcttctttaatctcatccatgcatttgtaaaggtgtaacattgttagatacatcactccaggttcatcagctgatcctagcatagtgtgggtcttcccagcaccagtggcaccataggcaagtactgagtttttaagaaagggatcaaaactctttgttatgaaatataactctattatatctactatctagtggatagttccatctggatatcctgaacagtatctttaaaccctacaaatccaaaattatagtcttatccttaaaacctgggaatgttaccacaaatctactcttactcctatgttctttactctgataaatggtatcactacccccggccccccactctaacaactactctttcaaagcaatcttaaaatctccctacagattagtcttcatcttccatcttctttatcctttgcaatttgtagtcatctttatgaaaaagaaattggaatacgttaaaccttcttaattgttccaggtttacataagttcaaattcctcactttggaatattataaagcactcagagggtgcctgcttattttgccagcctactgcttccttgctgtgatctagggatacttaattattcaatactctcagcatttgctgctcctttctactctgctgtctttgttctgaggagtccctctacttgatccagctggaaaacttccacctgcttctaaatgctcagcacaaatatcatttttctctgccttccctcattgtcctaggcagacacatccactctttcacttcagcccttagtatatgctgcatatccacatccctctcttctggcatgtatcacactgtatttccaattgagtgctccaccaaggcagaggccatgtatccttatcacctaacaaatcacttgccacaaagcaggtacttgaataaataccagatagatgccatttataagattcctttttaagtttttgttttaggtgaattactttgtttcctagattctattcagtacaagaaagtcatgtgaatttcttccaatgtataaaaaatccactctgtgggccaggctcggtggctcatccctacaatcccagtactttggacagctaaggtgggaggactgcttgagcccaggagtttgagaccagcctgggcaacacagtgagaccctgtatccacaaaaattttcaaaaaattagctgagggcagtggctcattcctgtagtccagctattcaggaggctaagatgggaggattgcttaagcccagcaggttgaggctgcagtgagctgtgattgtaccagcgcactccagcctgggcaacacagcaagacttcatcaaaaaaaaaaaaaagacaaagaaaaagaaaaaaatccactctgtgaattacatatggtaaatacccaatacaataatcagttaaggcaataacttctcatatcattgcagtgagctgtgattgcaccagcgcactccagcctgggcaacacagcaagacttcatcaaaaaaaaaaaaaaaaaaaaaaagacaaagagaaagaaaaaaatccactctgtgaattacatatggtaaacacccaatacaataatcagttaaggcaataacttctcatatcattattggttgaaaatatttttataaattcaatctgtctgctaaaccaaatgttaacaaaccacaaaaatacttttcagtaggatgaattaagatggtgtatattttactttgttctattttatgtaagctggtatataaggaaggtttaaaatggcttattcttaagcttattcttaagctgtcttaaagacagggtaagaatataaagaaagccaaaaagaaaccaaaaattcaatagtagtttgatagagagtctactttttttcttagagttaagaagatttcaaaatctctagagacatatgaagtttcatgaaagaggaaaagaatatcttcttaaaaatgggaatcacagtagtaccaatcaataaaccacactccatccttacttgatcaagagggtatgtcttccatgataaaactgaaatgaatcaggaaaagacagtgaaacatgttatagttgtgagctgtcttggggaaaatatcataaatactaataattacaaaaataagatattaataatggtgtttagtgcctggtaggcactgaaaaactggccattttgcatcttgggtatctctctctctctctctctttttttttttttaaaaaggaaatcaacatggctttcttgaaaggccaaaaaaagcactaaagatgcttcttgggctaacaatgagctaaagggcatgagataattaacatgggatcttaacacttttaccccctttccaatttttattttaggtttgggggtacagtgcaggtttgttacatgggtaaattgcatgctgcggggtttagtgtacagattttttcatcactcaggtaaggagtgtagtaccccataggtagttttttgatcctcagtctcctcctatccttcgccaacttaaacatttttatttcaatgtgccatgaagaaaaactgctgaaaagaaactgtcaaatgttcagaaatgtctgactactctccaggaatagataattacataggtaagctgtaggaaaagttataaatacaggttatgagaaaactgatgttaattgcaataagcatgtggtttaataacataggcaactgaaaggacagatgtctataggggccacagtaaggccctggaagagaaaggtttacaaatgatgtataagttatatatgataactaaaatttcaaagtctatatttacagttcaagtctattgaatttcttaagaatttttagcatacaagctccttaaatttgattatatttagaagggtcaagtagatgtccgtcctaatgcaaaagtgaaatcggatagagaaattaaatcccaaattgaaacaaatacctgtgcaattatatccattcaaaaaactacgaagaattggcttagtagtgtgttcaaaaacttctgactgagttgacgtttcatcaaaaacagcatcaaatacaaatttaagatccttattttgtttctttataacattttgatttgtagttttctttccatggaaaaaactgacttcttcttgtttgggatcaaaaactaggatatgtttatccacaacatgaaccactttatgaaatccagctgctttttctttagtgttttccggacgtacacgaactactactttcatatggtggcacaggtcttcctcagtgacagacattgttgattatcttgattcctatctgtataaatacttgaatacttctctgaaattaaaaaagaaaataaagttttaatatcagtttttactttcatgcaaaacaactggcatgtagtattcattcaaaaaagatgttaataattctgtttcactcactaggtaaacactgggggaaaaatacatacatacgtaggcacacacaacacacacacacaaatatatgcacacatacacacatatgcacttaagtaccctaaaacaaacaattaaatggcacatacgtacacacaactacgataaaaatctctttacctgaaatattagtaggatatttggcttaaactcaaatactatagctgaaccatatataatcacagatatgctatcctaaaacccagatttgattttcacaagccctccagcactatttcagcttaggtcatgggaatgaatagaaaaggatctcccagaaccatatcaaggtatgattggagagccttagcttgtgaactatttttagtaaaagtgatcttggctgcaagccccttggccgtggaaagtgatacacccaggctggtctctacctgtatctcagaagcccatagggttaaaaatagttccaaggatgcattcaactagatagctcaggtctgaaggtgatagagatggcatagactccttggtgtattcagagacctagtctctgaatattgatcctactctctgctttctaattgtgtgcaagtttaaggaatcaatgatagcaggtttaaaaattatggtctgattttatgactggttcacatgcaaagaagaaaagtaaagtaacaaagatcaataatttggaagtctcaactatccagaaaatactctagaatctgcagacccaaaaaccaaaacaaaaaaacaaacaacaacaacaacaaaaagacaaaaaaaaaccaaaccacaaaacaaaataaaacaccccaaaacaacaagatctgagtaaggaccaaaaggtgagacttaggacgatcctgcaatcccacttctgagtatatatccaaaggaattaaaatcagtatgttgaagagatatttgccctgccatgttcgctacaacattattcacaataactaagatatataaacaatctaagtgcctatcaacagataaatagatcgaaaaaatgtggtatatgcactcaatgaaatactattcagccttaaaaaagactaaaattctgtcatttatgacgacatgaatgaatctagggggcattatgctaagtaaaataagccaggtacaaaaagacaaatatcacatgatctcacttttatgcagaatctaaaaatatgaacacagagaagtagagtaatggttaccagaggctgaggagggtggggtgggtggtaaaaggggaaatgttggtcaaagggtacaaagtttcagttatacaagaggaatagttttggtgatctattgcacagcaaggtgaccatggttaattgctaagagtacattttaaatgttctcacaacaaaaaaatggtatgtgaggtgatggatatgttaattaattagcctgatatgctcattccactgtgtacacatgtatcaaaacattacattgtatcctataaacttacacattttttttgtcatttgtaagttaaaataaaagagacaaaaataataaattcacccaaaacaacatttaagtaaggacttgaaggaggtgagggagttagtcatacaggtattgtaggaagcttttccccatagagagaaggacccagggtgctactttatgttggccagaatggagtgagcaaaggggagtgtagtaagacatgagaggagaaatggaggccattaaaagaactttggcttgtactctgagtaagacaaagaacaactggaagtcgttttgagcacaggagtgacataatctatcactctcactgctatgtggataatataatagactagtctattgaagtaatgtagacaagagacaactctgtcagctcagaatagggtagtggcagcagaggtggtaagtggtcagattatgggtgtattctgagggtaaaggtgacagaatttactgacaggtagatgtagagtgaaagagagagacaggattgaagaataactccaaggtttttggcttgagaattagaaggataaagtagtcaccaactgagatggaaataagaagcagggcttttttcagcagggcgggaagtggtgggaggcagggtcagatcagttcagttttggatagcttaagtctcagagttttgacagacatccaggtggaaatgtcagggcaggtggaagttaagaaaataagaaaataaaggctgtcctcagtcttaatgcagtattaagctttaaaaactttcgggaaatcctaaatgaaggaggaaacagcgatctattgtgttaaatactgctaataggtcaagtacttactgagaattaaccatgggatttactaacacagaggccatcgagaaaagcaattttggggcagtagtggggaaaaaacccgaaaggagaagatccatagacaaggtatatggaaaggactgtttcaaagggaggagaaaaatggtgtgagtgaagggggggggtggtgtcaagatgttttgtttgtatttttttttaaatgggagaaatagcaatattcaagctgaaatatgtcacaaaaccacttagccatatactttcggacagaattcaaataaggacagaattcaaaggaatctaaaagcagtagatgctagacatatctttgcctttttaggactctgtctttggccaaaaatagaatactgaaaaggaagggatggtcatttggattctaagtcaggtcccaggtgaattactactttgtttttcatccctgagcttccgtctagccaagtgtcacaaagcacctacacctcacttttatgaccttggacaagttacttaaccccatgagtctcaggtttctatttgtataaaagagataataatacctcaatgtttgttgcaaaggctaaaaggggtaagtattaaggtattcagtaaataccaattctcttcctatctgccccactcctgcttcttcttgccaaagagactgagttcatcttttgagtttttctcaatgggcttatgactagcactttgggtaggccaattcttccttgcttagaactgccctgttatagggcatttagaactgccctattagaggatgtttggcatttctctcccctgtctattaaaggccagcagtgtcccatattccttgtgataacaaaaactatctcctatacacatttctaaaagcttttagaaatattcaacaatattttaacagatatactatttttaaatgatagattaaatatacataaatgaaattgttaacaaagtaaacataaaatactaagtgattatttcaaaataaaccttagaatctcaaggctggaggatatccctccctaattaattagttaattaattcatgacaactattacatgttggagccaggcagcctaggttaaaatctcagctctatgacgtactgactgtgatagtggataagttacttaacctccttgtggctcagcttccccatctgtaaagtaagactaacaacagtacctatctcataaagttgtaaagattacacacttagaacaatactttgtacagagtatgaactacaagtgtttgctattattatttacagtgtagtgagggaaacagatattaataaaatattgacacaaacatgtaaaggtataactgtgacaagtgccattatagggaaaaggttaacagtgctttataagactataatgaggggattatttaagtaacattccagggagaaatttggacaattacctaaaggaatgaggactaaactctgattttttattttgcccaaattcctatctaaggggtctggggagtcacgccctataaatcatggattttcattagatgcgttttatttgaccccgtatattgtgacttgcttttcaattgactctggcataacattatgagacaaagaaaaaatatttaacacccaaaatagatttacttgccaagccttgaaattgccctgcaaagtttcttgttggaaaagtccacgttctatagagaatccccttccctctttgttttcctcttttctttgcagatccagtagataatcaactaaaagccaggcacccttttagctctgcggagaaacattttacaacctgctctctctctctgaaatttgctgagagcttcctctgcaaaataaaacttggtctccacaattatttttaacctgaacattcctttctatcaatcccaggtctttagacaaactcaaccaattgtcaaccagaaaatgtttaaatttacctatagcttggaagcccccgctttgagttgtcccgcctttctgaaccaaaccaacgtatttcttaaatgtatttgattgatgtctcatgcctctctaaaatataaaaaaccacgctgtacactgaccactttgggcacatgctctcaggacctcctgagggctgagtcatgggccatggtcactcatatttggctcagaataaatctcttcaaatattttacagagtttgactctttcagtcaatagaatttaagtataatgtcaaatataactttgtttcacaactagttgcatattttaacaaagacatggatgttaatttttaaagtatcatgtatttccctatcttttaaaaacaaatatgtggatagaattttcttaataaaaatgcctaaggtcaacagaatcatcaaatgtaaaaattaatctatgttcaaatatacttacatctgcaggaattaatgagatcgttcctacattattatgtggtgccttctttgttttctaggatggctgactaaaagaaatgagcatttagttgattctgaataaggcacagagagagtaaattcttcaaccatgtttaatatgaagcagcaaaagaaaagcctaaatatcgttatctgcgcaagaatgtgtggattcacaaattcttttcagacagctgttcagaagatagtattatagcctctgatcaattttgtcctatctacccctaagtggtatttactgaacagaatcaaaagagcaaactggtaggatttcattggtggaactaaaacatgaaagacaaatacttccaacgtgattattcattataatcaggttgagcattccaaatttgaaaatctaaaatctaaaactttgtgagcaccaacatgacactcaaaggaaatgctcattagagtattttggattttcagattagagatcctcaaccagtaagcacaatgcaaaaattaaaaaaaaaaaaaaattccaaaattcaaaaaaacttctggtaccaagcattttatataatagatactaaacctgaagaagaatatttgtctagtctagagagagtagggtatcaaaaaagaagaatgaaggggctaaggaaaggggctttacttgctaatgtataaaacaaaagggttagattatatgatacggaaagtctttttctagctccaaaattttatgattattatgcatgattagaaaagggtacaagagaataagaaatgacagttaatagacttttactttatatccctattctctcatagagagtctagtatatagtaaattttcaaatataccaatgaatgaattaataaactataaaaaatgctcacagaccccaaaaagcaatttactcacttagagtagaaggaaaaagaaaatgtatatctccaagagaaacaacatattagagtacaatagttgacaacaacaggcaatcacaatgagaaccctgaggcatcaaaaagaaaaaaataagaaagaaataatgttcccaatttcttgtttagaaaattaaagaaatggcaagatcatgttttattttcatcattaagtgtggttaaaatactttattacttagcaaagctaatattcaattttctggaagattcatttatgtagaacagctcatgcctctgcaatgtttaatgaggtattaaaatctatttatcctgaatacttgcatggtaataagccaagttgaacaggtcaatttttaacttataacatatgtagctagtgctcatcccagattaaagattcacccctaattatatgcaagtacaataaattttatttaattcattgattgtatgcactgatactttcttcaaaagcattcagatcatgatatgtattgcatgtcatttacctgctccaaactatccaatgacttctcatcataagaataaaatacaaactccttaccatggcctataaattccacatatggccctgcctacctctctgacaatgacttctactgctgtctccccatttcattattctgcttcagctgcaatgactttcttagcattcctaaaacacttcagtcttgttttggcctcctggcttttttatttttgcttactggcaggcattacctccagatatttgcaaattgtaatccctcacttcattattcaggtctcttcaaatgccactttacagaggctttccctaaagaatctatctaagatagtgtcaccctgcctttgcttctccaatcccttactaggtttatttttctttcttgagtttagcactaattggaaagtatatgttatctggttacatatttattgcttgttttccctctagaatgtaagcactgtgagagcacggagttcatctgcacaccagcacctaggacactgcttgtcaaatatttaggacccaataaatacttgttgaataaatgaatgaataaaccagtacagttttaatagacttatagcatgttaccatttgtatttttaaaagatatatatgtatatgcatggtcattcccagaagggtacacaagaaattgataataccactttccctatagaagggaaatatgaaggtctgagtgagagacaggtttattttccatttcatattcttttgtaatatttaaattttaaacaaggtctaggtatttttatttcaaaacagacaaacaacaacaacaaaaccactattaaaatttaccttaatcgcaacatttaatttagtaacaataagttatattttctattgagctcatatcccacaaatagaaatgtactaacaattgacatactcttactaaatcgtcataacaacctatgacataaaccctgtactgtcattcctattttattaaggcagaaagtgtaatacagagaaattaactggctaaagtcacacaactgaggaagcacatgccatgggatttgaactcagactgactgaatctaaaggccaaatacttaatcactactctccctgatgtaaatatataaccctcagaaattttcttatagaagttaaaaatatatatattgcttacttttttagaaattttaaaacatttaattggcaaagattgtataaaacgaagatgtacaatgtgatggtctcatattcttatacacaatggaatgattaccacaattaaattaatacgtctatcaccattgatgctgtacattaaatgccatgaacttatttatcttataacttaaagtgtaccctttgaccaaaatctccctatttcccccaaaactcagcttctggtaccactctcttatatttccttacttttgatagaaacctcagggcctggcacaggagctcttgaatgtgaaaataaaactgattatgatttctctgtacttgtcagatatcacagagtggccattctcaataagtatgctttaacaatgcttagattaggttgatatggtgataattattaataatgaacaacaatgcttttcatttgagtatgtacactctgtctcatgcattatagtaaacacttgacattcgtcattgcattcaatccttataataatcacgtaaggtaggttgtagtagtatttccattttacaaaaacaaagacttagccttaggataagtaacttttccaagatgacgtaacaagtgggcaaccaaatgtcaaatataagtcaattcagtcaaagattcacccctaattttatgaaagtactataataaatttaattcattgattgtattcactgatacgttcatcaaaagcattcagatcatgtgatgcatcacatggcattcacctgctctaaagtctccaatgacttctcaccagaagaataaaacacaaactccttaacgggtacaaatctaaagtccatgttattaacagctaggttctactggaatcagaagagaatgcttccaaacaatgatacagaagtagagtgacagttatgatatattaaatacaaactcaaacctgaattagattcagattcaacaaacactgtataggactgaaaacgatactcaacactatcatctacagtccatcttaaagctctttttggaatatcagtcttgggggcactactcccaccaacttacaaccatcagggaagaataaaaggttcaactggagggaaggccggcactgacagctgagctattttttttcctcttagttttcacagttgtcagcaaaaaaaaaaaaaaaaaaaaaatcatttttatcgtgtctcttaattgatttggggtatttctatgatgggacaagatgcactttcaatataaaaatattatgtatttgtaaatacttttcaatagtagaagaggaagggctgggtgccctgcctcacacctgtaattcctgcactttaggaggccgaggcgggcagatggcttgaggtcaggagttcgagaccagcctggccaacacagtgaatccccgtctctaccaaagatacaaaaattagccgagtgtggtggcaaacgcctgtaatcccagctactcgggaggctgaggcacgagaattgcttgaacctgggaggtggaggttgcagtgagcagcgatcacgtcactgcactccagcttggaccacagagcgagactctgtctcaaataaatgaataaataatagaaaaggagattaattactactattaaaggcagataatcttttaagtttttttctttgtgattgtgctttgtagataatctttgtttattgaaatagttcttacaaactatgaacttaataagaagcactatctagtcattggatatcacattctctagcatcaaagtttataaaaaccttacaaaaacgaaacaggaggctagacgtggtggaatgacactaaaaattaaaatctgtagagtgcctaaatttctacctaattatccataatctaaactatatattataccacaaaatcagttcattctggtcaaatactctcttctcatataataaacagcagtcagtcttggcttctctgtaactgagtgactaatgagctcctaaaaaaaaaaaaaagtcctgcggaaaccaagaacagactataggactgattctaaatgtttacagtttaatttcatctgcagagcatttgccaaacagtatctcttccgtacctcatctgatcctcggcaatcccgttaggcagtcagagcggcttttatctcgatgctacctacaactggggctcaggaggggttcccgattgaccagtgcaagactaacagttaaagctcttcagacaaaaattttatgcagtgatgaacaaaagctcctcgtttgataggtaagcccctgggtacgcttatatgtgagtctgaaacaaaacactcccaatttggagtccggaaaaaagctgacactacaaggtgctgggcagttaaagttctctgccagtctgggaaaggggccaagatgccctttaaaacttaagacttcctctcaggacttaaggagatccctgcccttccagctgagcagtcgaggattacggtcaagcagagctgtgctcttagcaggttctcgaatccctttttcctttcactttctccccaggtaccctgatcccccacccctcaacgcttctgtgtttgtcaagaagcccacctggtcttctagacctgaagcggcaggaccctccgttcccaataatacaagtcaccaaaaggagcgtcccagagtccagttttaccttcatgtgcctctcactcacttcaggcccaggccaggttaccgcaaccacttcactttaatgtccgcctcccagcgcttcagactcaaccacaatattcaaacccaagccactttc

SEQ ID NO: 13 - DNA, 3435 bp, Homo sapiens
  source 1..3435: mol_type = genomic DNA
gtggttgagtctgaagcgctgggaggcggacattaaagtgaagtggttgcggtaacctggcctgggcctgaagtgagtgagaggcacatgaagagaagtattcaagtatttatacagataggaatcaagataatcaacaatgtctgtcactgaggaagacctgtgccaccatatgaaagtagtagttcgtgtacgtccggaaaacactaaagaaaaagcagctggatttcataaagtggttcatgttgtggataaacatatcctagtttttgatcccaaacaagaagaagtcagttttttccatggaaagaaaactacaaatcaaaatgttataaagaaacaaaataaggatcttaaatttgtatttgatgctgtttttgatgaaacgtcaactcagtcagaagtttttgaacacactactaagccaattcttcgtagttttttgaatggatataattgcacagtacttgcctatggtgccactggtgctgggaagacccacactatgctaggatcagctgatgaacctggagtgatgtatctaacaatgttacacctttacaaatgcatggatgagattaaagaagagaaaatatgtagtactgcagtttcatatctggaggtatataatgaacagattcgtgatctcttagtaaattcagggccacttgctgtccgggaagatacccaaaaaggggtggtcgttcatggacttactttacaccagcccaaatcctcagaagaaattttacatttattggataatggaaacaaaaacaggacacaacatcccactgatatgaatgccacatcttctcgttctcatgctgttttccaaatttacttgcgacaacaagacaaaacagcaagtatcaatcaaaatgtccgtattgccaagatgtcactcattgacctggcaggatctgagcgagcaagtacttccggtgctaaggggacccgatttgtagaaggcacaaatattaatagatcacttttagctcttgggaatgtcatcaatgccttagcagattcaaagagaaagaatcagcatatcccttacagaaatagtaagcttactcgcttgttaaaggattctcttggaggaaactgtcaaactataatgatagctgctgttagtccttcctctgtattctacgatgacacatataacactcttaagtatgctaaccgggcaaaggacattaaatcttctttgaagagcaatgttcttaatgtcaataatcatataactcaatatgtaaagatctgtaatgagcagaaggcagagattttattgttaaaagaaaaactaaaagcctatgaagaacagaaagccttcactaatgaaaatgaccaagcaaagttaatgatttcaaaccctcaggaaaaagaaatcgaaaggtttcaagaaatcctgaactgcttgttccagaatcgagaagaaattagacaagaatatctgaagttggaaatgttacttaaagaaaatgaacttaaatcattctaccaacaacagtgccataaacaaatagaaatgatgtgttctgaagacaaagtagaaaaggccactggaaaacgagatcatagacttgcaatgttgaaaactcgtcgctcctacctggagaaaaggagggaggaggaattgaagcaatttgatgagaatactaattggctccatcgtgtcgaaaaagaaatgggactcttaagtcaaaacggtcatattccaaaggaactcaagaaagatcttcattgtcaccatttgcacctccagaacaaagatttgaaagcacaaattagacatatgatggatctagcttgtcttcaggaacagcaacacaggcagactgaagcagtattgaatgctttacttccaaccctaagaaaacaatattgcacattaaaagaagccggcctgtcaaatgctgcttttgaatctgacttcaaagagatcgaacatttggtagagaggaaaaaagtggtagtttgggctgaccaaactgccgaacaaccaaagcaaaacgatctaccagggatttctgttcttatgacctttccacaacttggaccagttcagcctattccttgttgctcatcttcaggtggaactaatctggttaagattcctacagaaaaaagaactcggagaaaactaatgccatctcccttgaaaggacagcatactctaaagtctccaccatctcaaagtgtgcagctcaatgattctcttagcaaagaacttcagcctattgtatatacaccagaagactgtagaaaagcttttcaaaatccgtctacagtaaccttaatgaaaccatcatcatttactacaagttttcaggctatcagctcaaacataaacagtgataattgtctgaaaatgttgtgtgaagtagctatccctcataatagaagaaaagaatgtggacaggaggacttggactctacatttactatatgtgaagacatcaagagctcgaagtgtaaattacccgaacaagaatcactaccaaatgataacaaagacattttacaacggcttgatccttcttcattctcaactaagcattctatgcctgtaccaagcatggtgccatcctacatggcaatgactactgctgccaaaaggaaacggaaattaacaagttctacatcaaacagttcgttaactgcagacgtaaattctggatttgccaaacgtgttcgacaagataattcaagtgagaagcacttacaagaaaacaaaccaacaatggaacataaaagaaacatctgtaaaataaatccaagcatggttagaaaatttggaagaaatatttcaaaaggaaatctaagataaatcacttcaaaaccaagcaaaatgaagttgatcaaatctgcttttcaaagtttatcaataccctttcaaaaatatatttaaaatctttgaaagaagacccatcttaaagctaagtttacccaagtactttcagcaagcagaaaaatgaaactctttgttttcttcttttgtgttctaaaaaaataaaatttcaaaagaaaaggttgtcttttaagttttttaaatatttgttgccttttaaaatccctgagtgtaagttaccatggtggcagcttagttttactatgccacaacaagttgactaggacattttagtaaatggtagtgagttaaattatctttattattttttaaaaataagaatttagaagtggtaaaattatggcccaagatgtatttggttctctattatgttttgatacattattttaatcatatatatgactttccttttcaaaaatactttaatgtacaagtgtaaatatatgtgcccataaaatcattgtaaatattatttagtcatcacaaataaaatattgtcccttgctacttgatatattaaagatgtagattttaaagtggatgtagattttaaagtg